(19) United States
(12) Patent Application Publication
(10) Pub. No.: US 2026/0224758 A1
(43) Pub. Date: Aug. 6, 2026

(54) ALPHA-SYNUCLEIN BINDERS AND METHODS OF USE

(71) Applicant: Merck Sharp & Dohme LLC, Rahway, NJ (US)

(72) Inventors: Idriss BENNACEF, Ambler, PA (US); Maria V. FAWAZ, Hatfield, PA (US); Eric D. HOSTETLER, Collegeville, PA (US); Helen MITCHELL, Richboro, PA (US); Anthony J. ROECKER, Santee, CA (US); Anthony W. SHAW, Harleysville, PA (US); Craig A. STUMP, Pottstown, PA (US); Ling TONG, Warren, NJ (US)

(73) Assignee: Merck Sharp & Dohme LLC, Rahway, NJ (US)

(21) Appl. No.: 19/157,423

(22) PCT Filed: Feb. 29, 2024
(86) PCT No.: PCT/US2024/017922

§ 371 Date: Aug. 18, 2025

Related U.S. Application Data
(60) Provisional application No. 63/488,652, filed on Mar. 6, 2023.

(51) Int. Cl. A61K 51/04; A61B 5/00; C07B 59/00; C07D 401/14; C07D 403/14; C07D 413/14

(52) U.S. Cl. CPC A61K 51/0461; A61B 5/4848; A61K 51/0459; C07B 59/002; C07D 401/14; C07D 403/14; C07D 413/14; A61K 2123/00; C07B 2200/05

(57) ABSTRACT

The invention is directed to compounds of Formula I (I) or their pharmaceutically acceptable salts, which may be suitable for imaging alpha-synuclein pathology and hence are useful in binding and imaging alpha-synuclein aggregates in patients with Parkinson's Disease. More specifically, this invention relates to a method of using the compounds of this invention as tracers in positron emission tomography (PET) imaging to study alpha-synuclein in brain in vivo to allow diagnosis of Parkinson's Disease and other neurodegenerative diseases Specific characterized by alpha-synuclein pathology. The invention further relates to a method of measuring clinical efficacy of therapeutic agents for Parkinson's Disease and other neurodegenerative diseases characterized by alpha-synuclein pathology.

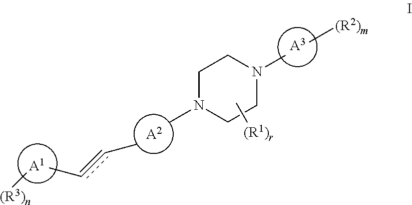

### CROSS-REFERENCE TO RELATED APPLICATIONS

This application claims the benefit of priority to U.S. Provisional Application No. 63/488,652, filed Mar. 6, 2023, the disclosure of which is incorporated herein by its entirety.

### BACKGROUND OF THE INVENTION

Neurodegenerative diseases such as Alzheimer's disease, Parkinson's disease (PD), Huntington's disease, amyotrophic lateral sclerosis and prion diseases are debilitating diseases which affect cognition and/or muscle control. These diseases are a subset of protein misfolding diseases. Protein folding is an essential process for protein function in all organisms, and conditions that disrupt protein folding present a threat to cell viability. In some cases, the disease arises because a specific protein is no longer functional when adopting a misfolded state. In other diseases, the pathological state originates because misfolding occurs concomitantly with aggregation, and the underlying aggregates are detrimental. Even though neurodegenerative diseases such as Alzheimer's and Parkinson's are caused by different proteins, both involve the accumulation of insoluble fibrous protein deposits, called amyloids. For example, Parkinson's Disease (PD), Dementia with Lewy Bodies (DLB), and multiple system atrophy (MSA), which are collectively referred to as “synucleinopathies,” have been linked to the accumulation of aggregated forms of the alpha-synuclein protein in neurons in the brain (see Nat. Rev. Neuro. 2013, 9, 13-24 and J. Parkinson's Disease 2013, 3, 565-567). As the primary neuropathologic change of PD, the degeneration of dopaminergic neurons occurs in the substantia nigra, as well as Lewy bodies (LB) and Lewy neurites (LN). To date, the pathogenic mechanism of PD has not been fully discovered.

Alpha-synuclein is a presynaptic terminal protein that consists of a140-amino acid protein that plays an important function in the central nervous system including synaptic vesicle recycling and synthesis, vesicular storage, and neurotransmitter release. It is specifically upregulated in a discrete population of presynaptic terminals of the brain during acquisition-related synaptic rearrangement. Alpha-synuclein naturally exists in a highly soluble, unfolded state. Evidence suggests that filamentous aggregates of alpha-synuclein accumulate at the pre-synaptic membrane and trigger synapse dysfunction and neuronal cell death in synucleinopathies and may be the cause of Parkinson's and DLB. Alpha-synuclein aggregation has been identified by antibody immunohistological studies as the major component of Lewy bodies, which are microscopic protein deposits in deteriorating nerve cells. Accumulation of misfolded, fibrillar alpha-synuclein in LewAy bodies (LB) and LewAy neurites (LN) is considered a hallmark of PD.

The diagnosis of PD is mainly based on the clinical symptoms such as rest tremor, bradykinesia, and rigidity, although these methods have their limitations (see J. Neurology 2019, 266, 1927-1936). The current desired treatment for PD is to slow the disease progression and minimize the disease symptoms in the patients. Therefore, a method of diagnosing PD in the very early stage can greatly help the physicians to design the therapeutic paradigm accordingly, and to slow the disease progression. There remains a need for improved diagnostic methods for identifying aggregations of misfolded proteins, including alpha-synuclein for early detection and ongoing monitoring of PD in subjects (see J. Parkinson's Disease 2013, 3, 565-567).

An alpha-synuclein positron emission tomography (PET) tracer would be a valuable non-invasive diagnostic biomarker for spatial and temporal quantification of aggregated pathological alpha-synuclein in human brain as a Parkinson's Disease biomarker. Additionally, an alpha-synuclein PET tracer could be useful for patient selection for PD clinical trials. In this mode, an alpha-synuclein tracer could be developed as a companion diagnostic for co-registration of a therapeutic agent. Additionally, an alpha-synuclein PET tracer could be a critical disease-relevant tool for quantifying a stabilization or decrease of alpha-synuclein formation for disease-modifying PD therapeutics.

Therefore, a need exists for neuroimaging radiotracers that would allow in vivo imaging of alpha-synuclein pathology thereby providing insight into the deposition of alpha-synuclein aggregates in the human brain. The successful neuroimaging radiotracer must cross the blood-brain barrier, have rapid clearance from tissue and plasma, and possess high affinity and specificity for alpha-synuclein aggregates with high selectivity over binding to beta-amyloid and tau aggregated proteins due to co-expression in many PD patient populations (see Biol Psychiatry 2015, 78, 672-683 and J Neuropath Exper Neurol 2003, 62, 389-397). While alpha synuclein binding ligands have been described that have reduced selectivity over aggregated beta-amyloid (WO 2019/121661), there is a need for compounds with high levels of selectivity over co-expressed aggregated proteins in PD in order to quantify an alpha synuclein specific signal in an in vivo imaging study for PD patients.

The present invention advances these interests by providing compounds of Formula I as aggregated alpha-synuclein binding ligands with high selectivity over binding of aggregated beta-amyloid pathology. The instant invention also relates to a method of using the compounds of Formula I as tracers in PET imaging to study alpha-synuclein deposits in brain in vivo to allow diagnosis of neurodegenerative diseases characterized by alpha-synuclein pathology. The invention further relates to a method of measuring clinical efficacy of therapeutic agents targeting alpha-synuclein pathology.

### SUMMARY OF THE INVENTION

The invention is directed to compounds of Formula I, pharmaceutical salts thereof, pharmaceutical compositions comprising them, diagnostic and therapeutic uses and processes for making such compounds. An embodiment of the invention provides a compound of Formula I:

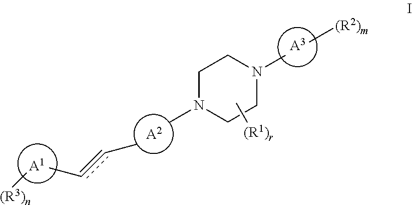

or a pharmaceutically acceptable salt thereof wherein;
[symbol] may be absent or may represent a bond;
R is independently selected from H or —C₁₋₆alkyl, where said alkyl is optionally substituted with one to three groups from —C₁₋₆alkyl, OR^{a} or halo;
R^{a} is independently selected from H, —C₁₋₆alkyl, —(CH₂)ₚOR, —(CH₂)ₚhalo, or —(CH₂)ₚO(CH₂)ₚhalo;
R^{b} is independently selected from H, —C₁₋₆alkyl, heterocyclyl, heteroaryl, —(CH₂)ₛOR, —CN,
—(CH₂)ₜhalo, —(CH₂)ₛNR₂ or —O(CH₂)ₚhalo;
R^{c} is independently selected from H, halo, OR, or —C₁₋₆alkyl, where said alkyl is optionally substituted with one to three groups from —C₁₋₆alkyl, OR^{a} or halo;
R¹ is selected —(CH₂)ₛOR, —(CH₂)ₛNR₂, —(CH₂)ₛ[O(R^{c}₂)ₚ]ₓ—^{R}c, or —(CH₂)ₛhalo;
R² independently selected from H, OR, CN, halo or —C₁₋₆alkyl, where said alkyl is optionally substituted with one to three groups from —C₁₋₆alkyl, OR^{a} or halo; R³ is independently selected from —C₁₋₆alkyl, heteroaryl or heterocyclyl, wherein said alkyl, heteroaryl or heterocyclyl is optionally substituted with one to three groups from OR^{a} or R^{b};
Ring A¹ is selected from pyridinyl, imidazo-pyrimidinyl, triazinyl, pyrimidinyl, imidazo-pyridinyl, pyrazinyl or pyridazinyl;
Ring A² is selected from pyrimidinyl, pyridinyl, pyrazinyl or phenyl, wherein said pyrimidinyl, pyridinyl, pyrazinyl or phenyl is optionally substituted with one to three groups from —C₁₋₆alkyl, OR^{a} or halo;
Ring A³ is selected from pyridinyl, pyrazinyl, pyrimidinyl, pyrrolopyrazinyl, triazinyl, indolyl, imidazolyl, oxadiazolyl, triazolyl, thiazolyl, isoxazolyl, oxazolyl. 3,4-dihydro-2H-pyrido[3,2,b][1,4]oxazine or phenyl;
m is selected from 0, 1, or 2;
n is selected from 1, 2, or 3;
p is independently selected from 1, 2 or 3;
r is selected from 1, 2 or 3;
s is independently selected from 0, 1, 2, 3, 4, 5 or 6;
t is independently selected from 0, 1, 2, 3, 4, 5 or 6; and
x is independently selected from 1, 2, 3, 4, 5 or 6.

The present invention is also directed to isotopically-labeled compounds of Formula I. Additionally, the present invention provides pharmaceutical compositions comprising a compound of Formula I and at least one pharmaceutically acceptable carrier.

The present invention is directed to compounds of Formula I which may be useful for binding alpha-synuclein aggregated proteins and/or tau aggregated proteins, and hence are useful in binding and imaging alpha-synuclein aggregated protein pathology in PD and non-PD synucleinopathy patients as well as aggregated Tau protein pathology Alzheimer's Disease (AD) and non-AD tauopathy patients via PET imaging techniques known commonly in the field (see J. Nucl. Med. 2019, 60, 93-99 and 107-114). This invention also relates to methods of using compounds of Formula I to identify patients with abnormal levels of aggregated alpha-synuclein pathology in the brain. This invention also relates to methods of using a compound of Formula I as a to measure progression of alpha-synuclein pathology over time as a biomarker in clinical assessment of potential therapeutic agents that can modify Parkinson's Disease progression.

Compounds of this invention may also be useful for imaging and detecting for other neurodegenerative diseases characterized by the deposition of alpha-synuclein aggregates such as multiple system atrophy (MSA) and dementia with Lewy Bodies (DLB).

### BRIEF DESCRIPTION OF THE DRAWINGS

FIG. 1: Saturation binding experiment in aggregated beta-amyloid rich AD tissue homogenate for [³H]-105.

FIG. 2: Specific [³H]-2 binding to alpha-synucleinopathy of a PD brain amygdala section.

FIG. 3: Coronal slice of a PET image of [¹¹C]-2 in rhesus monkey brain (averaged over 30-90 min post injection, fused with MRI).

### DETAILED DESCRIPTION OF THE INVENTION

The present invention provides novel compounds, synthetic methods for making the compounds, pharmaceutical compositions containing them, isotopically-labeled compounds and methods of using the compounds as imaging agents.

In an embodiment, the present invention is directed to a compound of Formula I:

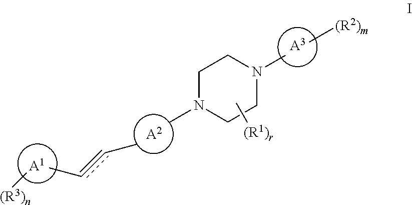

or a pharmaceutically acceptable salt thereof wherein;
[symbol] may be absent or may represent a bond;
R is independently selected from H or —C₁₋₆alkyl, where said alkyl is optionally substituted with one to three groups from —C₁₋₆alkyl, OR^{a} or halo;
R^{a} is independently selected from H, —C₁₋₆alkyl, —(CH₂)ₚOR, —(CH₂)ₚhalo, or —O(CH₂)ₚhalo;
R^{b} is independently selected from H, —C₁₋₆alkyl, heterocyclyl, heteroaryl, —(CH₂)ₚOR, —CN, —(CH₂)ₜhalo, —(CH₂)ₛNR₂ or —O(CH₂)ₚhalo;
R^{c} is independently selected from H, halo, OR, or —C₁₋₆alkyl, where said alkyl is optionally substituted with one to three groups from —C₁₋₆alkyl, OR^{a} or halo;
R¹ is selected —(CH₂)ₛOR, —(CH₂)ₛNR₂, —(CH₂)ₛ[O(R^{c}₂)ₚ]ₓ—R^{c}, or —(CH₂)ₛhalo;
R² is independently selected from H, OR, CN, halo or —C₁₋₆alkyl, where said alkyl is optionally substituted with one to three groups from —C₁₋₆alkyl, OR^{a} or halo;
R³ is independently selected from —C₁₋₆alkyl, heteroaryl or heterocyclyl, wherein said alkyl, heteroaryl or heterocyclyl is optionally substituted with one to three groups from OR^{a} or R^{b};

Ring A¹ is selected from pyridinyl, imidazo-pyrimidinyl, triazinyl, pyrimidinyl, imidazo-pyridinyl, pyrazinyl or pyridazinyl;
Ring A² is selected from pyrimidinyl, pyridinyl, pyrazinyl or phenyl, wherein said pyrimidinyl, pyridinyl, pyrazinyl or phenyl is optionally substituted with one to three groups from —C₁₋₆alkyl. OR^{a} or halo;
Ring A³ is selected from pyridinyl, pyrazinyl, pyrimidinyl, pyrrolopyrazinyl, triazinyl, indolyl, imidazolyl, oxadiazolyl, triazolyl, thiazolyl, isoxazolyl, oxazolyl. 3,4-dihydro-2H-pyrido[3,2,b][1,4]oxazine or phenyl;
m is selected from 0, 1, or 2;
n is selected from 1, 2, or 3;
p is independently selected from 1, 2 or 3;
r is selected from 1, 2 or 3;
s is independently selected from 0, 1, 2, 3, 4, 5 or 6;
t is independently selected from 0, 1, 2, 3, 4, 5 or 6; and
x is independently selected from 1, 2, 3, 4, 5 or 6.

A further embodiment of the invention provides a compound of Formula IA:

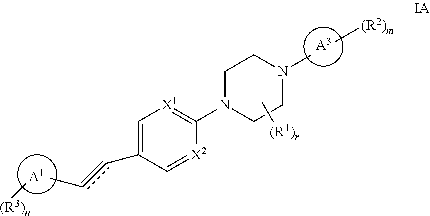

or a pharmaceutically acceptable salt thereof wherein;
[symbol] may be absent or may represent a bond;
R is independently selected from H or —C₁₋₆alkyl, where said alkyl is optionally substituted with one to three groups from —C₁₋₆alkyl, OR^{a} or halo;
R^{a} is independently selected from H, —C₁₋₆alkyl, —(CH₂)ₚOR, —(CH₂)ₚhalo, or —(CH₂)ₚO(CH₂)ₚhalo;
R^{b} is independently selected from H, —C₁₋₆alkyl, heterocyclyl, heteroaryl, —(CH₂)ₚOR, —CN, —(CH₂)ₜhalo, —(CH₂)ₛNR₂ or —O(CH₂)ₚhalo;
R^{c} is independently selected from H, halo, OR, or —C₁₋₆alkyl, where said alkyl is optionally substituted with one to three groups from —C₁₋₆alkyl, OR^{a} or halo;
R¹ is selected —(CH₂)ₛOR, —(CH₂)ₛNR₂, —(CH₂)[O(R^{c}₂)ₚ]ₓ—R^{c}, or —(CH₂)ₛhalo;
R² independently selected from H, OR, CN, halo or —C₁₋₆alkyl, where said alkyl is optionally substituted with one to three groups from —C₁₋₆alkyl, OR^{a} or halo;
R³ is independently selected from —C₁₋₆alkyl, heteroaryl or heterocyclyl, wherein said alkyl, heteroaryl or heterocyclyl is optionally substituted with one to three groups from OR^{a} or R^{b};
X¹ is N or CH;
X² is N or CH;
Ring A¹ is selected from pyridinyl, imidazo-pyrimidinyl, triazinyl, pyrimidinyl, imidazo-pyridinyl, pyrazinyl or pyridazinyl;
Ring A³ is selected from pyridinyl, pyrazinyl, pyrimidinyl, pyrrolopyrazinyl, triazinyl, indolyl, imidazolyl, oxadiazolyl, triazolyl, thiazolyl, isoxazolyl, oxazolyl, 3,4-dihydro-2H-pyrido[3,2,b][1,4]oxazine or phenyl;
m is selected from 0, 1, or 2;
n is selected from 1, 2, or 3;
p is independently selected from 1, 2 or 3;
r is selected from 1, 2 or 3;
s is independently selected from 0, 1, 2, 3, 4, 5 or 6;
t is independently selected from 0, 1, 2, 3, 4, 5 or 6; and
x is independently selected from 1, 2, 3, 4, 5, or 6.

In a further embodiment, the present invention is directed to a compound of Formula IB:

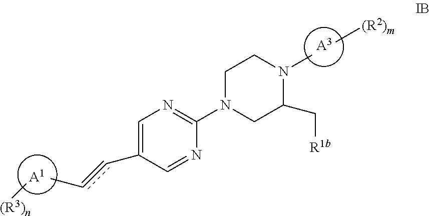

or a pharmaceutically acceptable salt thereof wherein;
[symbol] may be absent or may represent a bond;
R is independently selected from H or C₁₋₆alkyl, where said alkyl is optionally substituted with one to three groups from —C₁₋₆alkyl, OR^{a} or halo;
R^{a} is independently selected from H, —C₁₋₆alkyl, —(CH₂)ₚOR, —(CH₂)ₚhalo, halogen, or —(CH₂)ₚO(CH₂)ₚhalo;
R^{b} is independently selected from H, —C₁₋₆alkyl, heterocyclyl, heteroaryl, —(CH₂)ₚOR, —CN. —(CH₂)ₜhalo, —(CH₂)ₛNR₂ or —O(CH₂)ₚhalo;
R^{c} is independently selected from H, halo, OR, or —C₁₋₆alkyl, where said alkyl is optionally substituted with one to three groups from —C₁₋₆alkyl, OR^{a} or halo;
R^{1b} is selected from —[O(R^{c}₂)ₚ]ₓ—R^{c}, OR, NR₂ or halo;
R² is independently selected from H, OR, CN, halo or —C₁₋₆alkyl, where said alkyl is optionally substituted with one to three groups from —C₁₋₆alkyl, OR^{a} or halo;
R³ is independently selected from —C₁₋₆alkyl, imidazolyl, morpholinyl, triazolyl, oxazolyl, triazinyl, pyrazolyl, thiazolyl, pyrazinyl, isoxazolyl, azetidinyl, pyrrolidinyl, tetrahydrotriazolopyrazinyl, piperidinyl or pyrimidinyl, wherein said alkyl, imidazolyl, morpholinyl, triazolyl, oxazolyl, triazinyl, pyrazolyl, thiazolyl, pyrazinyl, isoxazolyl, azetidinyl, pyrrolidinyl, tetrahydrotriazolopyrazinyl, piperidinyl or pyrimidinyl is optionally substituted with one to three groups from OR³ or R^{b};
Ring A¹ is selected from pyridinyl, imidazo-pyrimidinyl, triazinyl, pyrimidinyl, imidazo-pyridinyl, pyrazinyl or pyridazinyl;
Ring A³ is selected from pyridinyl, pyrazinyl, pyrimidinyl, pyrrolopyrazinyl, triazinyl, indolyl, imidazolyl, oxadiazolyl, triazolyl, thiazolyl, isoxazolyl, oxazolyl. 3,4-dihydro-2H-pyrido[3,2,b][1,4]oxazine or phenyl;
m is selected from 0, 1, or 2;
n is selected from 1, 2, or 3;
p is independently selected from 1, 2 or 3;
s is independently selected from 0, 1, 2, 3, or 4;
t is independently selected from 0, 1, 2, 3, 4, 5 or 6; and
x is independently selected from 1, 2, 3, 4, 5 or 6.

In another embodiment, the present invention is directed to compounds of Formula I, having the structure of Formula IC:

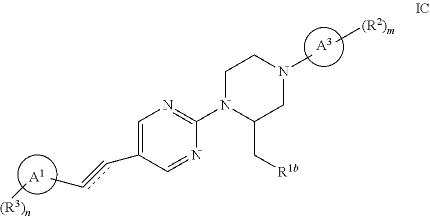

or a pharmaceutically acceptable salt thereof wherein;
[symbol] may be absent or may represent a bond;
R is independently selected from H or —C₁₋₆alkyl, where said alkyl is optionally substituted with one to three groups from —C₁₋₆alkyl, OR^{a} or halo;
R^{a} is independently selected from H, —C₁₋₆alkyl, —(CH₂)ₚOR, —(CH₂)ₚhalo, or —(CH₂)ₚO(CH₂)ₚhalo;
R^{b} is independently selected from H, —C₁₋₆alkyl, heterocyclyl, heteroaryl, —(CH₂)ₚOR, —CN. —(CH₂)ₜhalo, —(CH₂)ₛNR₂ or —O(CH₂)ₚhalo;
R^{c} is independently selected from H, halo, OR, or —C₁₋₆alkyl, where said alkyl is optionally substituted with one to three groups from —C₁₋₆alkyl, OR^{a} or halo; R^{1b} is selected from —[O(R^{c}₂)ₚ]ₓ—R^{c}, OR, NR₂ or halo;
R² is independently selected from H. OR, CN, halo or —C₁₋₆alkyl, where said alkyl is optionally substituted with one to three groups from —C₁₋₆alkyl, OR^{a} or halo;
R³ is independently selected from —C₁₋₆alkyl, imidazolyl, morpholinyl, triazolyl, oxazolyl, triazinyl, pyrazolyl, thiazolyl, pyrazinyl, isoxazolyl, azetidinyl, pyrrolidinyl, tetrahydrotriazolopyrazinyl, piperidinyl or pyrimidinyl, wherein said alkyl, imidazolyl, morpholinyl, triazolyl, oxazolyl, triazinyl, pyrazolyl, thiazolyl, pyrazinyl, isoxazolyl, azetidinyl, pyrrolidinyl, tetrahydrotriazolopyrazinyl, piperidinyl or pyrimidinyl is optionally substituted with one to three groups from OR^{a} or R^{b};
Ring A¹ is selected from pyridinyl, imidazo-pyrimidinyl, triazinyl, pyrimidinyl, imidazo-pyridinyl, pyrazinyl or pyridazinyl;
Ring A³ is selected from pyridinyl, pyrazinyl, pyrimidinyl, pyrrolopyrazinyl, triazinyl, indolyl, imidazolyl, oxadiazolyl, triazolyl, thiazolyl, isoxazolyl, oxazolyl, 3,4-dihydro-2H-pyrido[3,2,b][1,4]oxazine or phenyl;
m is selected from 0, 1, or 2;
n is selected from 1, 2, or 3;
p is independently selected from 1, 2 or 3;
s is independently selected from 0, 1, 2, 3, or 4;
t is independently selected from 0, 1, 2, 3, 4, 5 or 6; and
x is independently selected from 0, 1, 2, 3, 4, 5 or 6.

In another embodiment, the present invention is directed to compounds of Formula I, having the structure of Formula ID:

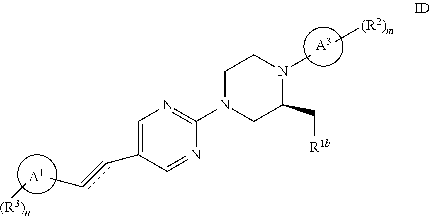

or a pharmaceutically acceptable salt thereof wherein;
[symbol] may be absent or may represent a bond;
R is independently selected from H or —C₁₋₆alkyl, where said alkyl is optionally substituted with one to three groups from —C₁₋₆alkyl, OR^{a} or halo;
R^{a} is independently selected from H, —C₁₋₆alkyl, —(CH₂)ₚOR, —(CH₂)ₚhalo, or —(CH₂)ₚO(CH₂)ₚhalo;
R is independently selected from H, —C₁₋₆alkyl, —(CH₂)ₚOR, —CN, —(CH₂)ₜhalo, —(CH₂)ₛNR₂ or —O(CH₂)ₚhalo;
R^{c} is independently selected from H, halo, OR, or —C₁₋₆alkyl, where said alkyl is optionally substituted with one to three groups from —C₁₋₆alkyl, OR^{a} or halo;
R^{1b} is selected from —[O(R^{c}₂)ₚ]ₓ—R^{c}, OR, NR₂ or halo;
R² is independently selected from H, OR, halo or —C₁₋₆alkyl, where said alkyl is optionally substituted with one to three groups from —C₁₋₆alkyl, OR^{a} or halo;
R³ is independently selected from —C₁₋₆alkyl, imidazolyl, morpholinyl, triazolyl, oxazolyl, triazinyl, pyrazolyl, thiazolyl, pyrazinyl, isoxazolyl, azetidinyl, pyrrolidinyl, tetrahydrotriazolopyrazinyl, piperidinyl or pyrimidinyl, wherein said alkyl, imidazolyl, morpholinyl, triazolyl, oxazolyl, triazinyl, pyrazolyl, thiazolyl, pyrazinyl, isoxazolyl, azetidinyl, pyrrolidinyl, tetrahydrotriazolopyrazinyl, piperidinyl or pyrimidinyl is optionally substituted with one to three groups from OR^{a} or R^{b};
Ring A¹ is selected from pyridinyl, imidazo-pyrimidinyl, triazinyl, pyrimidinyl, imidazo-pyridinyl, pyrazinyl or pyridazinyl;
Ring A³ is selected from pyridinyl, pyrazinyl, pyrimidinyl, pyrrolopyrazinyl, triazinyl, indolyl, imidazolyl, oxadiazolyl, triazolyl, thiazolyl, isoxazolyl, oxazolyl, 3,4-dihydro-2H-pyrido[3,2,b][1,4]oxazine or phenyl;
m is selected from 0, 1, or 2;
n is selected from 1, 2, or 3;
p is independently selected from 1, 2 or 3;
s is independently selected from 0, 1, 2, 3, or 4;
t is independently selected from 0, 1, 2, 3, 4, 5 or 6; and
x is independently selected from 0, 1, 2, 3, 4, 5 or 6.

In another embodiment, the present invention is directed to compounds of Formula I, having the structure of Formula IE:

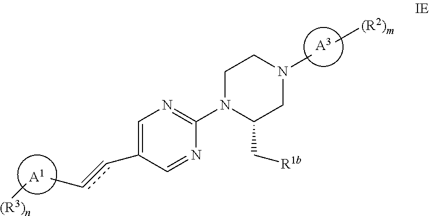

or a pharmaceutically acceptable salt thereof wherein;
[symbol] may be absent or may represent a bond;
R is independently selected from H or —C₁₋₆alkyl, where said alkyl is optionally substituted with one to three groups from —C₁₋₆alkyl, OR^{a} or halo;
R^{a} is independently selected from H, —C₁₋₆alkyl, —(CH₂)ₚOR, —(CH₂)ₚhalo, or —(CH₂)ₚO(CH₂)ₚhalo;
R^{b} is independently selected from H, —C₁₋₆alkyl, —(CH₂)ₚOR, —CN, —(CH₂)ₜhalo, —(CH₂)ₛNR₂ or —O(CH₂)ₚhalo;
R^{c} is independently selected from H, halo, OR, or —C₁₋₆alkyl, where said alkyl is optionally substituted with one to three groups from —C₁₋₆alkyl, OR^{a} or halo;
R^{1b} is selected from —[O(R²)ₚ]ₓ—R^{c}, OR, NR₂ or halo;
R² is independently selected from H, OR, halo or —C₁₋₆alkyl, where said alkyl is optionally substituted with one to three groups from —C₁₋₆alkyl, OR^{a} or halo;
R³ is independently selected from —C₁₋₆alkyl, imidazolyl, morpholinyl, triazolyl, oxazolyl, triazinyl, pyrazolyl, thiazolyl, pyrazinyl, isoxazolyl, azetidinyl, pyrrolidinyl, tetrahydrotriazolopyrazinyl, piperidinyl or pyrimidinyl, wherein said alkyl, imidazolyl, morpholinyl, triazolyl, oxazolyl, triazinyl, pyrazolyl, thiazolyl, pyrazinyl, isoxazolyl, azetidinyl, pyrrolidinyl, tetrahydrotriazolopyrazinyl, piperidinyl or pyrimidinyl is optionally substituted with one to three groups from OR^{a} or R^{b};
Ring A¹ is selected from pyridinyl, imidazo-pyrimidinyl, triazinyl, pyrimidinyl, imidazo-pyridinyl, pyrazinyl or pyridazinyl;
Ring A³ is selected from pyridinyl, pyrazinyl, pyrimidinyl, pyrrolopyrazinyl, triazinyl, indolyl, imidazolyl, oxadiazolyl, triazolyl, thiazolyl, isoxazolyl, oxazolyl, 3,4-dihydro-2H-pyrido[3,2,b][1,4]oxazine or phenyl;
m is selected from 0, 1, or 2;
n is selected from 1, 2, or 3;
p is independently selected from 1, 2 or 3;
s is independently selected from 0, 1, 2, 3, or 4;
t is independently selected from 0, 1, 2, 3, 4, 5 or 6; and.
x is independently selected from 0, 1, 2, 3, 4, 5 or 6.

In an embodiment, the present invention is directed to a compound of Formula IF:

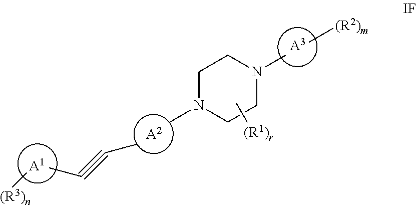

or a pharmaceutically acceptable salt thereof, and all substituents and variables are as defined above in Formula I.

In an embodiment, the present invention is directed to a compound of Formula IG:

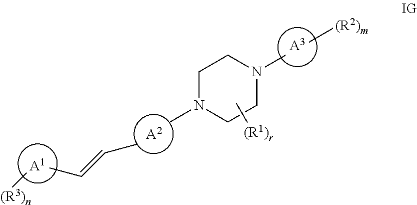

or a pharmaceutically acceptable salt thereof, and all substituents and variables are as defined above in Formula I.

In a further embodiment, the present invention is directed to a compound of Formula I, or a pharmaceutically acceptable salt thereof, wherein;
Ring A¹ is selected from pyrimidinyl or pyridinyl;
Ring A³ is selected from pyridinyl, pyrazinyl, pyrimidinyl, or triazinyl;
m is selected from 0 or 1;
n is selected from 1 or 2;
and all other substituents and variables are as defined above in Formula I.

In an embodiment, the invention provides a compound of Formula I, IA, IB. IC, ID, IE, IF, or IG wherein Ring A¹ is selected from pyridinyl, triazinyl or pyrimidinyl. In another embodiment, Ring A¹ is pyridinyl or pyrimidinyl. In a further embodiment, Ring A¹ is pyridinyl. In a further embodiment, Ring A¹ is triazinyl. In a further embodiment, Ring A¹ is pyrimidinyl.

In an embodiment, the invention provides a compound of Formula I, IF, or IG, wherein Ring A² is pyrimidinyl. In another embodiment, Ring A² is phenyl. In another embodiment. Ring A² is pyridinyl.

In an embodiment, the invention provides a compound of Formula I, IA, IB, IC, ID, IE, IF, or IG, wherein Ring A³ is selected from pyridinyl, pyrimidinyl, pyrazinyl or triazinyl. In another embodiment. Ring A³ is selected from pyridinyl or pyrimidinyl. In a further embodiment, Ring A³ is pyridinyl. In a further embodiment, Ring A³ is pyrimidinyl. In a further embodiment, Ring A³ is triazinyl.

In an embodiment, the invention provides a compound of Formula I, IA, IF, or IG, wherein R¹ is —(CH₂)ₛOR. In an embodiment, the invention provides a compound of Formula I, IA, IF, or IG, wherein, R¹ is —(CH₂)ₛ[O(R^{c}₂)ₚ]ₓ—R⁰.

In an embodiment, the invention provides a compound of Formula I, IA, IB, IC, ID, IE, IF, or IG, wherein R³ is independently selected from —C₁₋₆alkyl, imidazolyl, morpholinyl, triazolyl, oxazolyl, triazinyl, pyrazolyl, or isoxazolyl, wherein said alkyl, imidazolyl, morpholinyl, triazolyl, oxazolyl, triazinyl, pyrazolyl, or isoxazolyl is optionally substituted with one to three groups from OR^{a} or OR^{b}. In a further embodiment, R³ is independently selected from —C₁₋₆alkyl, triazinyl or imidazolyl, wherein said alkyl, triazinyl and imidazolyl is optionally substituted with one to three groups from OR^{a} or R^{b}. In a further embodiment, R³ is independently selected from —C₁₋₆alkyl or imidazolyl, wherein said alkyl, pyrazolyl or imidazolyl is optionally substituted with one to three groups from OR^{a} or R^{b}. In a further embodiment, R³ is independently selected from pyrazolyl or imidazolyl, wherein said pyrazolyl or imidazolyl is optionally substituted with one to three groups from OR^{a} or OR^{b}. In a further embodiment. R³ is pyrazolyl wherein said pyrazolyl is optionally substituted with one to three groups from OR^{a} or R^{b}. In a further embodiment, R³ is imidazolyl, wherein said imidazolyl is optionally substituted with one to three groups from OR³ or R^{b};

Representative compounds of the present invention include compounds selected from:

| Ex No. | Chemical Name |
|---|---|
| 1 | (R,E)-(4-(5-(2-(6-(1H-imidazol-1-yl)pyridin-3-yl)vinyl)pyrimidin-2-yl)-1-(pyrimidin- |
| | 2-yl)piperazin-2-yl)methanol; |
| 2 | (R,E)-5-(2-(6-(1H-imidazol-1-yl)pyridin-3-yl)vinyl)-2-(3-(methoxymethyl)-4- |
| | (pyrimidin-2-yl)piperazin-1-yl)pyrimidine; |
| 3 | (R,E)-5-(2-(6-(1H-imidazol-1-yl)pyridin-3-yl)vinyl)-2-(3-((2-fluoroethoxy)methyl)-4- |
| | (pyrimidin-2-yl)piperazin-1-yl)pyrimidine; |
| 4 | (R,E)-5-(2-(6-(1H-imidazol-1-yl)pyridin-3-yl)vinyl)-2-(3-((fluoromethoxy)methyl)-4- |
| | (pyrimidin-2-yl)piperazin-1-yl)pyrimidine; |
| 5 | (R,E)-5-(2-(6-(4H-1,2,4-triazol-4-yl)pyridin-3-yl)vinyl)-2-(2-(methoxymethyl)-4- |
| | (pyridin-2-yl)piperazin-1-yl)pyrimidine; |
| 6 | (R,E)-5-(2-(6-(1H-imidazol-1-yl)pyridin-3-yl)vinyl)-2-(2-((2-methoxyethoxy)methyl)- |
| | 4-(pyridin-2-yl)piperazin-1-yl)pyrimidine; |
| 7 | (R,E)-(2-(4-(5-(2-(6-(1H-imidazol-1-yl)pyridin-3-yl)vinyl)pyrimidin-2-yl)-2- |
| | (methoxymethyl)piperazin-1-yl)pyrimidin-5-yl)methanol; |
| 8 | (R,E)-5-(2-(6-(1H-imidazol-1-yl)pyridin-3-yl)vinyl)-2-(4-(5-(fluoromethyl)pyrimidin- |
| | 2-yl)-3-(methoxymethyl)piperazin-1-yl)pyrimidine; |
| 9 | (R,E)-5-(2-(6-(1H-imidazol-1-yl)pyridin-3-yl)vinyl)-2-(2-(methoxymethyl)-4-(5- |
| | (methoxymethyl)pyrimidin-2-yl)piperazin-1-yl)pyrimidine; |
| 10 | (R)-(4-(5-((6-(1H-imidazol-1-yl)pyridin-3-yl)ethynyl)pyrimidin-2-yl)-1-(pyrimidin-2- |
| | yl)piperazin-2-yl)methanol; |
| 11 | (R)-5-((6-(1H-imidazol-1-yl)pyridin-3-yl)ethynyl)-2-(3-(methoxymethyl)-4- |
| | (pyrimidin-2-yl)piperazin-1-yl)pyrimidine; |
| 12 | (R)-5-((6-(1H-imidazol-1-yl)pyridin-3-yl)ethynyl)-2-(4-(6-fluoropyrimidin-4-yl)-3- |
| | (methoxymethyl)piperazin-1-yl)pyrimidine; |
| 13 | (R,E)-5-(2-(6-(1H-imidazol-1-yl)pyridin-3-yl)vinyl)-2-(4-(6-fluoropyrimidin-4-yl)-2- |
| | (methoxymethyl)piperazin-1-yl)pyrimidine; |
| 14 | (R,E)-5-(2-(6-(1H-imidazol-1-yl)pyridin-3-yl)vinyl)-2-(4-(6-chloropyrimidin-4-yl)-2- |
| | (methoxymethyl)piperazin-1-yl)pyrimidine; |
| 15 | (R,E)-(1-(5-(2-(6-(1H-imidazol-1-yl)pyridin-3-yl)vinyl)pyrimidin-2-yl)-4-(6- |
| | fluoropyrimidin-4-yl)piperazin-2-yl)methanol; |
| 16 | (R,E)-4-(5-(2-(2-(4-(4-fluoropyridin-2-yl)-2-(methoxymethyl)piperazin-1- |
| | yl)pyrimidin-5-yl)vinyl)pyridin-2-yl)morpholine; |
| 17 | (R,E)-5-(2-(6-(1H-imidazol-1-yl)pyridin-3-yl)vinyl)-2-(4-(4-fluoropyridin-2-yl)-2- |
| | (methoxymethyl)piperazin-1-yl)pyrimidine; |
| 18 | (R,E)-5-(2-(6-(1H-imidazol-1-yl)pyridin-3-yl)vinyl)-2-(2-(methoxymethyl)-4- |
| | (pyridin-2-yl)piperazin-1-yl)pyrimidine; |
| 19 | (R,E)-6-(2-(2-(2-(methoxymethyl)-4-(pyridin-2-yl)piperazin-1-yl)pyrimidin-5- |
| | yl)vinyl)-2-methylimidazo[1,2-a]pyrimidine; |
| 20 | (R,E)-2-(5-(2-(2-(2-(methoxymethyl)-4-(pyridin-2-yl)piperazin-1-yl)pyrimidin-5- |
| | yl)vinyl)pyridin-2-yl)oxazole; |
| 21 | (R,E)-7-(4-(5-(2-(6-(1H-pyrazol-1-yl)pyridin-3-yl)vinyl)pyrimidin-2-yl)-3- |
| | (methoxymethyl)piperazin-1-yl)pyrrolo[1,2-a]pyrazine; |
| 22 | (R,E)-5-(2-(6-(1H-imidazol-1-yl)pyridin-3-yl)vinyl)-2-(3-(methoxymethyl)-4- |
| | (pyridin-2-yl)piperazin-1-yl)pyrimidine; |
| 23 | (R,E)-(1-(5-(2-(6-(1H-imidazol-1-yl)pyridin-3-yl)vinyl)pyrimidin-2-yl)-4-(pyrazin-2- |
| | yl)piperazin-2-yl)methanol; |
| 24 | (R,E)-(1-(5-(2-(6-(1H-imidazol-1-yl)pyridin-3-yl)vinyl)pyrimidin-2-yl)-4-(pyrimidin- |
| | 4-yl)piperazin-2-yl)methanol; |
| 25 | (R,E)-5-(2-(6-(1H-imidazol-1-yl)pyridin-3-yl)vinyl)-2-(2-(methoxymethyl)-4-(5- |
| | methylpyrimidin-2-yl)piperazin-1-yl)pyrimidine; |
| 26 | (R,E)-5-(5-(2-(2-(2-(methoxymethyl)-4-(pyridin-2-yl)piperazin-1-yl)pyrimidin-5- |
| | yl)vinyl)pyridin-2-yl)oxazole; |
| 27 | (R,E)-5-(2-(6-(1H-imidazol-1-yl)pyridin-3-yl)vinyl)-2-(4-(5-fluoropyrimidin-2-yl)-3- |
| | (methoxymethyl)piperazin-1-yl)pyrimidine; |
| 28 | (R,E)-5-(2-(6-(1H-imidazol-1-yl)pyridin-3-yl)vinyl)-2-(2-(methoxymethyl)-4- |
| | (pyrimidin-2-yl)piperazin-1-yl)pyrimidine; |
| 29 | (R,E)-5-(2-(6-(1H-imidazol-1-yl)pyridin-3-yl)vinyl)-2-(4-(2-fluoropyridin-4-yl)-2- |
| | (methoxymethyl)piperazin-1-yl)pyrimidine; |
| 30 | (R,E)-5-(2-(6-(1H-imidazol-1-yl)pyridin-3-yl)vinyl)-2-(2-((2-fluoroethoxy)methyl)-4- |
| | (pyrimidin-2-yl)piperazin-1-yl)pyrimidine; |
| 31 | (R,E)-5-(2-(6-(1H-imidazol-1-yl)pyridin-3-yl)vinyl)-2-(2-((2-fluoroethoxy)methyl)-4- |
| | (pyridin-4-yl)piperazin-1-yl)pyrimidine; |
| 32 | (R,E)-5-(2-(6-(1H-imidazol-1-yl)pyridin-3-yl)vinyl)-2-(4-(6-fluoropyridin-3-yl)-2- |
| | (methoxymethyl)piperazin-1-yl)pyrimidine; |
| 33 | (R,E)-(1-(5-(2-(6-(1H-imidazol-1-yl)pyridin-3-yl)vinyl)pyrimidin-2-yl)-4-(6- |
| | fluoropyridin-3-yl)piperazin-2-yl)methanol; |
| 34 | (R,E)-4-(4-(5-(2-(6-(1H-imidazol-1-yl)pyridin-3-yl)vinyl)pyrimidin-2-yl)-2- |
| | (methoxymethyl)piperazin-1-yl)-2-fluoropyrimidine; and |
| 35 | (R,E)-5-(2-(6-(1H-imidazol-1-yl)pyridin-3-yl)vinyl)-2-(4-(5-(2- |
| | fluoroethoxy)pyrimidin-2-yl)-3-(methoxymethyl)piperazin-1-yl)pyrimidine; |
| 36 | (R,E)-5-(5-(2-(2-(3-((2-fluoroethoxy)methyl)-4-(pyrimidin-2-yl)piperazin-1- |
| | yl)pyrimidin-5-yl)vinyl)pyridin-2-yl)oxazole |
| 37 | (R,E)-5-(2-(6-(1-(2-fluoroethyl)-1H-pyrazol-4-yl)pyridin-3-yl)vinyl)-2-(3- |
| | (methoxymethyl)-4-(pyrimidin-2-yl)piperazin-1-yl)pyrimidine |
| 38 | (R,E)-5-(5-(2-(2-(3-((2-(2-fluoroethoxy)ethoxy)methyl)-4-(1,3,5-triazin-2- |
| | yl)piperazin-1-yl)pyrimidin-5-yl)vinyl)pyridin-2-yl)oxazole |
| 39 | 1-((2-((R)-4-(5-((E)-2-(6-(1H-imidazol-1-yl)pyridin-3-yl)vinyl)pyrimidin-2-yl)-2- |
| | (methoxymethyl)piperazin-1-yl)pyrimidin-5-yl)oxy)-3-fluoropropan-2-ol |
| 40 | (R,E)-2-(3-((2-fluoroethoxy)methyl)-4-(pyrimidin-2-yl)piperazin-1-yl)-5-(2-(6-(4- |
| | methyl-1H-imidazol-1-yl)pyridin-3-yl)vinyl)pyrimidine |
| 41 | (R,E)-5-(5-(2-(2-(3-((2-fluoroethoxy)methyl)-4-(1,3,5-triazin-2-yl)piperazin-1- |
| | yl)pyrimidin-5-yl)vinyl)pyridin-2-yl)oxazole |
| 42 | (R,E)-5-(5-(2-(2-(3-((2-fluoroethoxy)methyl)-4-(pyrimidin-4-yl)piperazin-1- |
| | yl)pyrimidin-5-yl)vinyl)pyridin-2-yl)oxazole |
| 43 | (R,E)-2-(2-((2-fluoroethoxy)methyl)-4-(5-(2-(2-(1-methyl-1H-pyrazol-4-yl)pyrimidin- |
| | 5-yl)vinyl)pyrimidin-2-yl)piperazin-1-yl)-1,3,5-triazine |
| 44 | (R)-2-(2-((2-fluoroethoxy)methyl)-4-(5-((2-(1-methyl-1H-pyrazol-4-yl)pyrimidin-5- |
| | yl)ethynyl)pyrimidin-2-yl)piperazin-1-yl)-1,3,5-triazine |
| 45 | (R)-5-((6-(4H-1,2,4-triazol-4-yl)pyridin-3-yl)ethynyl)-2-(4-(6-fluoropyrimidin-4-yl)- |
| | 3-(methoxymethyl)piperazin-1-yl)pyrimidine |
| 46 | (R)-2-(4-(5-((2-(1-(2-fluoroethyl)-1H-pyrazol-4-yl)pyrimidin-5-yl)ethynyl)pyrimidin- |
| | 2-yl)-2-(methoxymethyl)piperazin-1-yl)-1,3,5-triazine |
| 47 | (R)-2-(4-(5-((6-(1-(2-fluoroethyl)-1H-pyrazol-4-yl)pyridin-3-yl)ethynyl)pyrimidin-2- |
| | yl)-2-(methoxymethyl)piperazin-1-yl)-1,3,5-triazine |
| 48 | (R)-5-(5-((2-(3-((2-(2-fluoroethoxy)ethoxy)methyl)-4-(1,3,5-triazin-2-yl)piperazin-1- |
| | yl)pyrimidin-5-yl)ethynyl)pyridin-2-yl)oxazole |
| 49 | (R)-2-(2-((2-fluoroethoxy)methyl)-4-(5-((2-(1-methyl-1H-pyrazol-3-yl)pyrimidin-5- |
| | yl)ethynyl)pyrimidin-2-yl)piperazin-1-yl)-1,3,5-triazine |
| 50 | (R)-2-(2-((2-fluoroethoxy)methyl)-4-(5-((6-(1-methyl-1H-pyrazol-4-yl)pyridin-3- |
| | yl)ethynyl)pyrimidin-2-yl)piperazin-1-yl)-1,3,5-triazine |
| 51 | (R)-2-(3-((2-fluoroethoxy)methyl)-4-(pyrimidin-2-yl)piperazin-1-yl)-5-((2-(1-methyl- |
| | 1H-pyrazol-4-yl)pyrimidin-5-yl)ethynyl)pyrimidine |
| 52 | (R)-2-(3-((2-(2-fluoroethoxy)ethoxy)methyl)-4-(pyrimidin-2-yl)piperazin-1-yl)-5-((2- |
| | (1-methyl-1H-pyrazol-4-yl)pyrimidin-5-yl)ethynyl)pyrimidine |
| 53 | (R)-2-(3-((2-fluoroethoxy)methyl)-4-(pyrimidin-4-yl)piperazin-1-yl)-5-((2-(1-methyl- |
| | 1H-pyrazol-4-yl)pyrimidin-5-yl)ethynyl)pyrimidine |
| 54 | (R)-2-(1-(2-fluoroethyl)-1H-pyrazol-4-yl)-5-((2-(3-(methoxymethyl)-4-(pyrimidin-4- |
| | yl)piperazin-1-yl)pyrimidin-5-yl)ethynyl)pyrimidine |
| 55 | (R)-5-(5-((2-(3-((2-fluoroethoxy)methyl)-4-(1,3,5-triazin-2-yl)piperazin-1- |
| | yl)pyrimidin-5-yl)ethynyl)pyrimidin-2-yl)oxazole |
| 56 | (R)-2-(4-(5-((2-(1-(fluoromethyl)-1H-pyrazol-4-yl)pyrimidin-5-yl)ethynyl)pyrimidin- |
| | 2-yl)-2-(methoxymethyl)piperazin-1-yl)-1,3,5-triazine |
| 57 | (R)-2-(4-(5-((6-(1H-imidazol-1-yl)pyridin-3-yl)ethynyl)-6-fluoropyrazin-2-yl)-2- |
| | (methoxymethyl)piperazin-1-yl)pyrimidine |
| 58 | (R)-5-((6-(4H-1,2,4-triazol-4-yl)pyridin-3-yl)ethynyl)-4-fluoro-2-(3- |
| | (methoxymethyl)-4-(pyrimidin-4-yl)piperazin-1-yl)pyrimidine |
| 59 | (R)-5-((6-(3-fluoro-4H-1,2,4-triazol-4-yl)pyridin-3-yl)ethynyl)-2-(3- |
| | (methoxymethyl)-4-(pyrimidin-2-yl)piperazin-1-yl)pyrimidine |
| 60 | (R)-2-(4-(5-((6-(1H-imidazol-1-yl)pyridin-3-yl)ethynyl)pyrimidin-2-yl)-2- |
| | (methoxymethyl)piperazin-1-yl)-4-fluoropyrimidine |
| 61 | (R)-4-(4-(5-((6-(1H-imidazol-1-yl)pyridin-3-yl)ethynyl)pyrimidin-2-yl)-2- |
| | (methoxymethyl)piperazin-1-yl)-2-fluoropyrimidine |
| 62 | (S)-5-(5-((2-(3-((2-fluoroethoxy)methyl)-4-(1,3,5-triazin-2-yl)piperazin-1- |
| | yl)pyrimidin-5-yl)ethynyl)pyridin-2-yl)oxazole |
| 63 | 3-(((R)-4-(5-((6-(1H-imidazol-1-yl)pyridin-3-yl)ethynyl)pyrimidin-2-yl)-1- |
| | (pyrimidin-2-yl)piperazin-2-yl)methoxy)-2-fluoropropan-1-ol |
| 64 | (R)-5-(5-((2-(4-(2-fluoropyridin-4-yl)-3-(methoxymethyl)piperazin-1-yl)pyrimidin-5- |
| | yl)ethynyl)pyridin-2-yl)oxazole |
| 65 | 1-(4-(6-fluoropyrimidin-4-yl)-1-(5-((6-(oxazol-5-yl)pyridin-3-yl)ethynyl)pyrimidin-2- |
| | yl)piperazin-2-yl)-N-methylmethanamine |
| 66 | (R)-5-(5-((2-(4-(5-(2-fluoroethoxy)pyrimidin-2-yl)-3-(methoxymethyl)piperazin-1- |
| | yl)pyrimidin-5-yl)ethynyl)pyridin-2-yl)oxazole |
| 67 | 1-fluoro-3-(((R)-4-(5-((6-(oxazol-5-yl)pyridin-3-yl)ethynyl)pyrimidin-2-yl)-1-(1,3,5- |
| | triazin-2-yl)piperazin-2-yl)methoxy)propan-2-ol |
| 68 | (R)-5-((6-(3-fluoroazetidin-1-yl)pyridin-3-yl)ethynyl)-2-(3-(methoxymethyl)-4- |
| | (pyrimidin-2-yl)piperazin-1-yl)pyrimidine |
| 69 | (R)-5-((6-(1H-imidazol-1-yl)pyridin-3-yl)ethynyl)-2-(4-(6-fluoropyrazin-2-yl)-3- |
| | (methoxymethyl)piperazin-1-yl)pyrimidine |
| 70 | (R)-2-(4-(5-((6-(4H-1,2,4-triazol-4-yl)pyridin-3-yl)ethynyl)pyrimidin-2-yl)-2-((2- |
| | fluoroethoxy)methyl)piperazin-1-yl)-1,3,5-triazine |
| 71 | (R)-5-((6-(4H-1,2,4-triazol-4-yl)pyridin-3-yl)ethynyl)-2-(3-((2-fluoroethoxy)methyl)- |
| | 4-(pyrimidin-2-yl)piperazin-1-yl)pyrimidine |
| 72 | 5-((6-((S)-3-fluoropyrrolidin-1-yl)pyridin-3-yl)ethynyl)-2-((R)-3-(methoxymethyl)-4- |
| | (pyrimidin-2-yl)piperazin-1-yl)pyrimidine |
| 73 | (R)-1-(5-((2-(3-((2-fluoroethoxy)methyl)-4-(1,3,5-triazin-2-yl)piperazin-1- |
| | yl)pyrimidin-5-yl)ethynyl)pyrimidin-2-yl)-1H-pyrazol-4-amine |
| 74 | (R)-5-(5-((2-(3-((3-fluoropropoxy)methyl)-4-(1,3,5-triazin-2-yl)piperazin-1- |
| | yl)pyrimidin-5-yl)ethynyl)pyridin-2-yl)oxazole |
| 75 | (R)-7-(5-((2-(3-((2-fluoroethoxy)methyl)-4-(1,3,5-triazin-2-yl)piperazin-1- |
| | yl)pyrimidin-5-yl)ethynyl)pyridin-2-yl)-5,6,7,8-tetrahydro-[1,2,4]triazolo[4,3- |
| | a]pyrazine |
| 76 | (R)-5-((6-(4-(2H-1,2,3-triazol-2-yl)piperidin-1-yl)pyridin-3-yl)ethynyl)-2-(3- |
| | (methoxymethyl)-4-(pyrimidin-2-yl)piperazin-1-yl)pyrimidine |
| 77 | (R)-2-(4-(5-((6-(1-(2-(2-fluoroethoxy)ethyl)-1H-pyrazol-4-yl)pyridin-3- |
| | yl)ethynyl)pyrimidin-2-yl)-2-(methoxymethyl)piperazin-1-yl)-1,3,5-triazine |
| 78 | (R)-2-(2-((2-(2-fluoroethoxy)ethoxy)methyl)-4-(5-((2-(1-methyl-1H-pyrazol-4- |
| | yl)pyrimidin-5-yl)ethynyl)pyrimidin-2-yl)piperazin-1-yl)-1,3,5-triazine |
| 79 | (R)-5-((2-(3-((2-(2-fluoroethoxy)ethoxy)methyl)-4-(1,3,5-triazin-2-yl)piperazin-1- |
| | yl)pyrimidin-5-yl)ethynyl)-2,5′-bipyrimidine |
| 80 | (R)-2-(4-(5-((6-(1H-imidazol-1-yl)pyridin-3-yl)ethynyl)-4-fluoropyrimidin-2-yl)-2- |
| | ((2-methoxyethoxy)methyl)piperazin-1-yl)-1,3,5-triazine |
| 81 | (R)-2-(4-(5-((6-(6-fluoropyrimidin-4-yl)pyridin-3-yl)ethynyl)pyrimidin-2-yl)-2- |
| | (methoxymethyl)piperazin-1-yl)pyrimidine-5-carbonitrile |
| 82 | (R)-2-(3-((2-(2-(2-fluoroethoxy)ethoxy)ethoxy)methyl)-4-(pyrimidin-2-yl)piperazin- |
| | 1-yl)-5-((2-(1-methyl-1H-pyrazol-4-yl)pyrimidin-5-yl)ethynyl)pyrimidine |
| 83 | (R)-2-(3-(13-fluoro-2,5,8,11-tetraoxatridecyl)-4-(pyrimidin-2-yl)piperazin-1-yl)-5-((2- |
| | (1-methyl-1H-pyrazol-4-yl)pyrimidin-5-yl)ethynyl)pyrimidine |
| 84 | (R)-2-(4-(5-((2-(1-(2-fluoroethyl)-1H-pyrazol-4-yl)pyrimidin-5-yl)ethynyl)pyrimidin- |
| | 2-yl)-3-(methoxymethyl)piperazin-1-yl)-1,3,5-triazine |
| 85 | (R)-2-(1-(2-fluoroethyl)-1H-pyrazol-4-yl)-5-((2-(4-(5-fluoropyridin-2-yl)-3- |
| | (methoxymethyl)piperazin-1-yl)pyrimidin-5-yl)ethynyl)pyrimidine |
| 86 | (S)-5-((2-(4H-1,2,4-triazol-4-yl)pyrimidin-5-yl)ethynyl)-2-(4-(5-fluoropyridin-2-yl)- |
| | 2-methylpiperazin-1-yl)pyrimidine |
| 87 | (S)-2-(4-(5-((2-(1-(2-fluoroethyl)-1H-pyrazol-4-yl)pyrimidin-5-yl)ethynyl)pyrimidin- |
| | 2-yl)-3-(methoxymethyl)piperazin-1-yl)-1,3,5-triazine |
| 88 | (R)-5-fluoro-2-(4-(5-((2-(1-(2-fluoroethyl)-1H-pyrazol-4-yl)pyrimidin-5- |
| | yl)ethynyl)pyrimidin-2-yl)-2-(methoxymethyl)piperazin-1-yl)pyrimidine |
| 89 | (R)-5-((2-(4H-1,2,4-triazol-4-yl)pyrimidin-5-yl)ethynyl)-2-(4-(5-fluoropyridin-2-yl)- |
| | 3-(methoxymethyl)piperazin-1-yl)pyrimidine |
| 90 | (R)-5-fluoro-2-(4-(5-((6-(1-(2-fluoroethyl)-1H-pyrazol-4-yl)pyridin-3- |
| | yl)ethynyl)pyrimidin-2-yl)-2-(methoxymethyl)piperazin-1-yl)pyrimidine |
| 91 | (R)-2-(4-(5-((2-(1-(3-fluoropropyl)-1H-pyrazol-4-yl)pyrimidin-5- |
| | yl)ethynyl)pyrimidin-2-yl)-2-(methoxymethyl)piperazin-1-yl)-1,3,5-triazine |
| 92 | (R)-5-(2-fluoroethoxy)-2-(2-(methoxymethyl)-4-(5-((6-(1-methyl-1H-pyrazol-4- |
| | yl)pyridin-3-yl)ethynyl)pyrimidin-2-yl)piperazin-1-yl)pyrimidine |
| 93 | (R)-5-(5-((2-(3-((2-fluoroethoxy)methyl)-4-(pyrimidin-2-yl)piperazin-1-yl)pyrimidin- |
| | 5-yl)ethynyl)pyrimidin-2-yl)oxazole |
| 94 | (R)-2-(4-(6-fluoropyrimidin-4-yl)-3-(methoxymethyl)piperazin-1-yl)-5-((2-(1-methyl- |
| | 1H-pyrazol-4-yl)pyrimidin-5-yl)ethynyl)pyrimidine |
| 95 | (R)-2-(2-((fluoromethoxy)methyl)-4-(5-((2-(1-methyl-1H-pyrazol-4-yl)pyrimidin-5- |
| | yl)ethynyl)pyrimidin-2-yl)piperazin-1-yl)-1,3,5-triazine |
| 96 | (S)-2-(3-((2-fluoroethoxy)methyl)-4-(5-((2-(1-methyl-1H-pyrazol-4-yl)pyrimidin-5- |
| | yl)ethynyl)pyrimidin-2-yl)piperazin-1-yl)-1,3,5-triazine |
| 97 | (R)-5-fluoro-2-(2-((2-fluoroethoxy)methyl)-4-(5-((2-(1-methyl-1H-pyrazol-3- |
| | yl)pyrimidin-5-yl)ethynyl)pyrimidin-2-yl)piperazin-1-yl)pyrimidine |
| 98 | (R)-2-(2-((2-fluoroethoxy)methyl)-4-(5-((2-(1-methyl-1H-pyrazol-5-yl)pyrimidin-5- |
| | yl)ethynyl)pyrimidin-2-yl)piperazin-1-yl)-1,3,5-triazine |

or a pharmaceutically acceptable salt thereof.

The present invention is directed to compound of Formula I for use as an imaging agent.

An embodiment of the invention comprises a compound selected from Ex. No. 1, 2, 3, 4, 12, 13, 17, 29, 31, 32 34, 38, 43, 44, 46, 48, 49, 51 or 52, or a pharmaceutically acceptable salt thereof. An embodiment of the invention comprises a compound selected from Ex. No. 1, 2, 3, 4, 12, 13, 17, 29, 31, 32 or 34, or a pharmaceutically acceptable salt. A further embodiment of the invention comprises a compound selected from Ex. No. 38, 43, 44, 46, 48, 49, 51 or 52, or a pharmaceutically acceptable salt thereof. A further embodiment of the invention comprises a compound selected from Ex. No. 1, 2, 3, 12, 13, 29 or 32, or a pharmaceutically acceptable salt thereof. A further embodiment of the invention comprises a compound selected from Ex. No. 1, 2, 3, 12, 13, 29, 32, 38, 44, 46 or 52, or a pharmaceutically acceptable salt thereof. A further embodiment of the invention comprises a compound selected from Ex. No. 2, 44 or 52, or a pharmaceutically acceptable salt thereof.

Another aspect of the invention is directed to compounds of Formula I, or a pharmaceutically acceptable salt thereof, that are labeled with an isotope selected from ²H, ³H, ¹¹C, ¹³C, ¹⁴C, ¹³N, ¹⁵N, ¹⁵O, ¹⁷O, ¹⁸O, ¹⁸F, ³⁵S, ³⁶CL, ⁸²Br, ⁷⁶Br, ⁷⁷Br, ¹²³I, ¹²⁴I or 131I. In a further aspect of the invention, the compounds of Formula I are isotopically labeled with ³H, ¹¹C or ¹⁸F. Examples of isotopically labeled a compound of Formula I, or pharmaceutically acceptable salts thereof, include, but are not limited to, ³H-2, ³H-29, ¹¹C-2, ¹⁸F-3, ¹⁸F-12, ¹⁸F-13, ¹⁸F-29, ¹⁸F-38, ¹⁸F-44, ¹⁸F-46, and ¹⁸F-52 and the like.

Another aspect of the invention is directed to compounds of Formula I, or a pharmaceutically acceptable salt thereof, that are labeled with an isotope selected from ²H, ³H, ¹¹C, ¹³C, ¹⁴C, ¹³N, ¹⁵N, ¹⁵O, ¹⁷O, ¹⁸O, ¹⁸F, ³⁵S, ³⁶CL, ⁸²Br, ⁷⁶Br, ⁷⁷Br, ¹²³I, ¹²⁴I or ¹³¹I, for use as an imaging agent.

It is understood that reference to “Formula I” also encompasses compounds of Formula I′, Formula IA, Formula IB, Formula IC, Formula ID, Formula IE, Formula IF and Formula IG, unless indicated otherwise.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Likewise, the present invention includes tautomeric forms of the compounds disclosed herein. Formula I shows the structure of the class of compounds without specific stereochemistry. At least some of the chemical names of compounds of the invention as set forth in this application may have been generated on an automated basis by use of commercially available chemical naming software programs, and have not been independently verified.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. Absolute stereochemistry may also be elucidated through other techniques known in the art, such as cryogenic electron microscopy. Relative stereochemistry may be determined using nuclear magnetic resonance with methods known in the art. Stereochemistry may be assigned by analogy to a set of isomers based on their relative biological activity following the same trend established by a similar stereochemically defined group of isomers. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Compounds of the present invention may also be separated by supercritical fluid chromatography (SFC) or reverse-phase HPLC or silica gel chromotography. Isomers are named according to the order they came off the column (first, second, etc eluting or alternatively with “A” and “B” or “1” and “2” being the first, second, etc eluting isomers) with examples named as example #A and example #B, etc according to the order eluting from the purification system. One with skill in the art would understand that sometimes the peaks may contain more than a single isomer and when cut in half or fractionated further result in multiple fractions of one peak and may not represent single isomers.

Furthermore, some separations required multiple rounds of purifications by the same method of purification and/or an alternative purification system. Additionally, a mixture may be a mixture of 2 to 8 stereoisomers. Alternatively, any enantiomer of a compound may be obtained by stereoselective synthesis using optically pure starting materials or reagents of known configuration by methods well known in the art.

In one embodiment the present invention provides pharmaceutical compositions comprising a compound of the invention, for example, a compound of Formula I, and at least one pharmaceutical excipient.

Compounds of Formula I are inhibitors and/or binders of aggregated alpha-synuclein or tau protein. Compounds of Formula I, and isotopically labeled variants thereof, may be useful for the diagnosis and/or treatment of Parkinson's disease and/or Alzheimer's disease. Means of detecting labels are well known to those skilled in the art. For example, isotopic labels may be detected using imaging techniques, photographic film or scintillation counters. In a preferred embodiment, the label is detected in vivo in the brain of the subject by imaging techniques, for example positron emission tomography (PET).

The compounds of Formula (I) may also form a component of bifunctional compounds that are targeted protein degrader compounds that bind aggregated alpha-synuclein proteins.

Such targeted alpha-synuclein protein degrader compounds contain a target protein binding moiety which is formed from a compound of Formula (I) and an E3 ubiquitin ligase-binding moiety. The targeted alpha-synuclein protein degrader compounds typically contain a linker group joining the alpha-synuclein protein binding moiety and the E3 ubiquitin ligase-binding moiety. The E3 ubiquitin ligase-binding moieties in the alpha-synuclein targeted protein degrader compounds can be, but are not limited to, binders to the E3 ligase von Hippel-Lindau protein, binders to the E3 ligase cereblon protein, or binders to the MDM2 protein. Such compounds can be administered in pharmaceutical compositions to treat disease conditions, including but not limited to, the conditions disclosed herein.

In the description that follows conventional structural representation is employed and includes conventional stereochemical notation for certain asymmetric carbon centers.

Thus, structural representation of compounds of the invention includes conventional stereochemical notation for some asymmetric carbon centers shown in the example compounds. Accordingly, in such instances, solid black “wedge” bonds represent bonds projecting from the plane of the reproduction medium. “hashed wedge” bonds representing descending bonds into the plane of the reproduction medium, and a “wavey” line appended to a carbon bearing a double bond indicates both possible cis and trans orientations are included. As is conventional, plain solid lines represent all spatial configurations for the depicted bonding. Accordingly, where no specific stereochemical notation is supplied the representation contemplates all stereochemical and spatial orientations of the structural features.

As is shown in the examples of the invention, and mentioned above, particular asymmetric carbon centers are structurally represented using conventional “Solid Wedge” and “Hash Wedge” bonding representation. For the most part, absolute configuration has not been determined for the example compounds, but has been assigned by analogy to specific example compounds of known stereochemical configurations (determined by X-ray crystallography) prepared using the same or analogous reaction conditions and starting reagents and isolated under the same chromatographic conditions. Accordingly, specific assignment of the configurations structurally represented herein is meant to identify the specific compounds prepared has having an excess of one particular stereoisomer and is not put forth herein necessarily as being a statement of the absolute determination of the stereochemical structure of said compound unless otherwise noted in the data presented.

It will be appreciated that where isomeric mixtures are obtained, the preparation of individual stereoisomers in significant percentages of enantiomeric excess can be carried out, if desired, by separation of the mixture using customary methods, for example by chromatography or crystallization, or by the use of stereochemically uniform starting materials for the synthesis described, or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product.

Where indicated herein, absolute stereochemistry is determined by X-ray crystallography of crystalline products or crystalline intermediates which are derivatized, if necessary, with a reagent containing a stereogenic center of known configuration. Unless a particular isomer, salt, solvate (including hydrates) or solvated salt of such racemate, enantiomer, or diastereomer is indicated, the present invention includes all such isomers, as well as salts, solvates (including hydrates) and solvated salts of such racemates, enantiomers, diastereomers and mixtures thereof.

Where a wavey line terminates a conventional bond (as opposed to connecting two atoms within a structure) it indicates a point of bonding to a structure, e.g.:

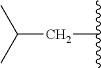 [SMILES: *CC(C)C]

indicates a the secondary-butyl moiety is bonded via the methylene group via the bond terminated with the wavey line. Where an alphabetical notation is used to depict a substituent moiety, a dash is employed to indicate the point of bonding to the indicated substrate, e.g.: —CH₂—C(O)—CH₂Cl indicates the acetyl chloride moiety is bonded via the methylene portion of the moiety.

Where compounds of Formula I are capable of tautomerization, all individual tautomers as well as mixtures thereof are included in the scope of this invention.

When any variable (e.g., R, R¹, n, heteroaryl, alkyl, etc.) occurs more than one time in any constituent or in Formula I, its definition on each occurrence is independent of its definition at every other occurrence unless otherwise specified at the point of definition. One of ordinary skill in the art will recognize that choice of combinations of the various substituents defined in a structural representation, i.e. R¹ R², etc., are to be chosen in conformity with well-known principles of chemical structure connectivity and stability, and combinations of substituents and/or variables are permissible only if such combinations result in stable compounds.

A “stable” compound is a compound which can be prepared and isolated and whose structure and properties remain or can be caused to remain essentially unchanged for a period of time sufficient to allow use of the compound for the purposes described herein (e.g., therapeutic administration to a subject). The compounds of the present invention are limited to stable compounds embraced by Formula I.

Where any variable or moiety is expressed in the form of a range, e.g. (—CH₂—)₁₋₄, both of the extrema of the specified range are included (i.e. 1 and 4 in the example) as well as all of the whole number values in between (i.e. 2 and 3 in the example).

It is understood that reference to “Formula I” also encompasses compounds of Formula IA, Formula IB and Formula IC, unless indicated otherwise.

As used herein, “alkyl” is intended to include both branched and straight-chain saturated aliphatic hydrocarbon groups having the specified number of carbon atoms.

“Halogen” or “halo” as used herein means fluoro, chloro, bromo and iodo.

As used herein, “cycloalkyl” is intended to include cyclic saturated aliphatic hydrocarbon groups having the specified number of carbon atoms. Preferably, cycloalkyl is C₃-C₁₀ cycloalkyl. Examples of such cycloalkyl elements include, but are not limited to, cyclopropyl, cyclobutyl, cyclopentyl, cyclohexyl and cycloheptyl.

As used herein, “aryl” is intended to mean any stable monocyclic or bicyclic carbon ring of up to 7 members in each ring, wherein at least one ring is aromatic. Examples of such aryl elements include phenyl, naphthyl, tetrahydronaphthyl, indanyl, biphenyl, phenanthryl, anthryl or acenaphthyl. In an embodiment of the instant invention, aryl is phenyl or naphthyl. In a further embodiment, aryl is phenyl.

The term heterocyclyl, heterocycle or heterocyclic, as used herein, represents a stable 5 to 7-membered monocyclic or stable 8- to 11-membered bicyclic heterocyclic ring which is either saturated or unsaturated, and which consists of carbon atoms and from one to four heteroatoms selected from the group consisting of N, O, and S. and including any bicyclic group in which any of the above-defined heterocyclic rings is fused to a benzene ring. The heterocyclic ring may be attached at any heteroatom or carbon atom which results in the creation of a stable structure. The term heterocyclyl, heterocycle or heterocyclic can include heteroaryl moieties when two rings are fused together. Examples of heterocyclic elements include, but are not limited to, azabicyclo[2,2,1]heptanyl, azepanyl, azetidinyl, benzodioxolyl, chromanyl, dihydrobenzofuryl, dihydrobenzothienyl, dihydrobenzothiopyranyl, dihydrobenzothiopyranyl sulfone, dihydro-pyrrolo[1,2-b]pyrazolyl, 1,3-dioxolanyl, imidazolidinyl, indolinyl, isochromanyl, isoindolinyl, morpholinyl, oxa-5-azabicyclo[2,2,1]heptanyl. 2-oxopiperazinyl, 2-oxopiperdinyl, 2-oxopyrrolidinyl, piperidyl, piperazinyl, pyrazolidinyl, pyrrolidinyl, tetrahydrofuryl, tetrahydroisoquinolinyl, tetrahydroquinolinyl, and thiamorpholinyl.

In an embodiment, heterocyclyl is selected from azabicyclo[2,2,1]heptanyl, azepanyl, azetidinyl, dihydro-pyrrolo[1,2-b]pyrazolyl, morpholinyl, oxa-5-azabicyclo[2,2,1]heptanyl, piperidyl, piperazinyl, pyrazolidinyl, pyrrolidinyl, pyrrolyl, and tetrahydrofuryl. In another embodiment, heterocyclyl is selected from azabicyclo[2,2,1]heptanyl, azepanyl, azetidinyl, dihydro-pyrrolo[1,2-b]pyrazolyl, oxa-5-azabicyclo[2,2,1]heptanyl, piperazinyl, and pyrrolidinyl.

“Heteroaryl” is intended to mean any stable monocyclic or bicyclic carbon ring of up to 7 members in each ring, wherein at least one ring is aromatic and wherein from one to four carbon atoms are replaced by heteroatoms selected from the group consisting of N, O, and S. Examples of such heterocyclic elements include, but are not limited to, azepinyl, furanyl, furyl, imidazolyl, indolinyl, indolyl, isochromanyl, isoindolinyl, isoxazolyl, isoquinolinyl, isothiazolyl, naphthyridinyl, oxazolyl, oxadiazolyl, pyridinyl, pyrazinyl, pyrazolyl, pyridazinyl, pyrimidinyl, 5H-pyrrolo[2,3-b]pyrazinyl, pyrrolyl, quinazolinyl, quinolinyl, tetrahydroisoquinolinyl, tetrahydroquinolinyl, tetrazolyl, thiazolyl, thienofuryl, thienothienyl, thienyl, triazinyl, triazolyl and the like. In an embodiment, heteroaryl is selected from furyl, imidazolyl, indolyl, isoxazolyl, oxazolyl, oxadiazolyl, pyridyl, pyrazinyl, pyrazolyl, pyridazinyl, pyrimidinyl, 5H-pyrrolo[2,3-b]pyrazinyl, tetrazolyl, thiazolyl, thienyl, triazinyl, triazolyl and the like.

For use in medicine, the salts of the compounds of Formula I will be pharmaceutically acceptable salts. Other salts may, however, be useful in the preparation of the compounds according to the invention or of their pharmaceutically acceptable salts. When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline. N,N′-dibenzylethylenediamine, diethylamine. 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

The preparation of the pharmaceutically acceptable salts described above and other typical pharmaceutically acceptable salts is more fully described by Berg et al., “Pharmaceutical Salts,”J. Pharm. Sci., 1977:66:1-19.

If the compounds of Formula I simultaneously contain acidic and basic groups in the molecule the invention also includes zwitterions, in addition to the salt forms described above.

The present invention also embraces isotopically-labeled compounds of the present invention which are structurally identical to those recited herein, but for the fact that a statistically significant percentage of one or more atoms in that form of the compound are replaced by an atom having an atomic mass or mass number different from the atomic mass or mass number of the most abundant isotope usually found in nature, thus altering the naturally occurring abundance of that isotope present in a compound of the invention. Another aspect of the invention relates to use of the isotopically labeled compounds as neuroimaging radiotracers for in vivo imaging of the brain for alpha-synuclein aggregates in the diagnosis, monitoring, and/or treatment of Parkinson's Disease (PD). Another aspect of the invention is use of the isotopically labeled compounds in PET, which is an in vivo analysis technique in the diagnosis, monitoring, and/or treatment of PD. The ³H, ¹¹C or ¹⁸F labeled compounds can be used in in vitro and in vivo methods for the determination of binding, receptor occupancy and metabolic studies including covalent labeling.

Another aspect of the invention relates to the use of the isotopically labeled compounds to screen for new chemical matter. In particular, various isotopically labeled compounds find utility in magnetic resonance imaging, autoradiography and other similar analytical tools. The present invention is meant to include all suitable isotopic variations of the compounds of Formula I. Examples of isotopes that can be preferentially incorporated into compounds of the invention include isotopes of hydrogen, carbon, nitrogen, oxygen, phosphorus, iodine, fluorine and chlorine, for example, but not limited to: ²H, ³H ¹¹C, ¹³C, ¹⁴C, ¹³N, ¹⁴N, ¹⁵O, ¹⁷O, ¹⁸O, ¹⁸F, ³⁵S, ³⁶Cl, ⁸²Br, ⁷⁶Br, ⁷⁷Br, ¹²³I, ¹²⁴I, ¹²⁵I or ¹³¹I isotopically labeled substituted heterocyclic derivative compounds of Formula I. It will be appreciated that other isotopes may be incorporated by known means also. In particular, the present invention is directed to ¹¹C, ¹³C, ¹⁴C, ¹⁸F, ¹⁵O, ¹³N, ³⁵S, ²H, and ³H isotopes of compounds of Formula I, compositions and methods of their preparation and use as radiotracers or PET tracers in diagnosing and measuring the effects of a compound in the treatment of PD. In a further embodiment, the present invention is directed to compounds of Formula I that are isotopically labeled with ³H, ¹¹C or ¹⁸F. along with compositions and methods of their preparation and use as PET tracers in diagnosing and measuring the effects of a compound in the treatment of PD. The present invention also relates to non-toxic alpha-synuclein protein binding compounds that can rapidly cross the blood brain barrier, have low non-specific binding properties and are rapidly cleared from the system. This and other aspects of the invention will be realized upon review of the specification in its entirety.

Isotopically-enriched compounds within Formula I can be prepared without undue experimentation by conventional techniques well known to those skilled in the art or by processes analogous to those described in the Schemes and Examples herein using appropriate isotopically-enriched reagents and/or intermediates.

As indicated herein the present invention includes isotopically labeled compounds of the invention. An “isotopically-labeled”, “radio-labeled”, “tracer”, “radiotracer”, “labeled tracer” or “radioligand” compound, is a compound where one or more atoms are replaced or substituted by an atom having an atomic mass or mass number different from the atomic mass or mass number typically found in nature (i.e., naturally occurring). Suitable radionuclides (i.e. “detectable isotopes”) that may be incorporated in compounds of the present invention include but are not limited to ²H, ³H, ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁶Br, ⁷⁷Br, ¹²³I, ¹²⁴I and 131I. The isotopically labeled compounds of the invention need only to be enriched with a detectable isotope to, or above, the degree which allows detection with a technique suitable for the particular application. The radionuclide that is incorporated in the instant radiolabeled compounds will depend on the specific application of that radiolabeled compound. In another embodiment of the invention the radionuclides are represented by ¹¹C, ¹³C, ¹⁴C, ¹⁸F, ¹⁵O, ¹³N, ³⁵S, 2H, and ³H, preferably ¹¹C, ³H, and ¹⁸F.

The isotopically labeled compounds of this invention are prepared by incorporating a selected isotope into the substrate molecule. This is accomplished by utilizing reagents that have had one or more of the atoms contained therein made radioactive by placing them in a source of radioactivity such as a nuclear reactor, a cyclotron and the like. Additionally, many isotopically labeled reagents, such as ²H₂O, ³H₃Cl, ¹⁴C₆H₅Br, ClCH₂¹⁴COCl and the like, are commercially available. The isotopically labeled reagents are then used in standard organic chemistry synthetic techniques to incorporate the isotope atom, or atoms, into a compound of Formula I as described below. The following Schemes illustrate how to make the compounds of Formula I.

This invention further relates to a pharmaceutical composition comprising an effective amount of at least one compound of Formula I and a pharmaceutically acceptable carrier. The composition may comprise, but is not limited to, one or more buffering agents, wetting agents, emulsifiers, suspending agents, lubricants, adsorbents, surfactants, preservatives and the like. The composition may be formulated as a solid, liquid, gel or suspension for oral administration (e.g., drench, bolus, tablet, powder, capsule, mouth spray, emulsion); parenteral administration (e.g., subcutaneous, intramuscular, intravenous, epidural injection); topical application (e.g., cream, ointment, controlled-released patch, spray); intravaginal, intrarectal, transdermal, ocular, or nasal administration. In a further embodiment, the pharmaceutical composition of the present invention may be formulated for parenteral administration, such as an intravenous formulation.

This invention provides radiolabeled compounds of Formula I as alpha-synulcein imaging agents and synthetic precursor compounds from which they are prepared. The compounds of Formula I bind aggregated alpha-synuclein to potentially track the progression of age-related diseases such as PD, as well as other synucleinopathies and neurodegenerative diseases, such as Multiple Systems Atrophy (MSA). Dementia with Lewv Bodies (DLB), etc. The compounds of this invention may also be used in combination with a broad range of cognition deficit enhancement agents. Thus, in another embodiment of this invention a compound of Formula (I) or a pharmaceutically acceptable salt thereof, or a pharmaceutical composition or formulation comprising a compound of Formula (I) is administered concurrently, simultaneously, sequentially or separately with another pharmaceutically active compound or compounds used in AD/PD therapies including for example donepezil, memantine, tacrine, carvidopa, levodopa, MOA-B inhibitors, catechol O-methyltransferase (COMT) inhibitors, etc. and equivalents and pharmaceutically active isomer(s) and metabolite(s) thereof.

An objective of the present invention is to provide a radiopharmaceutical agent, such as an isotopically labeled compound of Formula I, that is useful in alpha-synuclein imaging and has high specific radioactivity and high target tissue selectivity by virtue of its high affinity for alpha-synuclein aggregates.

In accordance with the present invention, a method for imaging alpha-synuclein deposits in a patient, wherein an isotopically-labeled compound of Formula I is employed as the imaging agent, comprises the steps of a) placing a human patient in a supine position in a PET camera; b) administering, intravenously, about 0.1 to about 10 mCi of an isotopically-labeled compound of Formula I to the patient; and c) performing an emission scan of the cerebral region of the patient's head to identify aggregations of alpha-synuclein in the brain tissue of the patient. The technique for performing an emission scan of the head is well known to those of skilled in the art. PET techniques are described in Freeman et al., Freeman and Johnson's Clinical Radionuclide Imaging, 3rd. Ed. Vol. 1 (1984): Grune & Stratton, New York; Ennis et Q. Vascular Radionuclide Imaging: A Clinical Atlas. John Wiley & Sons, New York (1983).

The term “labeled tracer” refers to any molecule which can be used to follow or detect a defined activity in vivo, for example, a preferred tracer is one that accumulates in the regions where alpha-synuclein aggregates may be found. Preferably, the labeled tracer is one that can be viewed in a living experimental animal, healthy human or patient (referred to as a subject), for example, by positron emission tomography (PET) scanning. Suitable labels include, but are not limited to radioisotopes, fluorochromes, chemiluminescent compounds, dyes, and proteins, including enzymes.

The present invention also provides methods of determining in vivo activity of an enzyme or other molecule. In an embodiment, an isotopically labeled compound of Formula I is used as a tracer to track the binding activity of aggregated alpha-synuclein protein in the brain and central nervous system.

Biomarkers of Parkinson's disease state, prognosis and progression will all be useful for general diagnostic utilities as well as for clinical development plans for therapeutic agents for Parkinson's disease. Compounds of Formula I may be used to provide biomarker information for patients in clinical trials for novel symptomatic and disease-modifying Parkinson's disease treatments and to assist in patient selection and assignment to cohorts. The present invention will serve as one of the biomarkers of disease state in order to get the correct patients into the proper PhIIb trial cohort. In addition, the present invention can serve as one marker of disease prognosis as an entry inclusion criterion in order to enhance the probability that the disease will progress in the placebo treatment arm, an issue that continues to plague Parkinson's disease clinical trials. Finally, the present invention can serve as one biomarker of disease progression to monitor the clinical course of patients on therapy and could provide an independent biomarker measure of treatment response by a therapeutic drug. The tracer can be selected in accordance with the detection method chosen. Before conducting the method of the present invention, a diagnostically effective amount of a labeled or unlabeled compound of the invention is administered to a living body, including a human.

The present invention also provides a method of measuring the clinical efficacy of therapeutic agents useful for treating Parkinson's Disease (PD) comprising the steps of a) administering an isotopically-labeled compound of Formula I to the patient diagnosed with PD before treatment with said therapeutic agent, b) measuring the amount of alpha-synuclein aggregate formation in the patient's brain tissue, c) administering an isotopically-labeled compound of Formula I to the patient after treatment with said therapeutic agent, d) measuring the amount of alpha-synuclein aggregate formation in the patient's brain tissue after treatment, and e) analyzing whether said therapeutic agent stopped or decreased the progression of alpha-synuclein aggregate formation in the patient's brain tissue.

The diagnostically effective amount of the labeled or unlabeled compound of the invention to be administered before conducting the in-vivo method for the present invention is within a range of from 0.1 ng to 100 mg per kg body weight, preferably within a range of from 1 ng to 10 mg per kg body weight.

The compounds of the present invention have utility in diagnosing, monitoring, and measuring Parkinson's disease and other non-PD synucleinopathies such as Multiple Systems Atrophy (MSA), Dementia with Lewy Bodies (DLB).

In preferred embodiments, the compounds of the invention are useful in diagnosing, monitoring or measuring Parkinson's Disease, non-PD synucleinopathies, neurodegenerative disease, cognitive disorders, schizophrenia, pain disorders and sleep disorders.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients.

In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active compound, which is a compound of Formula I, is included in an amount sufficient to produce the desired effect upon the process or condition of diseases. Accordingly, the pharmaceutical compositions of the present invention encompass any composition made by admixing a compound of the present invention and a pharmaceutically acceptable carrier.

As the term is used herein, “patients” (alternatively “subjects”) refers to an animal, preferably a mammal, and in particular a human, in need of assessment via an imaging study. As used herein, the term “administration” and variants thereof (e.g., “administering” a compound) in reference to a compound of Formula I means providing the compound, or a pharmaceutically acceptable salt thereof, to a subject in need of treatment.

The present invention also provides a method for the synthesis of compounds useful as intermediates in the preparation of compounds of the invention.

The compounds described herein can be prepared according to the procedures of the following schemes and examples, using appropriate materials and are further exemplified by the following specific examples. Deuterated versions of the compounds of the invention can be prepared by substituting an appropriate isotopically labelled reagent for a non-isotopically labelled reagent. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds. Reagents and starting materials for preparing the intermediates and example compounds are commercially available, unless indicated otherwise. All temperatures are degrees Celsius unless otherwise noted. Mass spectra (MS) were measured by electrospray ion-mass spectroscopy (ESI). ¹H NMR spectra were recorded at 300-500 MHz.

List of Abbreviations

| | |
|---|---|
| ACN= | acetonitrile |
| (^{ipc}ADI)NiBr₂= | N,N′-bis(1R,2R,3R,5S)-(−)-isopinocampheyl-2,3-butanediimine |
| | nickel(II) bromide |
| Anal.= | analytical |
| ARG= | autoradiography |
| Bestmann-Ohira reagent= | dimethyl (1-diazo-2-oxopropyl)phosphonate |
| Brett Phos Pd G3= | [(2-Di-cyclohexylphosphino-3,6-dimethoxy-2′,4′,6′-triisopropyl- |
| | 1,1′-biphenyl)-2-(2′-amino-1,1′-biphenyl)]palladium(II) |
| | methanesulfonate |
| BSA= | bovine serum albumin |
| calc.= | calculated |
| CPME= | cyclopentyl methyl ether |
| Cs₂CO₃= | cesium carbonate |
| DAST= | diethylaminosulfur trifluoride |
| Davephos G2 palladacycle= | Chloro[2-(dicyclohexylphosphino)-2′-(N,N-dimethylamino)-1,1′- |
| | biphenyl](2′-amino-1,1′-biphenyl-2-yl)palladium(II) |
| DEA= | diethylamine |
| DIPEA= | N,N-diisopropylethylamine |
| DMF= | dimethylformamide |
| DCM= | dichloromethane |
| DMSO= | dimethyl sulfoxide |
| DMA= | dimethylacetamide |
| EDTA= | ethylenediaminetetraacetic acid |
| ESI= | electrospray ionization |
| EtOAc= | ethyl acetate |
| EtOH= | ethanol |
| Et₂O= | diethyl ether |
| h= | hour(s) |
| HAr= | heteroaryl or aryl |
| HCl= | hydrochloric acid |
| HPLC= | high-pressure liquid chromatography |
| IHC= | immunohistochemistry |
| IPA= | iso-propyl alcohol |
| IPAc= | iso-propyl acetate |
| K₂CO₃= | potassium carbonate |
| LCMS= | Liquid Chromatography coupled to Mass Spectrometry |
| mCi= | millicurie |
| MeCN= | acetonitrile |
| MeI= | iodomethane |
| MeOH= | methyl alcohol |
| MRI= | magnetic resonance imaging |
| MgSO₄= | magnesium sulfate |
| MS= | mass spectroscopy |
| M/Z= | mass to charge ratio |
| NaBH₄= | sodium borohydride |
| NaH= | sodium hydride |
| NaHBEt₃= | sodium triethylborohydride |
| NaHCO₃= | sodium bicarbonate |
| Na₂SO₄= | sodium sulfate |
| NBS= | N-Bromosuccinimide |
| NCS= | N-Chlorosuccinimide |
| NH₄Cl= | ammonium chloride |
| NMP= | N-methylpyrrolidinone |
| NMR= | nuclear magnetic resonance spectroscopy |
| PD= | Parkinson's disease |
| Pd/C= | 10% palladium on carbon by weight |
| Pd₂(dba)₃= | Tris(dibenzylideneacetone)dipalladium(0) |
| PdCl₂(dppf)-CH₂Cl₂= | [1,1′-Bis(diphenylphosphino)ferrocene]dichloropalladium(II), |
| | complex with dichloromethane |
| PE (pet ether)= | petroleum ether |
| Ppm= | parts per million |
| rt= | room temperature |
| RCP= | radiochemical purity |
| RuPhos Pd G2= | chloro(2-dicyclohexylphosphino-2′,6′-diisopropoxy-1,1′- |
| | biphenyl)[2-(2′-amino-1,1′-biphenyl)]palladium(II) |
| SNAr= | nucleophilic aromatic substitution reaction |
| tBu X-phos Pd G3= | tBuXPhos-Pd-G3, [(2-Di-tert-butylphosphino-2′,4′,6′-triisopropyl- |
| | 1,1′-biphenyl)-2-(2′-amino-1,1′-biphenyl)]palladium(II) |
| | methanesulfonate |
| TEA= | triethylamine |
| TFA= | trifluoroacetic acid |
| TLC= | thin-layer chromatography |
| tR= | retention time |
| THF= | tetrahydrofuran |
| wt %= | percentage by weight |
| Xphos Pd G2= | chloro(2-dicyclohexylphosphino-2′,4′,6′-triisopropyl-1,1′- |
| | bipheny1)[2-(2′-amino-1,1′-biphenyl)]palladium(II) |

Compounds described herein were synthesized as a racemic mixture unless otherwise stated in the experimental procedures. In some cases, the final product may be further modified, for example, by manipulation of substituents. These manipulations may include, but are not limited to, reduction, oxidation, alkylation, acylation, and hydrolysis reactions which are commonly known to those skilled in the art. In some cases, the order of carrying out the foregoing reaction schemes may be varied to facilitate the reaction or to avoid unwanted reaction products. The following schemes and examples are provided so that the invention might be more fully understood. These examples are illustrative only and should not be construed as limiting the invention in any way.

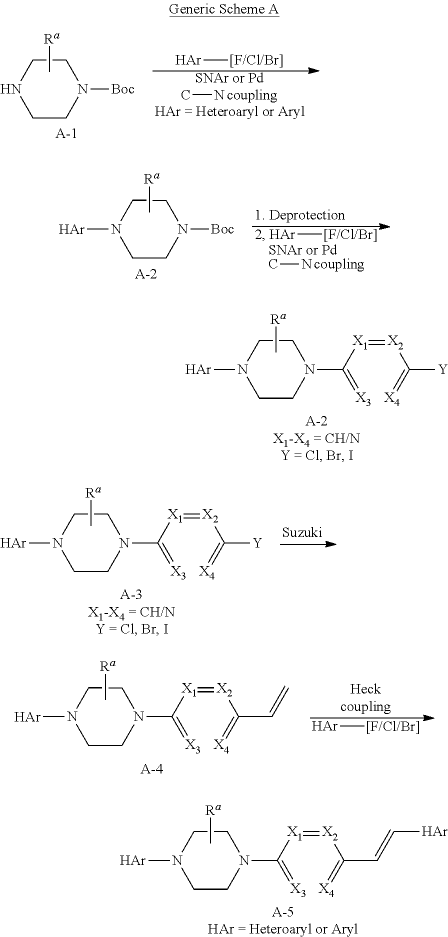

Substituted piperazines (A-1) can be transformed to heteroaryl- or arylpiperazines A-2 via SNAr or Pd-mediated C—N coupling reactions. Deprotection followed by SNAr or Pd-mediated C—N couplings with heteroaryl- or aryl halides provide intermediates A-3. Suzuki coupling utilizing vinyl equivalents can provide intermediates A-4 and subsequent Heck couplings with heteroaryl- or aryl halides can provide target molecules A-5.

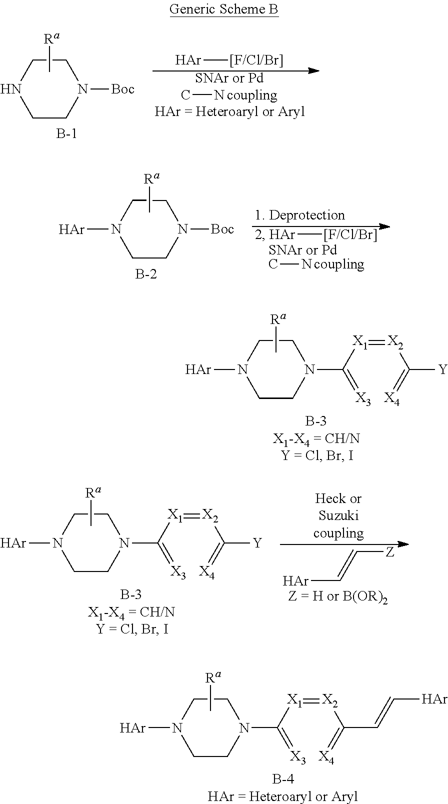

Substituted piperazines (B-1) can be transformed to heteroaryl- or arylpiperazines B-2 via SNAr or Pd-mediated C—N coupling reactions. Deprotection followed by SNAr or Pd-mediated C—N couplings with heteroaryl- or aryl halides provide intermediates B-3. Heck or Suzuki couplings with substituted vinylated aromatic motifs can provide target molecules B-4.

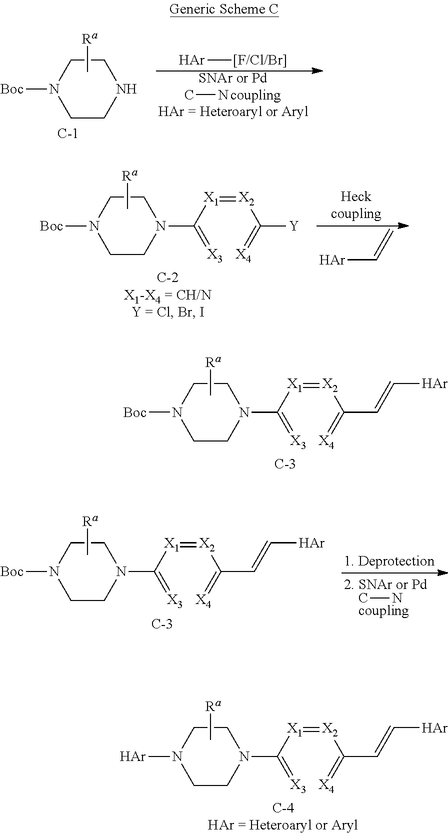

Substituted piperazines (C-1) can be transformed to heteroaryl- or arylpiperazines C-2 via SNAr or Pd-mediated C—N coupling reactions. Heck couplings with substituted vinylated aromatic motifs provide intermediates C-3. Deprotection followed by SNAr or Pd-mediated C—N coupling reactions can provide target molecules C-4.

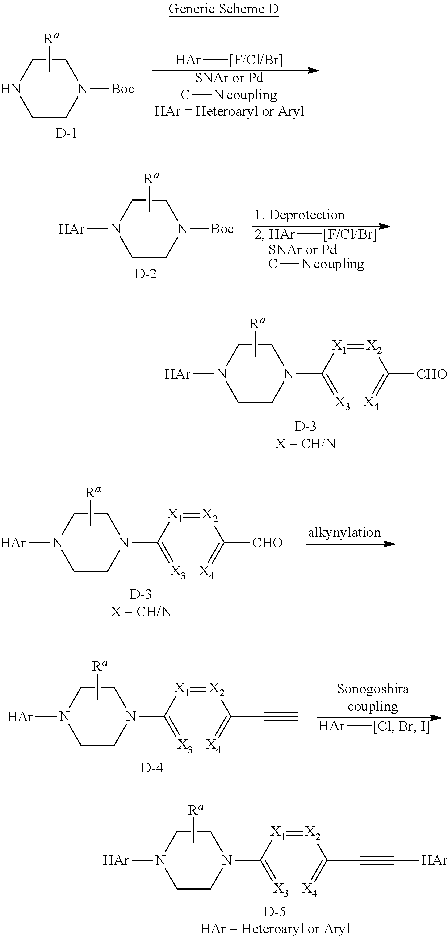

Substituted piperazines (D-1) can be transformed to heteroaryl- or arylpiperazines D-2 via SNAr or Pd-mediated C—N coupling reactions. Deprotection followed by SNAr or Pd-mediated C—N couplings with heteroaryl- or aryl halides provide intermediates D-3. Aldehyde to alkyne transformation can provide intermediates D-4 and subsequent Sonogoshira couplings with heteroaryl- or aryl halides can provide target molecules D-5.

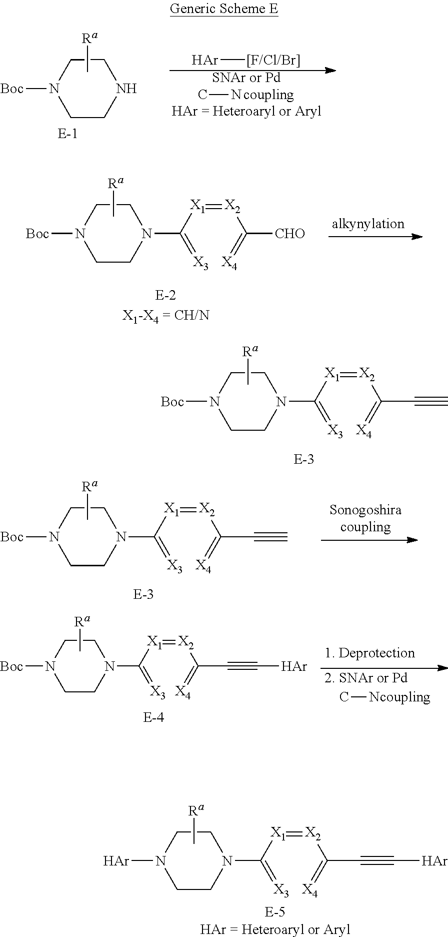

Substituted piperazines (E-1) can be transformed to heteroaryl- or arylpiperazines E-2 via SNAr or Pd-mediated C—N coupling reactions. Aldehyde to alkyne transformation can provide intermediates E-3. Sonogoshira couplings with heteroaryl- or aryl halides can produce intermediates E-4. Deprotection followed by SNAr or Pd-mediated C—N coupling reactions can provide target molecules E-5.

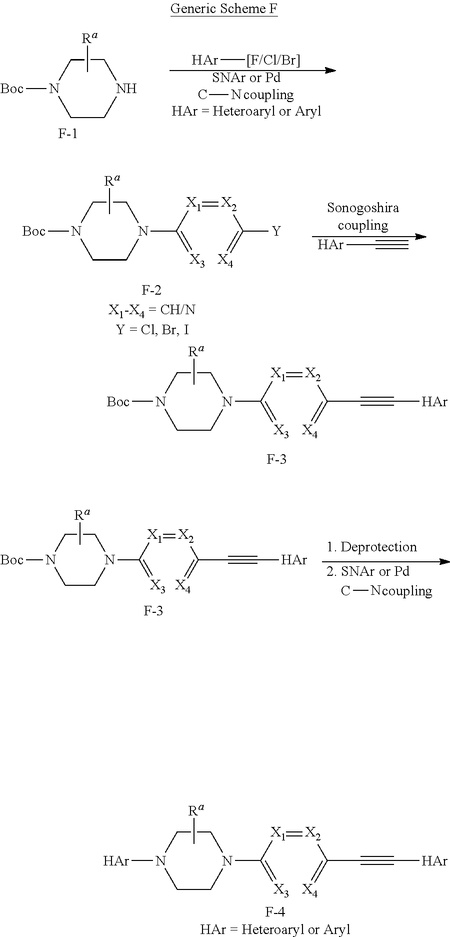

Substituted piperazines (F-1) can be transformed to heteroaryl- or arylpiperazines F-2 via SNAr or Pd-mediated C—N coupling reactions. Sonogoshira couplings with substituted acetylated aromatic motifs provide intermediates F-3. Deprotection followed by SNAr or Pd-mediated C—N coupling reactions can provide target molecules F-4.

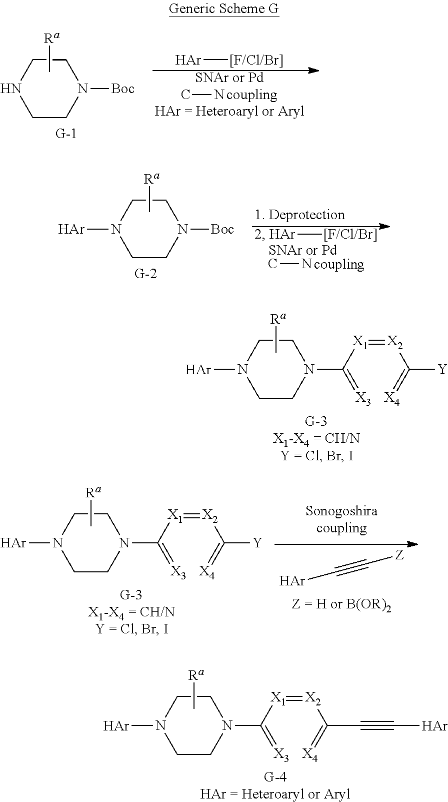

Substituted piperazines (G-1) can be transformed to heteroaryl- or arylpiperazines G-2 via SNAr or Pd-mediated C—N coupling reactions. Deprotection followed by SNAr or Pd-mediated C—N couplings with heteroaryl- or aryl halides provide intermediates B-3. Sonogoshira couplings with substituted acetylated aromatic motifs can provide target molecules B-4.

### Preparation of Intermediates

### Synthesis of Intermediate A: 2-(1H-imidazol-1-yl)-5-vinylpyridine

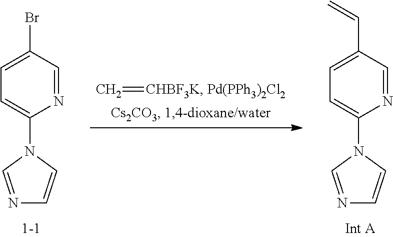

To a stirred solution of 1-1 (40 g, 179 mmol) in 1,4-dioxane (300 mL), water (300 mL) was added cesium carbonate (174 g, 536 mmol) at room temperature and degassed with argon for 20 minutes. Then potassium vinyltrifluoroborate (35.9 g, 268 mmol) and dichlorobis(triphenylphosphine)palladium(II) (6.27 g, 8.93 mmol) were added to the reaction mixture at room temperature. The resulting reaction mixture was stirred at 100° C. for 16 h. Reaction mixture was quenched with water (500 mL) and extracted with EtOAc (2×500 mL). The combined organic layer was washed with brine (500 mL), dried over sodium sulphate, filtered and the filtrate was concentrated under reduced pressure to afford crude product. The crude compound was purified by column chromatography on silica gel (100-200 mesh) and compound eluted with a gradient of 60% ethyl acetate in petroleum ether. Pure fractions were concentrated under reduced pressure to afford Int A. M/Z (ESI): 171.96 [M+H]⁺.

### Synthesis of Intermediate B: (R)-(4-(5-bromopyrimidin-2-yl)-1-(pyrimidin-2-yl)piperazin-2-yl)methanol

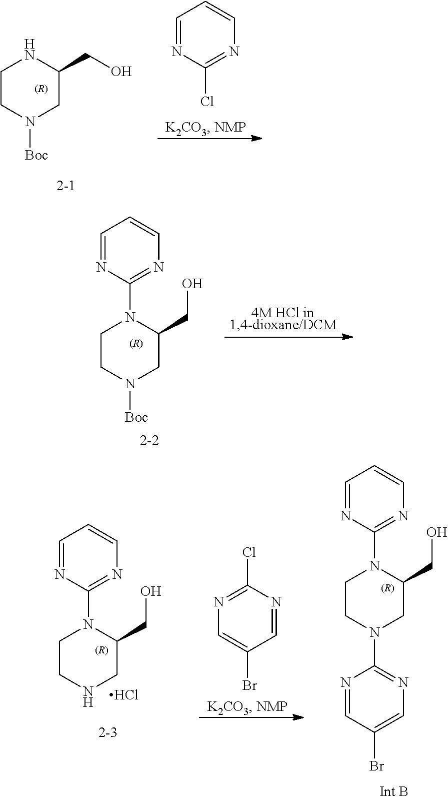

### Synthesis of 2-2: tert-butyl (R)-3-(hydroxymethyl)-4-(pyrimidin-2-yl)piperazine-1-carboxylate

To a stirred solution of 2-1 (15 g, 69.4 mmol) in NMP (150 mL) were added K₂CO₃ (19.17 g, 139 mmol) and 2-chloropyrimidine (7.94 g, 69.4 mmol) at room temperature. The reaction mixture was stirred under a nitrogen atmosphere at 80° C. for 24 h. The reaction mixture was quenched with ice cold water (200 mL) and extracted with EtOAc (3×200 mL). Combined organic layer was washed with ice cold water (100 mL), brine (100 mL), dried over Na₂SO₄, filtered, and concentrated under reduced pressure. The crude compound was purified by column chromatography over (100-200 mesh) silica gel and the compound was eluted with a gradient of 50% EtOAc in Pet ether. Pure fractions concentrated under reduced pressure to afford 2-2. M/Z (ESI): 295.16 [M+H]⁺.

### Synthesis of 2-3: (R)-(1-(pyrimidin-2-yl)piperazin-2-yl)methanol

To a stirred solution of 2-2 (9 g, 30.6 mmol) in DCM (45 mL) was added 4N HCl in 1,4-dioxane (38.2 mL, 153 mmol) at 0° C. The reaction mixture was stirred under nitrogen atmosphere at 25° C. for 16 h. Reaction mixture was concentrated under reduced pressure. The crude compound was triturated with diethyl ether (20 mL). Obtained solid was filtered and dried under reduced pressure to afford 2-3. M/Z (ESI): 195.06 [M+H]⁺.

### Synthesis of Int B: (R)-(4-(5-bromopyrimidin-2-yl)-1-(pyrimidin-2-yl)piperazin-2-yl)methanol

To a stirred solution of 2-3 (3 g, 13.00 mmol) in NMP (30 mL) were added K₂CO₃ (7.19 g, 52.0 mmol) and 5-bromo-2-chloropyrimidine (2.52 g, 13.00 mmol) at room temperature. The reaction mixture was stirred under nitrogen atmosphere at 80° C. for 16 h. Reaction mixture was quenched with ice cold water (100 mL) and extracted with EtOAc (3×100 mL). Combined organic layer was washed with ice cold water (100 mL), brine (100 mL), dried over Na₂SO₄, filtered, and concentrated under reduced pressure. The crude compound was purified by column chromatography over (100-200 mesh) silica and compound eluted with a gradient of 50% EtOAc in Pet ether. Pure fractions concentrated under reduced pressure and obtained compound was further re-purified by Prep HPLC purification (condition: mobile phase—10 mM Ammonium Bicarbonate in H₂O: MeCN column—X-Bridge C18 (19×250)mm 5 um Flow-18 ml/min gradient method-0/45, 4/50, 10.5/50, 10.6/100, 11.9/100, 12/45, 15/45). Pure fractions concentrated under reduced pressure and lyophilized to afford Int B. M/Z (ESI): 351.23 [M+H]⁺.

### Synthesis of Intermediate C: (R)-5-bromo-2-(3-(methoxymethyl)-4-(pyrimidin-2-yl)piperazin-1-yl)pyrimidine

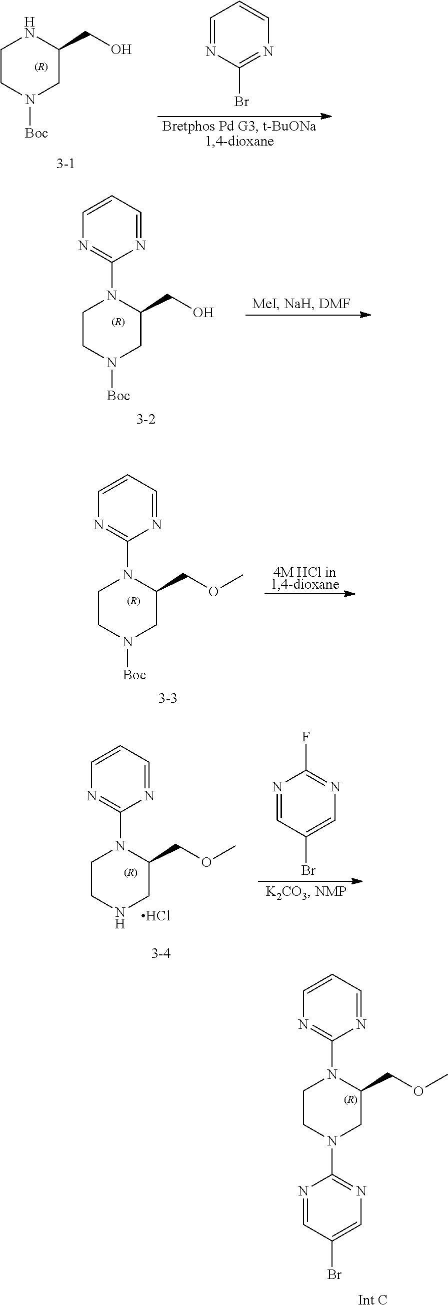

### Synthesis of 3-2: tert-butyl (R)-3-(hydroxymethyl)-4-(pyrimidin-2-yl)piperazine-1-carboxylate

To degassed argon, a stirred solution of 3-1 (1 g, 4.62 mmol) in dioxane (16 mL) were added sodium tert-butoxide (1.333 g, 13.87 mmol), [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate (BrettPhos Pd G3) (0.210 g, 0.231 mmol) and 2-bromopyrimidine (0.882 g, 5.55 mmol) at room temperature. The reaction mixture was stirred for 12 h at 100° C. The reaction mixture was quenched with ice water and extracted with EtOAc (50 mL×2). Combined organic layer was washed with brine solution, dried over anhydrous sodium sulphate, filtered, and concentrated under reduced pressure. The crude compound was purified by 100×200 silica gel Biotage flash column chromatography using 50% EtOAc/pet-ether as eluent. Pure fractions concentrated under reduced pressure to afford compound 3-2. M/Z (ESI): 295.19 [M+H]⁺.

### Synthesis of 3-3: tert-butyl (R)-3-(methoxymethyl)-4-(pyrimidin-2-yl)piperazine-1-carboxylate

To a stirred solution of compound 3-2 (800 mg, 2.72 mmol) in DMF (10 mL) were added methyl iodide (1929 mg, 13.59 mmol) and sodium hydride (98 mg, 4.08 mmol) at room temperature. Reaction mixture was stirred for 2 h at 0° C. Reaction mixture was quenched with ice water and extracted with EtOAc (10 mL×2). Combined organic layer was washed with brine solution, dried over anhydrous sodium sulphate, filtered, and evaporated under reduced pressure to afford compound 3-3. M/Z (ESI): 309.19 [M+H]⁺.

### Synthesis of 3-4: (R)-2-(2-(methoxymethyl)piperazin-1-yl)pyrimidine

To a stirred solution of compound 3-3 (800 mg, 2.59 mmol) in DCM (9 mL) was added 4M HCl in 1,4-dioxane (646 mg, 5.19 mmol) at 0° C. and stirred at 25° C. for 2 h. Reaction mixture was evaporated under reduced pressure, co-distilled with toluene, washed with pentane and dried to afford compound 3-4. M/Z (ESI): 209.19 [M+H]1.

### Synthesis of Int C: (R)-5-bromo-2-(3-(methoxymethyl)-4-(pyrimidin-2-yl)piperazin-1-yl)pyrimidine

To a stirred solution of compound 3-4 (700 mg, 2.86 mmol) in DMF (10 mL) at 0° C. were added potassium carbonate (0.813 ml, 14.30 mmol) followed by 5-bromo-2-fluoropyrimidine (607 mg, 3.43 mmol). Reaction mixture was stirred for 12 h at 80° C. Reaction mixture was quenched with ice water, resulting solid was filtered and dried under reduced pressure to afford compound Int C. M/Z (ESI): 365.06 [M+H]⁺.

### Synthesis of Intermediate D: (R)-2-(3-(methoxymethyl)-4-(pyrimidin-2-yl)piperazin-1-yl)-5-vinylpyrimidine

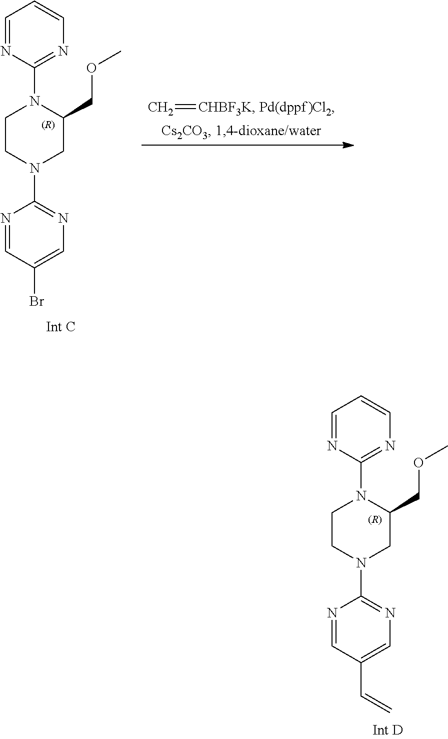

To a stirred solution of Int C (10 g, 27.4 mmol) in 14-dioxane (80 mL) and water (14 mL) were added Cs₂CO₃ (17.84 g, 54.8 mmol) and potassium vinyltrifluoroborate (7.33 g, 54.8 mmol) at room temperature and degassed with argon gas for 15 minutes. Then PdCl₂(dppf)-CH₂Cl₂ adduct (2.236 g, 2.74 mmol) was added to the reaction mixture at room temperature and the resulting reaction mixture was stirred under nitrogen atmosphere at 100° C. for 16 h in a sealed tube. Reaction mixture was quenched with water (50 mL) and extracted with EtOAc (2×200 mL). Combined organic layer was washed with brine (50 mL), dried over anhydrous Na₂SO₄, filtered and the filtrate was concentrated under reduced pressure to afford crude product. The crude compound was purified by column chromatography on silica gel (100-200 mesh) and compound was eluted with a gradient of 30% EtOAc in pet ether. Pure fractions were concentrated under reduced pressure to afford Int D. M/Z (ESI): 313.63 [M+H]⁺.

### Synthesis of Intermediate E: (R,E)-5-(2-(6-(1H-imidazol-1-yl)pyridin-3-yl)vinyl)-2-(3-(methoxymethyl)piperazin-1-yl)pyrimidine

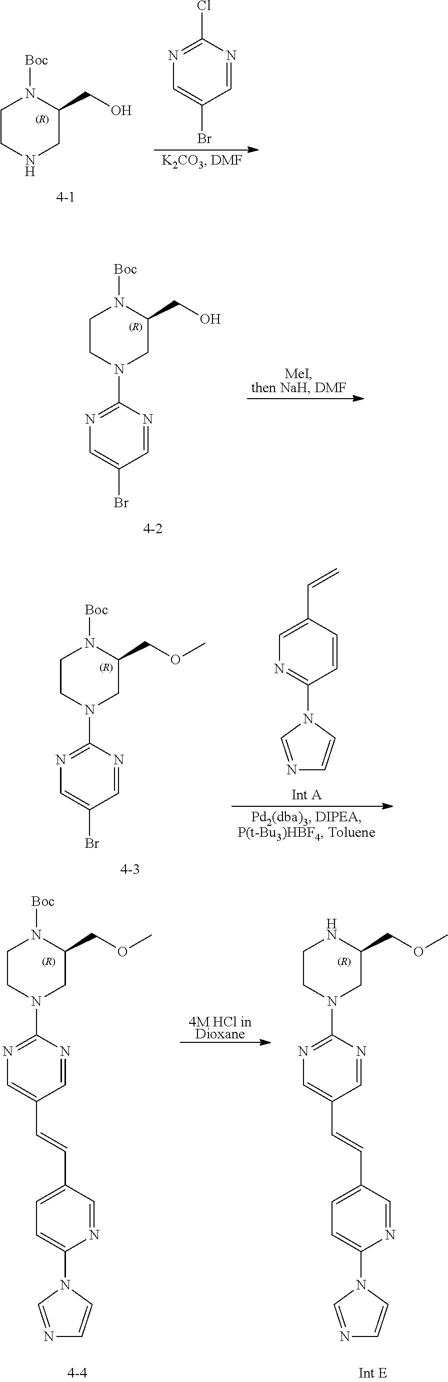

### Synthesis of 4-2: tert-butyl (R)-4-(5-bromopyrimidin-2-yl)-2-(hydroxymethyl)piperazine-1-carboxylate

To a stirred solution of tert-butyl 4-1 (25 g, 116 mmol) and 5-bromo-2-chloropyrimidine (26.8 g, 139 mmol) in DMF (250 mL) was added K₂CO₃ (39.9 g, 289 mmol) at room temperature. Reaction mixture was stirred at 80° C. for 2 h. The reaction mixture was diluted with ice water (100 mL), extracted with EtOAc (2×200 mL). Combined organic layer washed with brine solution (50 mL), dried over anhydrous Na₂SO₄ and filtered, dried and concentrated under reduced pressure to get crude compound. The crude compound was purified by silica column and eluted with 20% EtOAc in pet ether as gradient. Pure fractions concentrated under reduced pressure to afford compound 4-2. M/Z (ESI): 373.19 [M+H]⁺.

### Synthesis of 4-3: tert-butyl (R)-4-(5-bromopyrimidin-2-yl)-2-(methoxymethyl)piperazine-1-carboxylate

To a stirred solution of compound 4-2 (25 g, 67.0 mmol) in DMF (250 mL) were added Mel (20.94 mL, 335 mmol) and NaH (5.36 g, 134 mmol) at 0° C. Reaction mixture was stirred at room temperature for 2 h. The reaction mixture was diluted with water (100 mL), extracted with EtOAc (2×200 mL). Combined organic layer was washed with brine solution (50 mL), dried over anhydrous Na₂SO₄ and filtered, and concentrated under reduced pressure to get crude compound. The crude compound was purified by silica column, eluted with 15% EtOAc in pet ether as gradient. Pure fractions concentrated under reduced pressure to afford compound 4-3. M/Z (ESI): 387.14 [M+H]⁺.

### Synthesis of 4-4: tert-butyl (R,E)-4-(5-(2-(6-(1H-imidazol-1-yl)pyridin-3-yl)vinyl)pyrimidin-2-yl)-2-(methoxymethyl)piperazine-1-carboxylate

To a stirred solution of compound 4-3 (lg, 2.58 mmol) in Toluene (10 mL) were added compound Int A (0.442 g, 2.58 mmol), DIPEA (1.353 mL, 7.75 mmol) at room temperature and degassed with argon gas for 10 min, followed by Pd₂(dba)₃ (0.118 g, 0.129 mmol) and tri-tert-butylphosphonium tetrafluoroborate (0.075 g, 0.258 mmol) added under nitrogen atmosphere. Resulting reaction mixture was stirred at 120° C. for 16 h. The reaction mixture was filtered through celite pad and diluted with water (5 mL), extracted with EtOAc (2×10 ml). Combined organic layer was washed with brine solution (5 mL), dried over anhydrous Na₂SO₄ and filtered, concentrated under reduced pressure to get crude compound. The crude compound was purified by silica column, eluted with 5% MeOH in DCM as gradient. Pure fractions concentrated under reduced pressure to afford compound 4-4. M/Z (ESI): 478.35 [M+H]⁺.

### Synthesis of Int E: (R,E)-5-(2-(6-(1H-imidazol-1-yl)pyridin-3-ylvinyl)-2-(3-(methoxymethyl)piperazin-1-yl)pyrimidine

To a stirred solution of compound 4-4 (400 mg, 0.838 mmol) in DCM (5 mL) was added 4M HCl in 1,4-dioxane (0.419 mL, 1.675 mmol) at 0° C. The reaction mixture was stirred at room temperature for 16 h. The reaction mixture was concentrated under reduced pressure to afford compound Int E. M/Z (ESI): 378.23 [M+H]⁺.

### Synthesis of Intermediate F: (R)-2-(2-(methoxymethyl)-4-(pyridin-2-yl)piperazin-1-yl)-5-vinylpyrimidine

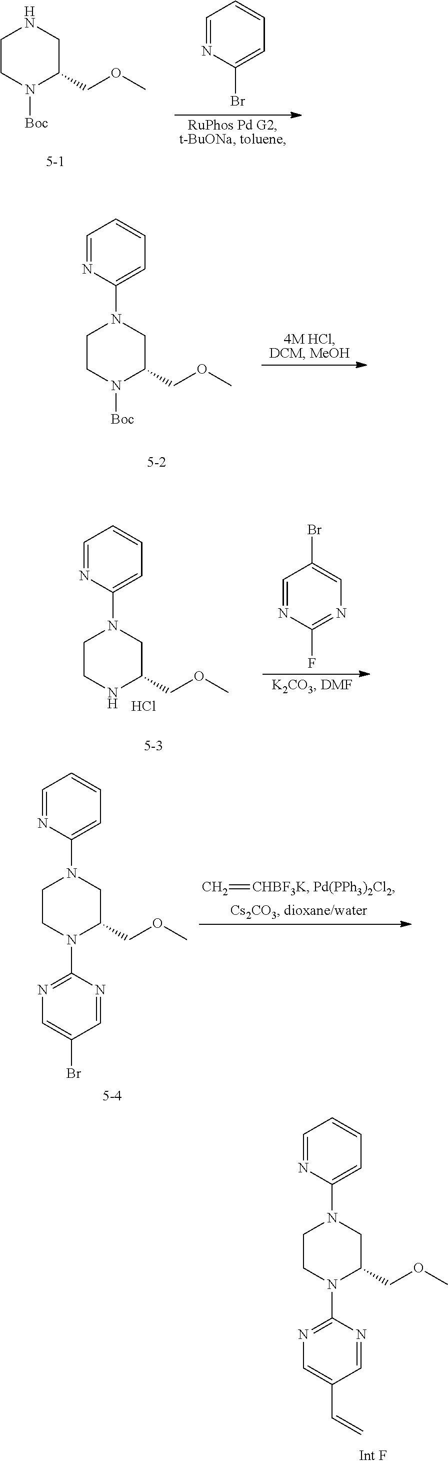

### Synthesis of 5-2: tert-butyl (R)-2-(methoxymethyl)-4-(pyridin-2-yl)piperazine-1-carboxylate

A stirred solution of 5-1 (1.50 g, 6.51 mmol) in toluene (60 mL) was purged with nitrogen for 10 minutes. 2-bromopyridine (0.932 mL, 9.77 mmol), sodium tert-butoxide (1.565 g, 16.28 mmol) and chloro(2-dicyclohexylphosphino-2′,6′-diisopropoxy-1,1′-biphenyl)[2-(2′-amino-1,1′-biphenyl)]palladium(II) (RuPhos Pd G2) (0.506 g, 0.651 mmol) were added and the mixture was heated at 110° C. under stirring for 18 h. The reaction mixture was filtered through a Celite pad and washed with EtOAc (3×20 ml). The filtrate was concentrated and purified by flash column chromatography (120 g Isco Gold column), eluting with Hexanes:EtOAc 100:0 to 50:50. The desired fractions were concentrated to afford compound 5-2. M/Z (ESI): 308.0 [M+H]⁺.

### Synthesis of 5-3: (R)-3-(methoxymethyl)-1-(pyridin-2-yl)piperazine hydrochloride

To a solution of 5-2 (1.97 g, 6.41 mmol)) in dichloromethane (5 mL) and MeOH (1 mL) was added 4M HCl in dioxane (4.81 mL, 19.23 mmol) and the reaction was aged at ambient temperature for 2 h. The reaction mixture was evaporated under reduced pressure and dried under high vacuum to afford compound 5-3. M/Z (ESI): 208.0 [M+H]⁺.

### Synthesis of 5-4: (R)-5-bromo-2-(2-(methoxymethyl)-4-(pyridin-2-yl)piperazin-1-yl)pyrimidine

A solution of 5-3 (1.86 g. 6.64 mmol), 5-bromo-2-fluoropyrimidine (2.350 g, 13.28 mmol), and potassium carbonate (2.75 g, 19.91 mmol) in DMF (25 mL) was heated at 70° C. for 18 h. The mixture was diluted with water (75 mL) and then extracted with Et₂O(3×30 mL).

The combined organic layers were dried over Na₂SO₄, filtered, and concentrated. The crude was purified by flash column chromatography (120 g Isco Gold column), eluting with Hexanes:EtOAc 100:0 to 40:60, to afford compound 5-4. M/Z (ESI): 364.2, 366.2 [M+H]⁺.

### Synthesis of Int F: (R)-2-(2-(methoxymethyl)-4-(pyridin-2-yl)piperazin-1-yl)-5-vinylpyrimidine

A nitrogen purged solution of 5-4 (1.30 g, 3.57 mmol), potassium vinyltrifluoroborate (0.717 g, 5.35 mmol), cesium carbonate (3.49 g. 10.71 mmol), and dichlorobis(triphenylphosphine) palladium(II) (0.125 g, 0.178 mmol) in dioxane (13.38 ml) was heated at 100° C. for 21 h. The mixture was diluted with water (30 mL) and then extracted with EtOAc (3×30 mL). The combined organic layers were dried over Na₂SO₄, filtered, and concentrated. The crude was purified by flash column chromatography (120 g Isco Gold column), eluting with Hexanes:EtOAc 100:0 to 50:50, to afford compound Int F. M/Z (ESI): 312.2 [M+H]⁺.

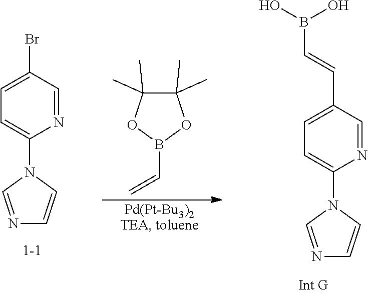

### Synthesis of Intermediate G: ((E)-(2-(6-(1H-imidazol-1-yl)pyridin-3-yl)vinyl)boronic acid

A solution of 5-bromo-2-(1H-imidazol-1-yl)pyridine (1-1, 1.00 g, 4.46 mmol) and TEA (1.24 mL, 8.93 mmol) in toluene (44.6 mL) was purged with nitrogen for 15 minutes. Vinylboronic acid pinacol ester (1.14 mL, 6.69 mmol) and bis(tri-t-butylphosphine)palladium(0) (0.114 g, 0.223 mmol) were added and the mixture was heated at 110° C. for 16 h. The mixture was filtered through a pad of Celite, concentrated, and purified by flash column chromatography (40 g Isco Gold column), eluting with Hexanes:EtOAc/EtOH(3:1) 100:0 to 0:100, to afford compound Int G. M/Z (ESI): 216.1 [M+H]+

### Synthesis of Intermediate H: (R,E)-5-(2-(6-(1H-imidazol-1-yl)pyridin-3-yl)vinyl)-2-(2-((2-methoxyethoxy)methyl)piperazin-1-yl)pyrimidine dihydrochloride

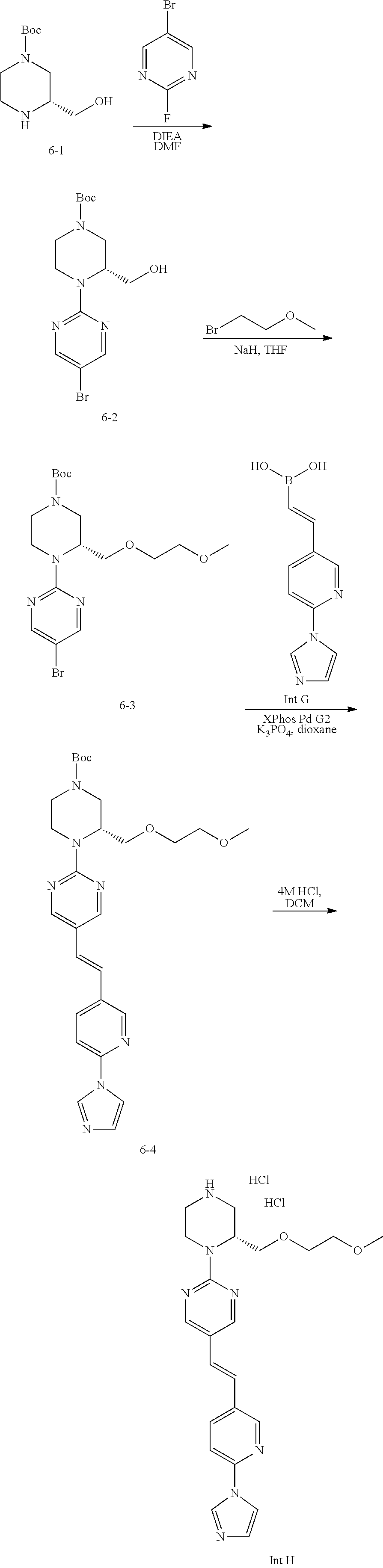

### Synthesis of 6-2: tert-butyl (R)-4-(5-bromopyrimidin-2-yl)-3-(hydroxymethyl)piperazine-1-carboxylate

A solution of 6-1 (2.74 g, 12.66 mmol), 5-bromo-2-fluoropyrimidine (2.00 g, 11.30 mmol), and DIEA (19.7 mL, 113 mmol) in DMF (40 mL) was heated at 100° C. for 16 h. The solution was poured onto water (100 mL) and extracted with Et₂O(3×30 mL). The combined organics were dried over Na₂SO₄, filtered, and concentrated. The crude was purified by flash column chromatography (120 g Isco Gold column), eluting with Hexanes:EtOAc 100:0 to 0:100, to afford compound 6-2. M/Z (ESI): 373.0, 375.0 [M+H]⁺.

### Synthesis of 6-3: tert-butyl (R)-4-(5-bromopyrimidin-2-yl)-3-((2-methoxyethoxy)methyl)piperazine-1-carboxylate

Sodium hydride (0.219 g, 5.47 mmol, 60 wot %) was added to a solution of 6-2 (1.02 g, 2.73 mmol) in THF (13.66 ml) at ambient temperature and the solution was stirred for 0.5 h. 1-Bromo-2-methoxyethane (0.514 ml, 5.47 mmol) was then added and the solution was stirred at 50° C. for 20 h. The solution was poured onto water (30 mL) and extracted with EtOAc (3×30 mL). The combined organics were dried over Na₂SO₄, filtered, and concentrated. The crude was purified by flash column chromatography (80 g Isco Gold column), eluting with Hexanes:EtOAc 100:0 to 0:100, to afford compound 6-3. M/Z (ESI): 431.3, 433.3 [M+H]⁺.

### Synthesis of 6-4: tert-butyl (R,E)-4-(5-(2-(6-(1H-imidazol-1-yl)pyridin-3-yl)vinyl)pyrimidin-2-yl)-3-((2-methoxyethoxy)methyl)piperazine-1-carboxylate

A solution of 6-3 (200 mg, 0.464 mmol), Int G (150 mg, 0.696 mmol), and 1M tripotassium phosphate (1391 μl, 1.391 mmol) in dioxane (4637 μl) was purged with nitrogen. Chloro(2-dicyclohexylphosphino-2′,4′,6′-triisopropyl-1,1′-biphenyl)[2-(2′-amino-1,1′-biphenyl)]palladium(II) (36.5 mg, 0.046 mmol) was added and the solution was stirred at 80° C. for 1.5 h. The mixture was filtered through a pad of Celite, concentrated, and purified by flash column chromatography (40 g Isco Gold column), eluting with Hexanes:EtOAc 100:0 to 0:100, to afford compound 6-4. M/Z (ESI): 522.4 [M+H]⁺.

### Synthesis of Int H: (R,E)-5-(2-(6-(1H-imidazol-1-yl)pyridin-3-yl)vinyl)-2-(2-((2-methoxyethoxy)methyl)piperazin-1-yl)pyrimidine dihydrochloride

A solution of 4M HCl in dioxane (1069 μl, 4.28 mmol) was added to a solution of 6-4 (223 mg, 0.428 mmol) in DCM (4275 μl) and the solution was aged at ambient temperature for 2 h. The mixture was concentrated and dried under high vacuum to afford compound Int H. M/Z (ESI): 422.3 [M+H]⁺.

### Synthesis of Intermediate I: (R)-(4-(5-ethynylpyrimidin-2-yl)-1-(pyrimidin-2-yl)piperazin-2-yl)methanol

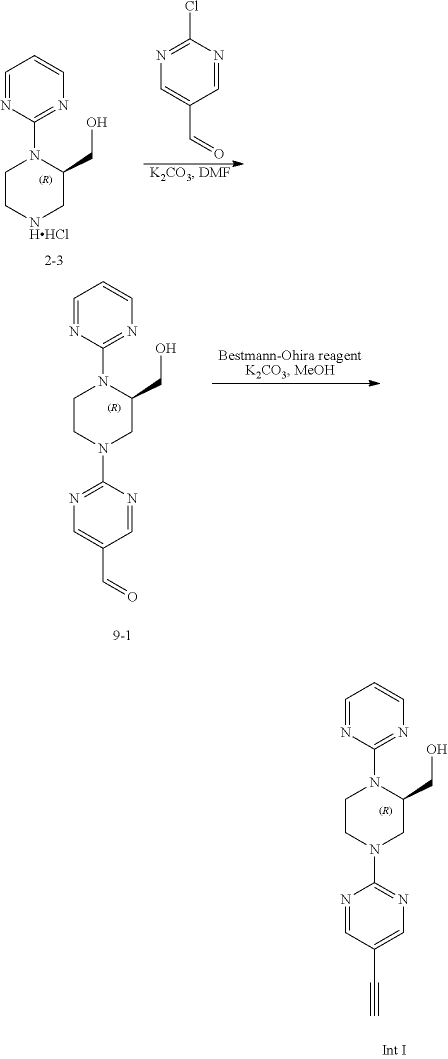

### Synthesis of 9-1: (R)-2-(3-(hydroxymethyl)-4-(pyrimidin-2-yl)piperazin-1-yl)pyrimidine-5-carbaldehyde

To a stirred solution of compound 2-3 (3 g. 13.0 mmol) in DMF (45 mL) were added potassium carbonate (8.99 g, 65.0 mmol) at room temperature followed by 2-chloropyrimidine-5-carbaldehyde (1.854 g, 13.00 mmol) added at room temperature. Resulting reaction mixture was stirred at 80° C. for 2 h. The reaction mixture was quenched with ice cold water, extracted with EtOAc (30 mL). The combined organic layer was separated, the aqueous layer was re-extracted with EtOAc (60 mL), and then washed with brine solution and dried over anhydrous sodium sulphate, filtered and evaporated under reduced pressure to afford crude compound (4.5 g) as brown solid. The crude compound was purified by 100-200 silica gel Biotage flash column chromatography, eluted with 50% EtOAc/pet-ether as gradient. Pure fractions concentrated under reduced pressure to afford compound 9-1. M/Z (LCMS) (M+H): 301.20.

### Synthesis of Int I: (R)-(4-(5-ethynylpyrimidin-2-yl)-1-(pyrimidin-2-yl)piperazin-2-yl)methanol

To a stirred solution of compound 9-1 (1.8 g. 5.99 mmol) in MeOH (20 mL) were added potassium carbonate (1.657 g. 11.99 mmol) and dimethyl (1-diazo-2-oxopropyl)phosphonate (Bestmann-Ohira reagent, 1.626 mL, 7.19 mmol) at 0° C., then the reaction mixture was stirred at room temperature for 16 h. The reaction mixture was diluted with water (20 mL), extracted with EtOAc (2×30 mL). The combined organic layer was washed with brine solution (10 mL), dried over anhydrous Na₂SO₄ and filtered and concentrated under reduced pressure to get crude compound. Crude compound was purified over silica column and product was eluted with 40% EtOAc in pet ether as gradient. Pure fractions concentrated under reduced pressure to afford compound Int I. M/Z (LCMS) (M+H): 297.25.

### Synthesis of Intermediate J: (R)-5-((6-(1H-imidazol-1-yl)pyridin-3-yl)ethynyl)-2-(3-(methoxymethyl)piperazin-1-yl)pyrimidine hydrochloride

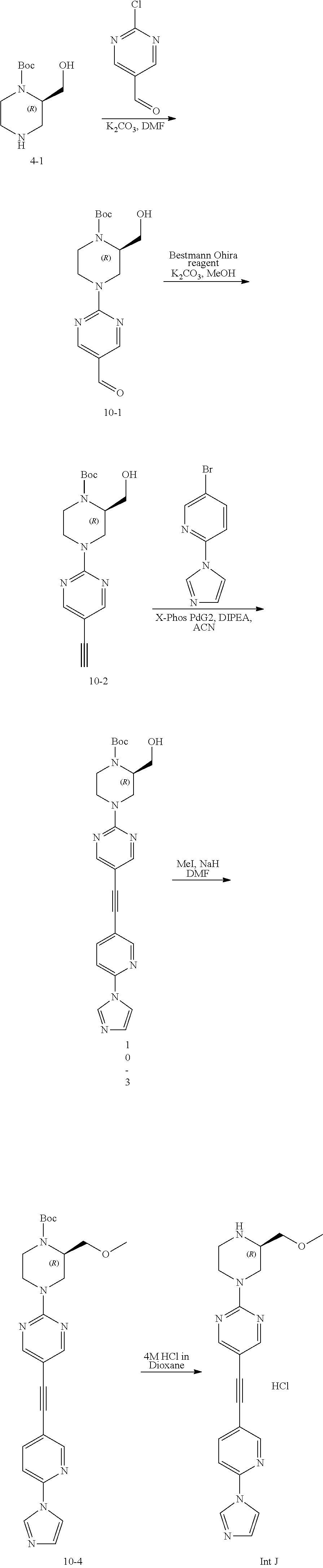

### Synthesis of 10-1: tert-butyl (R)-4-(5-formylpyrimidin-2-yl)-2-(hydroxymethyl)piperazine-1-carboxylate

To a stirred solution of 4-1 (5 g, 23.12 mmol) in DMF (80 mL) were added potassium carbonate (3.94 ml, 69.4 mmol) at room temperature, followed by 2-chloropyrimidine-5-carbaldehyde (4.28 g, 30.1 mmol) at room temperature. Resulting reaction mixture was stirred for 12 h at 80° C. Reaction mixture was quenched with Ice water, extracted with EtOAc (50 mL×2). The combined organic layer was washed with brine solution, dried over anhydrous sodium sulphate, filtered and evaporated under reduced pressure to get crude compound. The crude compound was purified over 100-200 silica gel Biotage flash column chromatography, eluted with 20% EtOAc/pet-ether as gradient. Pure fractions concentrated under reduced pressure to afford compound 10-1. M/Z (LCMS) (M+H): 323.17.

### Synthesis of 10-2: tert-butyl (R)-4-(5-ethynylpyrimidin-2-yl)-2-(hydroxymethyl)piperazine-1-carboxylate

To a stirred solution of compound 10-1 (6 g, 18.61 mmol) in MeOH (75 mL) were added potassium carbonate (5.14 g, 37.2 mmol) at room temperature, followed by dimethyl (1-diazo-2-oxopropyl)phosphonate (Bestmann-Ohira reagent; 5.36 ml. 22.33 mmol) added. Reaction mixture was stirred for 4 h at 25° C. Reaction mixture was quenched with saturated NH₄Cl solution, extracted with DCM (10 mL×3). The combined organic layer was washed with brine solution, dried over anhydrous sodium sulphate, filtered, evaporated under reduced pressure to get crude compound. The crude compound was purified over 100-200 silica gel Biotage flash column chromatography, eluted with 30% EtOAc/pet-ether as gradient. Pure fractions concentrated under reduced pressure to afford compound 10-2. M/Z (LCMS) (M+H): 319.22.

### Synthesis of 10-3: tert-butyl (R)-4-(5-((6-(1H-imidazol-1-yl)pyridin-3-yl)ethynyl)pyrimidin-2-yl)-2-(hydroxymethyl)piperazine-1-carboxylate

To a stirred solution of compound 10-2 (3.5 g, 10.99 mmol) in ACN (40 mL) (degassed with argon) were added N,N-Diisopropylethylamine (4.26 g, 33.0 mmol), followed by Chloro(2-dicyclohexylphosphino-2′,4′,6′-triisopropyl-1,1′-biphenyl)[2-(2′-amino-1,1′-biphenyl)]palladium(II) (0.865 g, 1.099 mmol), 5-bromo-2-(1H-imidazol-1-yl)pyridine (3.69 g, 16.49 mmol) at room temperature. The reaction mixture was stirred for 1 h at 80° C. Reaction mixture was quenched with Ice water, extracted with EtOAc (50 mL). The combined organic layer was washed with brine solution, dried over anhydrous sodium sulphate, filtered and evaporated under reduced pressure to get crude compound as brown solid. The crude compound was purified over 100-200 silica gel Biotage flash column chromatography, eluted with EtOAc as gradient. Pure fractions concentrated under reduced pressure to afford compound 10-3. M/Z (LCMS) (M+H): 462.37.

### Synthesis of 10-4: tert-butyl (R)-4-(5-((6-(1H-imidazol-1-yl)pyridin-3-yl)ethynyl)pyrimidin-2-yl)-2-(methoxymethyl)piperazine-1-carboxylate

To a stirred solution of compound 10-3 (2 g, 4.33 mmol) in DMF (15 mL) were added methyl iodide (1.230 g, 8.67 mmol) at 0° C. and stirred for 5 min at 0° C., followed by sodium hydride (0.416 g, 17.33 mmol) at 0° C. Reaction mixture was stirred for 2 h at 25° C. Reaction mixture was quenched with Ice water, diluted with EtOAc (15 mL×3). The combined organic layer was washed with brine solution, dried over anhydrous sodium sulphate, filtered and evaporated under reduced pressure to get crude compound. The crude compound was purified over 100-200 silica gel Biotage flash column chromatography, 20% EtOAc/pet-ether as eluent. All desired fractions were combined (determined by TLC) and concentrated under reduced pressure to get compound 10-4. M/Z (LCMS) (M+H): 476.30.

### Synthesis of Int J: (R)-5-((6-(1H-imidazol-1-yl)pyridin-3-yl)ethynyl)-2-(3-(methoxymethyl) piperazin-1-yl)pyrimidine hydrochloride

To a stirred solution of compound 10-4 (2 g, 4.21 mmol) in DCM (15 mL) were added 4M HCl in 1,4-dioxane (1.048 g, 8.41 mmol) at 0° C. Resulting reaction mixture was stirred at 2 h for 25° C. The reaction mixture was evaporated under reduced pressure and cold distilled with DCM. Obtained compound was washed with pentane, dried under reduced pressure to afford compound Int J. M/Z (LCMS) (M+H): 376.30.

### EXAMPLES

### Example 1

### (R,E)-(4-(5-(2-(6-(1H-imidazol-1-yl)pyridin-3-yl)vinyl)pyrimidin-2-yl)-1-(pyrimidin-2-yl)piperazin-2-yl)methanol

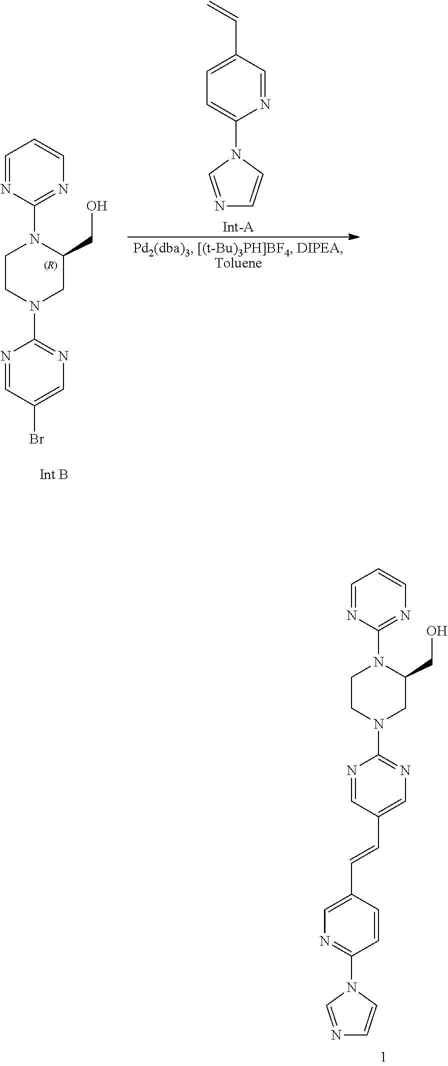

A stirred solution of Int B (1 g, 2.85 mmol) in toluene (10 mL) was purged with Argon gas for 10 min. To the reaction mixture was added Int A (0.487 g, 2.85 mmol), DIPEA (1.492 mL, 8.54 mmol). Pd₂(dba)₃ (0.261 g. 0.285 mmol) and tri-tert-butylphosphonium tetrafluoroborate (0.165 g, 0.569 mmol) at room temperature and again purged with Argon for another 10 min. The reaction mixture was stirred at 100° C. for 16 h. Reaction mixture was quenched with water (50 mL), extracted with 10% MeOH in DCM (2×100 mL). The combined organic layer was washed with brine (2×50 mL), dried over anhydrous Na₂SO₄, filtered, and concentrated under reduced pressure. The crude compound was purified by column chromatography over (100-200 mesh) silica and compound eluted with a gradient of 10% MeOH in DCM. Pure fractions concentrated under reduced pressure to afford 1. M/Z (ESI): 442.33 [M+H]⁻; ¹H NMR (DMSO-d₆, 400 MHz): δ (ppm) 8.67 (s, 2H), 8.59 (d, J=2.0 Hz, 1H), 8.55 (s, 1H), 8.39 (d, J=4.4 Hz, 2H), 8.22 (dd, J=8.6 Hz, 2.2 Hz, 1H), 7.96-8.0 (m, 1H), 7.84 (d, J=8.4 Hz, 1H), 7.20-7.34 (m, 2H), 7.13 (s, 1H), 6.65 (t, J=4.8 Hz, 1H), 4.71-4.84 (m, 3H). 4.41-4.55 (m, 2H). 3.40-3.53 (m, 2H). 3.18-3.30 (m, 3H).

### Example 2

### (R,E)-5-(2-(6-(1H-imidazol-1-yl)pyridin-3-yl)vinyl)-2-(3-(methoxymethyl)-4-(pyrimidin-2-yl)piperazin-1-yl)pyrimidine

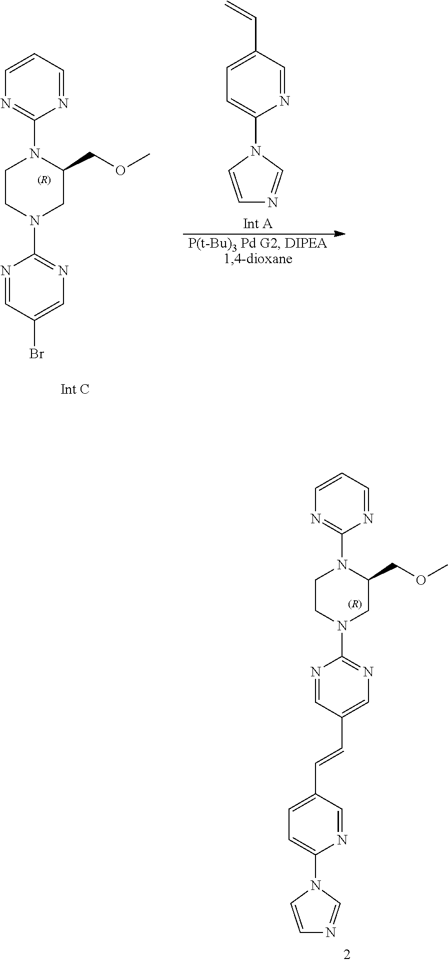

To a stirred solution of compound Int C (300 mg, 0.821 mmol) and Int A (211 mg, 1.232 mmol) in 1,4-dioxane (5 mL) was added DIPEA (0.430 mL, 2.464 mmol) at room temperature. The reaction mixture was purged with argon for 5 min, followed by Chloro[(tri-tert-butylphosphine)-2-(2-aminobiphenyl)]palladium(II) (42.1 mg, 0.082 mmol) added and again argon was purged for 5 min. Reaction mixture was stirred at 120° C. for 16 h. The reaction mixture was quenched with water (20 mL) and extracted with EtOAc (2×30 mL). Combined organic layer washed with brine solution (10 mL), dried over anhydrous Na₂SO₄, filtered, dried and concentrated under reduced pressure to get crude compound. The crude compound was purified by silica column and eluted with 5% MeOH in DCM as gradient. Pure fractions concentrated under reduced pressure to afford 2. M/Z (ESI): 456.39 [M+H]⁺.

¹H NMR (DMSO-d₆, 400 MHz): δ (ppm) 8.69 (s, 2H), 8.60 (d, J=2.4 Hz, 1H), 8.55 (s, 1H). 8.41 (d, J=4.4 Hz. 2H), 8.23 (dd, J=8.8 Hz, 2.4 Hz. 1H), 7.98 (s, 1H), 7.85 (d, J=8.8 Hz, 1H), 7.29 (d, J=16.8 Hz, 1H), 7.24 (d, J=16.4 Hz, 1H), 7.14 (s, 1H), 6.68 (t, J=4.8 Hz, 1H), 4.89-4.96 (m, 1H), 4.79 (d, J=13.2 Hz. 1H), 4.50-4.60 (m, 2H), 3.35-3.46 (m, 2H), 3.22-3.30 (m. 2H), 3.12-3.22 (m, 4H).

### Example 3

### (R,E)-5-(2-(6-(1H-imidazol-1-yl)pyridin-3-yl)vinyl)-2-(3-((2-fluoroethoxy)methyl)-4-(pyrimidin-2-yl)piperazin-1-yl)pyrimidine

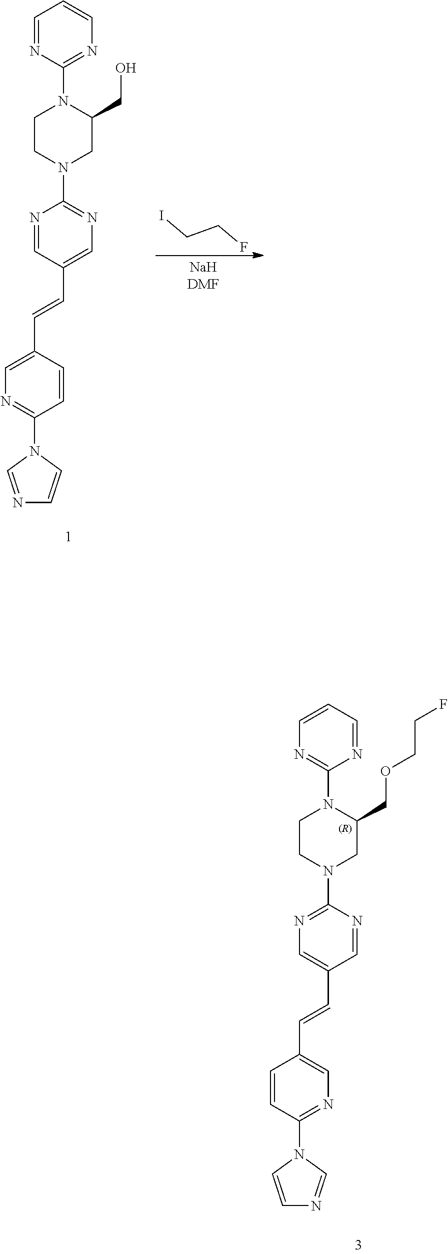

To a stirred solution of 1 (100 mg, 0.227 mmol) in DMF (1 ml) were added 1-fluoro-2-iodoethane (79 mg, 0.453 mmol) at 0° C., followed by 60% NaH (18.12 mg, 0.453 mmol). The reaction mixture was stirred at room temperature for 3 h. The reaction mixture was diluted with water (50 mL) and extracted with EtOAc (3×50 mL). Combined organic layer was dried over Na₂SO₄ and concentrated under reduced pressure to crude compound. The crude compound was purified by prep HPLC (mobile phase—10 mM Ammonium Bicarbonate in H₂O: MeCN, column—YMC Triart C18 (25×250) mm 5u Flow-7.0 ml/min, gradient method-0/35, 2/35, 11/68, 11.05/100, 13/100, 13.05/35, 16/35) to afford 3. M/Z (ELSD) (M+H): 488.35.

¹H NMR (DMSO-d₆, 400 MHz): δ (ppm) 8.68 (s, 2H), 8.60 (d, J=2.0 Hz, 1H), 8.55 (s, 1H). 8.41 (d, J=4.8 Hz. 2H). 8.23 (dd, J=8.6.2.4 Hz. 1H), 7.98 (t, J=1.4 Hz, 1H), 7.85 (d, J=8.5 Hz, 1H), 7.20-7.33 (m, 2H), 7.14 (s, 1H), 6.68 (t, J=4.8 Hz, 1H), 4.87-4.94 (m, 1H), 4.83 (br d, J=13.3 Hz, 1H), 4.46-4.62 (m, 3H), 4.39 (ddd, J=4.8, 3.3, 1.5 Hz, 1H), 3.65 (td, J=4.1, 2.4 Hz, 1H), 3.44-3.61 (m, 3H), 3.32-3.35 (m, 1H), 3.13-3.29 (m, 2H).

### Example 4

### (R,E)-5-(2-(6-(1H-imidazol-1-yl)pyridin-3-yl)vinyl)-2-(3-((fluoromethoxy)methyl)-4-(pyrimidin-2-yl)piperazin-1-yl)pyrimidine

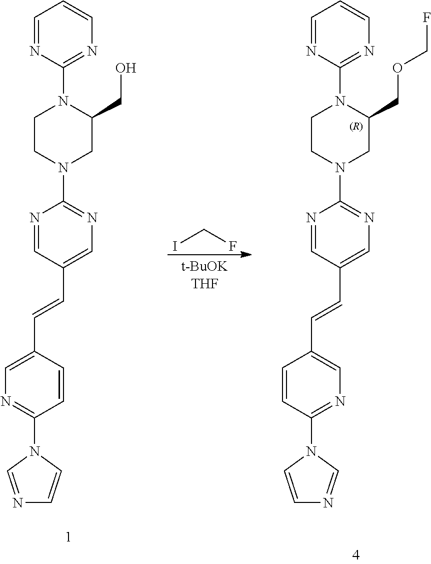

To a stirred solution of 1 (100 mg, 0.227 mmol) in THF (2 mL) were added fluoroiodomethane (72.4 mg, 0.453 mmol) and potassium tert-butoxide (50.8 mg, 0.453 mmol) at 0° C. Resulting reaction mixture was stirred for 4 h at room temperature. Reaction mixture was quenched with ice water, extracted with EtOAc (50 mL×2). Combined organic layer washed with brine solution, dried over anhydrous sodium sulphate, filtered and evaporated under reduced pressure to get crude compound. The crude compound was purified by prep HPLC (Method: MOBILE PHASE—10 mM Ammonium Bicarbonate in H₂O: MeCN, column—X-Select C18 (19×250) mm 5u Flow-16 ml/min, gradient method-0/45,2/45,12/80,12.05/99,15/99,15.05/45,18/45) to afford 4. M/Z (ELSD) (M+H): 473.44.

¹H NMR (DMSO-d₆, 400 MHz): δ (ppm) 8.69 (s, 2H), 8.60 (d, J=2.0 Hz, 1H), 8.55 (s, 1H). 8.42 (d, J=4.5 Hz, 2H), 8.23 (dd, J=8.8, 2.3 Hz, 1H), 7.98 (t, J=1.3 Hz, 1H), 7.80-7.90 (m, 1H), 7.27 (d, J=3.3 Hz, 2H), 7.14 (s, 1H), 6.70 (t, J=4.6 Hz, 1H), 5.30-5.39 (m, 1H), 5.16-5.26 (m, 1H), 4.95-5.02 (m, 1H), 4.77-4.84 (m, 1H), 4.48-4.61 (m, 2H), 3.78-3.85 (m, 1H), 3.68-3.75 (m, 1H), 3.36 (br d, J=4.3 Hz, 1H), 3.14-3.29 (m, 2H).

### Example 5

### (R,E)-5-(2-(6-(4H-1,2,4-triazol-4-yl)pyridin-3-yl)vinyl)-2-(2-(methoxymethyl)-4-(pyridin-2-yl)piperazin-1-yl)pyrimidine

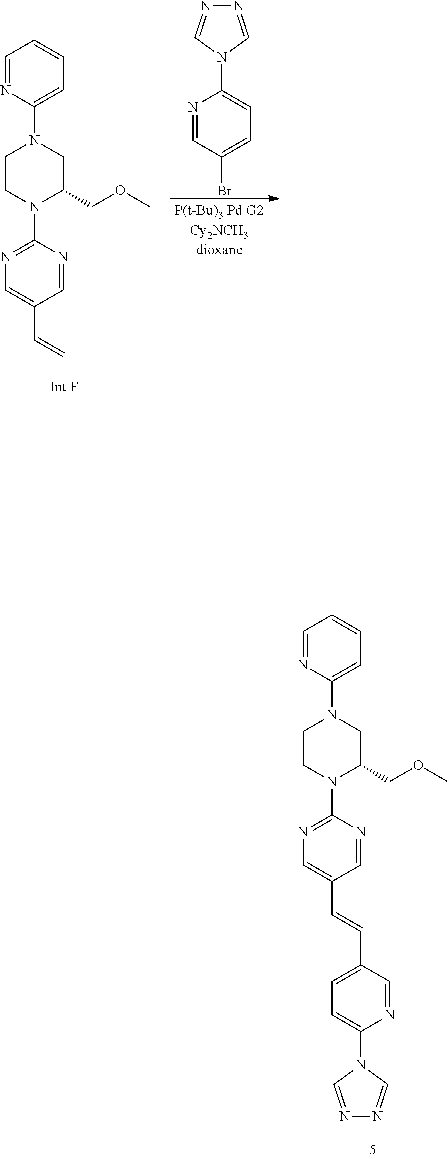 [SMILES: C=Cc1cnc(N2CCN(c3ccccn3)C[C@@H]2COC)nc1 ; Brc1ccc(-n2cnnc2)nc1 ; COC[C@H]1CN(c2ccccn2)CCN1c1ncc(/C=C/c2ccc(-n3cnnc3)nc2)cn1]

A nitrogen purged solution of Int F (10.0 mg, 0.032 mmol). 5-bromo-2-(4H-1,2,4-triazol-4-yl)pyridine (14.6 mg, 0.065 mmol), N,N-dicyclohexylmethylamine (19.0 mg, 0.097 mmol), and dichlorobis(triphenylphosphine)palladium(II) (1.66 mg, 0.003 mmol) in dioxane (0.5 ml) was heated at 120° C. for 18 h. The mixture was diluted with DMF (0.8 mL), filtered, and purified on a Gilson HPLC (10-100% ACN/H₂O w/0.1% NH₄OH Gemini-NX column) to afford compound 5. M/Z (ESI): 456.21 [M+H]⁺.

¹H NMR (DMSO-d₆, 500 MHz): δ (ppm) 9.31-9.26 (m, 2H), 8.78-8.58 (m, 3H), 8.31-8.24 (m, 1H), 8.14-8.08 (m, 1H), 7.88 (t, J=9.0 Hz, 1H), 7.63-7.46 (m, 1H), 7.39-7.18 (m, 2H), 6.82 (t, J=7.7 Hz, 1H), 6.71-6.55 (m, 1H), 4.97-4.89 (m, 1H), 4.73-4.45 (m, 1H), 4.39-4.30 (m, 1H), 4.27-4.17 (m, 1H), 3.47 (s, 3H), 3.35-3.26 (m, 1H), 3.25-3.18 (m, 2H), 3.17-3.09 (m, 1H), 3.01-2.91 (m, 1H).

### Example 6

### (R,E)-5-(2-(6-(1H-imidazol-1-yl)pyridin-3-yl)vinyl)-2-(2-((2-methoxyethoxy)methyl)-4-(pyridin-2-yl)piperazin-1-yl)pyrimidine

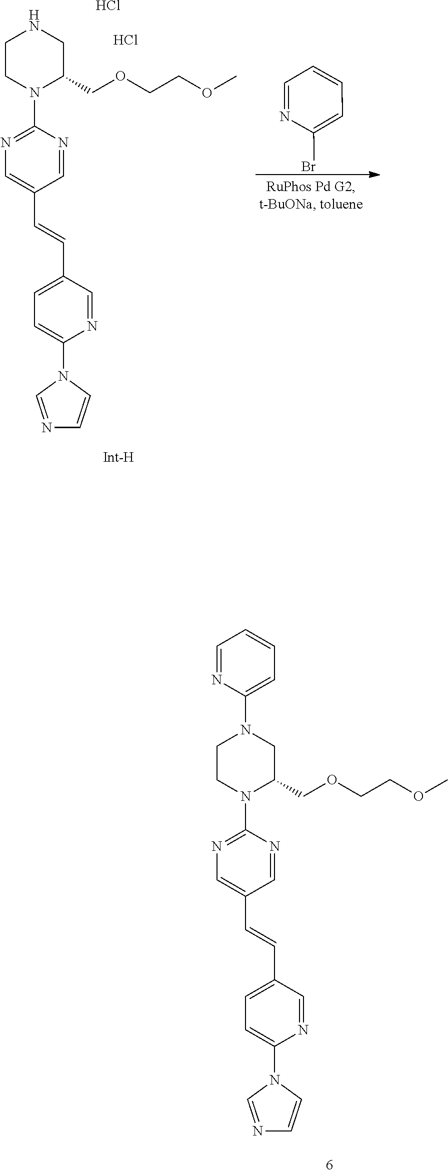 [SMILES: Cl ; COCCOC[C@H]1CNCCN1c1ncc(/C=C/c2ccc(-n3ccnc3)nc2)cn1 ; Cl ; Brc1ccccn1 ; COCCOC[C@H]1CN(c2ccccn2)CCN1c1ncc(/C=C/c2ccc(-n3ccnc3)nc2)cn1]

A stirred solution of Int H (211 mg, 0.427 mmol), 2-bromopyridine (52.9 μl, 0.555 mmol), and sodium tert-butoxide (205 mg, 2.134 mmol) in toluene (4268 μl) was purged with nitrogen for 10 minutes. Chloro(2-dicyclohexylphosphino-2′,6′-diisopropoxy-1,1′-biphenyl)[2-(2′-amino-1,1′-biphenyl)]palladium(II) (RuPhos Pd G2) (33.1 mg, 0.043 mmol) was added and the mixture was heated at 110° C. under stirring for 20 h. The reaction mixture was filtered through a Celite pad and washed with EtOAc (3×20 ml). The filtrate was concentrated and purified by flash column chromatography (40 g Isco Gold column), eluting with Hexanes:EtOAc 100:0 to 50:50, to afford compound compound 6. M/Z (ESI): 499.4 [M+H]⁺.

¹H NMR (CDCl₃, 500 MHz): δ (ppm) 8.54-8.52 (m, 1H), 8.53 (s, 2H), 8.35 (s, 1H), 8.19 (d, J=3.6 Hz, 1H), 7.94 (dd, J=8.5 Hz, 2.2 Hz, 1H), 7.65 (s, 1H), 7.50 (t. J=7.8 Hz. 1H), 7.35 (d, J=8.5 Hz, 1H), 7.23 (d, J=11.0 Hz, 1H), 6.95 (s, 2H), 6.76 (d, J=8.5 Hz, 1H), 6.69-6.0 (m, 1H), 5.08-5.02 (m, 1H), 4.71-4.52 (m, 1H), 4.50-4.28 (m, 2H), 3.78-3.45 (m, 6H), 3.45-3.29 (m, 1H), 3.36 (s, 3H), 3.25 (dd, J=13.1 Hz, 3.7 Hz, 1H), 3.16-2.96 (m, 1H).

### Examples 7 and 8

### (R,E)-(2-(4-(5-(2-(6-(1H-imidazol-1-yl)pyridin-3-yl)vinyl)pyrimidin-2-yl)-2-(methoxymethyl)piperazin-1-yl)pyrimidin-5-yl)methanol and (R,E)-5-(2-(6-(1H-imidazol-1-yl)pyridin-3-yl)vinyl)-2-(4-(5-(fluoromethyl)pyrimidin-2-yl)-3-(methoxymethyl)piperazin-1-yl)pyrimidine

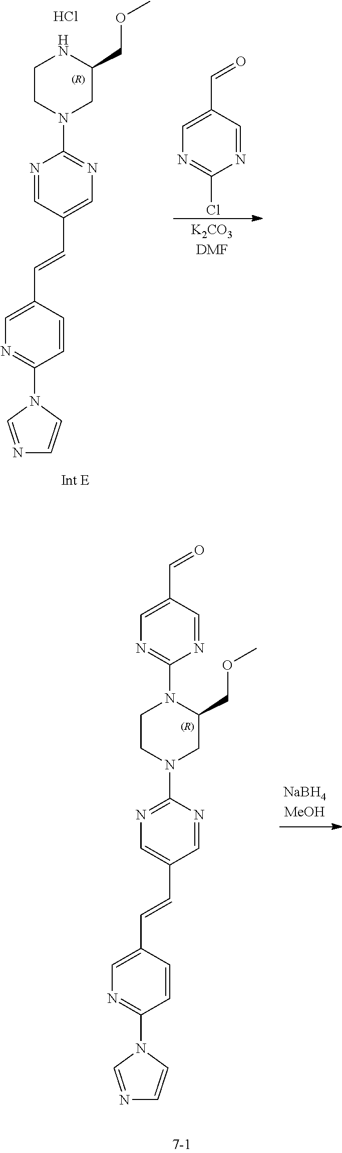

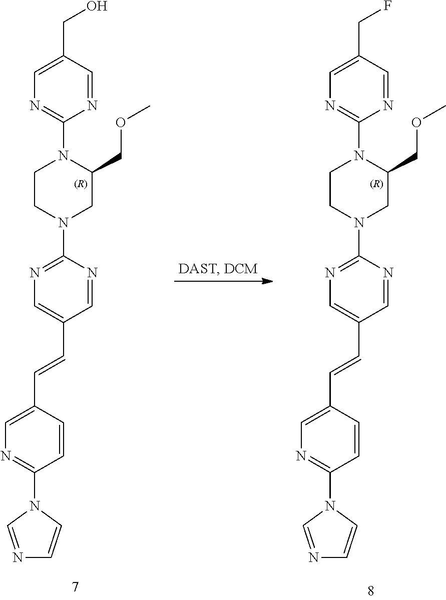

### Synthesis of 7-1: (R,E)-2-(4-(5-(2-(6-(1H-imidazol-1-yl)pyridin-3-yl)vinyl)pyrimidin-2-yl)-2-(methoxymethyl)piperazin-1-yl)pyrimidine-5-carbaldehyde

To a solution of Int E (2 g, 4.83 mmol) in DMF (20 mL) were added K₂CO₃ (4.01 g, 29.0 mmol), 2-chloropyrimidine-5-carbaldehyde (0.827 g, 5.80 mmol) at room temperature and stirred at 80° C. for 1 h under argon atmosphere. The reaction mixture was diluted with water (10 mL) and stirred for 10 min, obtained solid precipitate was filtered and dried to get crude compound. Crude compound was purified by biotage (48 g cartridge, 230-400 silica), eluted with 3% MeOH/DCM as gradient. Pure fractions concentrated under vacuum to afford compound 7-1. M/Z (ESI): 484.49 [M+H]⁺.

### Synthesis of Example 7: (R,E)-(2-(4-(5-(2-(6-(1H-imidazol-1-yl)pyridin-3-yl)vinyl)pyrimidin-2-yl)-2-(methoxymethyl)piperazin-1-yl)pyrimidin-5-yl)methanol

To a stirred solution of compound 7-1 (750 mg, 1.551 mmol) in MeOH (10 mL) was added NaBH₄ (117 mg, 3.10 mmol) at 0° C. and stirred for 30 min at same temperature. The reaction mixture was quenched with water and extracted with EtOAc (3×50 mL). Combined organic layer washed with brine solution (50 mL), dried over sodium sulphate, filtered and concentrated under reduced pressure and triturated with ACN (3×10 mL), acetone (10 mL) and diethyl ether (10 mL), and washed with pentane (10 mL) to afford 7. M/Z (ESI): 486.37 [M+H]⁺.

¹H NMR (400 MHz, DMSO-d₆): δ (ppm) 8.68 (s, 2H), 8.59 (d, J=2.0 Hz, 1H), 8.55 (s, 1H). 8.35 (s, 2H), 8.22 (dd, J=2.4 Hz, 8.8 Hz, 1H), 7.97 (s, 1H), 7.84 (d, J=8.8 Hz, 1H), 7.21-7.32 (m, 2H), 7.13 (s, 1H), 5.08 (t, J=5.6 Hz, 1H), 4.89-4.98 (m, 1H), 4.79 (d, J=13.6 Hz, 1H), 4.50-4.61 (m, 2H), 4.34 (d, J=5.6 Hz, 2H), 3.36-3.46 (m, 2H), 3.10-3.30 (m, 6H).

### Synthesis of Example 8: (R,E)-5-(2-(6-(1H-imidazol-1-yl)pyridin-3-yl)vinyl)-2-(4-(5-(fluoromethyl)pyrimidin-2-yl)-3-(methoxymethyl)piperazin-1-yl)pyrimidine

To a stirred solution of 7 (500 mg, 1.030 mmol) in DCM (10 mL) under argon atmosphere was added DAST (0.816 mL, 6.18 mmol) at 0° C. The reaction mixture was stirred at 0° C. for 30 min. The reaction mixture was quenched with water (10 mL), neutralized with saturated NaHCO₃ solution (pH was adjusted to neutral), and extracted with DCM (3×50 mL). The combined organic layer was washed with brine (30 mL), dried over sodium sulphate, filtered and concentrated under reduced pressure to afford crude product (500 mg). The crude compound (150 mg) was purified by Prep HPLC purification, and the rest amount was used in further steps Prep HPLC conditions: Mobile Phase—10 mM Ammonium Bicarbonate in H₂O: MeCN Column—YMC ODS C18 (20×100), 3 μm Flow—18.0 mL/min T/% B—0/55,10.50/81.2,10.55/100,12.50/100,12.55/55,15.50/55). Pure fractions were collected under freezing and directly lyophilized to afford 8. M/Z (SFC-MS): 488.32 [M+H]⁺.

¹H NMR (DMSO-d₆, 400 MHz): δ (ppm) 8.68 (s, 2H), 8.60 (d, J=2.0 Hz, 1H), 8.55 (s, 1H). 8.52 (d, J=2.0 Hz. 2H), 8.21-8.25 (m, 1H), 7.97 (s, 1H), 7.84 (d, J=8.4 Hz, 1H), 7.29 (d, J=16.8 Hz, 1H), 7.24 (d, J=16.4 Hz, 1H), 7.13 (s, 1H), 5.29 (d, J=48.8 Hz, 2H), 4.55-5.01 (m, 1H), 4.79 (d, J=13.2 Hz, 1H), 4.57 (d, J=10.4 Hz, 2H), 3.36-3.48 (m, 2H), 3.12-3.28 (m, 6H).

### Example 9

### (R,E)-5-(2-(6-(1H-imidazol-1-yl)pyridin-3-yl)vinyl)-2-(2-(methoxymethyl)-4-(5-(methoxymethyl)pyrimidin-2-yl)piperazin-1-yl)pyrimidine

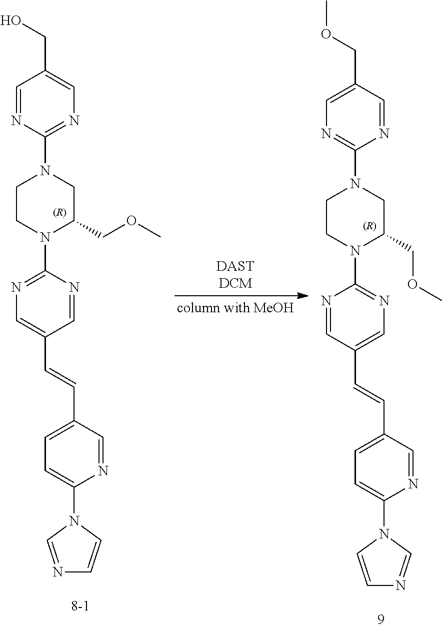

### Synthesis of Example 9: (R,E)-5-(2-(6-(1H-imidazol-1-yl)pyridin-3-yl)vinyl)-2-(2-(methoxymethyl)-4-(5-(methoxymethyl)pyrimidin-2-yl)piperazin-1-yl)pyrimidine

To a stirred solution of 8-1 [synthesized analogously to Example 7](300 mg, 0.618 mmol) in DCM (4 mL) was added DAST (0.163 ml, 1.236 mmol) at 0° C. drop wise. The reaction mixture was stirred at 25° C. for 2 h under nitrogen atmosphere. The reaction mixture was quenched with sodium bicarbonate solution (10 mL), extracted with 10% MeOH in DCM (3×25 mL). Combined organic layer was washed with brine solution (2×40 mL), dried over anhydrous Na₂SO₄ and filtered, and concentrated under reduced pressure to obtain the intermediate benzyl fluoride as a yellow solid. The above crude (280 mg) compound was purified by biotage using 80 g cartridge silica (230-400 mesh) column and eluted with 5% MeOH in DCM as gradient. Pure fractions concentrated under reduced pressure, again re-purified by Prep HPLC (Method: mobile phase—10 mM Ammonium Bicarbonate in H₂O: MeCN, column—X-Bridge C18 (19×250), Flow-12.0 ml/min, gradient method: 0/40, 2/40, 12/65, 14/65, 14.1/95, 17/95, 17.01/40, 20/40) to afford 9. M/Z (LCMS) (M+H): 500.41.

¹H NMR (chloroform-d, 400 MHz): δ (ppm) 8.69 (s, 2H), 8.55-8.60 (m, 2H), 8.37 (s, 2H), 8.23 (dd, J=2, 8.4 Hz, 1H), 7.97 (s, 1H), 7.85 (d, J=8.8 Hz, 1H), 7.22-7.31 (m, 2H), 7.13 (s, 1H), 4.97-4.98 (m, 1H), 4.76 (d, J=13.2 Hz, 1H), 4.54-4.59 (m, 2H), 4.25 (s, 1H), 3.36-3.45 (m, 2H), 3.15-3.26 (m, 8H), 3.08-3.14 (m 1H).

### Example 10

### (R)-(4-(5-((6-(1H-imidazol-1-yl)pyridin-3-yl)ethynyl)pyrimidin-2-yl)-1-(pyrimidin-2-yl)piperazin-2-yl)methanol

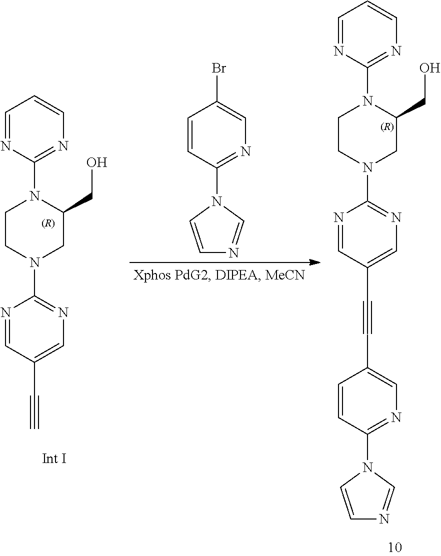

To a solution of compound Int I (1.1 g, 3.71 mmol) in ACN (30 mL) were added compound 5-bromo-2-(1H-imidazol-1-yl)pyridine (0.749 g, 3.34 mmol), DIPEA (1.945 mL, 11.14 mmol) and degassed with argon for 5 min, then chloro(2-dicyclohexylphosphino-2′,4′,6′-triisopropyl-1,1′-biphenyl)[2-(2′-amino-1,1′-biphenyl)]palladium(II) (0.292 g, 0.371 mmol) added and stirred at 80° C. for 1 h in a sealed tube. The reaction mixture was evaporated under reduced pressure, resulting precipitated solution was filtered and dried and submitted for Prep HPLC purification (method: MOBILE PHASE—10 mM Ammonium Bicarbonate in H₂O: MeCN column—X-Bridge C18 (19×250) Flow-15.0 ml/min GRADIENT METHOD—0/40,9.50/55,9.55/98,11.55/98.11.60/40,15.60/40) and lyophilized to afford 10. M/Z (LCMS) (M+H): 440.33.

¹H NMR (DMSO-d₆, 400 MHz): δ (ppm) 8.60-8.74 (m, 3H), 8.59 (s, 1H), 8.39 (d, J=4.8 Hz, 2H), 8.16 (dd, J=8.5, 2.3 Hz, 1H), 8.00 (t, J=1.3 Hz, 1H), 7.91 (d, J=8.5 Hz, 1H), 7.15 (s, 1H), 6.60-6.73 (m, 1H), 4.68-4.87 (m, 3H), 4.38-4.54 (m, 2H), 3.42-3.53 (m, 2H), 3.32-3.41 (m, 3H).

### Example 11

### (R)-5-((6-(1H-imidazol-1-yl)pyridin-3-yl)ethynyl)-2-(3-(methoxymethyl)-4-(pyrimidin-2-yl)piperazin-1-yl)pyrimidine

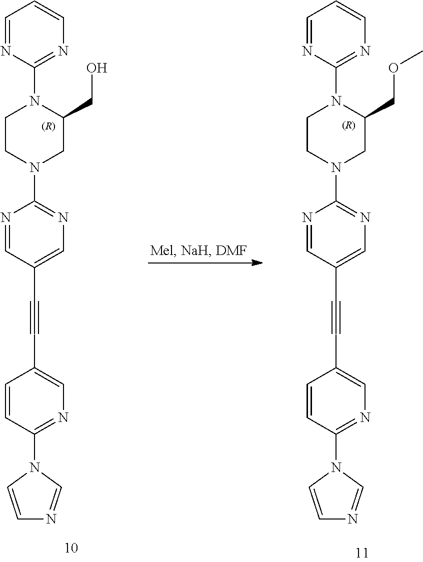

To a solution of 10 (70 mg, 0.088 mmol) in DMF (0.7 mL) were added methyl iodide (7.05 μl, 0.113 mmol) and NaH (7 mg, 0.175 mmol) at 0° C. Reaction mixture was stirred at room temperature for 1 h. The reaction mixture was quenched with cold water (10 mL), extracted with EtOAc (3×10 mL). Combined organic layer was washed with brine solution (10 mL), dried over Na₂SO₄ and concentrated under vacuum to get crude compound. Crude compound was purified by Prep HPLC (method: MOBILE PHASE—10 mM Ammonium Bicarbonate in H₂O: MeCN column—Kromosil C18(10×250) mm 5u Flow—8 ml/min gradient method-0/50, 10/85, 10.10/98, 12/98, 12.10/50, 15/50) and lyophilized to afford 11. M/Z (LCMS) (M+H): 454.36.

¹H NMR (DMSO-d₆, 400 MHz): δ (ppm) 8.66 (dd, J=2.3, 0.8 Hz, 1H), 8.64 (s, 2H), 8.59 (s, 1H), 8.41 (d, J=4.8 Hz, 2H), 8.16 (dd, J=8.5, 2.3 Hz, 1H), 8.00 (t, J=1.4 Hz, 1H), 7.88-7.93 (m, 1H), 7.16 (s, 1H), 6.68 (t, J=4.8 Hz, 1H), 4.87-4.98 (m, 1H), 4.72-4.82 (m, 1H), 4.48-4.60 (m, 2H), 3.33-3.41 (m, 3H), 3.14-3.27 (m, 5H).

### Example 12

### (R)-5-((6-(1H-imidazol-1-yl)pyridin-3-yl)ethynyl)-2-(4-(6-fluoropyrimidin-4-yl)-3-(methoxymethyl)piperazin-1-yl)pyrimidine

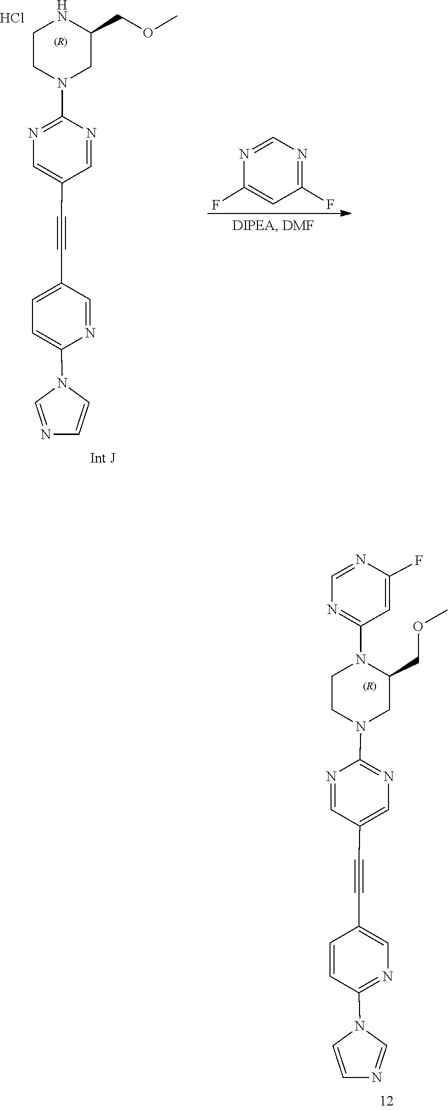

To a stirred solution of compound Int J (100 mg, 0.266 mmol) in DMF (1 mL) were added N,N-diisopropylethylamine (172 mg, 1.332 mmol) at room temperature, followed by 4,6-difluoropyrimidine (30.9 mg, 0.266 mmol) at room temperature. Reaction mixture was stirred for 1 h at 50° C. Reaction mixture was quenched with ice water, extracted with EtOAc (10 mL×2). Combined organic layer washed with brine solution, dried over anhydrous sodium sulphate, filtered and evaporated under reduced pressure to get crude compound. Crude compound was purified by prep HPLC (method: mobile phase—10 mM Ammonium Bicarbonate in H₂O: MeCN, column—X-Select C18 (19×250) Flow-18.0 ml/min, Gradient method: 0/50, 4/63, 9/63, 9.05/98, 11/98, 11.05/50, 15/50) and lyophilized to afford 12. M/Z (LCMS) (M+H): 472.43.

¹H NMR (DMSO-d₆, 400 MHz): δ (ppm) 8.64-8.65 (m, 3H), 8.58 (s, 1H), 8.35 (d, J=2.8 Hz, 1H), 8.16 (dd, J=2.4, 8.4 Hz, 1H), 8.00(s, 1H), 7.89 (d, J=8.4 Hz, 1H), 7.155 (s, 1H), 6.58 (s, 1H). 4.70-4.78 (m, 2H), 4.50-4.52 (m, 1H), 4.30 (br s, 1H), 3.37-3.44 (m, 3H), 3.24-3.29 (m, 2H), 3.21 (s, 3H).

The compounds contained in Table 1 were synthesized by analogous methods from synthetic sequences above as indicated in the last column in Table 1. Commercially available reagents were substituted where necessary to produce the examples below.

TABLE 1

| | | | Observed | | Example |
|---|---|---|---|---|---|
| Ex | | | Mass | Exact | Method |
| No. | Structure | Chemical Name | (M + H) | Mass | Used |
| 13 | 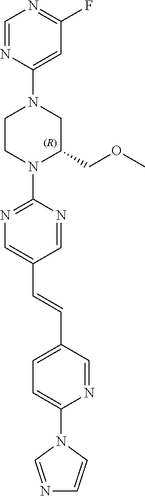 | (R,E)-5-(2-(6-(1H-imidazol-1- yl)pyridin-3-yl)vinyl)-2-(4-(6- fluoropyrimidin-4-yl)-2- (methoxymethyl)piperazin-1- yl)pyrimidine | 474.29 | 473.21 | 2 |
| 14 | 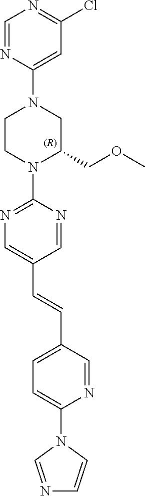 | (R,E)-5-(2-(6-(1H-imidazol-1- yl)pyridin-3-yl)vinyl)-2-(4-(6- chloropyrimidin-4-yl)-2- (methoxymethyl)piperazin-1- yl)pyrimidine | 489.97 | 489.18 | 2 |
| 15 | 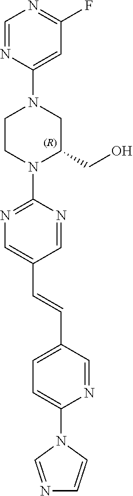 | (R,E)-(1-(5-(2-(6-(1H-imidazol-1- yl)pyridin-3-yl)vinyl)pyrimidin-2- yl)-4-(6-fluoropyrimidin-4- yl)piperazin-2-yl)methanol | 460.30 | 459.19 | 2 |
| 16 | 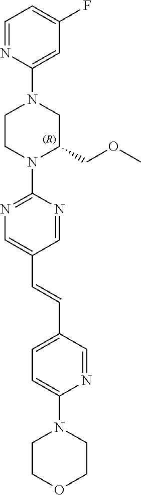 | (R,E)-4-(5-(2-(2-(4-(4-fluoropyridin- 2-yl)-2-(methoxymethyl)piperazin-1- yl)pyrimidin-5-yl)vinyl)pyridin-2- yl)morpholine | 492.55 | 491.24 | 2 |
| 17 | 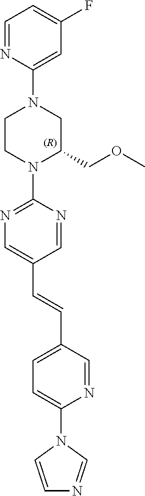 | (R,E)-5-(2-(6-(1H-imidazol-1- yl)pyridin-3-yl)vinyl)-2-(4-(4- fluoropyridin-2-yl)-2- (methoxymethyl)piperazin-1- yl)pyrimidine | 472.53 | 472.21 | 2 |
| 18 | 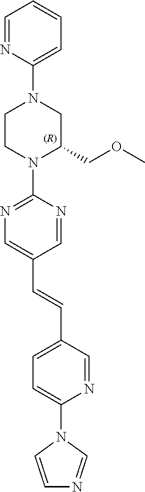 | (R,E)-5-(2-(6-(1H-imidazol-1- yl)pyridin-3-yl)vinyl)-2-(2- (methoxymethyl)-4-(pyridin-2- yl)piperazin-1-yl)pyrimidine | 455.24 | 454.22 | 2 |
| 19 | 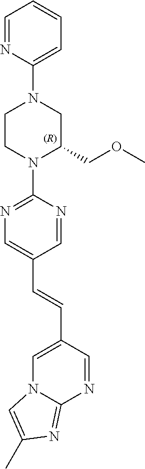 | (R,E)-6-(2-(2-(2-(methoxymethyl)-4- (pyridin-2-yl)piperazin-1- yl)pyrimidin-5-yl)vinyl)-2- methylimidazo[1,2-a]pyrimidine | 443.22 | 442.22 | 5 |
| 20 | 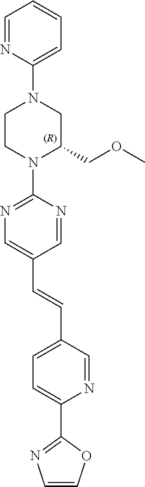 | (R,E)-2-(5-(2-(2-(2- (methoxymethyl)-4-(pyridin-2- yl)piperazin-1-yl)pyrimidin-5- yl)vinyl)pyridin-2-yl)oxazole | 456.21 | 455.21 | 5 |
| 21 | 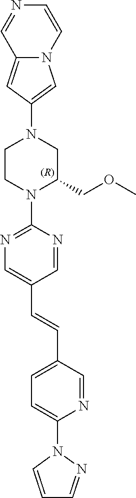 | (R,E)-7-(4-(5-(2-(6-(1H-pyrazol-1- yl)pyridin-3-yl)vinyl)pyrimidin-2- yl)-3-(methoxymethyl)piperazin-1- yl)pyrrolo[1,2-a]pyrazine | 494.22 | 493.23 | 2 |
| 22 | 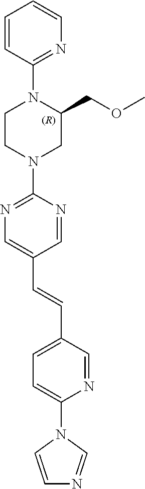 | (R,E)-5-(2-(6-(1H-imidazol-1- yl)pyridin-3-yl)vinyl)-2-(3- (methoxymethyl)-4-(pyridin-2- yl)piperazin-1-yl)pyrimidine | 455.28 | 454.22 | 2 |
| 23 | 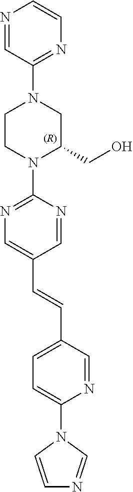 | (R,E)-(1-(5-(2-(6-(1H-imidazol-1- yl)pyridin-3-yl)vinyl)pyrimidin-2- yl)-4-(pyrazin-2-yl)piperazin-2- yl)methanol | 442.27 | 441.20 | 2 |
| 24 | 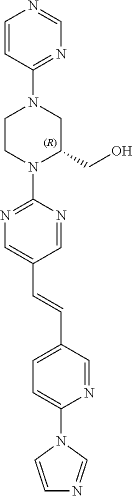 | (R,E)-(1-(5-(2-(6-(1H-imidazol-1- yl)pyridin-3-yl)vinyl)pyrimidin-2- yl)-4-(pyrimidin-4-yl)piperazin-2- yl)methanol | 442.33 | 441.20 | 2 |
| 25 | 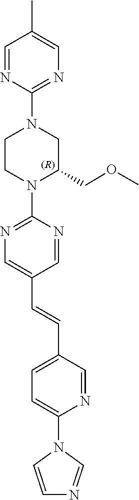 | (R,E)-5-(2-(6-(1H-imidazol-1- yl)pyridin-3-yl)vinyl)-2-(2- (methoxymethyl)-4-(5- methylpyrimidin-2-yl)piperazin-1- yl)pyrimidine | 470.33 | 469.23 | 2 |
| 26 | 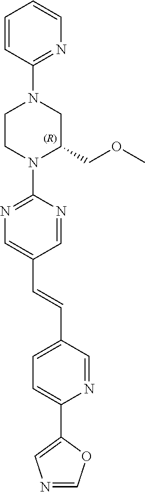 | (R,E)-5-(5-(2-(2-(2- (methoxymethyl)-4-(pyridin-2- yl)piperazin-1-yl)pyrimidin-5- yl)vinyl)pyridin-2-yl)oxazole | 456.30 | 455.21 | 2 |
| 27 | 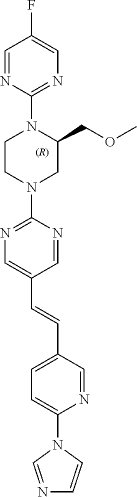 | (R,E)-5-(2-(6-(1H-imidazol-1- yl)pyridin-3-yl)vinyl)-2-(4-(5- fluoropyrimidin-2-yl)-3- (methoxymethyl)piperazin-1- yl)pyrimidine | 474.44 | 473.21 | 2 |
| 28 | 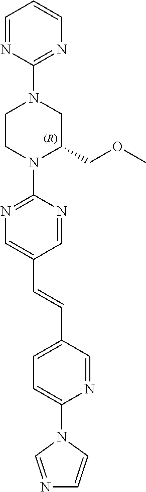 | (R,E)-5-(2-(6-(1H-imidazol-1- yl)pyridin-3-yl)vinyl)-2-(2- (methoxymethyl)-4-(pyrimidin-2- yl)piperazin-1-yl)pyrimidine | 456.38 | 455.22 | 2 |
| 29 | 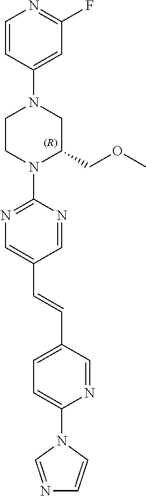 | (R,E)-5-(2-(6-(1H-imidazol-1- yl)pyridin-3-yl)vinyl)-2-(4-(2- fluoropyridin-4-yl)-2- (methoxymethyl)piperazin-1- yl)pyrimidine | 473.35 | 472.21 | 12 |
| 30 | 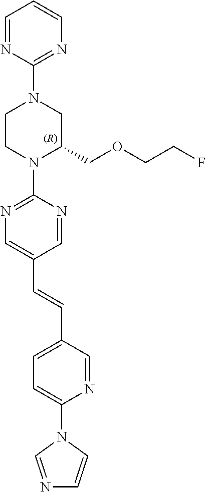 | (R,E)-5-(2-(6-(1H-imidazol-1- yl)pyridin-3-yl)vinyl)-2-(2-((2- fluoroethoxy)methyl)-4-(pyrimidin- 2-yl)piperazin-1-yl)pyrimidine | 488.38 | 487.22 | 2 |
| 31 | 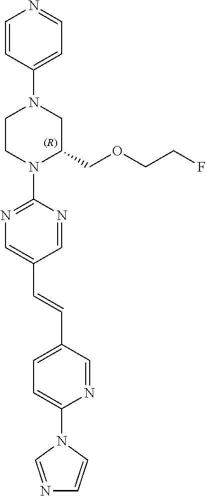 | (R,E)-5-(2-(6-(1H-imidazol-1- yl)pyridin-3-yl)vinyl)-2-(2-((2- fluoroethoxy)methyl)-4-(pyridin-4- yl)piperazin-1-yl)pyrimidine | 487.44 | 486.23 | 2 |
| 32 | 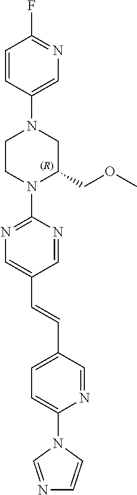 | (R,E)-5-(2-(6-(1H-imidazol-1- yl)pyridin-3-yl)vinyl)-2-(4-(6- fluoropyridin-3-yl)-2- (methoxymethyl)piperazin-1- yl)pyrimidine | 473.33 | 472.21 | 2 |
| 33 | 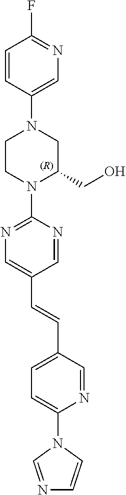 | (R,E)-(1-(5-(2-(6-(1H-imidazol-1- yl)pyridin-3-yl)vinyl)pyrimidin-2- yl)-4-(6-fluoropyridin-3- yl)piperazin-2-yl)methanol | 459.36 | 458.20 | 2 |
| 34 | 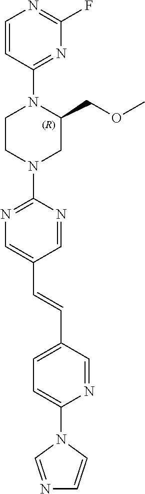 | (R,E)-4-(4-(5-(2-(6-(1H-imidazol-1- yl)pyridin-3-yl)vinyl)pyrimidin-2- yl)-2-(methoxymethyl)piperazin-1- yl)-2-fluoropyrimidine | 474.34 | 473.21 | 2 |
| 35 | 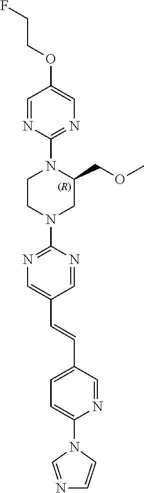 | (R,E)-5-(2-(6-(1H-imidazol-1- yl)pyridin-3-yl)vinyl)-2-(4-(5-(2- fluoroethoxy)pyrimidin-2-yl)-3- (methoxymethyl)piperazin-1- yl)pyrimidine | 518.43 | 517.23 | 2 |

### Example 36

### (R,E)-5-(5-(2-(2-(3-((2-fluoroethoxy)methyl)-4-(pyrimidin-2-yl)piperazin-1-yl)pyrimidin-5-yl)vinyl)pyridin-2-yl)oxazole

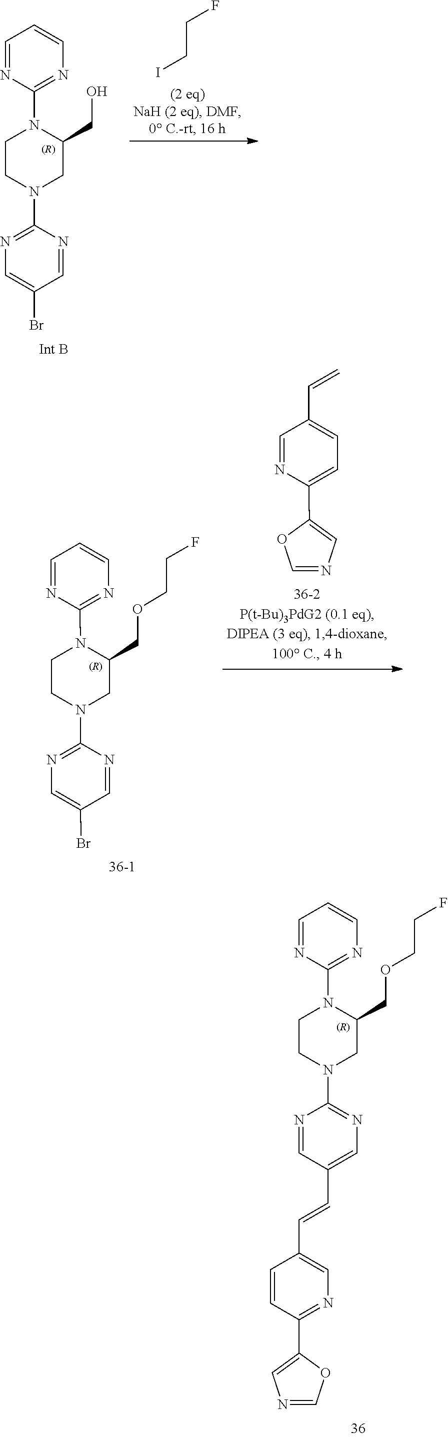

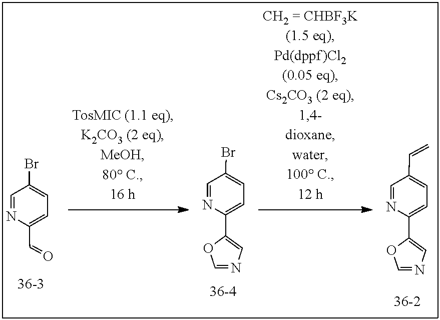

### Synthesis of 36-1: (R)-5-bromo-2-(3-((2-fluoroethoxy)methyl)-4-(pyrimidin-2-yl)piperazin-1-yl)pyrimidine

To a stirred solution of Int B (250 mg, 0.712 mmol) in DMF (3 mL) was added 1-fluoro-2-iodoethane (0.115 mL, 1.424 mmol) and NaH (56.9 mg, 1.424 mmol) at 0° C. The reaction mixture was stirred under nitrogen atmosphere at 25° C. for 16 h. Reaction mixture was quenched with water (40 mL) and extracted with EtOAc (2×30 mL). Combined organic layer was washed with brine (2×30 mL), dried over anhydrous Na₂SO₄, filtered and concentrated under reduced pressure. The crude compound was purified by biotage over 80 g silica (230-400 mesh) cartridge and compound eluted with a gradient of 30% EtOAc in Pet ether. Pure fractions concentrated under reduced pressure to afford 36-1. M/Z (ESI): 397.32 [M+H]⁻.

### Synthesis of 36-4: 5-(5-bromopyridin-2-yl)oxazole

To a stirred solution of tosylmethyl isocyanide (2 g, 10.24 mmol) in MeOH (35 mL) was added Potassium carbonate (4.25 g, 30.7 mmol at room temperature. Then to the reaction mixture was added 5-bromopicolinaldehyde (2.096 g, 11.27 mmol) at room temperature. The reaction mixture was stirred at 80° C. for 16 h. Reaction mixture was quenched with water (100 mL) and extracted with EtOAc (2×100 mL). Combined organic layer was washed with brine (100 mL), dried over anhydrous sodium sulphate, filtered and concentrated under reduced pressure.

The crude compound was purified by Biotage flash column chromatography over (100-200 mesh) silica gel and compound eluted with a gradient of 50% EtOAc in Pet ether. Pure fractions concentrated under reduced pressure to afford 36-4. M/Z (ESI): 225.10 [M+2H]⁺.

### Synthesis of 36-2: 5-(5-vinylpyridin-2-yl)oxazole

To an argon degassed stirred solution of 36-4 (1 g, 4.44 mmol) in 1,4-dioxane (10 mL), Water (5 mL) was added Potassium phosphate tribasic (2.83 g, 13.33 mmol) at room temperature. Then to the reaction mixture was added 1,1′-bis(diphenylphosphino)ferrocene-palladium(II)dichloride dichloromethane complex (0.363 g. 0.444 mmol) and potassium vinyltrifluoroborate (1.190 g, 8.89 mmol) at room temperature. The reaction mixture was stirred at 100° C. for 12 h. Reaction mixture was filtered on Buchner funnel through celite bed, washed with EtOAc (100 mL) and concentrated under reduced pressure. The crude compound was purified by Biotage flash column chromatography over (100-200 mesh) silica gel and compound eluted with a gradient of 50% EtOAc in Pet ether. Pure fractions concentrated under reduced pressure to afford 36-2. M/Z (ESI): 173.02 [M+H]⁺.

### Synthesis of 36: (R,E)-5-(5-(2-(2-(3-((2-fluoroethoxy methyl)-4-(pyrimidin-2-yl)piperazin-1-yl)pyrimidin-5-yl)vinyl)pyridin-2-yl)oxazole

To a stirred solution of 36-1 (120 mg, 0.302 mmol) in 1,4-dioxane (2 mL) was added DIPEA (0.158 mL, 0.906 mmol) and 36-2 (62.4 mg, 0.362 mmol) at room temperature. The reaction mixture was degassed with argon gas for 20 min. Then to the reaction mixture was added Chloro[(tri-tert-butylphosphine)-2-(2-aminobiphenyl)]palladium(II) (15.48 mg, 0.030 mmol) at room temperature. The reaction mixture was stirred under nitrogen atmosphere at 100° C. for 4 h in a sealed tube. Reaction mixture was quenched with water (20 mL) and extracted with EtOAc (2×20 mL). Combined organic layer was washed with brine (20 mL), dried over anhydrous Na₂SO₄, filtered and concentrated under reduced pressure. The crude compound was purified by biotage over 40 g silica (230-400 mesh) cartridge and compound eluted with a gradient of 3% MeOH in DCM. Pure fractions concentrated under reduced pressure to afford 36. M/Z (ESI): 489.36 [M+H]⁺.

¹H NMR (DMSO-d₆, 400 MHz): δ (ppm) 8.76 (d, J=1.6 Hz, 1H), 8.69 (s, 2H), 8.54 (s, 1H), 8.41 (d, J=4.4 Hz, 2H), 8.11 (dd, J=8.4 Hz. 2.0 Hz, 1H), 7.74-7.82 (m, 2H). 7.28 (q, J=14.8 Hz, 2H), 6.68 (t, J=4.8 Hz, 1H), 4.88-4.93 (m, 1H), 4.82 (d, J=13.6 Hz, 1H), 4.49-4.60 (m, 3H), 4.37-4.41 (m, 1H), 3.62-3.68 (m, 1H), 3.45-3.60 (m, 3H), 3.13-3.30 (m, 3H).

### Example 37

### (R,E)-5-(2-(6-(1-(2-fluoroethyl)-1H-pyrazol-4-yl)pyridin-3-yl)vinyl)-2-(3-(methoxymethyl)-4-(pyrimidin-2-yl)piperazin-1-yl)pyrimidine

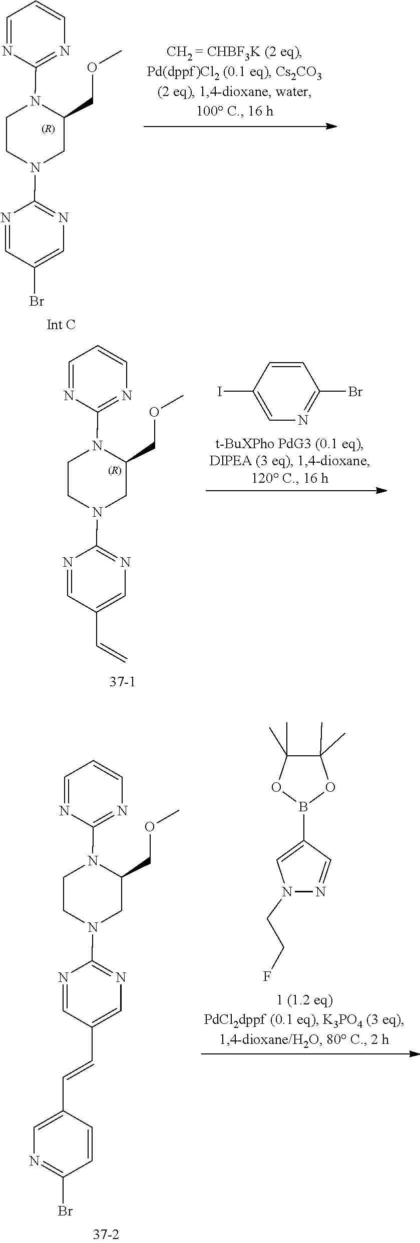

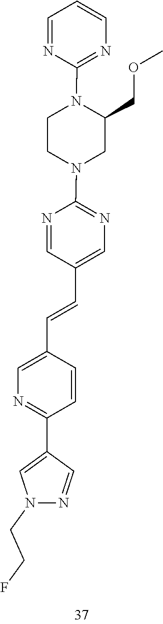 [SMILES: COC[C@H]1CN(c2ncc(/C=C/c3ccc(-c4cnn(CCF)c4)nc3)cn2)CCN1c1ncccn1]

### Synthesis of 37-1: (R)-2-(3-(methoxymethyl)-4-(pyrimidin-2-yl)piperazin-1-yl)-5-vinylpyrimidine

To a stirred solution of Int C (10 g, 27.4 mmol) in 1,4-dioxane (100 mL), water (20 mL) was added Cs₂CO₃ (17.84 g, 54.8 mmol) and potassium vinyltrifluoroborate (7.33 g, 54.8 mmol) at room temperature and degassed with argon gas for 15 min. Then PdCl₂(dppf)-CH₂Cl₂Adduct (2.236 g, 2.74 mmol) was added to the reaction mixture at room temperature. The reaction mixture was stirred under nitrogen atmosphere at 100° C. for 16 h in a sealed tube. Reaction mixture was quenched with water (200 mL) and extracted with EtOAc (2×200 mL). Combined organic layer was washed with brine (200 mL), dried over anhydrous Na₂SO₄, filtered and concentrated under reduced pressure. The crude compound was purified by silica column and compound eluted with a gradient of 30% EtOAc in pet ether. Pure fractions were concentrated under reduced pressure to afford 37-1. M/Z (ESI): 313.38 [M+H]⁻.

### Synthesis of 37-2: (R,E)-5-(2-(6-bromopyridin-3-yl)vinyl)-2-(3-(methoxymethyl)-4-(pyrimidin-2-yl)piperazin-1-yl)pyrimidine

To a stirred solution of 37-1 (4 g, 12.81 mmol) in 1,4-dioxane (50 mL) was added DIPEA (6.71 mL, 38.4 mmol) and 2-bromo-5-iodopyridine (18.18 g, 64.0 mmol) at room temperature and degassed with argon for 15 min. Then tBu X-phos Pd G3 (1.017 g, 1.281 mmol) was added at room temperature. The reaction mixture was stirred under nitrogen atmosphere at 120° C. for 16 h in a sealed tube. Reaction mixture was quenched with water (50 mL) and extracted with EtOAc (2×200 mL). Combined organic layer was washed with brine (50 mL), dried over anhydrous Na₂SO₄, filtered and concentrated under reduced pressure. The crude compound was purified by silica column and compound eluted with a gradient of 30% EtOAc in pet ether. Pure fractions were concentrated under reduced pressure and obtained compound was further re-purified by Prep-HPLC purification. Pure fractions were concentrated under reduced pressure and lyophilized to afford 37-2.M/Z (ESI): 516.30 [M+H]⁺.

¹H NMR (DMSO-d₆, 400 MHz): δ (ppm) 8.67 (s, 2H), 8.48 (d, J=2.4 Hz, 1H), 8.40 (d, J=4.8 Hz, 2H), 7.83 (d, J=8.0 Hz, 1H), 7.71 (dd, J=8.4 Hz, 2.4 Hz, 1H), 7.28 (d, J=16.4 Hz, 1H), 7.14 (d, J=16.4 Hz, 1H), 6.67 (t, J=4.8 Hz, 1H), 4.89-4.96 (m, 1H), 4.78 (d, J=13.2 Hz, 1H), 4.49-4.60 (m, 2H), 3.35-3.45 (m, 2H), 3.26 (d, J=4.0 Hz, 1H), 3.11-3.25 (m, 5H).

### Synthesis of 37: (R,E)-5-(2-(6-(I-(2-fluoroethyl)-1H-pyrazol-4-yl)pyridin-3-yl)vinyl)-2-(3-(methoxymethyl)-4-(pyrimidin-2-yl)piperazin-1-yl)pyrimidine

To a stirred solution of 37-2 (100 mg, 0.214 mmol) and 1-(2-fluoroethyl)-4-(4,4,5,5-tetramethyl-1,3,2-dioxaborolan-2-yl)-1H-pyrazole (61.5 mg, 0.256 mmol) in 1.4 dioxane (1 mL) and H₂O (0.2 mL) was added tripotassium phosphate (136 mg, 0.641 mmol) at room temperature. The reaction mixture was degassed and purged with argon gas for 5 min. Then to this reaction mixture PdCl₂(dppf)-CH₂Cl₂adduct (17.44 mg, 0.021 mmol) was added at room temperature. The reaction mixture was stirred at 80° C. for 2 h under nitrogen atmosphere in a sealed tube. The reaction mixture was diluted with water (10 mL), extracted with EtOAc (2×20 mL). Combined organic layer was washed with brine (10 mL), dried over anhydrous Na₂SO₄ filtered, and concentrated under reduced pressure. Crude compound was purified by prep-HPLC purification. Pure fractious were combined and lyophilized to afford 37. M/Z (ESI): 502.41 [M+H]⁻.

### Prep-HPLC Purification Conditions:

Mobile Phase—10 mM Ammonium Bicarbonate IN H₂O: MeCN Column—X-Bridge C18 (19×150), 5μ Flow-15.0 ml/min Gradient Method 0/40,7/50,9/56,9.1/100,11.9/100,12/40,15/40.

¹H NMR (400 MHz, DMSO-d₆) δ=8.68 (s, 2H), 8.62 (d, J=1.6 Hz, 1H), 8.41 (d, J=4.6 Hz, 2H), 8.38-8.36 (m, 1H), 8.08 (s, 1H), 7.99 (dd, J=8.4 Hz, 2.3 Hz, 1H), 7.68 (d, J=8.1 Hz, 1H), 7.20 (s, 2H), 6.67 (t, J=4.7 Hz, 1H), 4.95-4.85 (m, 2H), 4.82-4.73 (m, 2H), 4.58-4.50 (m, 3H), 4.45 (t. J=4.7 Hz. 1H), 3.45-3.35 (m, 2H), 3.30-3.23 (m, 2H). 3.23-3.20 (m, 3H), 3.19-3.15 (m, 1H).

### Example 38

### (R,E)-5-(5-(2-(2-(3-((2-(2-fluoroethoxy)ethoxy)methyl)-4-(1,3,5-triazin-2-yl)piperazin-1-yl)pyrimidin-5-yl)vinyl)pyridin-2-yl)oxazole

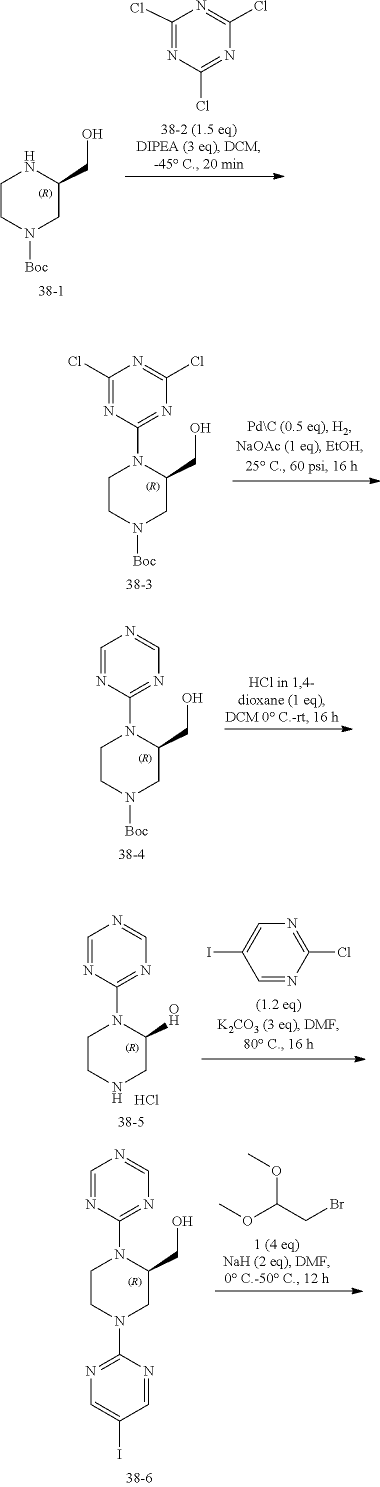

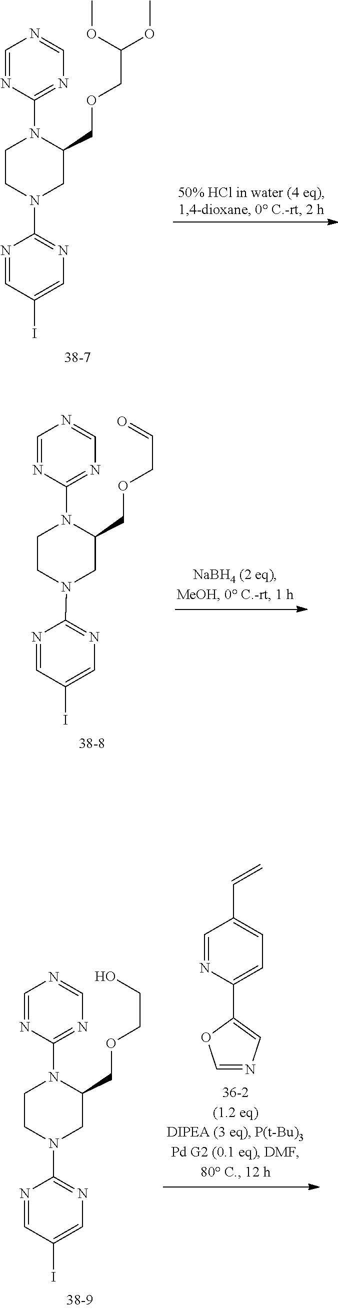 [SMILES: COC(COC[C@H]1CN(c2ncc(I)cn2)CCN1c1ncncn1)OC ; O=CCOC[C@H]1CN(c2ncc(I)cn2)CCN1c1ncncn1 ; OCCOC[C@H]1CN(c2ncc(I)cn2)CCN1c1ncncn1 ; C=Cc1ccc(-c2cnco2)nc1]

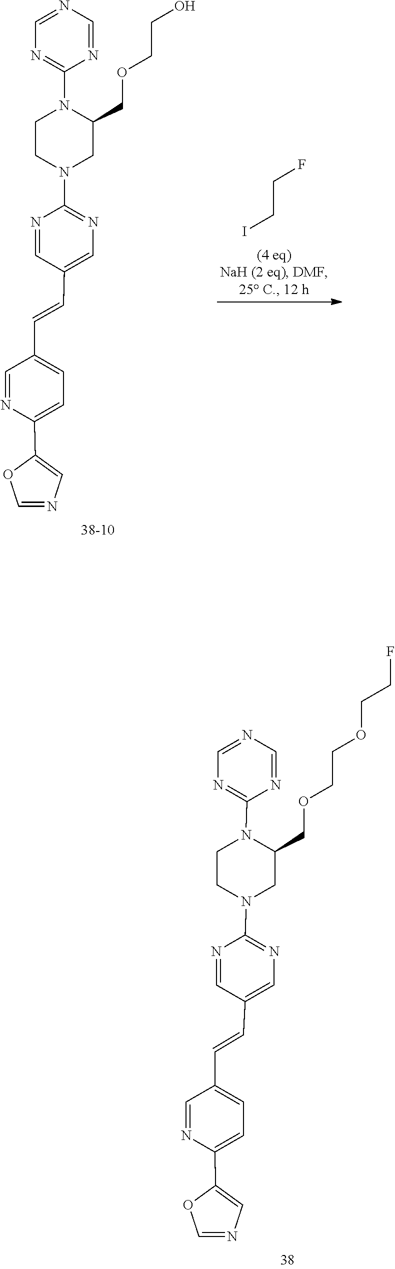

### Synthesis of 38-3: tert-butyl (R)-4-(4,6-dichloro-1,3,5-triazin-2-yl)-3-(hydroxymethyl)piperazine-1-carboxylate

To a stirred solution of tert-butyl (R)-3-(hydroxymethyl)piperazine-1-carboxylate (38-1) (30 g, 139 mmol) in DCM (800 mL) was added DIPEA (72.7 mL, 416 mmol) at −45° C. and stirring continued for 15 min. Then to this reaction mixture 2,4,6-trichloro-1,3,5-triazine (38-2) (38.4 g, 208 mmol) was added at −45° C. The reaction mixture was stirred −45° C. for 20 min.

The reaction mixture was diluted with water (500 mL) and with EtOAc (3×200 mL). The combined organic layer was washed with water (2×200 mL), dried over Na₂SO₄ and concentrated under reduced pressure to afford 38-3. M/Z (ESI): 364.19 [M+H]⁻.

### Synthesis of 38-4: tert-butyl (R)-3-(hydroxymethyl)-4-(1,3,5-triazin-2-yl)piperazine-1-carboxylate

To a stirred solution of 38-3 (20 g, 54.9 mmol) in EtOH (500 mL) was added sodium acetate (4.50 g, 54.9 mmol) and 10% Pd-C(5.84 g, 27.5 mmol) at room temperature. The reaction mixture was degassed and purged with nitrogen gas for 3 times. The reaction mixture was stirred at 60 psi H₂ gas pressure at 25° C. for 16 h. Reaction mixture was filtered through ciliate bed, washed with EtOAc (500 mL) and concentrated under reduced pressure to afford 38-4. M/Z (ESI): 296.34 [M+H]⁺.

### Synthesis of 38-5: (R)-(1-(1,3,5-triazin-2-yl)piperazin-2-yl)methanol hydrochloride

To a stirred solution of 38-4 (15 g, 50.8 mmol) in DCM (200 mL) was added HCl in 1,4-dioxane (6.03 mL, 50.8 mmol) at 0° C. The reaction mixture was stirred at 25° C. for 16 h. The reaction mixture was concentration under reduced pressure. Crude compound was triturated with diethyl ether (2×50 mL) and dried under reduced pressure to afford 38-5. M/Z (ESI): 196.13 [M+H]⁺.

### Synthesis of 38-6: (R)-(4-(5-iodopyrimidin-2-yl)-1-(1,3,5-triazin-2-yl)piperazin-2-yl)methanol

To a stirred solution of 38-5 (9.5 g, 41.0 mmol) in DMF (200 mL) were added 2-chloro-5-iodopyrimidine (11.83 g, 49.2 mmol) and K₂CO₃ (17.00 g, 123 mmol) at room temperature. The reaction mixture was stirred 80° C. for 16 h. Reaction mixture was quenched with water (200 mL) and with EtOAc (3×150 mL). Combined organic layer was dried over Na₂SO₄ and concentrated under reduced pressure. Crude compound was purified by 300 g (100-200 mesh) silica gel cartridge and compound eluted with 50% EtOAc in pet ether. Pure fractions were combined and concentrated under reduced pressure to afford 38-6. M/Z (ESI): 400.22 [M+H]⁺.

### Synthesis of 38-7: (R)-2-(2-((2,2-dimethoxyethoxy)methyl)-4-(5-iodopyrimidin-2-yl)piperazin-1-yl)-1,3,5-triazine

To a stirred solution of 38-6 (2 g, 5.01 mmol) in DMF (30 mL) was added 60% NaH in oil (0.401 g, 10.02 mmol) at 0° C. The reaction mixture was stirred at 0° C. for 30 min. Then to this reaction mixture 2-bromo-1,1-dimethoxyethane (3.39 g, 20.04 mmol) was added at 0° C. The reaction mixer was stirred at 50° C. for 12 h. Reaction mixture was diluted with water (100 mL) and extracted with EtOAc (3×100 mL). Combined organic layer was dried over Na₂SO₄ and concentrated under reduced pressure. Crude compound was purified by 100 g (100-200 mesh) silica gel cartridge and compound eluted with 25% EtOAc in pet ether. Pure fractions were combined and concentrated under reduced pressure to afford 38-7. M/Z (ESI): 488.29 [M+H]⁻.

### Synthesis of 38-8: (R)-2-((4-(5-iodopyrimidin-2-yl)-1-(1,3,5-triazin-2-yl)piperazin-2-yl)methoxy)acetaldehyde

To a stirred solution of 38-7 (1.9 g, 3.90 mmol) in 1,4 dioxane (30 mL) was added 50% in water HCl (1.281 mL, 15.60 mmol) at 0° C. The reaction mixture was stirred 25° C. for 2 h. Reaction mixture was diluted with aqueous saturated NaHCO₃ solution (100 mL) and extracted with EtOAc (3×100 mL). Combined organic layer was dried over Na₂SO₄ and concentrated under reduced pressure to afford 38-8. M/Z (ESI): 442.22 [M+H]⁺.

### Synthesis of 38-9: (R)-2-((4-(5-iodopyrimidin-2-yl)-1-(1,3,5-triazin-2-yl)piperazin-2-yl)methoxy)ethan-1-ol

To a stirred solution of 38-8 (1.2 g, 2.72 mmol) in MeOH (20 mL) was added NaBH₄ (0.206 g, 5.44 mmol) at 0° C. The reaction mixture was stirred at 25° C. for 1 h. Reaction mixture was concentrated under reduced pressure and diluted with water (100 mL). Aqueous layer was extracted with EtOAc (3×150 mL). Combined organic layer was dried over Na₂SO₄ and concentrated under reduced pressure to afford 38-9. M/Z (ESI): 444.29 [M+H]⁻.

### Synthesis of 38-10: (R,E)-2-((4-(5-(2-(6-(oxazol-5-yl)pyridin-3-yl)vinyl)pyrimidin-2-yl)-1-(1,3,5-triazin-2-yl)piperazin-2-yl)methoxy)ethan-1-ol

The stirred solution of 38-9 (500 mg, 1.128 mmol) in DMF (8 mL) was purged with argon gas for 10 min. Then to this reaction mixture 36-2 (233 mg, 1.354 mmol), DIPEA (0.591 mL, 3.38 mmol), and chloro[(tri-tert-butylphosphine)-2-(2-aminobiphenyl)]palladium(II) (57.8 mg, 0.113 mmol) were added at room temperature. The reaction mixture was again purged with argon for another 10 min. The reaction mixture was stirred at 80° C. for 12 h. Reaction mixture was diluted with water (300 mL) and extracted with EtOAc (3×150 mL). Combined organic layer was dried over Na₂SO₄ and concentrated under reduced pressure. Crude compound was purified by 100 g (100-200 mesh) silica gel cartridge and compound eluted with 8% MeOH in DCM. Pure fractions were combined and concentrated under reduced pressure to afford 38-10. M/z (ESI)=488.36 [M+H]f

### Synthesis of 38: (R,E)-5-(5-(2-(2-(3-((2-(2-fluoroethoxy)ethoxy)methyl)-4-(1,3,5-triazin-2-yl)piperazin-1-yl)pyrimidin-5-yl)vinyl)pyridin-2-yl)oxazole

To a stirred solution of 38-10 (350 mg, 0.718 mmol) in DMF (4 mL) was added 60% NaH in oil (57.4 mg, 1.436 mmol) at 0° C. The reaction mixture was stirred at 0° C. for 30 min. Then to this reaction mixture 1-fluoro-2-iodoethane (500 mg, 2.87 mmol) was added at 0° C. The reaction mixture was stirred at 25° C. for 12 h. Reaction mixture was diluted with water (200 mL) and extracted with EtOAc (3×180 mL). Combined organic layer was dried over Na₂SO₄ and concentrated under reduced pressure. Crude compound was purified by prep HPLC purification. Pure fractions were combined and lyophilized to afford 38. M/z (ESI)=534.31 [M+H]⁻.

### Purification: Prep HPLC Method.

Mobile Phase—10 mM Ammonium Bicarbonate in H₂O: MeCN COLUMN—Princetonsphere (21.2×250) mm, 5μ Flow-19.0 ml/min/Gradient Method—0/40,8/45,14/45,14.05/100,17/100,17.05/40,20/40.

¹H NMR (400 MHz, DMSO-d₆) δ=8.76 (d, J=1.8 Hz, 1H), 8.70 (s, 2H), 8.64 (s, 2H), 8.54 (s, 1H), 8.11 (dd, J=2.1, 8.4 Hz, 1H), 7.83-7.76 (m, 2H). 7.35-7.22 (m, 2H), 5.02-4.96 (m, 1H), 4.83-4.77 (m, 1H), 4.65-4.38 (m, 4H), 3.63-3.44 (m, 8H), 3.35-3.33 (m, 1H), 3.30 (br s, 1H), 3.21-3.14 (m, 1H).

### Example 39-a and 39-B

### 1-((2-((R)-4-(5-((E)-2-(6-(1H-imidazol-1-yl)pyridin-3-yl)vinyl)pyrimidin-2-yl)-2-(methoxymethyl)piperazin-1-yl)pyrimidin-5-yl)oxy)-3-fluoropropan-2-ol

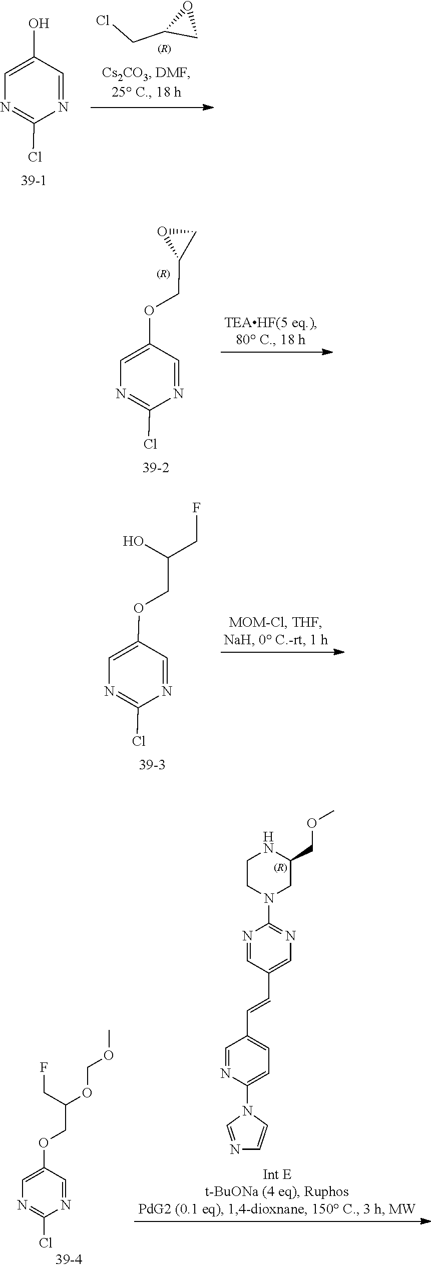

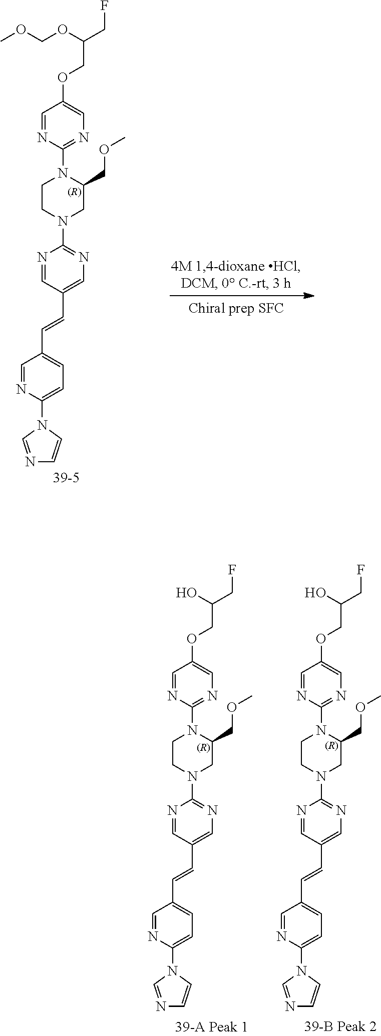

### Synthesis of 39-2: (R)-2-chloro-5-(oxiran-2-ylmethoxy)pyrimidine

To a stirred solution of 2-chloropyrimidin-5-ol (2.0 g, 15.32 mmol) in DMF (20 mL) under nitrogen atmosphere was added Cesium carbonate (7.49 g, 22.98 mmol) and (R)-2-(chloromethyl)oxirane (7.09 g, 77 mmol) at room temperature. The reaction mixture was stirred at 25° C. for 18 h. Reaction mixture was concentrated under reduced pressure and diluted with 10% MeOH: DCM (20 mL) and filtered. Reaction mixture was concentrated under reduced pressure. The crude compound was purified by biotage column chromatography over 25 g silica cartridge and compound eluted with a gradient of 10% MeOH in DCM. Pure fractions concentrated under reduced pressure to afford 39-2. M/Z (ESI): 186.96 [M+H]⁻.

### Synthesis of 39-3: 1-((2-chloropyrimidin-5-yl)oxy)-3-fluoropropan-2-ol

To a 39-2 (900 mg, 4.82 mmol) was added triethylamine trihydrofluoride (3 mL, 4.82 mmol) at room temperature. The reaction mixture was stirred at 80° C. for 18 h. Reaction mixture was cooled to room temperature. Reaction mixture was basified with sat. NaHCO₃ solution (PH~8) and extracted with EtOAc (100 mL). Combined organic layer was washed with water (100 mL) and dried over Na₂SO₄, filtered and concentrated under reduced pressure. The crude compound was purified by biotage column chromatography over 25 g silica (230-400 mesh) cartridge and compound eluted with a gradient of 40% EtOAc in Pet ether. Pure fractions concentrated under reduced pressure to afford 39-3. M/Z (ESI): 207.02 [M+H]⁻.

### Synthesis of 39-4: 2-chloro-5-(3-fluoro-2-(methoxymethoxy)propoxy)pyrimidine

To a stirred solution of 39-3 (0.3 g, 1.452 mmol) in THF (15 mL) under nitrogen atmosphere was added sodium hydride (0.116 g, 2.90 mmol) at 0° C. Then to the reaction mixture was added MOM-Cl (0.165 mL, 2.178 mmol) dropwise at 0° C. The reaction mixture was stirred at 25° C. for 1 h. Reaction mixture was quenched with ice water (20 mL) and extracted with EtOAc (20 mL). Combined organic layer was washed with water (20 mL), dried with Na₂SO₄, filtered and concentrated under reduced pressure. The crude compound was purified by biotage column chromatography over 25 g silica column (100-200 mesh) cartridge and compound eluted with a gradient of 40% EtOAc in Pet ether. Pure fractions concentrated under reduced pressure to afford 39-4. M/Z (ESI): 251.03[M+H]⁺.

### Synthesis of 39-5: 5-((E)-2-(6-(1H-imidazol-1-yl)pyridin-3-yl)vinyl)-2-((3R)-4-(5-(3-fluoro-2-(methoxymethoxy)propoxy)pyrimidin-2-yl)-3-(methoxymethyl)piperazin-1-yl)pyrimidine

To a stirred solution of (R,E)-5-(2-(6-(1H-imidazol-1-yl)pyridin-3-yl)vinyl)-2-(3-(methoxymethyl)piperazin-1-yl)pyrimidine (Int E) (140 mg, 0.371 mmol) and 39-4 (307 mg, 1.224 mmol) in 1,4-dioxane (10 mL) was added sodium tert-butoxide (143 mg, 1.484 mmol) at room temperature and degassed with nitrogen for 2 min. Then to the reaction mixture was added under nitrogen atmosphere Chloro(2-dicyclohexylphosphino-2′,6′-diisopropoxy-1,1′-biphenyl)[2-(2′-amino-1,1′-biphenyl)]palladium(II) (28.8 mg, 0.037 mmol) at room temperature. The reaction mixture was stirred at 150° C. for 3 h under microwave irradiation. Reaction mixture was quenched with water (10 mL) and extracted with EtOAc (2×10 mL). Combined organic layer was washed with water (20 mL), dried over Na₂SO₄, filtered and concentrated under reduced pressure. The crude compound was mixed and purified by biotage column chromatography over 12 g silica (100-200 mesh) cartridge and compound eluted with a gradient of 10% MeOH in DCM. Pure fractions concentrated under reduced pressure and obtained compound was washed with diethyl ether (10 mL) to afford 39-5. M/Z (ESI): 592.45 [M+H]⁺.

### Synthesis of 39-A and 39-B: 1-((2-((R)-4-(5-((E)-2-(6-(1H-imidazol-1-yl)pyridin-3-yl)vinyl)pyrimidin-2-yl)-2-(methoxymethyl)piperazin-1-yl)pyrimidin-5-yl)oxy)-3-fluoropropan-2-ol

To a stirred solution of 39-5 (40 mg, 0.068 mmol) in DCM (10 mL) under nitrogen atmosphere was added 1,4-dioxane hydrochloride (0.017 mL, 0.068 mmol) at 0° C. The reaction mixture was stirred at 25° C. for 3 h. Reaction mixture was concentrated under reduced pressure. The crude compound was purified by SFC purification. Pure fractions concentrated under reduced pressure and lyophilized separately to afford 39-A peak-1 and 39-B peak-2. M/Z (ESI): 548.37 [M+H]⁺.

¹H NMR (DMSO-d₆, 400 MHz): δ (ppm) 8.68 (s, 2H), 8.54-8.63 (m, 2H), 8.26 (s, 2H), 8.20-8.25 (m, 1H), 8.01-8.13 (m, 1H), 7.98 (s, 1H), 7.84 (d, J=8.8 Hz, 1H), 7.21-7.33 (m, 2H), 7.13 (s, 1H), 6.52 (s, 5H), 5.45 (d, J=4.8 Hz, 1H), 4.79 (d, J=11.2 Hz, 2H), 4.35-4.60 (m, 4H), 3.92-4.08 (m, 2H), 3.38-3.42 (m, 2H), 3.21-3.28 (m, 1H), 3.10-3.21 (m, 3H).

Analytical NP-HPLC Conditions:

| Column/dimensions | Chiralpak IA (250 × 4.6 × 5 μ) | |
|---|---|---|
| Mobile Phase | Methanol:DCM:DIPA(80:20:0.2%) | |
| Flow | 0.5 | mL/min |
| Temperature | Ambient | |
| Wave Length | 340 | nm |

Preparative NP-HPLC Conditions

| | |
|---|---|
| Column/Dimensions | Chiralpak IA (250 × 10 × 5 μ) |
| Mobile Phase | Methanol:DCM:DIPA(80:20:0.2%) |
| Flow | 3.0 mL/min |

### Example 40

### (R,E)-2-(3-((2-fluoroethoxy)methyl)-4-(pyrimidin-2-yl)piperazin-1-yl)-5-(2-(6-(4-methyl-1H-imidazol-1-yl)pyridin-3-yl)vinyl)pyrimidine

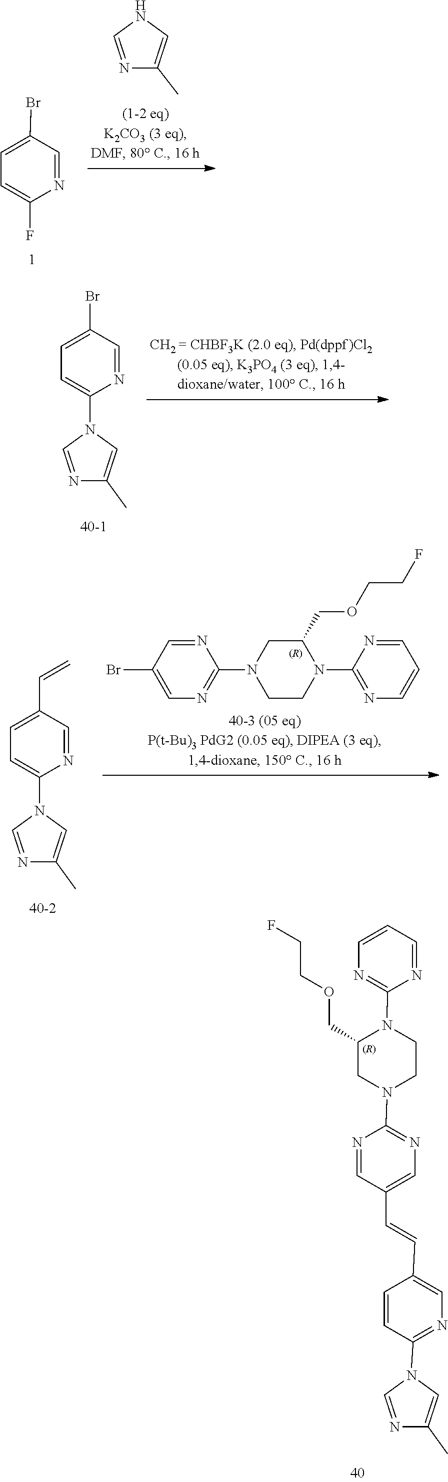

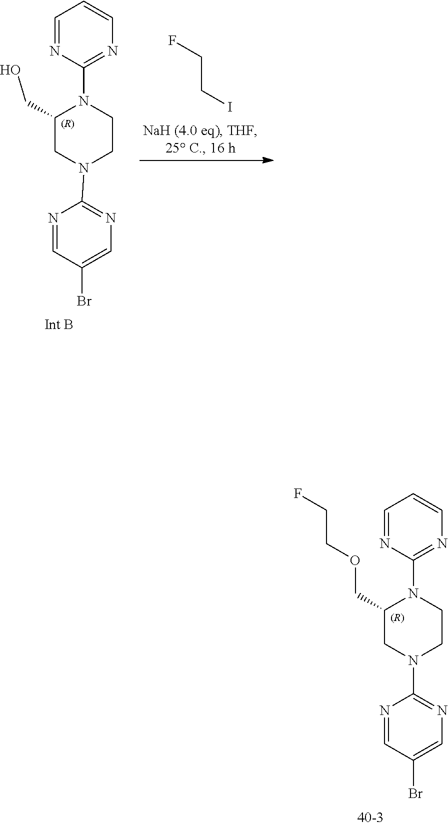

### Synthesis of 40-1: 5-bromo-2-(4-methyl-1H-imidazol-1-yl)pyridine

To a stirred solution of 5-bromo-2-fluoropyridine (1 g, 5.68 mmol) in DMF (20 mL) were added potassium carbonate (2.356 g, 17.05 mmol) and 4-methyl-TH-imidazole at room temperature. The reaction mixture was stirred at 80° C. for 16 h. Reaction mixture was quenched with ice water (50 mL), solid was filtered through Buchner funnel and dried under reduced pressure to afford 40-1. M/Z (ESI): 237.98 [M+H]⁺.

### Synthesis of 40-2: 2-(4-methyl-1H-imidazol-1-yl)-5-vinylpyridine

The stirred solution of 40-1 (1 g, 4.20 mmol) in 1,4-dioxane (10 mL) and water (10 mL) was degassed and purged with argon gas. Then to this degassed stirred solution were added potassium phosphate tribasic (2.67 g, 12.60 mmol) and potassium vinyltrifluoroborate (1.125 g, 8.40 mmol), [1,1′-bis(diphenylphosphino)ferrocene]dichloropalladium(II) (0.154 g, 0.210 mmol) at room temperature. The reaction mixture was stirred at 100° C. for 16 h. Reaction mixture was quenched with ice water (10 mL) and extracted with EtOAc (2×10 mL). The combined organic layer was washed with brine (10 mL), dried over anhydrous sodium sulphate, filtered and concentrated under reduced pressure. Crude compound was purified by (100-200 mesh) silica gel column and compound eluted with 100% EtOAc. Pure fraction was concentrated under reduced pressure to afford 40-2. M/Z (ESI): 186.02 [M+H]⁺.

### Synthesis of 40-3: (R)-5-bromo-2-(3-((2-fluoroethoxy)methyl)-4-(pyrimidin-2-yl)piperazin-1-yl)pyrimidine

To a stirred solution of int B (1 g, 2.85 mmol) in THF (10 mL) were added 1-fluoro-2-iodoethane (0.381 mL, 5.69 mmol) and sodium hydride (0.273 g, 11.39 mmol) at room temperature. The reaction mixture was stirred at 25° C. for 16 h. Reaction mixture was quenched with ice water (10 mL) and aqueous layer was extracted with EtOAc (3×20 mL). Combined organic layer was washed with brine (10 mL), dried over anhydrous sodium sulphate, filtered and concentrated under reduced pressure. Crude compound was purified by (100-200 mesh) silica gel column and compound eluted with 50% EtOAc in pet ether. Pure fractions were combined and concentrated under reduced pressure to afford 40-3. M/Z (ESI): 399.13 [(M+2)+H]⁺.

### Synthesis of 40: (R,E)-2-(3-((2-fluoroethoxy)methyl)-4-(pyrimidin-2-yl)piperazin-1-yl)-5-(2-(6-(4-methyl-1H-imidazol-1-yl)pyridin-3-yl)vinyl)pyrimidine

The stirred solution of 40-2 (200 mg, 1.080 mmol) in 1,4-dioxane (5 mL) was degassed and purged with argon gas. Then to this reaction mixture N,N-diisopropylethylamine (0.581 mL. 3.24 mmol), 40-3 (214 mg, 0.540 mmol) and chloro[(tri-tert-butylphosphine)-2-(2-aminobiphenyl)]palladium(II) (27.7 mg, 0.054 mmol) were added at room temperature. The reaction mixture was stirred at 150° C. for 16 h in microwave oven. Reaction mixture was quenched with ice water (10 mL) and aqueous layer was extracted with EtOAc (3×5 mL). Combined organic layer was washed with brine (IOmL), dried over anhydrous sodium sulphate, filtered and concentrated under reduced pressure. Crude product was purified by prep HPLC purification. Pure fractions were combined and lyophilized to afford 40. M/Z (ESI): 502.39 [M+H]⁻.

Prep HPLC conditions

| Instrument ID | ANL-MCL-5-PREP-021 |
|---|---|
| Column Name | X-SELECT C18 (19*250) mm 5 μ |
| Column No# | X-SELECT C18 (19*250) mm 5 μ |
| Mobile Phase-A | 10 mM Ammonium BiCarbonate in water |
| Mobile Phase-B | Acetonitrile |
| Gradient program | 0/50, 2/50, 11/74, 11.1/100, 13/100, 13.,/50, |
| (T/% B) | 16/50 |
| Flow Rate (mL/minute) | 19 |

¹H NMR (400 MHz, DMSO-d₆) δ=8.67 (s, 2H), 8.57-8.56 (m, 1H), 8.45-8.38 (m, 3H), 8.19 (dd, J=8.8 Hz, 2.3 Hz, 1H), 7.77 (d, J=8.6 Hz, 1H), 7.67 (s, 1H), 7.25 (d, J=2.4 Hz, 2H), 6.68 (t, J=4.7 Hz, 1H), 4.94-4.87 (m, 1H), 4.86-4.79 (m, 1H), 4.59-4.39 (m, 4H), 3.67-3.47 (m, 4H), 3.30-3.12 (m, 3H), 2.20-2.15 (m, 3H).

### Example 41

### (R,E)-5-(5-(2-(2-(3-((2-fluoroethoxy)methyl)-4-(1,3,5-triazin-2-yl)piperazin-1-yl)pyrimidin-5-yl)vinyl)pyri din-2-yl)oxazole

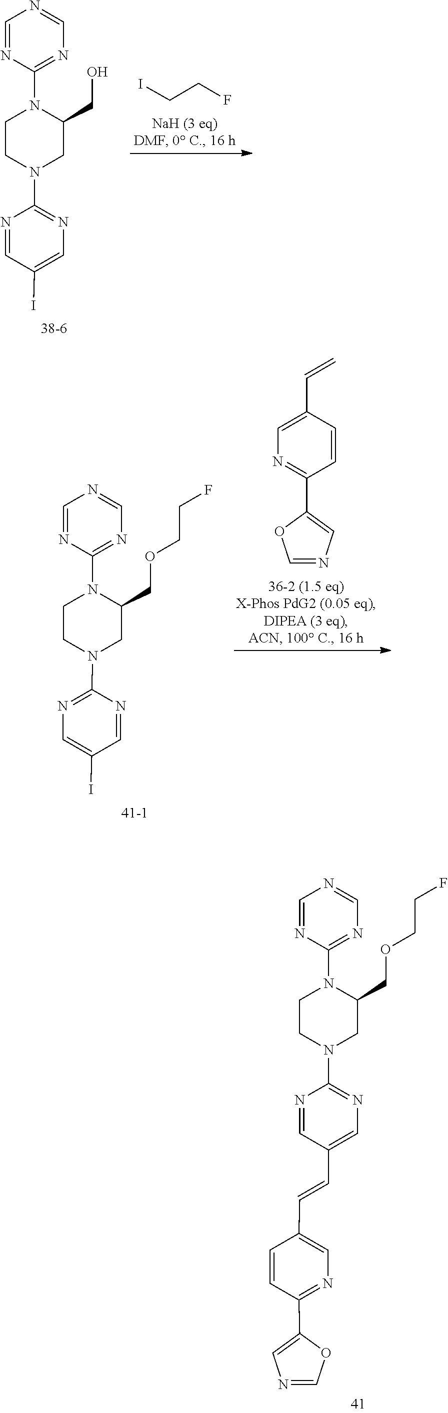

### Synthesis of 41-1: (R)-2-(2-((2-fluoroethoxy)methyl)-4-(5-iodopyrimidin-2-yl)piperazin-1-yl)-1,3,5-triazine

To a stirred solution of 38-6 (1.00 g, 2.51 mmol) in DMF (10 mL) were added 60% NaH in oil (301 mg, 7.52 mmol) and 1-fluoro-2-iodoethane (872 mg, 2 Eq, 5.01 mmol) at 0° C. The reaction mixture was stirred at 25° C. for 16 h. Reaction mixture was quenched with water (20 mL) and extracted with EtOAc (2×50 mL). Combined organic layer was washed with brine (20 mL), dried over anhydrous Na₂SO₄, filtered, and concentrated under reduced pressure. Crude compound was purified by Biotage using silica column and compound eluted with 40% EtOAc in pet ether. Pure fractions were combined and concentrated under reduced pressure to afford 41-1. M/Z (ESI): 446.19 [M+H]⁺.

### Synthesis of 41: (R,E)-5-(5-(2-(2-(3-((2-fluoroethoxy methyl)-4-(1,3,5-triazin-2-yl)piperazin-1-yl)pyrimidin-5-yl)vinyl)pyridin-2-yl)oxazole

A stirred solution of 41-1 (80 mg, 0.151 mmol) in ACN (1 mL) was purged with argon gas for 10 min. Then the stirred solution was added 36-2 (51.9 mg, 0.301 mmol), DIPEA (0.105 mL, 0.603 mmol) and chloro(2-dicyclohexylphosphino-2′,4′,6′-triisopropyl-1,1′-biphenyl)[2-(2′-amino-1,1′-biphenyl)]palladium(II) (15.81 mg, 0.02 mmol) at room temperature and again purged with argon gas for another 10 mi. The reaction mixture was stirred at 100° C. for 12 h. Reaction mixture was quenched with water (80 mL) and extracted with EtOAc (3×80 mL). Combined organic layer was dried over anhydrous Na₂SO₄, filtered and concentrated under reduced pressure. Crude compound was purified by prep-HPLC purification (conditions: Mobile Phase—10 mM Ammonium Bicarbonate IN H₂O: MeCN COLUMN—X-Bridge C18 (19×250) Flow-18.0 ml/min Gradient Method-0/30,7/42,12.44.5,12.05/100,14/100,14.05/30,17/30). Pure fractions concentrated under reduced pressure and lyophilized to afford 41. M/Z (ESI): 490.42 [M+H]⁺.

¹H NMR (DMSO-d₆, 400 MHz): δ (ppm) 8.76-8.75 (m, 1H), 8.70 (s, 2H), 8.64 (m, 2H), 8.54 (s, 1H), 8.11 (dd, J=8.4 Hz, 2.4 Hz, 1H), 7.80-7.77 (m, 2H). 7.34-7.23 (m, 2H), 4.99-4.98 (m, 1H), 4.81 (d, J=13.6 Hz, 1H), 4.62-4.56 (m, 2H), 4.50-4.36 (m, 2H), 3.66-3.64 (m, 1H), 3.58-3.56 (m, 3H), 3.34 (m, 1H), 3.27-3.26 (m, 1H), 3.20-3.17 (m, 1H).

### Example 42

### (R,E)-5-(5-(2-(2-(3-((2-fluoroethoxy)methyl)-4-(pyrimidin-4-yl)piperazin-1-yl)pyrimidin-5-yl)vinyl)pyridin-2-yl)oxazole

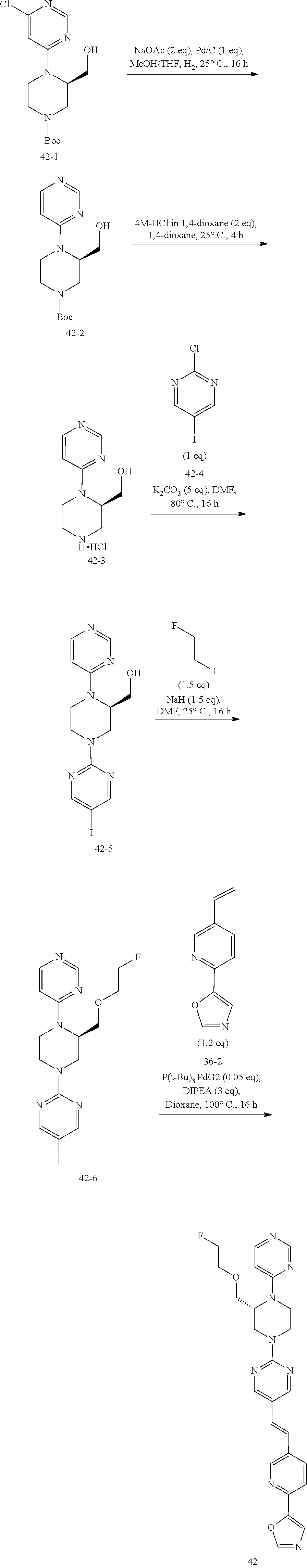

### Synthesis of 42-2: tert-butyl (R)-3-(hydroxymethyl)-4-(pyrimidin-4-yl)piperazine-1-carboxylate

To a stirred solution of 42-1 (2 g, 6.08 mmol) in MeOH (11 mL) and THF (11 mL) was added sodium acetate (0.998 g, 12.17 mmol) at room temperature. Then to this reaction mixture 10% Pd/C (0.647 g, 6.08 mmol) was added at room temperature. The reaction mixture was stirred at 25° C. for 16 h under hydrogen atmosphere. Reaction mixture was quenched with ice water (10 mL) and solid was filtered through Buchner funnel. Filtered solid was dried under vacuum. Crude compound was purified by Biotage flash column chromatography using (100-200 mesh) silica gel column and compound eluted with 10% EtOAc in pet-ether. Pure fractions were combined and concentrated under reduced pressure to afford 42-2. M/Z (ESI): 295.11 [M+H]⁻

### Synthesis of 42-3: (R)-(1-(pyrimidin-4-yl)piperazin-2-yl)methanol

To a stirred solution of 42-2 (1.5 g, 5.10 mmol) in DCM (20 mL) was added 4M HCl in 1,4-dioxane (1.209 mL, 10.19 mmol) at room temperature. The reaction mixture was stirred at 25° C. for 4 h. Reaction mixture was concentrated under reduced pressure and cold distilled with toluene. Crude compound was triturated with pentane (5 mL) and dried under reduced pressure to afford 42-3. M/Z (ESI): 195.10 [M+H]⁺

### Synthesis of 42-5: (R)-(4-(5-iodopyrimidin-2-yl)-1-(pyrimidin-4-yl)piperazin-2-yl)methanol

To a stirred solution of 42-3 (1 g, 5.15 mmol) in DMF (25 mL) were added potassium carbonate (3.56 g, 25.7 mmol) and 2-chloro-5-iodopyrimidine (42-4) (1.238 g, 5.15 mmol) at room temperature. The reaction mixture was stirred at 80° C. for 16 h. Reaction mixture was quenched with ice water (20 mL) and extracted with ethyl acetate (3×20 mL). Combined organic layer was washed with brine (10 mL), dried over anhydrous sodium sulphate, filtered and concentrated under reduced pressure. Crude compound was purified by Biotage flash chromatography using (100-200 mesh) silica gel column and compound eluted with 50% EtOAc in pet ether. Pure fractions were combined and concentrated under reduced pressure to afford 42-5. M/Z (ESI): 399.13 [M+H]⁺

### Synthesis of 42-6: (R)-2-(3-((2-fluoroethoxy)methyl)-4-(pyrimidin-4-yl)piperazin-1-yl)-5-iodopyrimidine

To a stirred solution of 42-5 (5060079-0374-002) (500 mg. 1.256 mmol) in THF (5 mL) were added 1-fluoro-2-iodoethane (0.126 mL, 1.883 mmol) and sodium hydride (90 mg, 3.77 mmol) at room temperature. The reaction mixture was stirred at 25° C. for 16 h. Reaction mixture was quenched with ice water (10 mL), and extracted with ethyl acetate (3×20 mL). Combined organic layer was washed with brine (10 mL), dried over anhydrous sodium sulphate, filtered and concentrated under reduced pressure. Crude compound was purified by Biotage using (100-200 mesh) silica gel column and compound eluted with 50% EtOAc in pet ether. Pure fractions were combined and concentrated under reduced pressure to afford 42-6. M/Z (ESI): 445.12 [M+H]⁻

### Synthesis of 42: (R,E)-5-(5-(2-(2-(3-((2-fluoroethoxy)methyl)-4-(pyrimidin-4-yl)piperazin-1-yl)pyrimidin-5-yl)vinyl)pyridin-2-yl)oxazole

The stirred solution of 42-6 (100 mg, 0.225 mmol) in 1,4-dioxane (1 mL) was degassed and purged with argon gas. Then to this reaction mixture N,N-diisopropylethylamine (0.121 mL. 0.675 mmol), 5-(5-vinylpyridin-2-yl)oxazole (36-2) (46.5 mg, 0.270 mmol), chloro[(tri-tert-butylphosphine)-2-(2-aminobiphenyl)]palladium(II) (11.53 mg, 0.023 mmol) were added at room temperature. The reaction mixture was stirred at 100° C. for 16 h. Reaction mixture was quenched with ice water (10 mL) and extracted with ethyl acetate (3×10 mL). Combined organic layer was washed with brine (5 mL), dried over anhydrous sodium sulphate, filtered and concentrated under reduced pressure. Crude product was purified by prep HPLC purification. Pure fractions were combined and lyophilized to afford 42. M/Z (ESI): 489.34 [M+H]⁺

### Prep HPLC Purification Condition;

Mobile Phase—10 mM Ammonium Bicarbonate in H₂O: MeCN
COLUMN—LUNA C18 (21.2×250) mm 5 um Flow-18 ml/min.
Gradient:_0/38,2/38,15/40,16/40.3,16.05/100,18.0/100,18.05/38,21.0/38.

¹H NMR (400 MHz, DMSO-d₆) δ=8.76 (d, J=1.8 Hz, 1H), 8.70 (s, 2H), 8.54 (s, 2H), 8.22 (d, J=6.3 Hz, 1H), 8.11 (dd, J=8.4 Hz, 2.1 Hz, 1H), 7.82-7.77 (m, 2H), 7.34-7.22 (m, 2H), 6.88-6.86 (m, 1H), 4.79-4.48 (m, 4H), 4.37 (br d, J=4.1 Hz, 2H), 3.67-3.51 (m, 4H), 3.40-3.36 (m, 1H), 3.29-3.22 (m, 2H).

### Example 43

### (R,E)-2-(2-((2-fluoroethoxy)methyl)-4-(5-(2-(2-(1-methyl-1H-pyrazol-4-yl)pyrimidin-5-yl)vinyl)pyrimidin-2-yl)piperazin-1-yl)-1,3,5-triazine

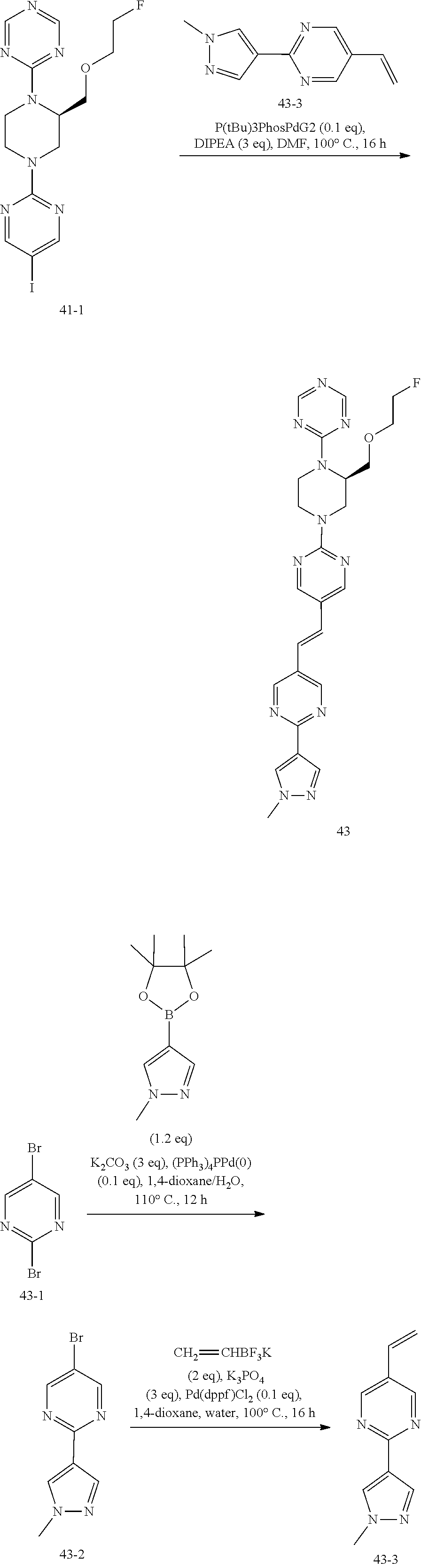

### Synthesis of 43-2: 5-bromo-2-(1-methyl-1H-pyrazol-4-yl)pyrimidine

To a stirred solution of 2,5-dibromopyrimidine (43-1) (5 g, 21.02 mmol) in 1,4-dioxane (50 mL) and H₂O (10 mL) were added 1-methyl-4-(4.4,5,5-tetramethyl-1,3,2-dioxaborolan-2-yl)-1H-pyrazole (5.25 g, 25.2 mmol), potassium carbonate (8.71 g, 63.1 mmol) at room temperature. The reaction mixture was degassed and purged with argon gas for 10 min. Then to this reaction mixture tetrakis(triphenylphosphine)palladium(0) (2.429 g, 2.102 mmol) was added at room temperature. The reaction mixture was stirred at 110° C. at 12 h. Reaction mixture was diluted with water (50 mL) and extracted with EtOAc (2×50 mL). Combined organic layer was dried over Na₂SO₄, concentrated under reduced pressure. Crude compound was purified by silica gel column chromatography and compound eluted with 20% EtOAc in pet ether. Pure fractions were combined and concentrated under reduced pressure to afford 43-2. M/z (ESI)=239.01 [M+H]⁻

### Synthesis of 43-3: 2-(1-methyl-1H-pyrazol-4-yl)-5-vinylpyrimidine

To a stirred solution of 43-2 (1.00 g, 4.18 mmol) in 1,4-dioxane (10 mL) and water (10 mL) were added trifluoro(vinyl)-4-borane, potassium salt (1.12 g, 8.37 mmol) and tripotassium phosphate (2.66 g, 12.5 mmol) at room temperature and degassed with argon at room temperature for 5 min. Then PdCl₂(dppf)-CH₂Cl₂ adduct (342 mg, 418 μmol) was added to the reaction mixture at room temperature. The reaction mixture was stirred at 100° C. for 16 h. Reaction mixture was quenched with water (20 mL) and extracted with EtOAc (2×30 mL). Combined organic layer was washed with brine (10 mL), dried over anhydrous Na₂SO₄, filtered and concentrated under reduced pressure. Crude compound was purified by Biotage using silica column and compound eluted with 30% EtOAc in pet ether. Pure fractions were combined and concentrated under reduced pressure to afford 43-3. M/Z (ESI): 187.00 [M+H]⁻.

### Synthesis of 43: (R,E)-2-(2-((2-fluoroethoxy)methyl)-4-(5-(2-(2-(1-methyl-1H-pyrazol-4-yl)pyrimidin-5-yl)vinyl)pyrimidin-2-yl)piperazin-1-yl)-1,3,5-triazine

To a stirred solution of 41-1 (100 mg, 225 mol) and 43-3 (50.2 mg, 270 μmol) in 1,4-dioxane (2 mL) was added DIPEA (117 μL, 674 μmol) at room temperature and degassed with argon at room temperature for 5 min. Then chloro(tri-t-butylphosphine)(2′-amino-1,1′-biphenyl-2-yl)palladium(II) (11.5 mg, 22.5 mol) was added to the reaction mixture at room temperature and again degassed with argon at room temperature for 5 min. The reaction mixture was stirred at 100° C. for 16 h. Reaction mixture was quenched with water (10 mL) and extracted with EtOAc (2×20 mL). Combined organic layer was washed with brine (10 mL), dried over anhydrous Na₂SO₄, filtered and concentrated under reduced pressure. Crude compound was purified by Prep HPLC purification (conditions: Mobile Phase—10 mM Ammonium Bicarbonate IN H₂O: MeCN COLUMN—X-Bridge C18 (19×250) mm, 5μ Flow-15.0 ml/min Gradient Method:-0/28,2/28,11/59.4,11.05/100,13/100,13.05/28,16/28). Pure fractions were combined, concentrated under reduced pressure and lyophilized to afford 43. M/Z (ESI): 504.39 [M+H]⁺.

¹H NMR (400 MHz, DMSO-d₆): δ (ppm) 8.91 (s, 2H), 8.68 (s, 2H), 8.64 (s, 2H), 8.38 (s, 1H), 8.03 (d, J=0.4 Hz, 1H), 7.32 (d, J=16.8 Hz, 1H), 7.16 (d, J=16.4 Hz, 1H), 4.95-5.04 (m, 1H), 4.81 (d, J=13.6 Hz, 1H), 4.55-4.67 (m, 2H), 4.33-4.52 (m, 2H), 3.91 (s, 3H), 3.63-3.69 (m, 1H), 3.58 (dd, J=6.6 Hz, 3.0 Hz, 3H). 3.33-3.36 (m, 1H), 3.25-3.30 (m, 1H), 3.14-3.23 (m, 1H).

### Example 44

### (R)-2-(2-((2-fluoroethoxy)methyl)-4-(5-((2-(1-methyl-1H-pyrazol-4-yl)pyrimidin-5-yl)ethynyl)pyrimidin-2-yl)piperazin-1-yl)-13,5-triazine

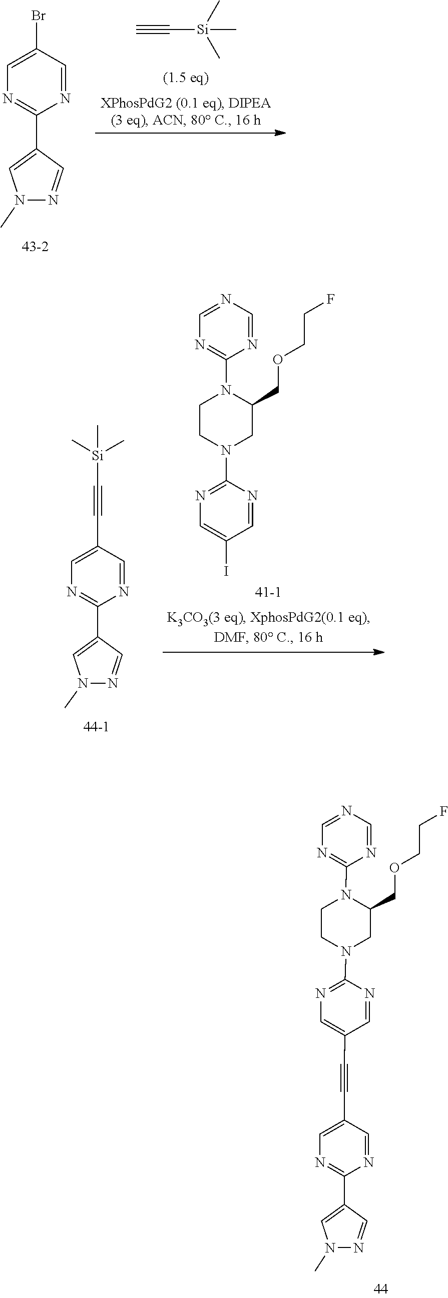

### Synthesis of 44-1: 2-(1-methyl-1H-pyrazol-4-yl)-5-((trimethylsilyl)ethynyl)pyrimidine

To a stirred solution of 43-2 (2 g, 8.37 mmol) in ACN (24 mL) was added DIPEA (4.38 mL, 25.10 mmol) at room temperature. The reaction mixture was degassed and purged with argon gas for 15 min. Then to the reaction mixture ethynyltrimethylsilane (1.786 mL, 12.55 mmol) and XPhos Pd G2 (0.658 g, 0.837 mmol) were added at room temperature. The reaction mixture was stirred at 80° C. for 16 h under nitrogen atmosphere in a sealed tube. Reaction mixture was diluted with water (10 mL), extracted with EtOAc (2×30 mL). Combined organic layer was washed with brine (10 mL), dried over anhydrous Na₂SO₄, filtered, and concentrated under reduced pressure. Crude compound was purified by biotage using 40 g (230-400 mesh) silica gel cartridge and compound eluted with 30% EtOAc in pet ether. Pure fractions were combined and concentrated under reduced pressure to afford 44-1. M/z (ESI)=257.23 [M+H]⁺

### Synthesis of 44: (R)-2-(2-((2-fluoroethoxy)methyl)-4-(5-((2-(1-methyl-1H-pyrazol-4-yl)pyrimidin-5-yl)ethynyl)pyrimidin-2-yl)piperazin-1-yl)-1,3,5-triazine

To a stirred solution of 41-1 (100 mg, 0.225 mmol) in DMF (1 mL) were added 44-1 (63.3 mg. 0.247 mmol) and potassium carbonate (93 mg, 0.674 mmol) at room temperature. The reaction mixture was degassed and purged with argon gas for 15 min. Then to this reaction mixture X-Phos Pd G2 (17.67 mg, 0.022 mmol) was added at room temperature. The reaction mixture was stirred at 80° C. for 16 h under nitrogen atmosphere in a sealed tube. Reaction mixture was diluted with water (10 mL), extracted with EtOAc (2×30 mL). Combined organic layer was washed with brine (10 mL), dried over anhydrous Na₂SO₄, filtered, and concentrated under reduced pressure. Crude compound was purified by prep HPLC purification (method: Mobile Phase—10 mM Ammonium Bicarbonate IN H₂O: MeCN COLUMN—YMC HYDROSPHER C18(20*250)mm, 5μ Flow-18.0 ml/min Gradient Method 0/45,10.5/66,10.51/100,13/100,13.1/45,16/45). Pure fractions were combined and lyophilized to afford 44. M/z (ESI)=502.35 [M+H]+

¹H NMR (400 MHz, DMSO-d₆) δ=8.89 (s, 2H). 8.63 (d, J=4.4 Hz, 4H), 8.42 (s, 1H), 8.05 (s, 1H), 4.98-4.96 (m, 1H), 4.80 (d, J=13.6 Hz, 1H), 4.59-4.55 (m, 2H), 4.44 (dt, J=4, 8.0 Hz, 2H), 3.92 (s, 3H), 3.65-3.55 (m, 4H), 3.40-3.36 (m, 1H), 3.29-3.20 (m, 2H).

### Example 45

### (R)-5-((6-(4H-1,2,4-triazol-4-yl)pyridin-3-yl)ethynyl)-2-(4-(6-fluoropyrimidin-4-yl)-3-(methoxymethyl)piperazin-1-yl)pyrimidine

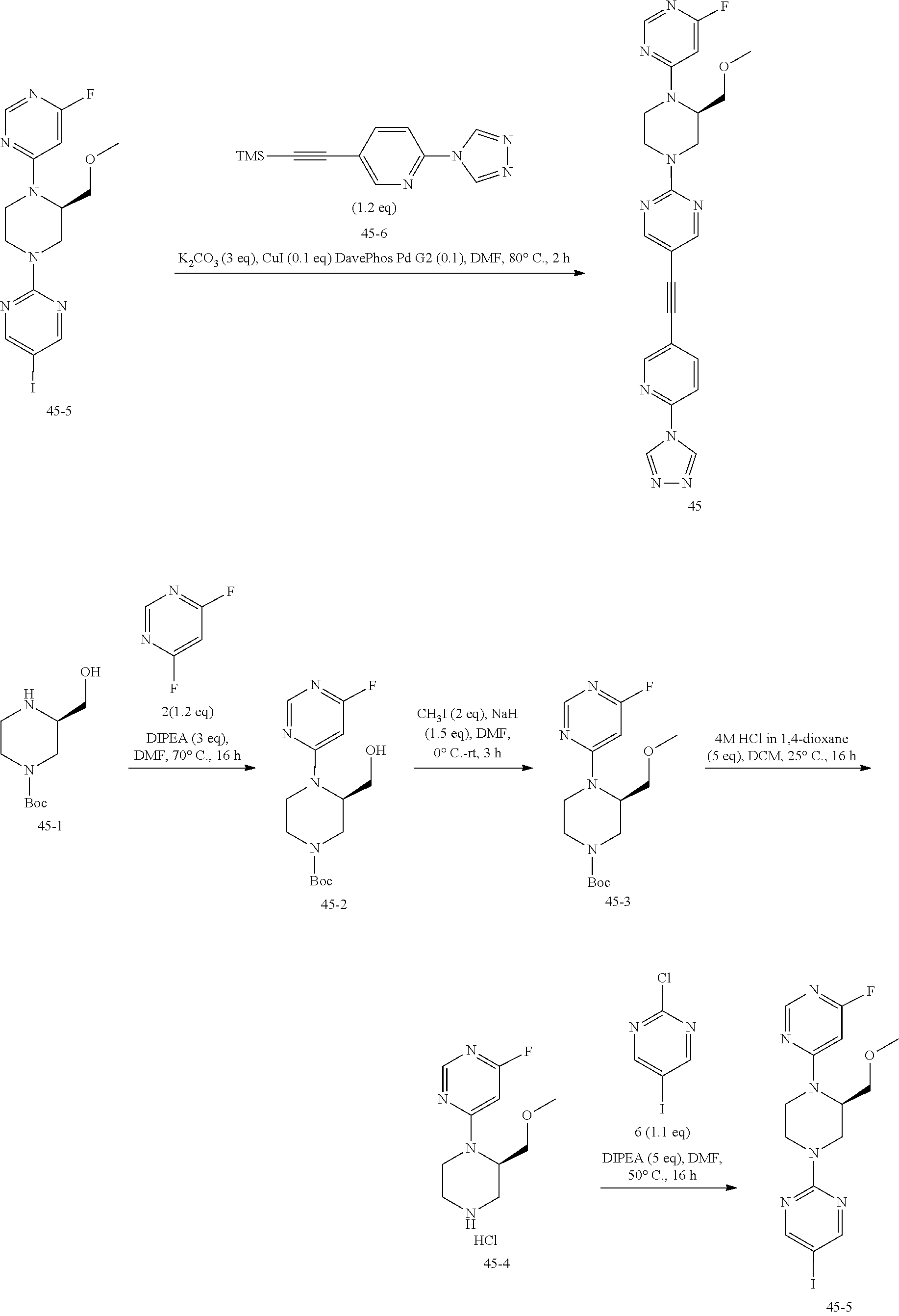

### Synthesis of 45-2: tert-butyl (R)-4-(6-fluoropyrimidin-4-yl)-3-(hydroxymethyl)piperazine-1-carboxylate

To a stirred solution of tert-butyl (R)-3-(hydroxymethyl)piperazine-1-carboxylate (5 g, 23.12 mmol) in DMF (80 mL) were added 4,6-difluoropyrimidine (3.22 g, 27.7 mmol), DIPEA (12.11 mL, 69.4 mmol) at room temperature. The reaction mixture was stirred at 70° C. for 16 h under argon atmosphere. Reaction mixture was diluted with water (70 mL), extracted with EtOAc (3×70 mL). Combined organic layer was washed with brine (70 mL), dried over anhydrous Na₂SO₄ and concentrated under reduced pressure. Crude compound was purified by biotage using 120 g (230-400 mesh) silica gel cartridge and compound eluted with 35% EtOAc in pet ether. Pure fractions were combined and concentrated under reduced pressure to afford 45-2. M/z (ESI)=313.31 [M+H]⁺.

### Synthesis of 45-3: tert-butyl (R)-4-(6-fluoropyrimidin-4-yl)-3-(methoxymethyl)piperazine-1-carboxylate

To a stirred solution of 45-2 (5.5 g, 17.61 mmol) in DMF (70 mL) was added methyl iodide (2.202 mL, 35.2 mmol) at 0° C. Then to this reaction mixture was added NaH (1.056 g, 26.4 mmol) portion-wise at 0° C. The reaction mixture was stirred at 25° C. for 3 h under argon atmosphere. Reaction mixture was diluted with water (100 mL), extracted with EtOAc (2×100 mL). Combined organic layer was washed with brine (100 mL), dried over anhydrous Na₂SO₄ filtered and concentrated under reduced pressure. Crude compound was purified by silica gel column chromatography and compound eluted with 15% EtOAc in pet ether. Pure fractions were combined and concentrated under reduced pressure to afford 45-3. M/z (ESI)=327.34 [M+H]⁺.

### Synthesis of 45-4: (R)-4-fluoro-6-(2-(methoxymethyl)piperazin-1-yl)pyrimidine

To a stirred solution of 45-3 (4.5 g. 13.79 mmol) in DCM (60 mL) was added 4M HCl (17.23 mL, 68.9 mmol) in 1,4-dioxane at room temperature. The reaction mixture was stirred at 25° C. for 16 h under argon atmosphere. Reaction mixture was concentrated under reduced pressure to afford 45-4. M/z (ESI)=227.13 [M+H]⁺.

### Synthesis of 45-5: (R)-2-(4-(6-fluoropyrimidin-4-yl)-3-(methoxymethyl)piperazin-1-yl)-5-iodopyrimidine

To a stirred solution of 45-4 (4.5 g, 17.13 mmol) in DMF (70 mL) were added 2-chloro-5-iodopyrimidine (4.53 g, 18.84 mmol), DIPEA (14.96 mL, 86 mmol) at room temperature. The reaction mixture was stirred at 50° C. for 16 h under argon atmosphere. Reaction mixture was diluted with water (200 mL). Precipitation was appeared which was stirred at room temperature for 10 min. Then solid was filtered and dried under vacuum. Crude compound was purified by biotage using 120 g (230-400 mesh) silica gel cartridge and compound elute with 35% EtOAc in pet ether. Pure fractions were combined and concentrated under reduced pressure to afford 45-5. M/z (ESI)=431.14 [M+H]⁺.

### Synthesis of 45: (R)-5-((6-(4H-1,2,4-triazol-4-yl)pyridin-3-yl)ethynyl)-2-(4-(6-fluoropyrimidin-4-yl)-3-(methoxymethyl)piperazin-1-yl)pyrimidine

To a stirred solution of 45-5 (200 mg, 0.465 mmol) in DMF (2 mL) were added 45-6 (135 mg, 0.558 mmol). K₂CO₃ (193 mg, 1.395 mmol), copper(i) iodide (9 mg, 0.047 mmol) at room temperature. The reaction mixture was degassed and purged with argon for 10 min. Then to this reaction mixture was added davephos G2 palladacycle (33 mg, 0.047 mmol) at room temperature. The reaction mixture was stirred at 80° C. for 2 h in a sealed tube. Reaction mixture was diluted with cold water (10 mL), filtered through celite pad, washed with DCM (20 mL) and organic layer was kept aside. Aqueous layer was then extracted with DCM (2×20 mL). Combined organic layer was washed with brine (20 mL), dried over anhydrous Na₂SO₄ and concentrated under reduced pressure. Crude compound was triturated with diethyl ether (10 mL) and the compound was purified by prep HPLC purification. Pure fractions were combined and lyophilized to afford 45. M/z (ESI)=473.38 [M+H]⁺

### Prep HPLC Purification:

### X-Bridge CIS PACK: Mobile Phase—10 mM Ammonium Bicarbonate IN H₂O: MeCN Column—

X-Bridge PACK, C18 (25×250) mm, 5μ Flow-20.0 ml/min Gradient Method—0/35,4/35,10.5/60,10.51/100,13/100,13.05/35,16/35 ¹H NMR (400 MHz, DMSO-d₆) δ=9.34 (s, 2H), 8.71 (d, J=1.6 Hz, 1H), 8.65 (s, 2H), 8.36 (d, J=2.6 Hz, 1H), 8.25 (dd, J=2.1, 8.5 Hz, 1H), 7.97 (d, J=8.5 Hz, 1H), 6.58 (s, 1H), 5.04-4.67 (m, 2H), 4.60-4.45 (m, 1H), 4.42-4.11 (m, 1H), 3.36 (br s, 4H), 3.30-3.28 (m, 1H), 3.20 (s, 3H).

### Example 46

### (R)-2-(4-(5-((2-(1-(2-fluoroethyl)-1H-pyrazol-4-yl)pyrimidin-5-yl)ethynyl)pyrimidin-2-yl)-2-(methoxymethyl)piperazin-1-yl)-1,3,5-triazine

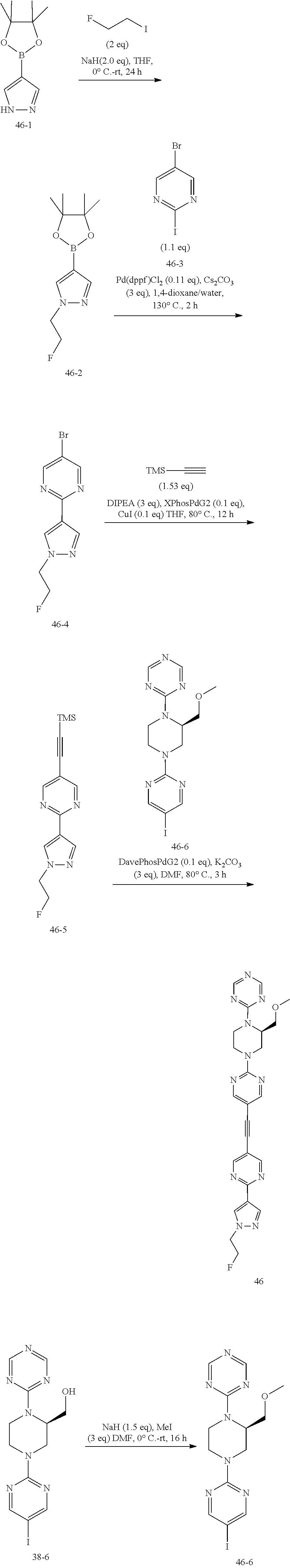

### Synthesis of 46-2: 1-(2-fluoroethyl)-4-(4,4,5,5-tetramethyl-1,3,2-dioxaborolan-2-yl)-1H-pyrazole

To a stirred solution of 4-(4,4,5,5-tetramethyl-1,3,2-dioxaborolan-2-yl)-1H-pyrazole (46-1) (5 g, 25.8 mmol) and 1-fluoro-2-iodoethane (8.96 g, 51.5 mmol) in THF (100 mL) under argon was added NaH (2.061 g, 51.5 mmol) portion wise at 0° C. The reaction mixture was stirred at 25° C. for 24 h. Reaction mixture was poured into crushed ice water (50 mL) and extracted with EtOAc (2×50 mL). Combined organic layer was washed with brine (100 mL), dried over anhydrous sodium sulphate, filtered and concentrated under reduced pressure. Crude compound was washed with diethyl ether (10 mL) and dried under vacuum to afford 46-2. M/z (ESI)=241.06 [M+H]⁺

### Synthesis of 46-4: 5-bromo-2-(1-(2-fluoroethyl)-1H-pyrazol-4-yl)pyrimidine

To a stirred solution of 46-2 (600 mg, 2.499 mmol) in 1,4-dioxane (15 mL) and water (3 mL) was added 5-bromo-2-iodopyrimidine (783 mg, 2.75 mmol) at 25° C. The reaction mixture was degassed and purged with argon for 10 min. Then to this reaction mixture were added Cs₂CO₃ (2443 mg, 7.50 mmol) and Pd(dppf)Cl₂ (201 mg, 0.275 mmol) at 25° C. The reaction mixture was stirred at 130° C. for 2 h. Reaction mixture was partitioned between water (20 mL) and EtOAc (20 mL). Organic layer was dried over sodium sulfate, filtered and concentrated under reduced pressure. Crude compound was purified by Biotage using 12 g silica gel cartridge and compound eluted with 50% EtOAc in pet ether. Pure fractions were combined and concentrated under reduced pressure to afford 46-4. M/z (ESI)=271.18 [M+H]+

### Synthesis of 46-5: 2-(1-(2-fluoroethyl)-1H-pyrazol-4-yl)-5-((trimethylsilyl)ethynyl)pyrimidine

The stirred solution of 46-4 (180 mg, 0.664 mmol) in THF (6 mL) was degassed and purged with argon for 5 min. Then to this reaction mixture were added XPhos Pd G2 (52.2 mg, 0.066 mmol), DIPEA (0.348 mL, 1.992 mmol), copper(i) iodide (12.65 mg, 0.066 mmol) and trimethylsilylacetylene (100 mg, 1.018 mmol) at 25° C. The reaction mixture was stirred at 80° C. for 12 h. Reaction mixture was diluted with EtOAc (20 mL) and washed with water (20 mL). Organic layer was dried over anhydrous sodium sulphate, filtered and concentrated under reduced pressure. Crude compound was purified by Biotage using 4 g silica gel cartridge and compound eluted with 30% EtOAc in pet ether. Pure fractions were combined and concentrated under reduced pressure to afford 46-5. M/z (ESI)=289.28 [M+H]⁺

### Synthesis of 46-6: (R)-2-(4-(5-iodopyrimidin-2-yl)-2-(methoxymethyl)piperazin-1-yl)-1,3,5-triazine

To a stirred solution of 38-6 (1.00 g, 2.51 mmol) in DMF (20 mL) was added NaH (90.2 mg, 3.76 mmol) at 0° C. and stirred at 0° C. for 30 min. Then methyl iodide (470 μL, 7.52 mmol) was added dropwise to the reaction mixture at 0° C. The reaction mixture was stirred at 25° C. for 16 h. Reaction mixture was quenched with cold water (50 mL) and extracted with EtOAc (2×50 mL). Combined organic layer was washed with brine (100 mL), dried over anhydrous Na₂SO₄, filtered and concentrated under reduced pressure. Crude compound was purified by Biotage using 120 g silica cartridge and compound eluted with 15% EtOAc in hexane. Pure fractions were combined and concentrated under reduced pressure to afford 46-6. M/Z (ESI): 414.10 [M+H]⁺.

### Synthesis of 46: (R)-2-(4-(5-((2-(1-(2-fluoroethyl)-1H-pyrazol-4-yl)pyrimidin-5-yl)ethynyl)pyrimidin-2-yl)-2-(methoxymethyl)piperazin-1-yl)-1,3,5-triazine

To a stirred solution of 46-6 (200 mg, 0.484 mmol) in DMF (4 mL) was added 46-5 (100 mg, 0.347 mmol) at 25° C. The reaction mixture was degassed and purged with argon for 10 min. Then to this reaction mixture were added K₂CO₃ (201 mg, 1.452 mmol) and davephos g2 palladacycle (34.1 mg, 0.048 mmol) at 25° C. Reaction mixture was stirred at 80° C. for 3 h. Reaction mixture was diluted with EtOAc (10 mL), water (10 mL), and filtered through celite bed. Organic layer was dried over anhydrous sodium sulphate, filtered and concentrated under reduced pressure. Crude compound was purified by Biotage using 4 g silica gel cartridge and compound was eluted with 70% EtOAc in pet ether. Pure fractions were combined and concentrated under reduced pressure. Obtained compound was again purified by prep HPLC purification. Pure fractions were combined and lyophilized to afford 46. M/z (ESI)=502.29 [M+H]⁻

### Prep HPLC Purification;

X-Bridge: Mobile Phase—10 mM Ammonium Bicarbonate IN H₂O: MeCN
COLUMN—X-Bridge, C18 (10×250) mm, 5μ Flow-19.0 ml/min
Gradient Method—0/35,2/35,7.0,40,13.0/51,1.1/100,15.9/100,16/35,21/35

¹H NMR (400 MHz, DMSO-d₆) δ=8.91 (s, 2H), 8.74-8.68 (m, 4H), 8.50 (s, 1H), 8.13 (s, 1H), 5.02-4.97 (m, 1H), 4.89-4.85 (m, 1H), 4.79-4.73 (m, 2H), 4.61-4.46 (m, 4H), 3.44 (d, J=6.8 Hz, 2H), 3.38-3.34 (m, 1H), 3.30-3.18 (m, 5H).

### Example 47

### (R)-2-(4-(5-((6-(1-(2-fluoroethyl)-1H-pyrazol-4-yl)pyridin-3-yl)ethynyl)pyrimidin-2-yl)-2-(methoxymethyl)piperazin-1-yl)-1,3,5-triazine

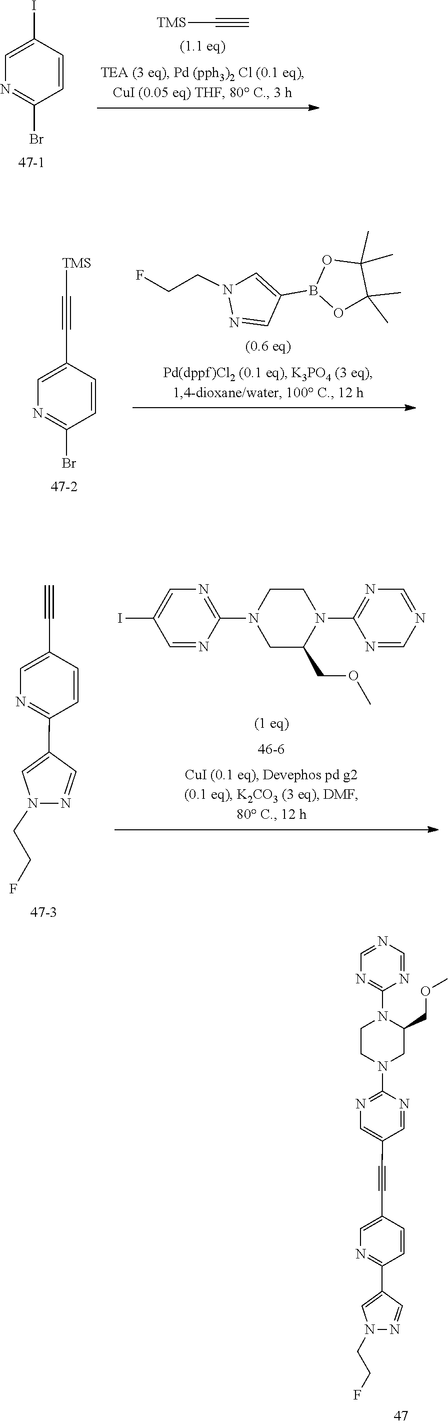

### Synthesis of 47-2: 2-bromo-5-((trimethylsilyl)ethynyl)pyridine

The stirred solution of 2-bromo-5-iodopyridine (47-1) (5 g, 17.61 mmol) in THF (75 mL) was purged with argon gas for 10 min. Then to this reaction mixture were added TEA (7.36 mL, 52.8 mmol), copper(i) iodide (0.168 g, 0.881 mmol), dichlorobis(triphenylphosphine)palladium(ii) (1.236 g, 1.761 mmol) and trimethylsilylacetylene (1.903 g, 19.37 mmol) at room temperature and again purged with argon for another 10 min. The reaction mixture was stirred at 80° C. for 3 h. The reaction mixture was diluted with water (200 mL) and extracted with EtOAc (3×180 mL). Combined organic layer was dried over Na₂SO₄ and concentrated under reduced pressure. Crude compound was purified by 100 g (100-200 mesh) silica gel cartridge and compound eluted with 20% EtOAc in pet ether. Pure fractions were combined and concentrated under reduced pressure to afford 47-2. M/Z (ESI): 254.12 [M+H]⁻.

### Synthesis of 47-3: 5-ethynyl-2-(1-(2-fluoroethyl)-1H-pyrazol-4-yl)pyridine

The stirred solution of 47-2 (1.2 g, 4.72 mmol) in 1,4 dioxane (12 mL), water (5 mL) was purged with argon gas for 10 min. Then to this reaction mixture was added 1-(2-fluoroethyl)-4-(4,4,5,5-tetramethyl-1,3,2-dioxaborolan-2-yl)-1H-pyrazole (0.680 g. 2.83 mmol), potassium phosphate tribasic (3.01 g, 14.16 mmol) and [1,1′-bis(diphenylphosphino)ferrocene]dichloropalladium(II)](0.345 g, 0.472 mmol) at room temperature and again purged with argon for another 10 min. The reaction mixture was stirred at 100° C. for 12 h. Reaction mixture was diluted with water (100 mL) and extracted with EtOAc (3×100 mL). Combined organic layer was dried over Na₂SO₄ and concentrated under reduced pressure. Crude compound was purified by 100 g (100-200 mesh) silica gel cartridge and compound eluted with 20% EtOAc in pet ether. Pure fractions were combined and concentrated under reduced pressure to afford 47-3. M/Z (ESI): 216.23 [M+H]⁺.

### Synthesis of 47: (R)-2-(4-(5-((6-(1-(2-fluoroethyl)-1H-pyrazol-4-yl)pyridin-3-yl)ethynyl)pyrimidin-2-yl)-2-(methoxymethyl)piperazin-1-yl)-1,3,5-triazine

The stirred solution of 46-6 (600 mg, 1.452 mmol) in DMF (5 mL) was purged with argon gas for 10 min. Then to this reaction mixture were added 47-3 (344 mg, 1.597 mmol), K₂CO₃ (602 mg, 4.36 mmol), copper(I) iodide (27.7 mg, 0.145 mmol) and davephos g2 palladacycle (102 mg, 0.145 mmol) at room temperature and again purged with argon for another 10 min. The reaction mixture was stirred at 80° C. for 12 h. The reaction mixture was diluted with water (100 mL) and extracted with EtOAc (3×100 mL). Combined organic layer was dried over Na₂SO₄ and concentrated under reduced pressure. Crude compound was purified by 100 g (100-200 mesh) silica gel column and compound eluted with 60% EtOAc in pet ether. Pure fractions were combined and concentrated under reduced pressure. Obtained compound was purified by prep HPLC purification. Pure fractions were combined and lyophilized to afford 47. M/Z (ESI): 501.35 [M+H]⁺.

### Prep HPLC Method.

Mobile Phase—10 mM Ammonium Bicarbonate IN H2O: MeCN
COLUMN—X-Bridge C18 (19×250) Flow-18.0 ml/mm
Gradient Method—0/30,7/42,12.44.5,12.05/100,14/100,14.05/30,17/30

¹H NMR (400 MHz, DMSO-d₆) δ (ppm)=8.88-8.57 (m, 5H), 8.42 (s, 1H), 8.12 (s, 1H), 7.91 (dd, J=2.1, 8.3 Hz, 1H), 7.73 (d, J=8.4 Hz, 1H), 5.03-4.95 (m, 1H), 4.89-4.73 (m, 3H), 4.64-4.54 (m, 2H), 4.54-4.43 (m, 2H), 3.44 (d, J=7.0 Hz, 2H), 3.36 (br d, J=4.4 Hz, 2H), 3.28-3.14 (m, 5H).

### Example 48

### (R)-5-(5-((2-(3-((2-(2-fluoroethoxy)ethoxy)methyl)-4-(1,3,5-triazin-2-yl)piperazin-1-yl)pyrimidin-5-yl)ethynyl)pyridin-2-yl)oxazole

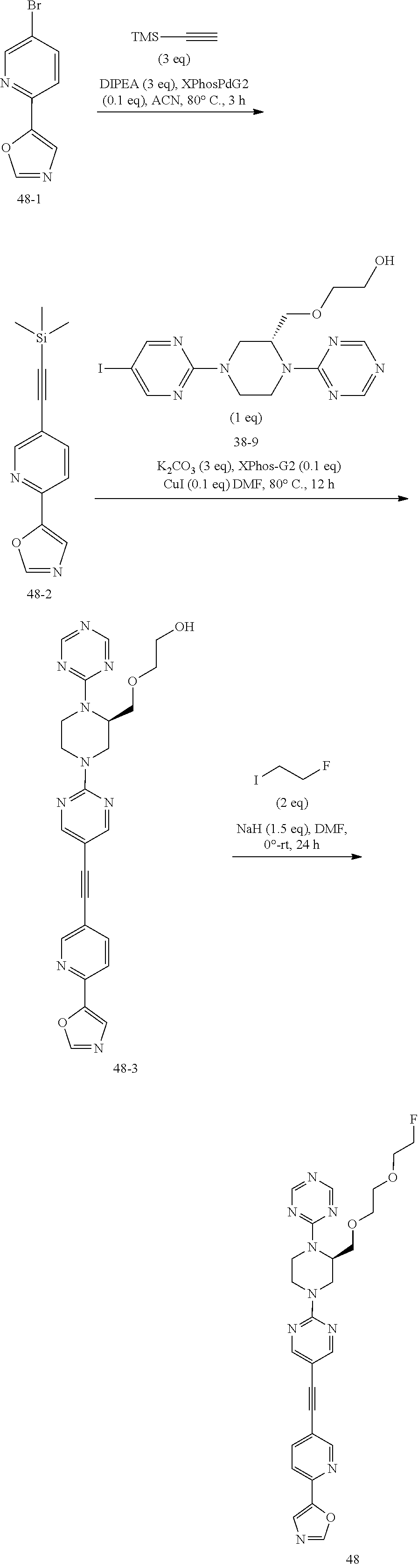

### Synthesis of 48-2: 5-(5-((trimethylsilyl)ethynyl)pyridin-2-yl)oxazole

The stirred solution of 48-1 (1.0 g, 4.44 mmol) in ACN (20 mL) was degassed and purged with argon gas for 10 min. Then to this reaction mixture were added DIPEA (2.328 mL, 13.33 mmol), chloro(2-dicyclohexylphosphino-2′,4′,6′-triisopropyl-1,1′-biphenyl)[2-(2′-amino-1,1′-biphenyl)]palladium(II) (0.350 g, 0.444 mmol), and ethynyltrimethylsilane (1.309 g, 13.33 mmol) at room temperature and again purged with argon gas for another 10 min. The reaction mixture was stirred at 80° C. for 3 h. The reaction mixture was diluted with water (200 mL) and extracted with EtOAc (3×180 mL). Combined organic layer was dried over Na₂SO₄ and concentrated under reduced pressure. Crude compound was purified by 100 g (100-200 mesh) silica gel cartridge and compound eluted with 20% EtOAc in pet ether. Pure fractions were combined and concentrated under reduced pressure to afford 48-2. M/Z (ESI): 243.46 [M+H]⁺.

### Synthesis of 48-3: (R)-2-((4-(5-((6-(oxazol-5-yl)pyridin-3-yl)ethynyl)pyrimidin-2-yl)-1-(1,3,5-triazin-2-yl)piperazin-2-yl)methoxy)ethan-1-ol

The stirred solution of 38-9 (500 mg, 1.128 mmol) in DMF (10 mL) was purged with argon gas for 10 min. Then to this reaction mixture were added 48-2 (328 mg, 1.354 mmol), K₂CO₃ (468 mg, 3.38 mmol), copper(i) iodide (21.48 mg, 0.113 mmol) and chloro(2-dicyclohexylphosphino-2′,4′,6′-triisopropyl-1,1′-biphenyl)[2-(2′-amino-1,1′-biphenyl)]palladium(II) (89 mg, 0.113 mmol) at room temperature. The reaction mixture was again purged with argon gas for another 10 min and stirred at 80° C. for 12 h. Reaction mixture was diluted with water (200 mL) and extracted with EtOAc (3×150 mL). Combined organic layer was dried over Na₂SO₄ and concentrated under reduced pressure. Crude compound was triturated with diethyl eater (2×50 mL) and concentrated under reduced pressure to afford 48-3. M/Z (ESI): 486.35 [M+H]⁺.

¹H NMR (400 MHz, DMSO-d₆) δ (ppm)=8.78-8.76 (m, 1H), 8.64 (s, 4H), 8.61-8.57 (m, 1H), 8.06 (dd, J=2.1, 8.3 Hz, 1H), 7.88 (s, 1H), 7.82 (d, J=8.1 Hz, 1H), 4.98-4.92 (m, 1H), 4.81-4.76 (m, 1H), 4.62-4.48 (m, 3H), 3.52 (d, J=7.0 Hz, 2H), 3.42-3.36 (m, 5H), 3.30-3.15 (m, 2H).

### Synthesis of 48: (R)-5-(5-((2-(3-((2-(2-fluoroethoxy)ethoxy)methyl)-4-(1,3,5-triazin-2-yl)piperazin-1-yl)pyrimidin-5-yl)ethynyl)pyridin-2-yl)oxazole

To a stirred solution of 48-3 (50 mg, 0.103 mmol) in DMF (1 mL) was added 60% in oil NaH (6.18 mg, 0.154 mmol) at 0° C. The reaction mixture was stirred at 0° C. for 30 min followed by addition of 1-fluoro-2-iodoethane (35.8 mg, 0.206 mmol) at 0° C. The reaction mixture was stirred at 25° C. for 24 h. The reaction mixture was diluted with water (50 mL) and extracted with EtOAc (3×80 mL). Combined organic layer was dried over Na₂SO₄ and concentrated under reduced pressure. Crude compound was purified by prep HPLC purification. Pure fractions were combined and lyophilized to afford 48. M/Z (ESI): 532.38 [M+H]⁺.
Prep HPLC method.
Mobile Phase—10 mM Ammonium Bicarbonate IN H₂O: MeCN
COLUMN—X-Bridge C18 (19×250) Flow-18.0 ml/min
Gradient Method—0/30,7/42,12.44.5,12.05/100.14/100.14.05/30,17/30

¹H NMR (400 MHz, DMSO-d₆) δ (ppm)=8.78 (s, 1H), 8.64 (s, 4H), 8.59 (s, 1H), 8.06 (dd, J=1.8, 8.2 Hz, 1H), 7.88 (s, 1H), 7.83 (d, J=8.3 Hz, 1H), 5.01-4.93 (m, 1H), 4.79 (br d, J=13.4 Hz, 1H), 4.65-4.48 (m, 4H), 3.65-3.49 (m, 9H), 3.22 (br s, 2H).

### Example 49

### (R)-2-(2-((2-fluoroethoxy)methyl)-4-(5-((2-(1-methyl-1H-pyrazol-3-yl)pyrimidin-5-yl)ethynyl)pyrimidin-2-yl)piperazin-1-yl)-1,3,5-triazine

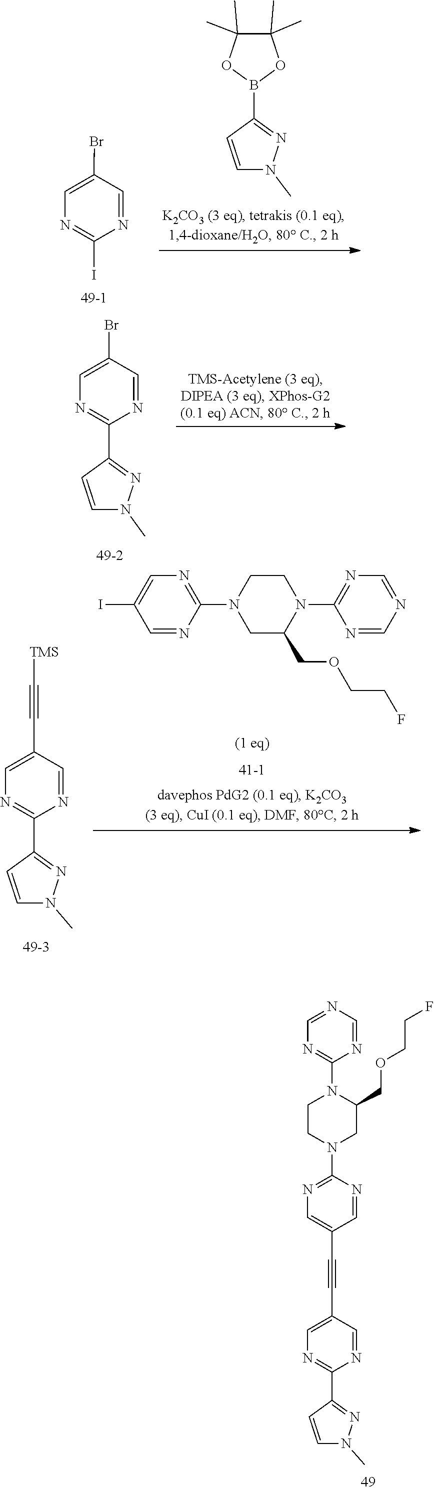

### Synthesis of 49-2: 5-bromo-2-(1-methyl-1H-pyrazol-3-yl)pyrimidine

To a stirred solution of 5-bromo-2-iodopyrimidine (500 mg, 1.755 mmol) in 1,4-dioxane (10 mL) and water (2 mL) were added 1-methyl-3-(4,4,5,5-tetramethyl-1,3,2-dioxaborolan-2-yl)-1H-pyrazole (548 mg, 2.63 mmol) and K₂CO₃ (728 mg, 5.27 mmol) at room temperature. The reaction mixture was degassed and purged with argon gas for 15 min. Then to this reaction mixture tetrakis(triphenylphosphine)palladium (203 mg, 0.176 mmol) was added at room temperature. The reaction mixture was stirred at 80° C. for 2 h under nitrogen atmosphere in a sealed tube. The reaction mixture was diluted with water (10 mL), extracted with EtOAc (2×20 mL). Combined organic layer was washed with brine (10 mL), dried over anhydrous Na₂SO₄, filtered and concentrated under reduced pressure. Crude compound was purified by Biotage using silica gel column and compound eluted with 40% EtOAc in pet ether. Pure fractions were combined and concentrated under reduced pressure to afford 49-2. M/Z (ESI): 238.92 [M+H]⁺.

### Synthesis of 49-3: 2-(1-methyl-1H-pyrazol-3-yl)-5-((trimethylsilyl)ethynyl)pyrimidine

To a stirred solution of 49-2 (350 mg, 1.464 mmol) in acetonitrile (5 mL) was added DIPEA (0.767 mL, 4.39 mmol) at room temperature. The reaction mixture was degassed and purged with argon gas for 15 min. Then to this reaction mixture trimethylsilylacetylene (431 mg, 4.39 mmol) and chloro(2-dicyclohexylphosphino-2′,4′,6′-triisopropyl-1,1′-biphenyl)[2-(2′-amino-1,1′-biphenyl)]palladium(II) (115 mg, 0.146 mmol) were added at room temperature. The reaction mixture was stirred at 80° C. for 2 h under nitrogen atmosphere in a sealed tube. The reaction mixture was diluted with water (10 mL), extracted with EtOAc (2×20 mL). Combined organic layer was washed with brine (10 mL), dried over anhydrous Na₂SO₄, filtered and concentrated under reduced pressure. Crude compound was purified by Biotage using silica gel column and compound eluted with 50% EtOAc in pet ether. Pure fractions were combined and concentrated under reduced pressure to afford 49-3. M/Z (ESI): 257.35 [M+H]⁺.

### Synthesis of 49: (R)-2-(2-((2-fluoroethoxy)methyl)-4-(5-((2-(1-methyl-1H-pyrazol-3-yl)pyrimidin-5-yl)ethynyl)pyrimidin-2-yl)piperazin-1-yl)-1,3,5-triazine

To a stirred solution of 41-1 (130 mg, 0.292 mmol) in DMF (2 mL) were added 49-3 (112 mg, 0.438 mmol), K₂CO₃ (121 mg, 0.876 mmol) and copper (I) iodide (5.56 mg, 0.029 mmol) at room temperature. The reaction mixture was degassed and purged with argon gas for 10 min. Then to this reaction mixture was added davephos G2 palladacycle (20.54 mg, 0.029 mmol) at room temperature. The reaction mixture was stirred at 80° C. for 2 h in a sealed tube. The reaction mixture was diluted with water (20 mL), extracted with EtOAc (2×30 mL). Combined organic layer was washed with brine (10 mL), dried over anhydrous Na₂SO₄, filtered and concentrated under reduced pressure. Crude compound was purified by prep-HPLC purification (conditions: Mobile Phase—10 mM Ammonium Bicarbonate IN H₂O: MeCN COLUMN—Betasil Phenyl hexyl C18 (20×250), 5μ Flow-18.0 ml/min. Gradient Method:-0/38, 2/38, 15.50/42, 15.60/100, 19.90/100, 20.0/38, 24.0/38). Pure fractions were combined and lyophilized to afford 49. M/Z (ESI): 502.22 [M+H]⁺.

¹H NMR (400 MHz, DMSO-d₆) δ (ppm)=8.96 (s, 2H). 8.67-8.63 (m, 4H), 7.84 (d, J=2.1 Hz, 1H), 6.94 (d, J=2.3 Hz, 1H), 5.01-4.93 (m, 1H), 4.80 (br d, J=13.6 Hz, 1H), 4.64-4.52 (m, 2H), 4.51-4.34 (m, 2H), 3.95 (s, 3H), 3.68-3.54 (m, 4H), 3.44-3.34 (m, 2H), 3.28-3.22 (m, 1H).

### Example 50

### (R)-2-(2-((2-fluoroethoxy)methyl)-4-(5-((6-(1-methyl-1H-pyrazol-4-yl)pyridin-3-yl)ethynyl)pyrimidin-2-yl)piperazin-1-yl)-1,3,5-triazine

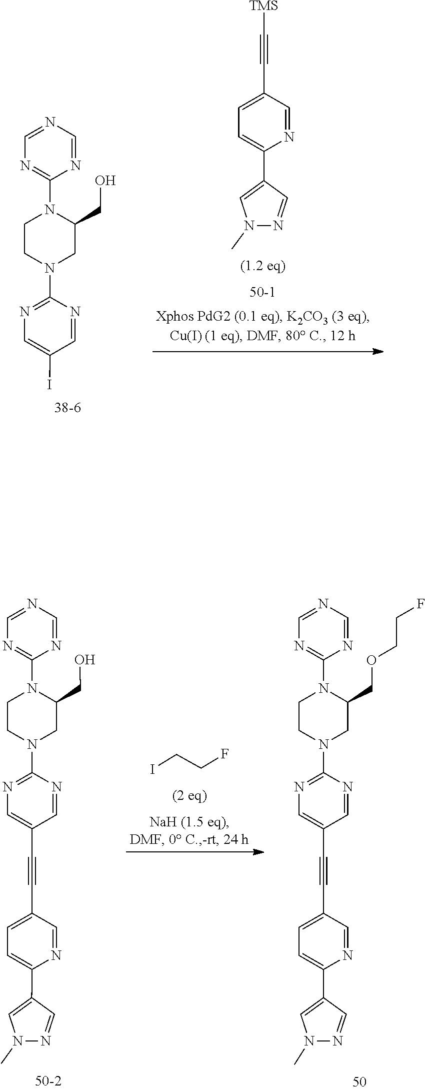

### Synthesis of 50-2: (R)-(4-(5-((6-(1-methyl-1H-pyrazol-4-yl)pyridin-3-yl)ethynyl)pyrimidin-2-yl)-1-(1,3,5-triazin-2-yl)piperazin-2-yl)methanol

To a stirred solution of 38-6 (150 mg, 0.376 mmol) in DMF (3 mL) was degassed and purged with argon gas for 10 min. Then to this reaction mixture were added 50-1 (made in an analogous manner as 44-3) (115 mg, 0.451 mmol), K₂CO₃ (156 mg, 1.127 mmol), copper (I) iodide (71.6 mg, 0.376 mmol) and chloro(2-dicyclohexylphosphino-2′,4′,6′-triisopropyl-1,1′-biphenyl)[2-(2′-amino-1,1′-biphenyl)]palladium(II) (29.6 mg, 0.038 mmol) at room temperature and again purged with argon gas for another 10 min. The reaction mixture was stirred at 80° C. for 12 h. The reaction mixture was quenched with water (40 mL) and extracted with EtOAc (3×30 mL). Combined organic layer was dried over anhydrous Na₂SO₄, filtered and concentrated under reduced pressure. Crude compound was washed with diethyl ether (2×30 mL), dried over Na₂SO₄ and concentrated under reduced pressure. Obtained compound was further re-purified by prep HPLC purification (conditions: Mobile Phase—10 mM Ammonium Bicarbonate IN H₂O: MeCN COLUMN—X-Bridge C18 (19×250) mm, 5μ Flow-14.0 ml/min Gradient Method-0/25,2/25.11/60.9,11.05/100,17/100,17.05/25,20/25). Pure fractions were combined and concentrated under reduced pressure to afford 50-2. M/Z (ESI): 455.19 [M+H]⁺.

¹H NMR (400 MHz, DMSO-d₆) δ (ppm)=8.58-8.68 (m, 5H), 8.34 (s, 1H), 8.04 (s, 1H), 7.90 (dd, J=8.2 Hz, 2.2 Hz, 1H), 7.69 (d, J=8.4 Hz, 1H), 4.89 (t, J=5.0 Hz, 1H), 4.72-4.85 (m, 2H), 4.46-4.61 (m, 2H), 3.89 (s, 3H), 3.45-3.57 (m, 2H), 3.36-3.42 (m, 2H), 3.22-3.28 (m, 1H).

### Synthesis of 50: (R)-2-(2-((2-fluoroethoxy)methyl)-4-(5-((6-(1-methyl-1H-pyrazol-4-yl)pyridin-3-yl)ethynyl)pyrimidin-2-3l)piperazin-1-yl)-1,3,5-triazine

To a stirred solution of 50-2 (70 mg, 0.154 mmol) in DMF (1 mL) was added 60% NaH in oil (9.24 mg. 0.231 mmol) at 0° C. The reaction mixture was stirred at 0° C. for 30 min. Then to this reaction mixture was added 1-fluoro-2-iodoethane (53.6 mg, 0.308 mmol) at 0° C. The reaction mixture was stirred at room temperature for 24 h. The reaction mixture was quenched with water (30 mL) and extracted with EtOAc (3×40 mL). Combined organic layer was dried over anhydrous Na₂SO₄, filtered and concentrated under reduced pressure. Crude compound was purified by prep HPLC purification (conditions: Mobile Phase—10 mM Ammonium Bicarbonate IN H₂O: MeCN COLUMN—X-Bridge C18 (10×250 mm), 5μ Flow-7 ml/min Gradient Method-0/52,2/52,7.5/55.5,10/55.5,10.05/100,12/100,12.05/52,16/52). Pure fractions were combined and concentrated under reduced pressure to afford 50. M/Z (ESI): 501.21 [M+H]⁺.

¹H NMR (400 MHz, DMSO-d₆) δ (ppm)=8.63 (d, J=10.8 Hz, 5H), 8.34 (s, 1H), 8.04 (s, 1H), 7.89 (dd, J=8.2 Hz, 2.2 Hz, 1H), 7.69 (d, J=8.0 Hz, 1H), 4.43-5.04 (m, 1H), 4.79 (d, J=13.6 Hz, 1H), 4.54-4.67 (m, 2H), 4.43 (dt, J=48.0 Hz, 4.0 Hz, 2H), 3.89 (s, 3H), 3.51-3.60 (m, 4H), 3.35-3,43 (m, 2H), 3.18-3.26 (m, 1H).

### Example 51

### (R)-2-(3-((2-fluoroethoxy)methyl)-4-(pyrimidin-2-yl)piperazin-1-yl)-5-((2-(1-methyl-1H-pyrazol-4-yl)pyrimidin-5-yl)ethynyl)pyrimidine

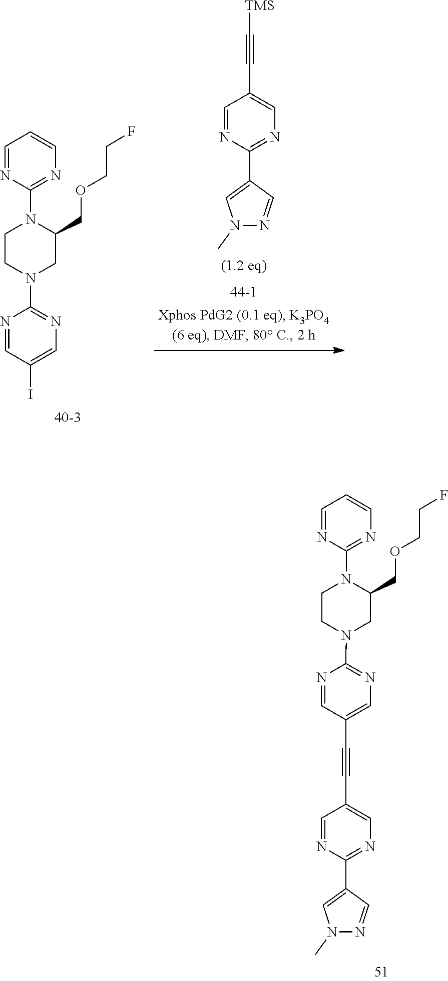 [SMILES: FCCOC[C@H]1CN(c2ncc(I)cn2)CCN1c1ncccn1 ; Cn1cc(-c2ncc(C#C[Si](C)(C)C)cn2)cn1 ; Cn1cc(-c2ncc(C#Cc3cnc(N4CCN(c5ncccn5)[C@@H](COCCF)C4)nc3)cn2)cn1]

### Synthesis of 51: (R)-2-(3-((2-fluoroethoxy)methyl)-4-(pyrimidin-2-yl)piperazin-1-yl)-5-((2-(1-methyl-1H-pyrazol-4-yl)pyrimidin-5-yl)ethynyl)pyrimidine

To a solution of 40-3 (100 mg, 0.301 mmol) in DMF (2 mL) were added 44-1 (93 mg, 0.361 mmol), tripotassium phosphate (383 mg, 1.805 mmol) at room temperature. The reaction mixture was degassed and purged with argon for 10 min. Then to this reaction mixture was added chloro(2-dicyclohexylphosphino-2′,4′,6′-triisopropyl-1,1′-biphenyl)[2-(2′-amino-1,1′-biphenyl)]palladium(II) (24 mg, 0.031 mmol) at room temperature. The reaction mixture was stirred at 80° C. for 2 h in a sealed tube. Reaction mixture was quenched with water (20 mL), filtered through celite pad and washed with DCM (2×20 mL). Combined organic layer was washed with brine (20 mL), dried over anhydrous Na₂SO₄, filtered and concentrated under reduced pressure. Obtained compound was triturated with diethyl ether (2×10 mL) and concentrated under reduced pressure. Obtained compound was purified by prep HPLC purification (conditions: Mobile Phase—10 mM Ammonium Bicarbonate IN H₂O: MeCN COLUMN—X-Bridge C18 (19×250), 5μ Flow-15.0 ml/min Gradient Method: 0/45, 2/45, 7/58, 12/58, 12.1/100, 15/100, 15.1/45, 18/45). Pure fractions were combined and concentrated under reduced pressure to afford 51. M/Z (ESI): 501.21 [M+H]⁺.

¹H NMR (400 MHz, DMSO-d₆) δ=8.89 (s, 2H), 8.63 (d, J=4.4 Hz, 4H), 8.42 (s, 1H), 8.05 (s, 1H), 4.98-4.96 (m, 1H), 4.80 (d, J=13.6 Hz, 1H), 4.59-4.55 (m, 2H), 4.44 (dt, J=4, 8.0 Hz, 2H), 3.92 (s, 3H), 3.65-3.55 (m, 4H), 3.40-3.36 (m, 1H), 3.29-3.20 (m, 2H).

### Example 52

### (R)-2-(3-((2-(2-fluoroethoxy)ethoxy)methyl)-4-(pyrimidin-2-yl)piperazin-1-yl)-5-((2-(1-methyl-1H-pyrazol-4-yl)pyrimidin-5-yl)ethynyl)pyrimidine

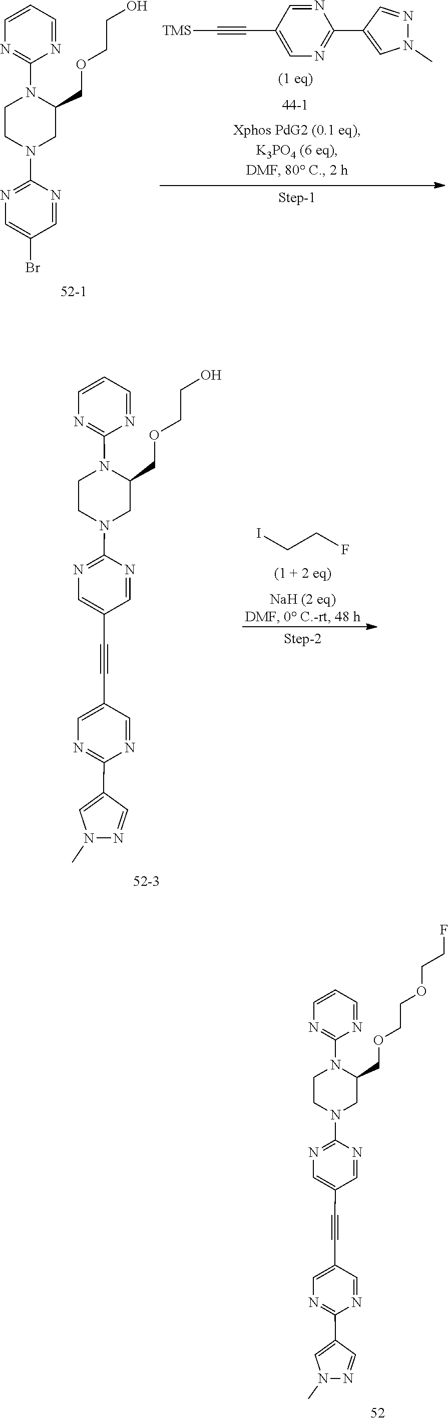

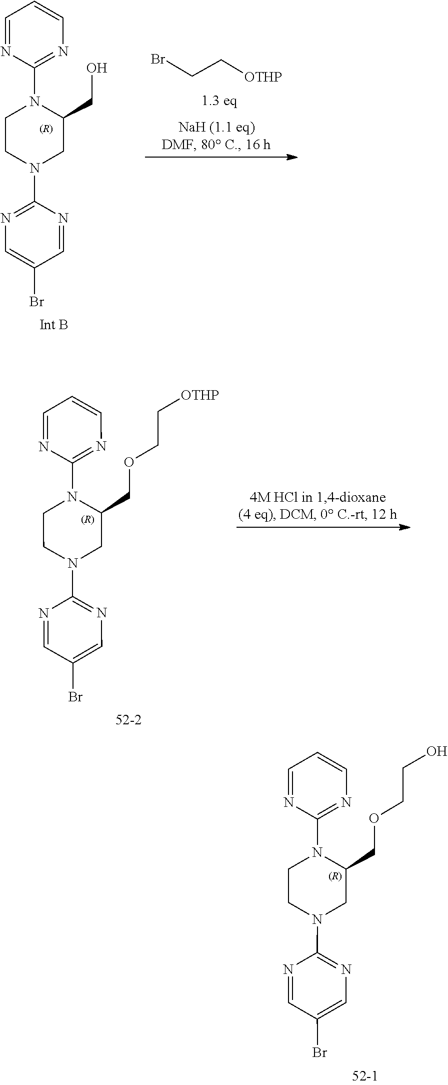

### Synthesis of 52-2: 5-bromo-2-((3R)-4-(pyrimidin-2-yl)-3-((2-((tetrahydro-2H-pyran-2-yl)oxy)ethoxy)methyl)piperazin-1-yl)pyrimidine

To a stirred solution of (R)-(4-(5-bromopyrimidin-2-yl)-1-(pyrimidin-2-yl)piperazin-2-yl)methanol (Int B) (3 g, 8.54 mmol) in DMF (80 mL) was added 60% in oil NaH (0.376 g, 9.40 mmol) at 0° C. and stirred at 0° C. for 30 min. Then 2-(2-bromoethoxy)tetrahydro-2 h-pyran (2.322 g, 11.10 mmol) was added to the reaction mixture at 0° C. The reaction mixture was stirred at 80° C. for 16 h. Reaction mixture was quenched with water (100 mL) and extracted with EtOAc (3×100 mL). Combined organic layer was dried over Na₂SO₄, filtered and concentrated under reduced pressure. Crude compound was purified by Biotage using 25 g silica gel cartridge and compound eluted with 30% EtOAc in pet ether. Pure fractions were combined and concentrated under reduced pressure to afford 52-2. M/Z (ESI): 479.13 [M+H]⁺.

### Synthesis of 52-1: (R)-2-((4-(5-bromopyrimidin-2-yl)-1-(pyrimidin-2-yl)piperazin-2-yl)methoxy)ethan-1-ol

To a stirred solution of 52-2 (2.5 g. 4.75 mmol) in DCM (30 mL) was added 4M 1,4-dioxane hydrochloride (4.75 mL, 19.00 mmol) at 0° C. The reaction mixture was stirred at 25° C. for 12 h. Reaction mixture was concentrated under reduced pressure. The residue was diluted with aq. NaHCO₃ solution (100 mL) and extracted with EtOAc (3×100 mL). Combined organic layer was dried over Na₂SO₄, filtered and concentrated under reduced pressure. Crude compound was purified by Biotage using 25 g silica gel cartridge and compound eluted with 40% EtOAc in pet ether. Pure fractions were combined and concentrated under reduced pressure to afford 52-1. M/Z (ESI): 395.24 [M+H]⁺.

### Synthesis of 52-3: (R)-2-((4-(5-((2-(1-methyl-1H-pyrazol-4-yl)pyrimidin-5-yl)ethynyl)pyrimidin-2-yl)-1-(pyrimidin-2-yl)piperazin-2-yl)methoxy)ethan-1-ol

To a solution of 52-1 (prepared in an analogous manner to 38-9) (200 mg, 0.506 mmol) in DMF (2 mL) were added 44-1 (130 mg, 0.506 mmol), tripotassium phosphate (644 mg, 3.04 mmol) at room temperature. The reaction mixture was degassed and purged with argon gas for 10 min. Then to this reaction mixture was added chloro (2-dicyclohexylphosphino-2′,4′,6′-triisopropyl-1,1′-biphenyl)[2-(2′-amino-1,1′-biphenyl)]palladium(II) (39.8 mg, 0.051 mmol) and stirred at 80° C. for 2 h in a sealed tube. The reaction mixture was quenched with water (20 mL) and extracted with EtOAc (2×30 mL). Combined organic layer was washed with brine (10 mL), dried over anhydrous Na₂SO₄, filtered and concentrated under reduced pressure. Crude compound was purified by prep-HPLC purification (conditions: Mobile Phase—10 mM Ammonium Bicarbonate IN H₂O: MeCN COLUMN—X-Bridge C18 (19×250), 5μ Flow-15.0 ml/min Gradient Method: 0/35, 2/35, 7/40, 11.7/40, 11.75/100, 14.95/100, 15/35, 18/35). Pure fractions were combined and concentrated under reduced pressure and lyophilized to afford 52-3. M/Z (ESI): 499.16 [M+H]⁺.

¹H NMR (400 MHz, DMSO-d₆) δ=8.89 (s, 2H), 8.62 (s, 2H), 8.37-8.44 (m, 3H), 8.05 (s, 1H), 6.91 (br s, 1H), 6.68 (t, J=4.8 Hz, 1H), 4.78-4.96 (m, 2H), 4.48-4.59 (m, 2H), 3.92 (s, 3H), 3.35-3.53 (m, 7H), 3.20-3.30 (m, 2H).

### Synthesis of 52: (R)-2-(3-((2-(2-fluoroethoxy)ethoxy)methyl)-4-(pyrimidin-2-yl)piperazin-1-yl)-5-((2-(1-methyl-1H-pyrazol-4-yl)pyrimidin-5-yl)ethynvllpyrimidine

To a solution of 52-3 (70 mg, 0.140 mmol) in DMF (1 mL) were added 1-fluoro-2-iodoethane (73.3 mg. 0.421 mmol), NaH (11.23 mg. 0.281 mmol) at 0° C. The reaction mixture was stirred at room temperature for 48 h. The reaction mixture was quenched with water (20 mL) and extracted with EtOAc (2×30 mL). Combined organic layer was washed with brine (10 mL), dried over anhydrous Na₂SO₄, filtered and concentrated under reduced pressure. Crude compound was purified by Prep-HPLC purification (conditions: Mobile Phase—10 mM Ammonium Bicarbonate IN H₂O: MeCN Column Name Betasil Phenyl Hexyl (21.2×250)MM, 5μ Gradient Method: 0/45, 5/50, 15/50, 15.05/100, 18.05/100, 18.10/45, 21/45). Pure fractions were combined and lyophilized to afford 52. M/Z (ESI): 545.26 [M+H]⁺.

¹H NMR (400 MHz, DMSO-d₆) δ=8.89 (s, 2H), 8.63 (s, 2H), 8.38-8.44 (m, 3H), 8.05 (s, 1H), 6.68 (t. J=4.8 Hz, 1H), 4.85 (br s, 1H), 4.77-4.85 (m, 1H), 4.38-4.60 (m, 4H), 3.92 (s, 3H), 3.61-3.67 (m, 1H), 3.53-3.59 (m, 1H), 3.44-3.56 (m, 6H), 3.36-3.39 (m, 1H), 3.22-3.29 (m, 2H).

### Example 53

### (R)-2-(3-((2-fluoroethoxy)methyl)-4-(pyrimidin-4-yl)piperazin-1-yl)-5-((2-(1-methyl-1H-pyrazol-4-yl)pyrimidin-5-yl)ethynyl)pyrimidine

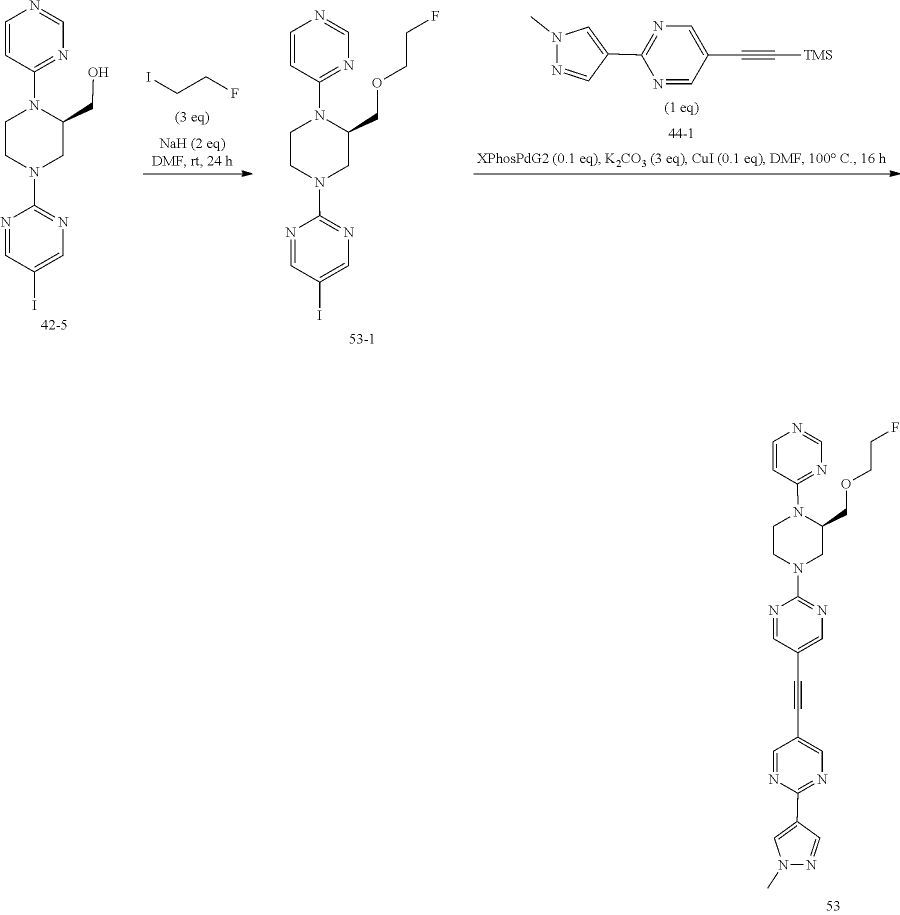

### Synthesis of 53-1: (R)-2-(3-((2-fluoroethoxy)methyl)-4-(pyrimidin-4-yl)piperazin-1-yl)-5-iodopyrimidine

To a stirred solution of 42-5 (2 g, 5.02 mmol) in DMF (30 mL) were added NaH (241 mg, 10 mmol) and 1-fluoro-2-iodoethane (2.62 g, 15.1 mmol) at room temperature. The reaction mixture was stirred at room temperature for 24 h. Reaction mixture was quenched with water (50 mL) and extracted with EtOAc (2×75 mL). Combined organic layer was washed with brine (2×30 mL), dried over anhydrous Na₂SO₄, filtered and concentrated under reduced pressure. Crude compound was purified by Biotage using 80 g silica (230-400 mesh) cartridge and compound eluted with 50% EtOAc in pet ether. Pure fractions were combined and concentrated under reduced pressure to afford 53-1. M/Z (ESI): 445.18 [M+H]⁺.

### Synthesis of 53: (R)-2-(3-((2-fluoroethoxy)methyl)-4-(pyrimidin-4-yl)piperazin-1-yl)-5-((2-(1-methyl-1H-pyrazol-4-yl)pyrimidin-5-yl)ethynyl)pyrimidine

To a stirred solution of 53-1 (60 mg, 135 mol) in DMF (1 mL) were added 44-1 (34.6 mg. 135 mol), K₂CO₃ (56.0 mg, 405 mol) and CuI (2.57 mg, 13.5 mol) at room temperature. Reaction mixture was degassed and purged with argon gas for 15 min. Then to this reaction mixture was added XPhos Palladacycle (10.6 mg, 13.5 mol) at room temperature. The reaction mixture was stirred at 100° C. for 16 h in a sealed tube. Reaction mixture was quenched with aqueous saturated Na₂CO₃ (20 mL) and extracted with 10% MeOH in DCM (2×35 mL). Combined organic layer was washed with brine (2×20 mL), dried over anhydrous Na₂SO₄, filtered and concentrated under reduced pressure. Crude compound was purified by prep-HPLC purification (conditions: Mobile Phase—10 mM Ammonium Bicarbonate IN H₂O: MeCN, COLUMN—X-Bridge C18 (19×250) mm 5u, Flow-16 ml/min, Gradient Method-0/35,4/45,8.6/45,8.65/100,11/100,11.05/45,14/45). Pure fractions were combined and concentrated under reduced pressure to afford 53. M/Z (ESI): 501.33 [M+H]⁺.

¹H NMR (400 MHz, DMSO-d₆) δ (ppm)=7.30-10.60 (m, 8H), 6.99 (br s, 1H), 4.55-4.87 (m, 2H), 4.19-4.56 (m, 4H), 3.92 (s, 3H), 3.49-3.71 (m, 4H), 3.42 (dd, J=13.4 Hz, 3.8 Hz, 1H), 3.32 (s, 2H).

### Example 54

### (R)-2-(1-(2-fluoroethyl)-1H-pyrazol-4-yl)-5-((2-(3-(methoxymethyl)-4-(pyrimidin-4-yl)piperazin-1-yl)pyrimidin-5-yl)ethynyl)pyrimidine

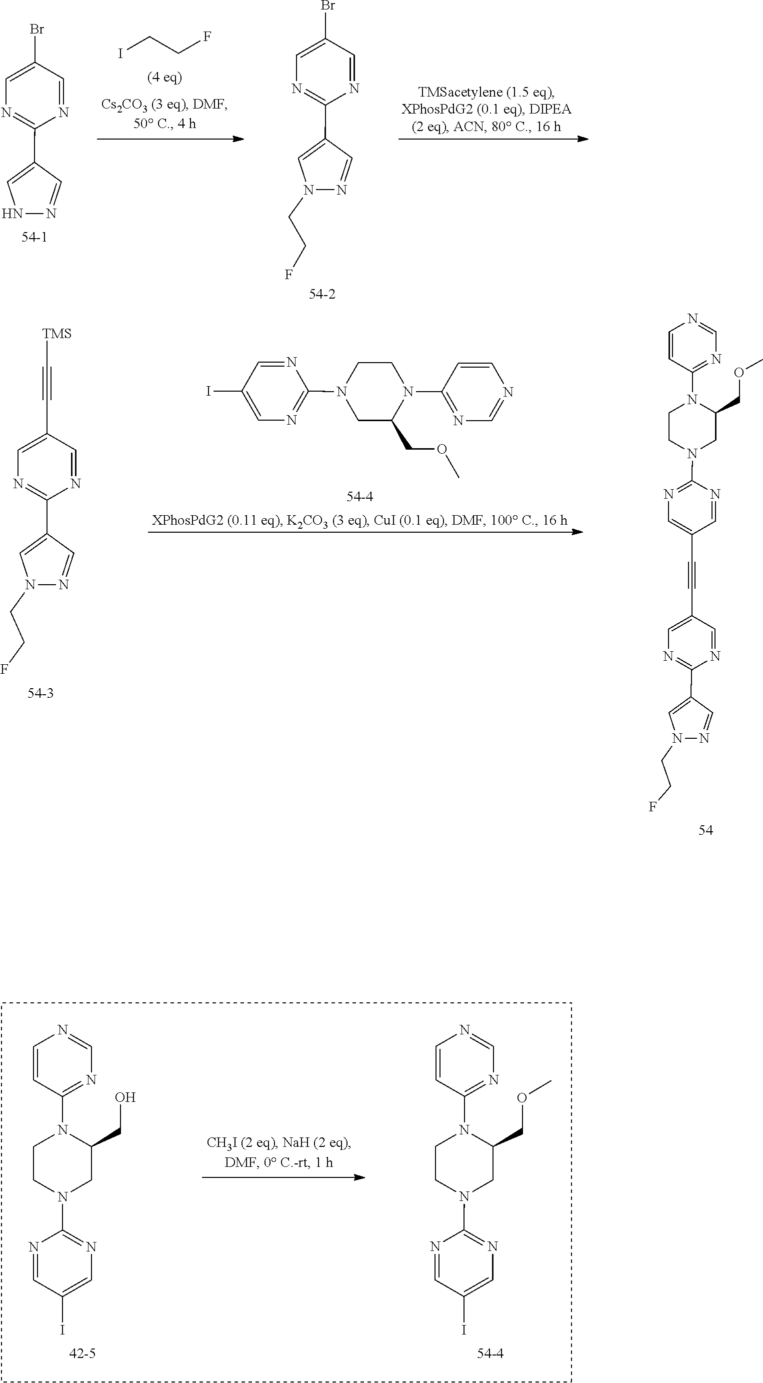

### Synthesis of 54-2: 5-bromo-2-(1-(2-fluoroethyl)-1H-pyrazol-4-ylpyrimidine

To a stirred solution of 5-bromo-2-(1H-pyrazol-4-yl)pyrimidine (54-1) (1 g, 3.82 mmol) in DMF (15 mL) were added 1-fluoro-2-iodoethane (2.66 g, 15.3 mmol) and Cs₂CO₃ (3.74 g, 11.5 mmol) at room temperature. The reaction mixture was stirred at 50° C. for 4 h under nitrogen atmosphere in a sealed tube. Reaction mixture was quenched with water (20 mL) and extracted with EtOAc (2×25 mL). Combined organic layer was washed with brine (2×20 mL), dried over anhydrous Na₂SO₄, filtered and concentrated under reduced pressure. Crude compound was purified by Biotage using 24 g silica (230-400 mesh) cartridge and compound eluted with 50% EtOAc in hexane. Pure fractions were combined and concentrated under reduced pressure to afford 54-2. M/Z (ESI): 273.00 [M+H]⁺.

### Synthesis of 54-3: 2-(1-(2-fluoroethyl)-1H-pyrazol-4-yl)-5-((trimethylsilyl)ethynyl)pyrimidine

To a stirred solution of 54-2 (1 g. 3.69 mmol) in ACN (50 mL) were added ethynyl-trimethyl-silane (773 μL, 5.53 mmol) and DIPEA (1.29 mL, 7.38 mmol) at room temperature. Reaction mixture was degassed and purged with argon gas for 15 min. Then to this reaction mixture was added XPhos Palladacycle (290 mg, 369 mol) at room temperature. The reaction mixture was stirred at 80° C. for 16 h under nitrogen atmosphere in a sealed tube. Reaction mixture was concentrated under reduced pressure. Crude compound was purified by Biotage using 40 g silica (230-400 mesh) cartridge and compound eluted with 50% of EtOAc in hexane. Pure fractions were combined and concentrated under reduced pressure to afford 54-3. M/Z (ESI): 289.14 [M+H]⁺.

### Synthesis of 54-4: (R)-5-iodo-2-(3-(methoxymethyl)-4-(pyrimidin-4-yl)piperazin-1-yl)pyrimidine

To a stirred solution of 42-5 (5 g. 12.6 mmol) in DMF (50 mL) were added NaH (603 mg, 25.1 mmol) and methyl iodide (1.62 mL, 25.1 mmol) at 0° C. The reaction mixture was stirred at room temperature for 1 h. Reaction mixture was quenched with water (50 mL), precipitated solid was filtered, washed with water and dried with under reduced pressure to afford 54-4. M/Z (ESI): 413.18 [M+H]⁺.

### Synthesis of 54: (R,E)-2-(2-((2-fluoroethoxy)methyl)-4-(5-(2-(2-(1-methyl-1H-pyrazol-4-yl)pyrimidin-5-yl)vinyl)pyrimidin-2-3l)piperazin-1-yl)-1,3,5-triazine

To a stirred solution of 54-3 (50 mg, 121 mol) in DMF (1 mL) were added K₂CO₃ (50.3 mg. 364 mol). 54-4 (42 mg, 146 mol) and CuI (2.31 mg, 12.1 mol) at room temperature. Reaction mixture was degassed and purged with argon gas for 15 min. Then to this added XPhos Palladacycle (9.54 mg, 12.1 μmol) at room temperature. The reaction mixture was stirred at 100° C. for 16 h in a sealed tube. Reaction mixture was quenched with water (20 mL) and extracted with EtOAc (2×40 mL). Combined organic layer was washed with brine (2×20 mL), dried over anhydrous Na₂SO₄, filtered and concentrated under reduced pressure. Crude compound was purified by prep-HPLC purification (conditions: Mobile Phase—10 mM Ammonium Bicarbonate IN H₂O: MeCN, COLUMN—X-Bridge C18 (19×250) mm 5u, Flow-16 ml/min, Gradient Method- 0/35,4/45,8.6/45,8.65/100,11/100,11.05/45,14/45). Pure fractions were combined and concentrated under reduced pressure to afford 54. M/Z (ESI): 501.34 [M+H]⁻.

¹H NMR (400 MHz, DMSO-d₆) δ (ppm)=8.91 (s, 2H), 8.65 (s, 2H), 8.54 (br s, 1H), 8.50 (s, 1H), 8.26 (br s, 1H), 8.13 (s, 1H), 6.87 (s, 1H), 4.55-4.94 (m, 5H), 4.50 (t, J=4.6 Hz. 2H), 4.30 (br s, 1H), 3.37-3.45 (m, 3H), 3.27-3.30 (m, 2H), 3.21 (s, 3H).

### Example 55

### (R)-5-(5-((2-(3-((2-fluoroethoxy)methyl)-4-(1,3,5-triazin-2-yl)piperazin-1-yl)pyrimidin-5-yl)ethynyl)pyrimidin-2-yl)oxazole

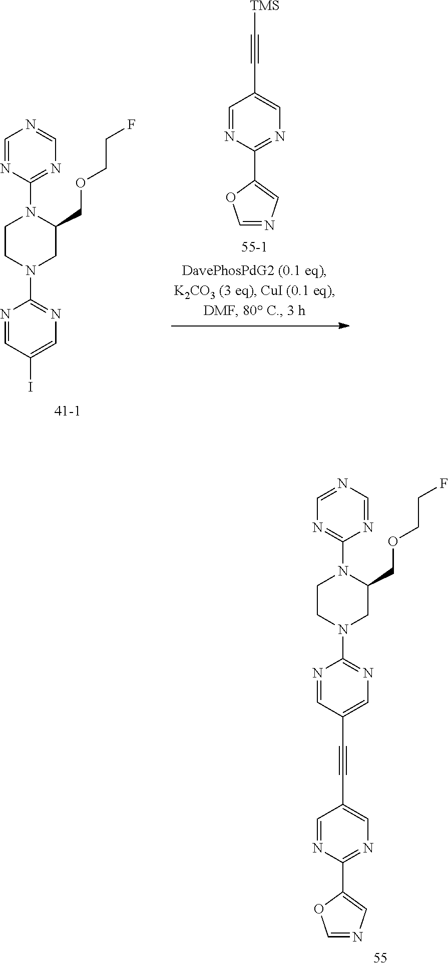 [SMILES: FCCOC[C@H]1CN(c2ncc(I)cn2)CCN1c1ncncn1 ; C[Si](C)(C)C#Cc1cnc(-c2cnco2)nc1 ; FCCOC[C@H]1CN(c2ncc(C#Cc3cnc(-c4cnco4)nc3)cn2)CCN1c1ncncn1]

### Synthesis of 55: (R)-5-(5-((2-(3-((2-fluoroethoxy)methyl)-4-(1,3,5-triazin-2-yl)piperazin-1-yl)pyrimidin-5-yl)ethynyl)pyrimidin-2-yl)oxazole

To a stirred solution of 41-1 (150.0 mg, 336.9 mol) and 55-1 (made in an analogous manner as 48-2) (81.98 mg, 336.9 mol) in DMF (3 mL) was added K₂CO₃ (139.7 mg. 1.011 mmol) at room temperature and degassed with N₂ gas at room temperature for 5 min. Then copper(I) iodide (6.416 mg, 33.69 μmol) and 2-dicyclohexylphosphino-2-(N,N-dimethylamino)biphenyl(2′-amino-1,1′-biphenyl-2-yl) palladium(II) (23.74 mg, 33.69 μmol) were added to the reaction mixture at room temperature. The reaction mixture was stirred at 80° C. for 3 h. Reaction mixture was quenched with water (10 mL) and extracted with DCM (2×10 mL). Combined organic layer was washed with brine (20 mL), dried over anhydrous Na₂SO₄, filtered and concentrated under reduced pressure. Crude compound was purified by Biotage using 24 g silica cartridge and compound eluted with 40% EtOAc in hexane. Pure fractions were combined, concentrated under reduced pressure. Obtained compound was re-purified by Prep HPLC purification (conditions: Mobile Phase—10 mM Ammonium Bicarbonate IN H₂O: MeCN COLUMN—X-Bridge C18 (19×250) mm, 5μ Flow-15.0 ml/min Gradient Method-0/40, 2/40, 7/53, 10/53, 10.05/100, 12/100, 12.05/40, 15/40). Pure fractions were combined, concentrated under reduced pressure and lyophilized to afford 55. M/Z (ESI): 489.26 [M+H]⁺.

¹H NMR (400 MHz, DMSO-d₆): δ (ppm) 9.04 (s, 2H), 8.58-8.75 (m, 5H), 8.04 (s, 1H), 4.91-5.02 (m, 1H), 4.80 (d, J=13.6 Hz, 1H), 4.54-4.64 (m, 2H), 4.30-4.52 (m, 2H), 3.54-3.69 (m, 4H), 3.40-3.45 (m, 1H), 3.22-3.30 (m, 2H).

### Example 56

### (R)-2-(4-(5-((2-(1-(fluoromethyl)-1H-pyrazol-4-yl)pyrimidin-5-yl)ethynyl)pyrimidin-2-yl)-2-(methoxymethyl)piperazin-1-yl)-1,3,5-triazine

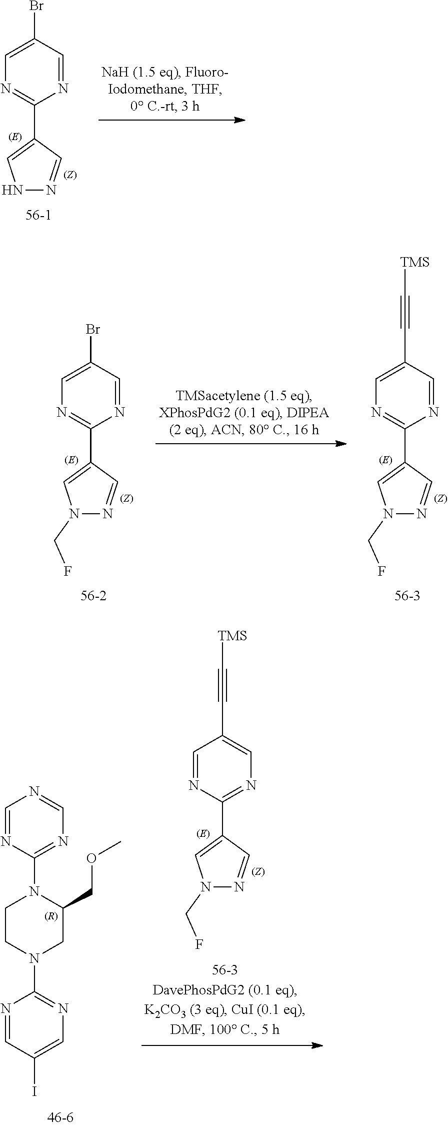

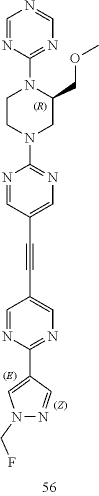

### Synthesis of 56-2: 5-bromo-2-(1-(fluoromethyl)-1H-pyrazol-4-yl)pyrimidine

To a solution of 5-bromo-2-(1H-pyrazol-4-yl)pyrimidine (54-1) (1.00 g. 4.44 mmol) in anhydrous THF (20 mL) was added NaH (160 mg, 6.67 mmol) at 0° C. The reaction mixture was stirred at 0° C. for 30 min. Then fluoroiodomethane (330 μL, 4.89 mmol) was added dropwise to the reaction mixture at 0° C. The reaction mixture was stirred at 25° C. for 3 h. Reaction mixture was quenched with water (50 mL) and extracted with ethyl acetate (2×50 mL). Combined organic layer was dried over anhydrous Na₂SO₄, filtered and concentrated under reduced pressure. Crude compound was purified by Biotage using silica gel column and compound eluted with 20% ethyl acetate in hexane. Pure fractions were combined and concentrated under reduced pressure to afford 56-2. M/Z (ESI): 257.04 [M+H]⁺.

### Synthesis of 56-3: 2-(1-(fluoromethyl)-1H-pyrazol-4-yl)-5-((trimethylsilyl)ethynyl)pyrimidine

To a stirred solution of 56-2 (400 mg, 1.56 mmol) in acetonitrile (8 mL) was added DIPEA (542 μL, 3.11 mmol) at room temperature. The reaction mixture was stirred at room temperature and degassed with N₂ gas at room temperature. Then XPhos Palladacycle (122 mg, 156 μmol) and ethynyl-trimethyl-silane (326 μL, 2.33 mmol) were added to the reaction mixture at room temperature. The reaction mixture was stirred at 80° C. for 16 h. Reaction mixture was filtered through celite pad, washed with EtOAc and concentrated under reduced pressure. The residue was diluted with water (50 mL) and extracted with EtOAc (2×50 mL). Combined organic layer was washed with brine (50 mL), dried over anhydrous Na₂SO₄, filtered and concentrated under reduced pressure. Crude compound was purified by Biotage using 120 g silica cartridge and compound eluted with 15% EtOAc in hexane. Pure fractions were combined and concentrated under reduced pressure to afford 56-3. M/Z (ESI): 275.02 [M+H]⁺.

### Synthesis of 56: (R)-2-(4-(5-((2-(1-(fluoromethyl)-1H-pyrazol-4-yl)pyrimidin-5-yl)ethynyl)pyrimidin-2-yl)-2-(methoxymethyl)piperazin-1-yl)-1,3,5-triazine

To a stirred solution of 46-6 (150.0 mg, 363.0 mol) and 56-3 (99.60 mg, 363.0 mol) in DMF (4 mL) was added K₂CO₃ (150.5 mg, 1.089 mmol) at room temperature and degassed with N₂ gas at room temperature for 5 min. Then copper(I) iodide (6.913 mg. 36.30 mol) and 2-dicyclohexylphosphino-2-(N,N-dimethylamino)biphenyl(2′-amino-1,1′-biphenyl-2-yl) palladium(II) (25.58 mg, 36.30 μmol) were added to the reaction mixture at room temperature. The reaction mixture was stirred at 100° C. for 5 h. Reaction mixture was filtered through celite pad, washed with DCM and concentrated under educed pressure. The residue was diluted with water (50 mL) and extracted with EtOAc (2×50 mL). Combined organic layer was washed with brine (50 mL), dried over anhydrous Na₂SO₄, filtered and concentrated under reduced pressure. Crude compound was purified by Biotage using 80 g silica cartridge and compound eluted with 8% MeOH in DCM. Pure fractions were combined, concentrated under reduced pressure and obtained compound was further re-purified by Prep HPLC purification (conditions: Mobile Phase—10 mM Ammonium Bicarbonate IN H₂O: MeCN COLUMN—X-Bridge C18 (19×250) mm, 5μ Flow-15.0 ml/min Gradient Method-0/42,6/50,10.45/50,10.5/100.14.5/100,14.55/42,17/42. APMS-008). Pure fractions were combined, concentrated under reduced pressure and lyophilized to afford 56. M/Z (ESI): 488.17 [M+H]⁻.

¹H NMR (400 MHz, DMSO-d₆): δ (ppm) 8.96 (s, 2H), 8.82 (s, 1H), 8.65 (d, J=4.4 Hz, 4H), 8.28 (s, 1H), 6.23 (d, J=52.4 Hz, 2H), 4.97-5.03 (m, 1H), 4.77 (d, J=13.6 Hz, 1H), 4.55-4.63 (m, 2H), 3.45 (d, J=7.2 Hz, 2H), 3.33-3.40 (m, 2H), 3.16-3.26 (m, 4H).

### Example 57

### (R)-2-(4-(5-((6-(1H-imidazol-1-yl)pyridin-3-yl)ethynyl)-6-fluoropyrazin-2-yl)-2-(methoxymethyl)piperazin-1-yl)pyrimidine

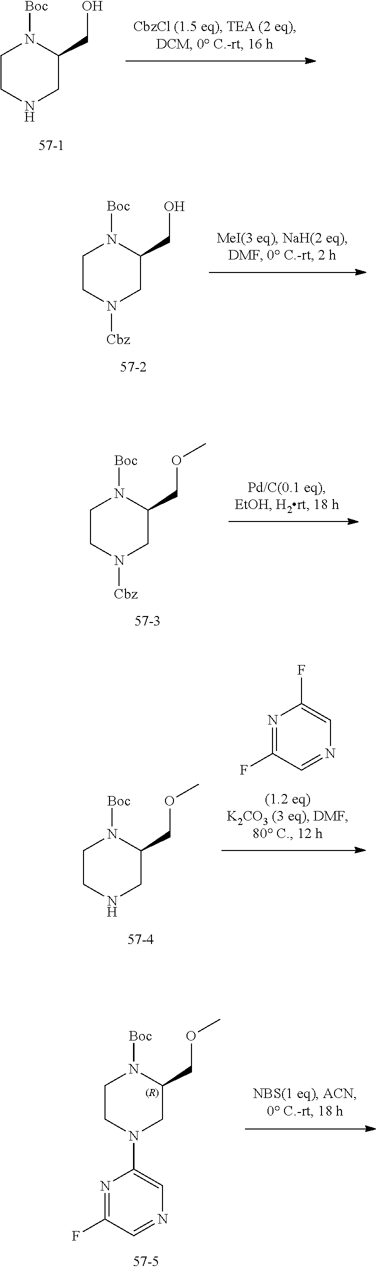

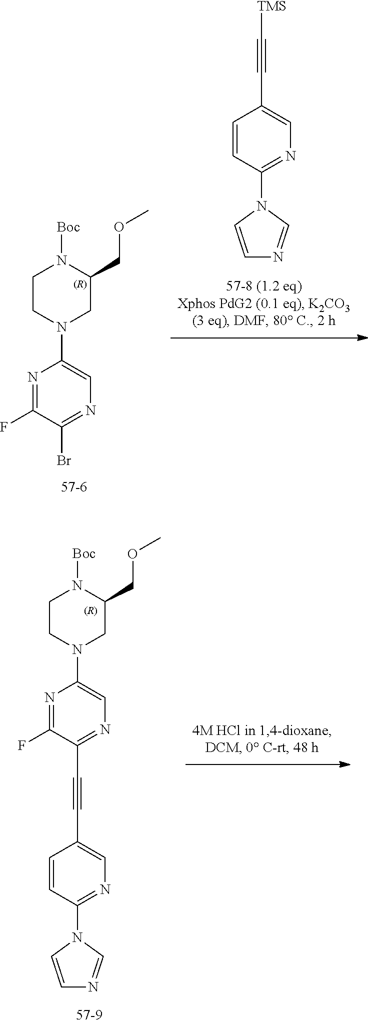

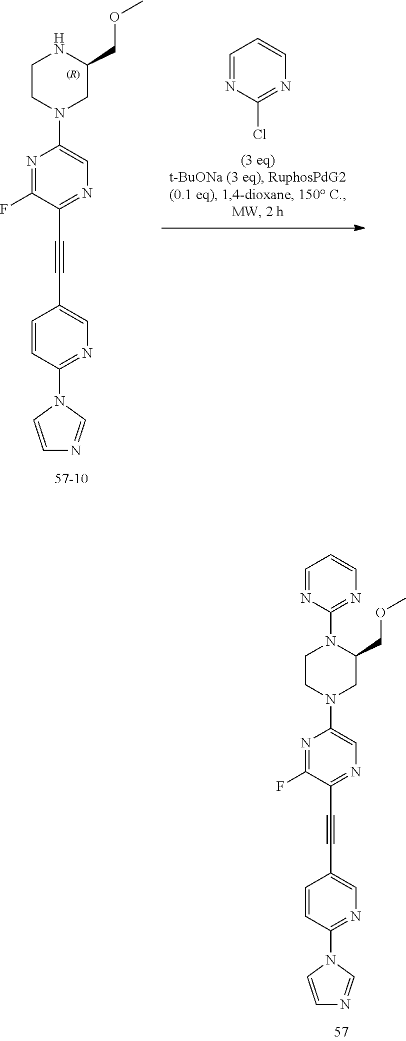

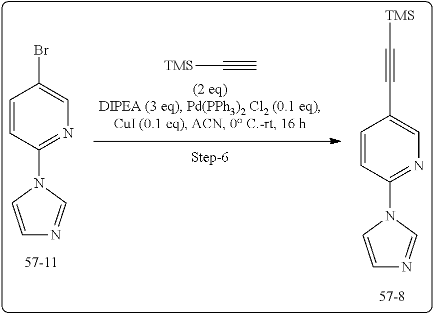

### Synthesis of 57-2: 4-benzyl 1-(tert-butyl) (R)-2-(hydroxymethyl)piperazine-1,4-dicarboxylate

To a stirred solution of tert-butyl (R)-2-(hydroxymethyl)piperazine-1-carboxylate (57-1) (50 g, 231 mmol) in DCM (700 mL) were added TEA (64.4 mL, 462 mmol) and Cbz-Cl (49.5 mL, 347 mmol) at 0° C. The reaction mixture was stirred at room temperature for 16 h under nitrogen atmosphere. The reaction mixture was quenched with water (250 mL) and extracted with EtOAc (2×500 mL). Combined organic layer was washed with aqueous saturated NaHCO₃ (2×100 mL) and brine (2×100 mL), was dried over anhydrous Na₂SO₄, filtered and concentrated under reduced pressure. Crude compound was purified by Biotage using 330 g silica (230-400 mesh) cartridge and compound eluted with 20% EtOAc in pet ether. Pure fraction were combined and concentrated under reduced pressure to afford 57-2.

¹H NMR (400 MHz, DMSO-d₆) δ (ppm)=7.28-7.42 (m, 5H), 5.09 (s, 2H), 4.81 (br s, 1H), 3.90-4.17 (m, 2H), 3.85 (d, J=10.0 Hz, 1H), 3.73 (d, J=10.8 Hz, 1H), 3.34-3.48 (m, 2H), 2.76-3.15 (m, 3H), 1.40 (s, 9H).

### Synthesis of 57-3: 4-benzyl 1-(tert-butyl) (R)-2-(methoxymethylI)piperazine-1,4-dicarboxylate

To a stirred solution of 57-2 (40 g, 114 mmol) in DMF (300 mL) were added NaH (9.13 g. 228 mmol) and Mel (21.41 mL, 342 mmol) at 0° C. The reaction mixture was stirred at 0° C. for 2 h under nitrogen atmosphere. The reaction mixture was quenched with ice cold water (250 mL) and extracted with EtOAc (2×650 ml). Combined organic layer was washed with brine (2×150 mL), dried over anhydrous Na₂SO₄, filtered and concentrated under reduced pressure. Crude compound was purified by Biotage using 330 g silica (230-400 mesh) cartridge and compound eluted with 20% EtOAc in pet ether. Pure fractions were combined and concentrated under reduced pressure to afford 57-3. M/Z (ESI): 309.17 [M+H]⁺. (tert-butyl breakage mass) Synthesis of 57-4: tert-butyl (R)-2-(methoxymethyl)piperazine-1-carboxylate

To a stirred solution of 57-3 (20 g, 54.9 mmol) in EtOH (200 mL) was added Pd-C (5.84 g. 5.49 mmol) at room temperature. The reaction mixture was stirred at room temperature for 18 h under hydrogen atmosphere. Reaction mixture was filtered through celite pad and residue was washed with ethyl acetate (2×200 mL). Filtrate was dried over anhydrous Na₂SO₄, filtered and concentrated under reduced pressure to afford 57-4. M/Z (ESI): 231.16 [M+H]⁺.

### Synthesis of 57-5: tert-butyl (R)-4-(6-fluoropyrazin-2-yl)-2-(methoxymethyl)piperazine-1-carboxylate

To a stirred solution of 57-4 (2 g, 8.68 mmol) in DMF (35 mL) were added K₂CO₃ (3.60 g, 26.1 mmol) and 2,6-difluoropyrazine (0.889 mL, 10.42 mmol) at room temperature. The reaction mixture was stirred at 80° C. for 12 h under nitrogen atmosphere. The reaction mixture was quenched with water (100 mL) and extracted with EtOAc (2×200 mL). Combined organic layer was dried over anhydrous Na₂SO₄, filtered and concentrated under reduced pressure. Crude compound was purified by Biotage using 120 g silica (230-400 mesh) cartridge and compound eluted with 30% EtOAc in pet ether. Pure fractions were combined and concentrated under reduced pressure to afford 57-5. M/Z (ESI): 327.25 [M+H]⁺.

### Synthesis of 57-6: tert-butyl (R)-4-(5-bromo-6-fluoropyrazin-2-yl)-2-(methoxymethyl)piperazine-1-carboxylate

To a stirred solution of 57-5 (1.5 g, 4.60 mmol) in ACN (45 mL) was added NBS (0.818 g, 4.60 mmol) at 0° C. The reaction mixture was stirred at room temperature for 18 h under nitrogen atmosphere. The reaction mixture was quenched with water (50 mL) and extracted with EtOAc (2×100 mL). Combined organic layer was washed with brine (2×50 mL), dried over anhydrous Na₂SO₄, filtered and concentrated under reduced pressure. Crude compound purified by Biotage using 80 g silica (230-400 mesh) cartridge and compound eluted with 18% EtOAc in pet ether. Pure fractions were combined and concentrated under reduced pressure to afford 57-6. M/Z (ESI): 405.09 [M+H]⁺.

### Synthesis of 57-8: 2-(1H-imidazol-1-yl)-5-((trimethylsilyl)ethynyl)pyridine

To a stirred solution of 5-bromo-2-(1H-imidazol-1-yl)pyridine (57-11) (1 g, 4.46 mmol) in ACN (40 mL) were added DIPEA (2.339 mL, 13.39 mmol) and copper (I) iodide (0.085 g, 0.446 mmol) at 0° C. The reaction mixture was degassed and purged with argon gas for 20 min. Then to this reaction mixture were added trimethylsilylacetylene (1.271 mL, 8.93 mmol) and bis-(triphenylphosphino)-palladous chloride (0.297 g. 0.446 mmol) at 0° C. The reaction mixture was stirred at room temperature for 16 h under nitrogen atmosphere. The reaction mixture was quenched with water (50 mL) and extracted with EtOAc (2×100 mL). Combined organic layer was washed with brine (2×50 mL), dried over anhydrous Na₂SO₄, filtered and concentrated under reduced pressure. Crude compound was purified by Biotage using 80 g silica (230-400 mesh) cartridge and compound was eluted with 60% EtOAc in pet ether. Pure fractions were combined and concentrated under reduced pressure to afford 57-8. M/Z (ESI): 242.42 [M+H]⁺.

### Synthesis of 57-9: tert-butyl (R)-4-(5-((6-(1H-imidazol-1-yl)pyridin-3-yl)ethynyl)-6-fluoropyrazin-2-yl)-2-(methoxymethyl)piperazine-1-carboxylate

To a stirred solution of 57-6 (1.5 g, 3.70 mmol) in DMF (15 mL) were added K₂CO₃ (1.535 g, 11.10 mmol) and 57-8 (1.072 g, 4.44 mmol) at room temperature. The reaction mixture was degassed and purged with argon gas for 25 min. Then to this reaction mixture was added XPhos Pd G2 (0.291 g, 0.370 mmol) at room temperature. The reaction mixture was stirred at 80° C. for 2 h under nitrogen atmosphere in a sealed tube. The reaction mixture was quenched with water (60 mL) and extracted with EtOAc (2×85 mL). Combined organic layer was washed with brine (2×30 mL), dried over anhydrous Na₂SO₄, filtered and concentrated under reduced pressure. Crude compound was purified by Biotage using 80 g silica (230-400 mesh) cartridge and compound eluted with 6% MeOH in DCM. Pure fractions were combined and concentrated under reduced pressure to afford 57-9. M/Z (ESI): 494.45 [M+H]⁺.

### Synthesis of 57-10: (R)-2-((6-(1H-imidazol-1-yl)pyridin-3-yl)ethynyl)-3-fluoro-5-(3-(methoxymethyl)piperazin-1-yl)pyrazine

To a stirred solution of 57-9 (750 mg, 1.520 mmol) in DCM (15 mL) was added 4M HCl in 1,4-dioxane (0.760 mL, 3.04 mmol) at 0° C. The reaction mixture was stirred at room temperature for 48 h under nitrogen atmosphere. Reaction mixture was concentrated under reduced pressure. Obtained compound was dissolved in 10% MeOH in DCM (50 mL) and added MP carbonate resin (2 g) for 20 min. The reaction mixture was filtered through celite pad and washed with 10% MeOH in DCM (2×20 mL). Combined organic layer was dried over anhydrous Na₂SO₄, filtered and concentrated under reduced pressure. Crude compound was purified by Biotage using 80 g silica (230-400 mesh) cartridge and compound eluted with 12% MeOH in DCM. Pure fractions were combined and concentrated under reduced pressure to afford 57-10. M/Z (ESI): 394.21[M+H]⁻.

### Synthesis of 57: (R)-2-(4-(5-((6-(1H-imidazol-1-yl)pyridin-3-yl)ethynyl)-6-fluoropyrazin-2-yl)-2-(methoxymethyl)piperazin-1-yl)pyrimidine

To a stirred solution of 57-10 (100 mg, 0.254 mmol) in 1,4-dioxane (1.5 mL) were added 2-chloropyrimidine (87 mg, 0.763 mmol) and sodium tert-butoxide (73.3 mg, 0.763 mmol) at room temperature. Reaction mixture was degassed and purged with argon gas for 20 min. Then to this reaction mixture was added RuPhos Pd G2 (19.74 mg, 0.025 mmol) at room temperature. The reaction mixture was stirred in a microwave at 150° C. for 2 h. Reaction mixture was quenched with water (25 mL) and extracted with EtOAc (2×45 mL). Combined organic layer was washed with brine (2×20 mL), dried over anhydrous Na₂SO₄, filtered and concentrated under reduced pressure. Crude compound was purified by 40 g silica (230-400 mesh) cartridge and compound eluted with 4% MeOH in DCM. Pure fractions were combined and concentrated under reduced pressure. Obtained compound was again purified by prep-HPLC purification (conditions: Mobile Phase—10 mM Ammonium Bicarbonate IN H₂O: MeCN, COLUMN—X-Bridge, C18 (10×250) mm, 5p, Flow-6.0 ml/min, Gradient Method:-0/40,5/55,10.3/58,10.4/100,11.9/100,12/40,16/40). Pure fractions were combined, concentrated under reduced pressure and lyophilized to afford 57. M/Z (ESI): 472.11 [M+H]+¹H NMR (400 MHz, DMSO-d₆) δ (ppm)=8.68-8.73 (m, 1H), 8.60 (s, 1H), 8.42 (d, J=4.8 Hz, 2H), 8.30 (d, J=5.6 Hz, 1H), 8.21 (dd, J=8.6 Hz, 2.2 Hz, 1H), 8.01 (t, J=1.2 Hz, 1H), 7.92 (d, J=8.8 Hz, 1H), 7.16 (s, 1H), 6.70 (t, J=4.6 Hz, 1H), 4.85-4.99 (m, 1H), 4.40-4.54 (m, 2H), 4.27 (d, J=12.0 Hz, 1H), 3.37-3.52 (m, 5H), 3.22 (s, 3H).

### Example 58

### (R)-5-((6-(4H-1,2,4-triazol-4-yl)pyridin-3-yl)ethynyl)-4-fluoro-2-(3-(methoxymethyl)-4-(pyrimidin-4-yl)piperazin-1-yl)pyrimidine

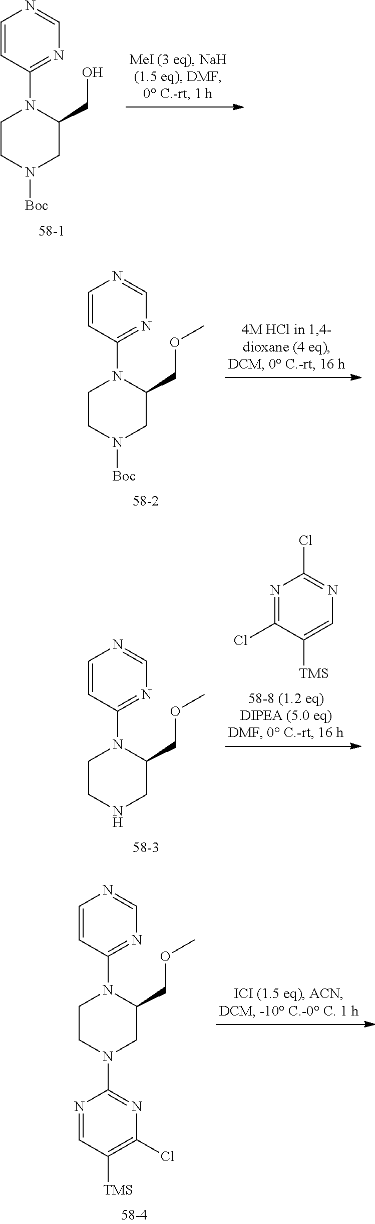 [SMILES: CC(C)(C)OC(=O)N1CCN(c2ccncn2)[C@@H](CO)C1 ; COC[C@H]1CN(C(=O)OC(C)(C)C)CCN1c1ccncn1 ; COC[C@H]1CNCCN1c1ccncn1 ; C[Si](C)(C)c1cnc(Cl)nc1Cl ; COC[C@H]1CN(c2ncc([Si](C)(C)C)c(Cl)n2)CCN1c1ccncn1]

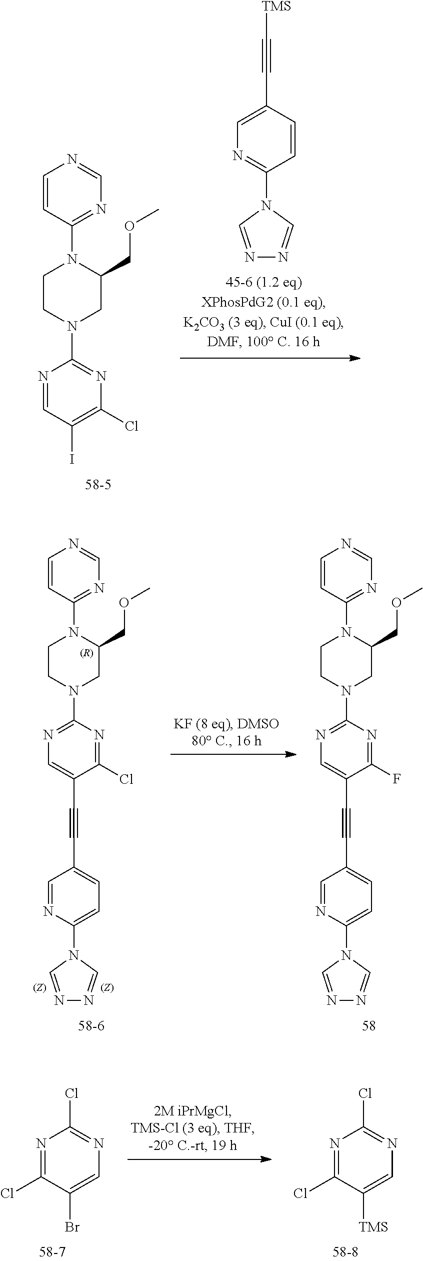

### Synthesis of 58-2: tert-butyl (R)-3-(methoxymethyl-4-(pyrimidin-4-yl)piperazine-1-carboxylate

To a stirred solution of tert-butyl (R)-3-(hydroxymethyl)-4-(pyrimidin-4-yl)piperazine-1-carboxylate (58-1) (5.00 g. 17.0 mmol) in DMF (20 mL) were added Mel (3.19 mL, 51.0 mmol) and 60% NaH in oil (612 mg, 25.5 mmol)) at 0° C. The reaction mixture was stirred at 25° C. for 1 h. The reaction mixture was quenched with water (100 mL) and extracted with EtOAc (2×100 mL). Combined organic layer was washed with brine (2×50 mL), dried over anhydrous Na₂SO₄, filtered and concentrated under reduced pressure. Crude compound was purified by Biotage using 80 g silica cartridge and compound eluted with 20% EtOAc in pet ether. Pure fractions were combined and concentrated under reduced pressure to afford 58-2. M/Z (ESI): 309.23 [M+H]⁺.

### Synthesis of 58-3: (R)-4-(2-(methoxymethyl)piperazin-1-yl)pyrimidine

To a stirred solution of 58-2 (2.10 g, 6.81 mmol) in DCM (20 mL) were added 4M hydrogen chloride in 1,4-dioxane (6.81 mL, 27.2 mmol) at 0° C. The reaction mixture was stirred at 25° C. for 16 h. Reaction mixture was concentrated under reduced pressure and residue was washed with diethyl ether (2×10 mL) and dried under reduced pressure to afford 58-3. M/Z (ESI): 209.18 [M+H]⁺.

### Synthesis of 58-8: 2,4-dichloro-5-(trimethylsilyl)pyrimidine

To a stirred solution of 5-bromo-2,4-dichloropyrimidine (10.0 g, 43.9 mmol) in THF (150 mL) were added 2M isopropyl magnesium chloride (21.9 mL, 43.9 mmol) at −20° C. Reaction mixture was stirred at 0° C., to 25° C. for 3 h. Then TMS-Cl (16.7 mL, 132 mmol) was added to the reaction mixture at −20° C. Then the reaction mixture was stirred at 0° C., to 25° C. for 16 h under nitrogen atmosphere. Reaction mixture was quenched with NH₄Cl (100 mL), extracted with EtOAc (2×120 mL). Combined organic layer was washed with brine (2×50 mL), dried over anhydrous Na₂SO₄, filtered and concentrated under reduced pressure. Crude compound was purified by Biotage using 120 g silica cartridge and compound eluted with 10% EtOAc in pet ether. Pure fractions were combined and concentrated under reduced pressure to afford 58-8. M/Z (ESI): 220.97 [M+H]⁺.

### Synthesis of 58-4: (R)-4-chloro-2-(3-(methoxymethyl)-4-(pyrimidin-4-yl)piperazin-1-yl)-5-(trimethylsilyl)pyrimidine

To a stirred solution of 58-3 (1.20 g, 5.76 mmol) in DMF (30 mL) were added 58-8 (1.53 g, 6.91 mmol) and DIPEA (5.02 mL, 28.8 mmol) at room temperature. The reaction mixture was stirred at 25° C. for 16 h under nitrogen atmosphere. Reaction mixture was quenched with water (100 mL), extracted with EtOAc (2×100 mL). Combined organic layer was washed with brine (2×50 mL), dried over anhydrous Na₂SO₄, filtered and concentrated under reduced pressure. Crude compound was purified by Biotage using 40 g silica cartridge and compound was eluted with 20% EtOAc in pet ether. Pure fractions were combined and concentrated under reduced pressure to afford 58-4. M/Z (ESI): 393.30 [M+H]⁺.

### Synthesis of 58-5: (R)-4-chloro-5-iodo-2-(3-(methoxymethyl)-4-(pyrimidin-4-yllpiperazin-1-yl)pyrimidine

To a stirred solution of 58-4 (800 mg, 2.04 mmol) in ACN (20 mL) and DCM (5 mL) were added iodine monochloride (197 μL, 3.05 mmol) at −10° C.-0° C. The reaction mixture was stirred at 0° C. for 1 h under nitrogen atmosphere. Reaction mixture was quenched with water (80 mL) and extracted with EtOAc (2×80 mL). Combined organic layer was washed with brine (2×10 mL), dried over anhydrous Na₂SO₄, filtered and concentrated under reduced pressure. Crude compound was purified by Biotage using 40 g silica cartridge and compound was eluted with 30% EtOAc in pet ether. Pure fractions were combined and concentrated under reduced pressure to afford 58-5. M/Z (ESI): 446.98 [M+H]⁺.

### Synthesis of 58-6: (R)-5-((6-(4H-1,2,4-triazol-4-yl)pyridin-3-yl)ethynyl)-4-chloro-2-(3-(methoxymethyl)-4-(pyrimidin-4-yl)piperazin-1-yl)pyrimidine

To a stirred solution of 58-5 (70.0 mg, 157 mol) in DMF (1 mL) were added 45-6 (45.6 mg, 188 mol). K₂CO₃ (65.0 mg, 470 mol) and CuI (2.98 mg, 15.7 mol) at room temperature and degassed with argon gas for 15 min. Then XPhos Palladacycle (12.3 mg, 15.7 μmol) was added to this reaction mixture at room temperature. The reaction mixture was stirred at 100° C. for 16 h in a sealed tube. Reaction mixture was quenched with water (80 mL) and extracted with EtOAc (2×80 mL). Combined organic layer was washed with brine (2×20 mL), dried over anhydrous Na₂SO₄, filtered and concentrated under reduced pressure. Crude compound was washed with diethyl ether (2×10 mL), dried under reduced pressure. Crude compound was purified by Prep HPLC purification (condition: Mobile Phase—10 mM Ammonium Bicarbonate IN H₂O: MeCN COLUMN—X-Bridge C18 (19×250) mm 5u Flow-16 ml/min Gradient Method- 0/35,4/45,8.6/45,8.65/100,11/100,11.05/45,14/45). Pure fractions were combined, concentrated under reduced pressure and lyophilized to afford 58-6. M/Z (ESI): 489.14 [M+H]⁺.

¹H NMR (400 MHz, DMSO-d₆) 3 (ppm)=9.34 (s, 2H), 8.64-8.78 (m, 2H), 8.54 (s, 1H), 8.19-8.31 (in, 2H), 7.98 (d, J=8.4 Hz. 1H), 6.86 (d, J=6.0 Hz, 1H), 4.19-4.90 (m, 4H), 3.38-3.50 (m, 5H), 3.21 (s, 3H).

### Synthesis of 58: (R)-5-((6-(4H-1,2,4-triazol-4-yl)pyridin-3-yl)ethynyl)-4-fluoro-2-(3-(methoxymethyl)-4-(pyrimidin-4-yl)piperazin-1-yl)pyrimidine

To a stirred solution of 58-6 (100.0 mg, 204.5 mol) in DMSO (2 mL) were added potassium fluoride (38.32 μL, 1.636 mmol) at room temperature. The reaction mixture was stirred at 80° C. for 16 h under nitrogen atmosphere. Reaction mixture was quenched with water (50 mL) and extracted with EtOAc (2×50 mL). Combined organic layer was washed with brine (2×20 mL), dried over anhydrous Na₂SO₄, filtered and concentrated under reduced pressure.

Crude compound was purified by prep HPLC purification (condition: Mobile Phase—10 mM Ammonium Bicarbonate IN H₂O: MeCN COLUMN—X-Bridge C18 (19×250) mm 5u Flow-16 ml/min Gradient Method- 0/35,4/45,8.6/45,8.65/100,11/100,11.05/45,14/45). Pure fractions were combined, concentrated under reduced pressure and lyophilized to afford 58. M/Z (ESI): 473.14 [M+H]⁺.

¹H NMR (400 MHz, DMSO-d₆) δ (ppm)=9.35 (d, J=3.6 Hz, 2H), 8.68-8.82 (m, 2H), 8.54 (s, 1H), 8.16-8.35 (m, 2H), 7.97 (d, J=8.4 Hz, 1H), 6.86 (d, J=6.0 Hz, 1H), 4.20-4.92 (m, 4H), 3.36-3.50 (m, 5H), 3.20 (s, 3H).

### Example 59

### (R)-5-((6-(3-fluoro-4H-1,2,4-triazol-4-yl)pyridin-3-yl)ethynyl)-2-(3-(methoxymethyl)-4-(pyrimidin-2-yl)piperazin-1-yl)pyrimidine

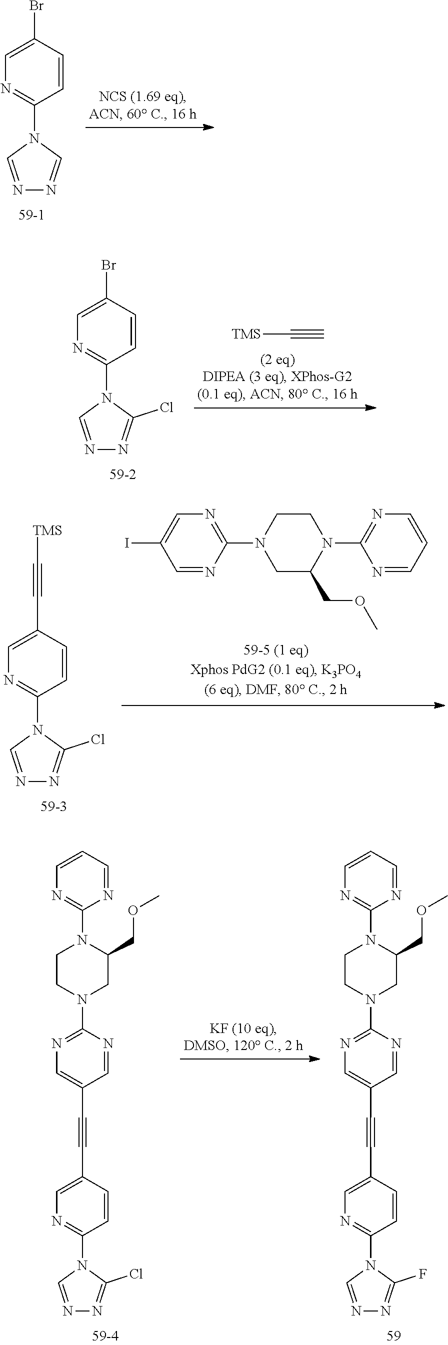

### Synthesis of 59-2: 5-bromo-2-(3-chloro-4H-1,2,4-triazol-4-yl)pyridine

To a solution of 5-bromo-2-(4H-1,2,4-triazol-4-yl)pyridine (59-1) (3 g, 13.33 mmol) in ACN (50 mL) was added NCS (3 g, 22.47 mmol) at room temperature. The reaction mixture was stirred at 60° C. for 16 h under argon atmosphere. Reaction mixture was concentrated under reduced pressure. Crude compound was purified by Biotage using 48 g silica (230-400 mesh) cartridge and compound eluted with 45% EtOAc in pet ether. Pure fractions were combined and concentrated under reduced pressure to afford 59-2. M/Z (ESI): 261.00 [M+H]⁻.

### Synthesis of 59: 2-(3-chloro-4H-1,2,4-triazol-4-yl)-5-((trimethylsilyl)ethynyl)pyridine

To a stirred solution of 59-2 (2.5 g, 9.63 mmol) in ACN (40 mL) was added DIPEA (5 mL, 28.6 mmol) at room temperature. Reaction mixture was degassed and purged with argon gas for 15 min. Then to this reaction mixture were added ethynyltrimethylsilane (2.75 mL, 19.32 mmol) and XPhos Pd G2 (0.758 g. 0.963 mmol) at room temperature. The reaction mixture was stirred at 80° C. for 16 h in a sealed tube. Reaction mixture was concentrated under reduced pressure. Crude compound was purified by Biotage using 80 g silica (230-400 mesh) cartridge and compound eluted with 50% EtOAc in pet ether. Pure fractions were combined and concentrated under reduced pressure to afford 59-3. M/Z (ESI): 276.95 [M+H]⁻.

### Synthesis of 59-4: (R)-5-((6-(3-chloro-4H-1,2,4-triazol-4-yl)pyridin-3-yl)ethynyl)-2-(3-(methoxymethyl-4-(pyrimidin-2-yl)piperazin-1-yl)pyrimidine

To a solution of 59-5 (made in an analogous manner as Int C) (700 mg, 1.698 mmol) in DMF (10 mL) were added 59-3 (611 mg, 2.207 mmol), tripotassium phosphate (2163 mg, 10.19 mmol) at room temperature. Reaction mixture was degassed and purged with argon gas for 10 min. Then to this reaction mixture was added chloro(2-dicyclohexylphosphino-2′,4′,6′-triisopropyl-1,1′-biphenyl)[2-(2′-amino-1,1′-biphenyl)]palladium(II) (134 mg, 0.170 mmol) at room temperature. The reaction mixture was stirred at 80° C. for 2 h in a sealed tube. Reaction mixture was quenched with water (50 mL), filtered through celite pad, washed with DCM (50 mL). Aqueous layer was extracted with DCM (2×50 mL), combined organic layer was washed with brine (50 mL), dried over Na₂SO₄, filtered and concentrated under reduced pressure. Crude compound was purified by Biotage using 48 g silica (230-400 mesh) cartridge and compound eluted with 5% MeOH in DCM. Pure fractions were combined and concentrated under reduced pressure to afford 59-4. M/Z (ESI): 489.20 [M+H]⁺.

### Synthesis of 59: (R)-5-((6-(3-fluoro-4H-1,2,4-triazol-4-yl)pyridin-3-yl)ethynyl)-2-(3-(methoxymethyl)-4-(pyrimidin-2-yl)piperazin-1-yl)pyrimidine

To a solution of 59-4 (100 mg, 0.205 mmol) in DMSO (1 mL) was added potassium fluoride (119 mg, 2.045 mmol) at room temperature. The reaction mixture was stirred at 120° C. for 12 h. Reaction mixture was quenched with ice cold water (5 mL), filtered and dried under reduced pressure. Crude compound was purified by prep-HPLC purification. Relatively pure fractions were combined, concentrated under reduced pressure. Obtained compound was re-purified by achiral SFC purification. M/Z (ESI): 473.16 [M+H]⁺.

¹H NMR (400 MHz, DMSO-d₆) S (ppm)=9.05 (s, 1H), 8.75 (dd, J=2.2 Hz, 0.6 Hz, 1H), 8.64 (s, 2H), 8.41 (d, J=4.8 Hz, 2H), 8.25 (dd, J=8.4 Hz, 2.4 Hz, 1H), 7.78 (d, J=7.6 Hz, 1H), 6.68 (t, J=4.8 Hz, 1H), 4.89-4.98 (m, 1H), 4.78 (d, J=13.6 Hz, 1H), 4.48-4.61 (m, 2H), 3.39-3.45 (m, 3H), 3.20 (s, 5H).

### Prep-HPLC Purification Conditions;

Instrument ID ANL-MCL5-PREP-023
Column Name XBRIDGE ODS 19*250.5 um
Column No #XBRIDGE ODS 19*250.5 um
Mobile Phase-A 10 mM Ammonium BiCarbonate in water
Mobile Phase-B Acetonitrile
Gradient program (T/% B) 0/48,2/48,10.20/65.10.25/100,13/100,13.05/48,17/48

Achiral SFC Purification Conditions:

| Column | YMC DIOL (4.6*250 mm)5 μm | |
|---|---|---|
| Co-solvent | METHANOL | |
| Total flow | 3 | mL/min |
| % of CO2 | 90 | |
| % of Co-Solvent | 10 | |
| ABPR | 1500 | psi |
| Temperature | 30° | C. |

The compounds contained in Table 2 were synthesized by analogous methods from synthetic sequences above as indicated in the last column in Table 2. Commercially available reagents were substituted where necessary to produce the examples below.

TABLE 2

| | | | Observed | | Example |
|---|---|---|---|---|---|
| Ex | | | Mass | Exact | Method |
| No. | Structure | Chemical Name | (M + H) | Mass | Used |
| 60 | 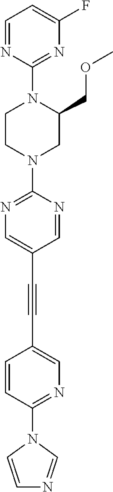 [SMILES: COC[C@H]1CN(c2ncc(C#Cc3ccc(-n4ccnc4)nc3)cn2)CCN1c1nccc(F)n1] | (R)-2-(4-(5-((6-(1H-imidazol-1- yl)pyridin-3-yl)ethynyl)pyrimidin-2- yl)-2-(methoxymethyl)piperazin-1- yl)-4-fluoropyrimidine | 472.37 | 471.50 | 12 |
| 61 | 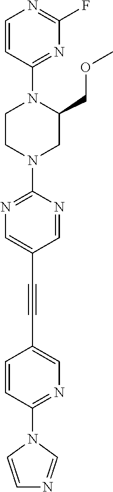 [SMILES: COC[C@H]1CN(c2ncc(C#Cc3ccc(-n4ccnc4)nc3)cn2)CCN1c1ccnc(F)n1] | (R)-4-(4-(5-((6-(1H-imidazol-1- yl)pyridin-3-yl)ethynyl)pyrimidin-2- yl)-2-(methoxymethyl)piperazin-1- yl)-2-fluoropyrimidine | 472.41 | 471.50 | 12 |
| 62 | 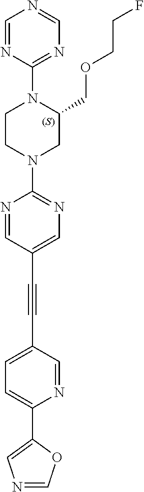 | (S)-5-(5-((2-(3-((2- fluoroethoxy)methyl)-4-(1,3,5- triazin-2-yl)piperazin-1- yl)pyrimidin-5-yl)ethynyl)pyridin-2- yl)oxazole | 488.31 | 487.19 | 44 |
| 63 | 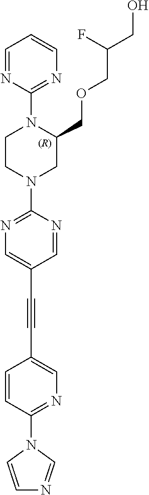 | 3-(((R)-4-(5-((6-(1H-imidazol-1- yl)pyridin-3-yl)ethynyl)pyrimidin-2- yl)-1-(pyrimidin-2-yl)piperazin-2- yl)methoxy)-2-fluoropropan-1-ol | 516.41 | 515.22 | 44 |
| 64 | 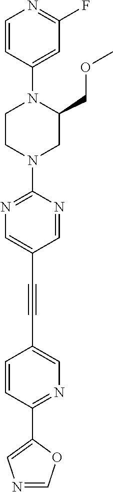 [SMILES: COC[C@H]1CN(c2ncc(C#Cc3ccc(-c4cnco4)nc3)cn2)CCN1c1ccnc(F)c1] | (R)-5-(5-((2-(4-(2-fluoropyridin-4- yl)-3-(methoxymethyl)piperazin-1- yl)pyrimidin-5-yl)ethynyl)pyridin-2- yl)oxazole | 472.39 | 471.18 | 44 |
| 65 | 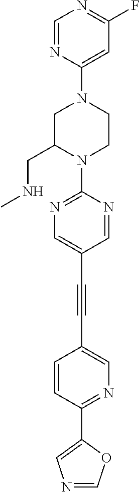 [SMILES: CNCC1CN(c2cc(F)ncn2)CCN1c1ncc(C#Cc2ccc(-c3cnco3)nc2)cn1] | 1-(4-(6-fluoropyrimidin-4-yl)-1-(5- ((6-(oxazol-5-yl)pyridin-3- yl)ethynyl)pyrimidin-2-yl)piperazin- 2-yl)-N-methylmethanamine | 472.31 | 471.19 | 44 |
| 66 | 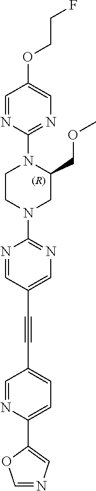 | (R)-5-(5-((2-(4-(5-(2- fluoroethoxy)pyrimidin-2-yl)-3- (methoxymethyl)piperazin-1- yl)pyrimidin-5-yl)ethynyl)pyridin-2- yl)oxazole | 517.35 | 516.20 | 44 |
| 67 | 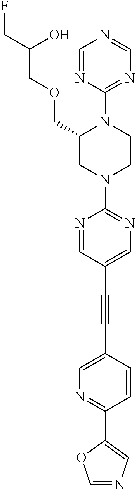 [SMILES: OC(CF)COC[C@H]1CN(c2ncc(C#Cc3ccc(-c4cnco4)nc3)cn2)CCN1c1ncncn1] | 1-fluoro-3-(((R)-4-(5-((6-(oxazol-5- yl)pyridin-3-yl)ethynyl)pyrimidin-2- yl)-1-(1,3,5-triazin-2-yl)piperazin-2- yl)methoxy)propan-2-ol | 518.31 | 517.20 | 44 |
| 68 | 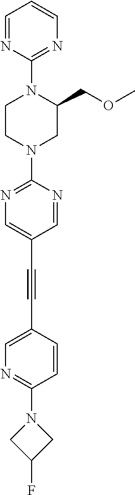 [SMILES: COC[C@H]1CN(c2ncc(C#Cc3ccc(N4CC(F)C4)nc3)cn2)CCN1c1ncccn1] | (R)-5-((6-(3-fluoroazetidin-1- yl)pyridin-3-yl)ethynyl)-2-(3- (methoxymethyl)-4-(pyrimidin-2- yl)piperazin-1-yl)pyrimidine | 461.4 | 460.21 | 44 |
| 69 | 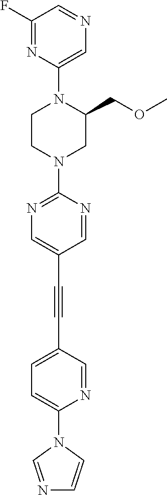 [SMILES: COC[C@H]1CN(c2ncc(C#Cc3ccc(-n4ccnc4)nc3)cn2)CCN1c1cncc(F)n1] | (R)-5-((6-(1H-imidazol-1-yl)pyridin- 3-yl)ethynyl)-2-(4-(6-fluoropyrazin- 2-yl)-3-(methoxymethyl)piperazin-1- yl)pyrimidine | 472.4 | 471.19 | 12 |
| 70 | 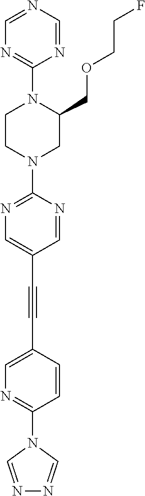 [SMILES: FCCOC[C@H]1CN(c2ncc(C#Cc3ccc(-n4cnnc4)nc3)cn2)CCN1c1ncncn1] | (R)-2-(4-(5-((6-(4H-1,2,4-triazol-4- yl)pyridin-3-yl)ethynyl)pyrimidin-2- yl)-2-((2- fluoroethoxy)methyl)piperazin-1-yl)- 1,3,5-triazine | 488.23 | 487.20 | 44 |
| 71 | 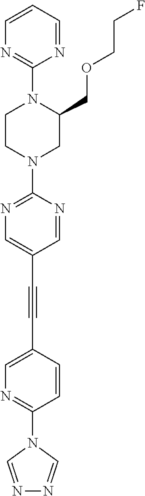 [SMILES: FCCOC[C@H]1CN(c2ncc(C#Cc3ccc(-n4cnnc4)nc3)cn2)CCN1c1ncccn1] | (R)-5-((6-(4H-1,2,4-triazol-4- yl)pyridin-3-yl)ethynyl)-2-(3-((2- fluoroethoxy)methyl)-4-(pyrimidin- 2-yl)piperazin-1-yl)pyrimidine | 487.28 | 486.20 | 44 |
| 72 | 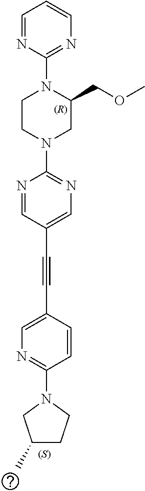 | 5-((6-((S)-3-fluoropyrrolidin-1- yl)pyridin-3-yl)ethynyl)-2-((R)-3- (methoxymethyl)-4-(pyrimidin-2- yl)piperazin-1-yl)pyrimidine | 475 | 474.23 | 44 |
| 73 | 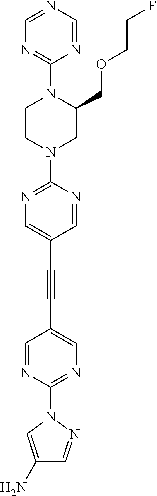 [SMILES: Nc1cnn(-c2ncc(C#Cc3cnc(N4CCN(c5ncncn5)[C@@H](COCCF)C4)nc3)cn2)c1] | (R)-1-(5-((2-(3-((2- fluoroethoxy)methyl)-4-(1,3,5- triazin-2-yl)piperazin-1- yl)pyrimidin-5-yl)ethynyl)pyrimidin- 2-yl)-1H-pyrazol-4-amine | 503.28 | 502.21 | 44 |
| 74 | 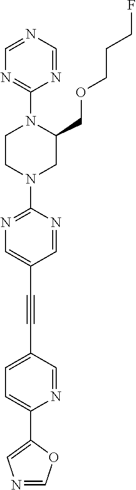 [SMILES: FCCCOC[C@H]1CN(c2ncc(C#Cc3ccc(-c4cnco4)nc3)cn2)CCN1c1ncncn1] | (R)-5-(5-((2-(3-((3- fluoropropoxy)methyl)-4-(1,3,5- triazin-2-yl)piperazin-1- yl)pyrimidin-5-yl)ethynyl)pyridin-2- yl)oxazole | 502.31 | 501.20 | 44 |
| 75 | 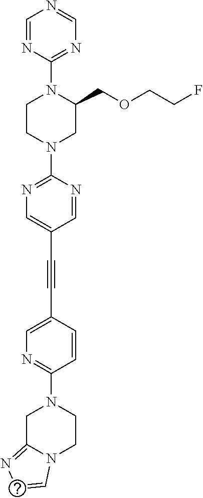 [SMILES: FCCOC[C@H]1CN(c2ncc(C#Cc3ccc(N4CCn5ccnc5C4)nc3)cn2)CCN1c1ncncn1] | (R)-7-(5-((2-(3-((2- fluoroethoxy)methyl)-4-(1,3,5- triazin-2-yl)piperazin-1- yl)pyrimidin-5-yl)ethynyl)pyridin-2- yl)-5,6,7,8-tetrahydro- [1,2,4]triazolo[4,3-a]pyrazine | 543.5 | 542.24 | 44 |
| 76 | 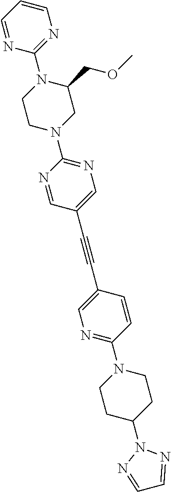 [SMILES: COC[C@H]1CN(c2ncc(C#Cc3ccc(N4CCC(n5nccn5)CC4)nc3)cn2)CCN1c1ncccn1] | (R)-5-((6-(4-(2H-1,2,3-triazol-2- yl)piperidin-1-yl)pyridin-3- yl)ethynyl)-2-(3-(methoxymethyl)-4- (pyrimidin-2-yl)piperazin-1- yl)pyrimidine | 538 | 537.27 | 44 |
| 77 | 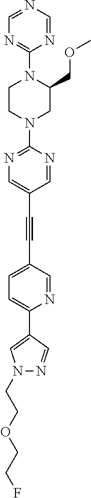 [SMILES: COC[C@H]1CN(c2ncc(C#Cc3ccc(-c4cnn(CCOCCF)c4)nc3)cn2)CCN1c1ncncn1] | (R)-2-(4-(5-((6-(1-(2-(2- fluoroethoxy)ethyl)-1H-pyrazol-4- yl)pyridin-3-yl)ethynyl)pyrimidin-2- yl)-2-(methoxymethyl)piperazin-1- yl)-1,3,5-triazine | 545.22 | 544.25 | 44 |
| 78 | 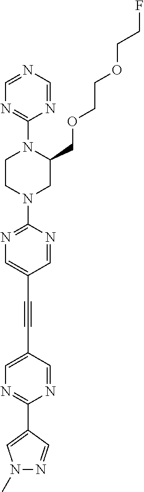 [SMILES: Cn1cc(-c2ncc(C#Cc3cnc(N4CCN(c5ncncn5)[C@@H](COCCOCCF)C4)nc3)cn2)cn1] | (R)-2-(2-((2-(2- fluoroethoxy)ethoxy)methyl)-4-(5- ((2-(1-methyl-1H-pyrazol-4- yl)pyrimidin-5-yl)ethynyl)pyrimidin- 2-yl)piperazin-1-yl)-1,3,5-triazine | 546.27 | 545.20 | 52 |
| 79 | 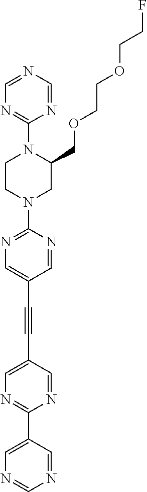 [SMILES: FCCOCCOC[C@H]1CN(c2ncc(C#Cc3cnc(-c4cncnc4)nc3)cn2)CCN1c1ncncn1] | (R)-5-((2-(3-((2-(2- fluoroethoxy)ethoxy)methyl)-4- (1,3,5-triazin-2-yl)piperazin-1- yl)pyrimidin-5-yl)ethynyl)-2,5′- bipyrimidine | 544.34 | 543.23 | 52 |
| 80 | 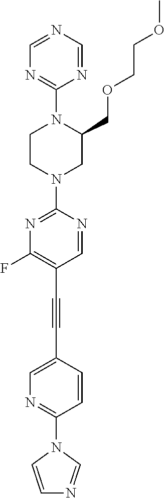 [SMILES: COCCOC[C@H]1CN(c2ncc(C#Cc3ccc(-n4ccnc4)nc3)c(F)n2)CCN1c1ncncn1] | (R)-2-(4-(5-((6-(1H-imidazol-1- yl)pyridin-3-yl)ethynyl)-4- fluoropyrimidin-2-yl)-2-((2- methoxyethoxy)methyl)piperazin-1- yl)-1,3,5-triazine | 517.28 | 516.21 | 96 |
| 81 | 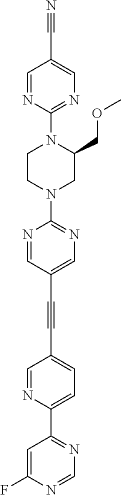 [SMILES: COC[C@H]1CN(c2ncc(C#Cc3ccc(-c4cc(F)ncn4)nc3)cn2)CCN1c1ncc(C#N)cn1] | (R)-2-(4-(5-((6-(6-fluoropyrimidin-4- yl)pyridin-3-yl)ethynyl)pyrimidin-2- yl)-2-(methoxymethyl)piperazin-1- yl)pyrimidine-5-carbonitrile | 509.29 | 508.19 | 44 |
| 82 | 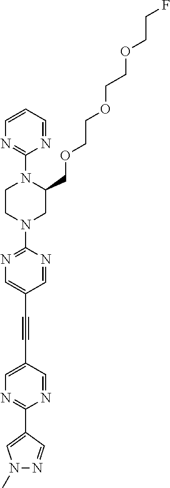 [SMILES: Cn1cc(-c2ncc(C#Cc3cnc(N4CCN(c5ncccn5)[C@@H](COCCOCCOCCF)C4)nc3)cn2)cn1] | (R)-2-(3-((2-(2-(2- fluoroethoxy)ethoxy)ethoxy)methyl)- 4-(pyrimidin-2-yl)piperazin-1-yl)-5- ((2-(1-methyl-1H-pyrazol-4- yl)pyrimidin-5-yl)ethynyl)pyrimidine | 589.47 | 588.27 | 44 |
| 83 | 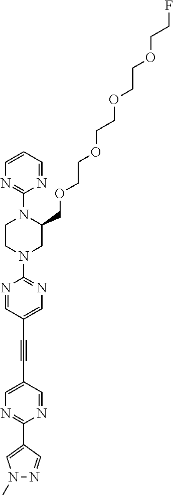 [SMILES: Cn1cc(-c2ncc(C#Cc3cnc(N4CCN(c5ncccn5)[C@@H](COCCOCCOCCOCCF)C4)nc3)cn2)cn1] | (R)-2-(3-(13-fluoro-2,5,8,11- tetraoxatridecyl)-4-(pyrimidin-2- yl)piperazin-1-yl)-5-((2-(1-methyl- 1H-pyrazol-4-yl)pyrimidin-5- yl)ethynyl)pyrimidine | 633.35 | 632.30 | 48 |
| 84 | 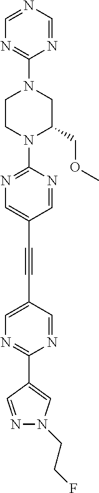 [SMILES: COC[C@H]1CN(c2ncncn2)CCN1c1ncc(C#Cc2cnc(-c3cnn(CCF)c3)nc2)cn1] | (R)-2-(4-(5-((2-(1-(2-fluoroethyl)- 1H-pyrazol-4-yl)pyrimidin-5- yl)ethynyl)pyrimidin-2-yl)-3- (methoxymethyl)piperazin-1-yl)- 1,3,5-triazine | 502.16 | 501.21 | 46 |
| 85 | 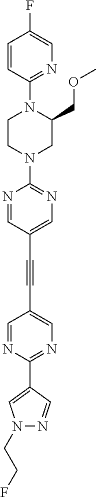 [SMILES: COC[C@H]1CN(c2ncc(C#Cc3cnc(-c4cnn(CCF)c4)nc3)cn2)CCN1c1ccc(F)cn1] | (R)-2-(1-(2-fluoroethyl)-1H-pyrazol- 4-yl)-5-((2-(4-(5-fluoropyridin-2-yl)- 3-(methoxymethyl)piperazin-1- yl)pyrimidin-5-yl)ethynyl)pyrimidine | 518.24 | 517.22 | 46 |
| 86 | 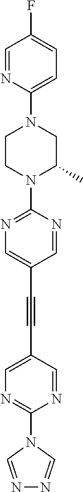 [SMILES: C[C@H]1CN(c2ccc(F)cn2)CCN1c1ncc(C#Cc2cnc(-n3cnnc3)nc2)cn1] | (S)-5-((2-(4H-1,2,4-triazol-4- yl)pyrimidin-5-yl)ethynyl)-2-(4-(5- fluoropyridin-2-yl)-2- methylpiperazin-1-yl)pyrimidine | 443.2 | 442.18 | 10 |
| 87 | 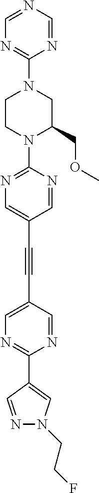 [SMILES: COC[C@@H]1CN(c2ncncn2)CCN1c1ncc(C#Cc2cnc(-c3cnn(CCF)c3)nc2)cn1] | (S)-2-(4-(5-((2-(1-(2-fluoroethyl)- 1H-pyrazol-4-yl)pyrimidin-5- yl)ethynyl)pyrimidin-2-yl)-3- (methoxymethyl)piperazin-1-yl)- 1,3,5-triazine | 502.16 | 501.21 | 46 |
| 88 | 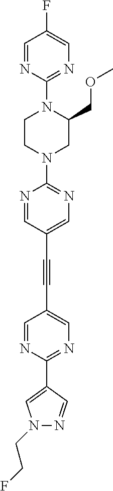 [SMILES: COC[C@H]1CN(c2ncc(C#Cc3cnc(-c4cnn(CCF)c4)nc3)cn2)CCN1c1ncc(F)cn1] | (R)-5-fluoro-2-(4-(5-((2-(1-(2- fluoroethyl)-1H-pyrazol-4- yl)pyrimidin-5-yl)ethynyl)pyrimidin- 2-yl)-2-(methoxymethyl)piperazin-1- yl)pyrimidine | 519.30 | 518.21 | 46 |
| 89 | 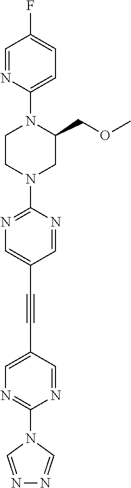 [SMILES: COC[C@H]1CN(c2ncc(C#Cc3cnc(-n4cnnc4)nc3)cn2)CCN1c1ccc(F)cn1] | (R)-5-((2-(4H-1,2,4-triazol-4- yl)pyrimidin-5-yl)ethynyl)-2-(4-(5- fluoropyridin-2-yl)-3- (methoxymethyl)piperazin-1- yl)pyrimidine | 473.2 | 472.19 | 10 |
| 90 | 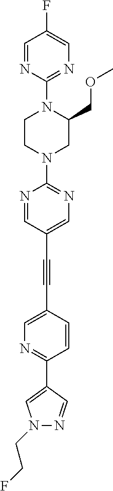 [SMILES: COC[C@H]1CN(c2ncc(C#Cc3ccc(-c4cnn(CCF)c4)nc3)cn2)CCN1c1ncc(F)cn1] | (R)-5-fluoro-2-(4-(5-((6-(1-(2- fluoroethyl)-1H-pyrazol-4- yl)pyridin-3-yl)ethynyl)pyrimidin-2- yl)-2-(methoxymethyl)piperazin-1- yl)pyrimidine | 518.33 | 517.22 | 46 |
| 91 | 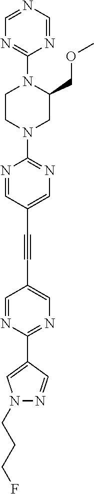 [SMILES: COC[C@H]1CN(c2ncc(C#Cc3cnc(-c4cnn(CCCF)c4)nc3)cn2)CCN1c1ncncn1] | (R)-2-(4-(5-((2-(1-(3-fluoropropyl)- 1H-pyrazol-4-yl)pyrimidin-5- yl)ethynyl)pyrimidin-2-yl)-2- (methoxymethyl)piperazin-1-yl)- 1,3,5-triazine | 516.36 | 515.23 | 46 |
| 92 | 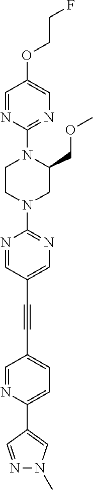 [SMILES: COC[C@H]1CN(c2ncc(C#Cc3ccc(-c4cnn(C)c4)nc3)cn2)CCN1c1ncc(OCCF)cn1] | (R)-5-(2-fluoroethoxy)-2-(2- (methoxymethyl)-4-(5-((6-(1-methyl- 1H-pyrazol-4-yl)pyridin-3- yl)ethynyl)pyrimidin-2-yl)piperazin- 1-yl)pyrimidine | 530.21 | 529.58 | 44 |
| 93 | 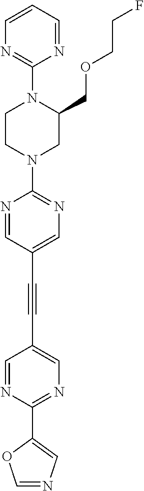 [SMILES: FCCOC[C@H]1CN(c2ncc(C#Cc3cnc(-c4cnco4)nc3)cn2)CCN1c1ncccn1] | (R)-5-(5-((2-(3-((2- fluoroethoxy)methyl)-4-(pyrimidin- 2-yl)piperazin-1-yl)pyrimidin-5- yl)ethynyl)pyrimidin-2-yl)oxazole | 488.29 | 487.19 | 55 |
| 94 | 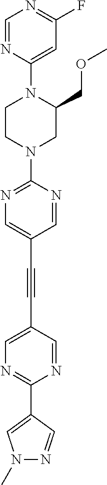 | (R)-2-(4-(6-fluoropyrimidin-4-yl)-3- (methoxymethyl)piperazin-1-yl)-5- ((2-(1-methyl-1H-pyrazol-4- yl)pyrimidin-5-yl)ethynyl)pyrimidine | 487.27 | 486.20 | 44 |
| 95 | 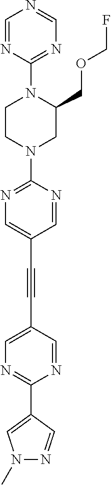 [SMILES: Cn1cc(-c2ncc(C#Cc3cnc(N4CCN(c5ncncn5)[C@@H](COCF)C4)nc3)cn2)cn1] | (R)-2-(2-((fluoromethoxy)methyl)-4- (5-((2-(1-methyl-1H-pyrazol-4- yl)pyrimidin-5-yl)ethynyl)pyrimidin- 2-yl)piperazin-1-yl)-1,3,5-triazine | 488.13 | 487.20 | 44 |
| 96 | 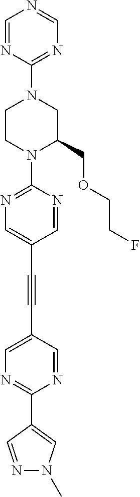 [SMILES: Cn1cc(-c2ncc(C#Cc3cnc(N4CCN(c5ncncn5)C[C@H]4COCCF)nc3)cn2)cn1] | (S)-2-(3-((2-fluoroethoxy)methyl)-4- (5-((2-(1-methyl-1H-pyrazol-4- yl)pyrimidin-5-yl)ethynyl)pyrimidin- 2-yl)piperazin-1-yl)-1,3,5-triazine | 502.16 | 501.21 | 44 |
| 97 | 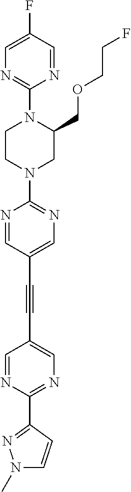 [SMILES: Cn1ccc(-c2ncc(C#Cc3cnc(N4CCN(c5ncc(F)cn5)[C@@H](COCCF)C4)nc3)cn2)n1] | (R)-5-fluoro-2-(2-((2- fluoroethoxy)methyl)-4-(5-((2-(1- methyl-1H-pyrazol-3-yl)pyrimidin-5- yl)ethynyl)pyrimidin-2-yl)piperazin- 1-yl)pyrimidine | 519.26 | 518.21 | 44 |
| 98 | 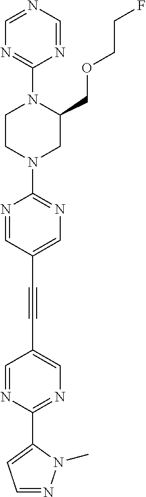 [SMILES: Cn1nccc1-c1ncc(C#Cc2cnc(N3CCN(c4ncncn4)[C@@H](COCCF)C3)nc2)cn1] | (R)-2-(2-((2-fluoroethoxy)methyl)-4- (5-((2-(1-methyl-1H-pyrazol-5- yl)pyrimidin-5-yl)ethynyl)pyrimidin- 2-yl)piperazin-1-yl)-1,3,5-triazine | 502.32 | 501.21 | 44 |

### Radiolabeling Procedures

### Synthesis of Radiolabeling Precursors

### Synthesis of 44-2: (R)-2-((4-(5-((2-(1-methyl-1H-pyrazol-4-yl)pyrimidin-5-yl)ethynyl)pyrimidin-2-yl)-1-(1,3,5-triazin-2-yl)piperazin-2-yl)methoxy)ethyl4-methylbenzenesulfonate

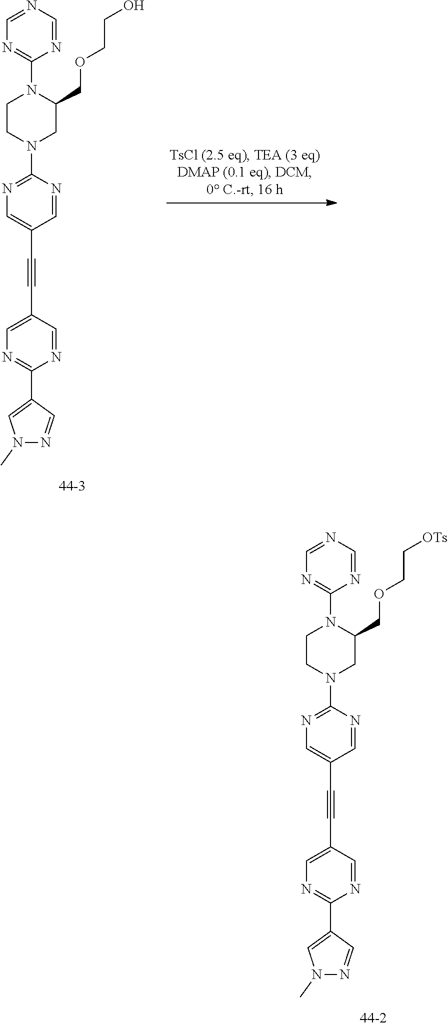 [SMILES: Cn1cc(-c2ncc(C#Cc3cnc(N4CCN(c5ncncn5)[C@@H](COCCO)C4)nc3)cn2)cn1 ; Cc1ccc(S(=O)(=O)OCCOC[C@H]2CN(c3ncc(C#Cc4cnc(-c5cnn(C)c5)nc4)cn3)CCN2c2ncncn2)cc1]

### Synthesis of 44-2: (R)-2-((4-(5-((2-(1-methyl-1H-pyrazol-4-yl)pyrimidin-5-yl)ethynyl)pyrimidin-2-yl)-1-(1,3,5-triazin-2-yl)piperazin-2-yl)methoxy)ethyl4-methylbenzenesulfonate

To a stirred solution of 44-3 (made in an analogous manner as 52-3) (500 mg, 1.001 mmol) in DCM (5 mL) were added TEA (0.419 mL, 3.00 mmol). DMAP (12.23 mg. 0.100 mmol) and p-toluenesulfonyl chloride (477 mg, 2.502 mmol) at 0° C. The reaction mixture was stirred at room temperature for 16 h. Reaction mixture was quenched with water (100 mL) and extracted with EtOAc (3×100 mL). Combined organic layer was dried over Na₂SO₄, filtered and concentrated under reduced pressure. Crude compound was purified by prep-HPLC purification (conditions: Mobile Phase—10 mM Ammonium Bicarbonate IN H₂O: MeCN COLUMN—Betasil phenyl hexyl (20×250) mm, 5μ Flow-18.0 ml/min, Gradient Method-0/49,16/52,16.05/100,19/100,19.05/49,23/49). Pure fractions were combined, concentrated under reduced pressure and lyophilized to afford 44-2. M/Z (ESI): 654.35 [M+H]⁺.

¹H NMR (400 MHz, DMSO-d₆) δ (ppm)=8.90 (s, 2H). 8.62 (d, J=5.2 Hz, 4H), 8.42 (s, 1H), 8.06 (s, 1H), 7.75 (d, J=8.4 Hz, 2H), 7.45 (d, J=8.0 Hz, 2H), 4.85-4.94 (m, 1H), 4.70 (d, J=13.6 Hz, 1H), 4.48-4.64 (m, 2H), 4.03 (t, J=4.4 Hz, 2H), 3.92 (s, 3H), 3.50-3.60 (m, 2H), 3.44 (d, J=6.8 Hz, 2H), 3.36-3.40 (m, 1H), 3.18-3.27 (m, 2H), 2.40 (s, 3H).

### Synthesis of 46-7: (R)-2-(4-(5-((2-(3-(methoxymethyl)-4-(1,3,5-triazin-2-yl)piperazin-1-yl)pyrimidin-5-yl)ethynyl)pyrimidin-2-yl)-1H-pyrazol-1-yl)ethyl 4-methylbenzenesulfonate

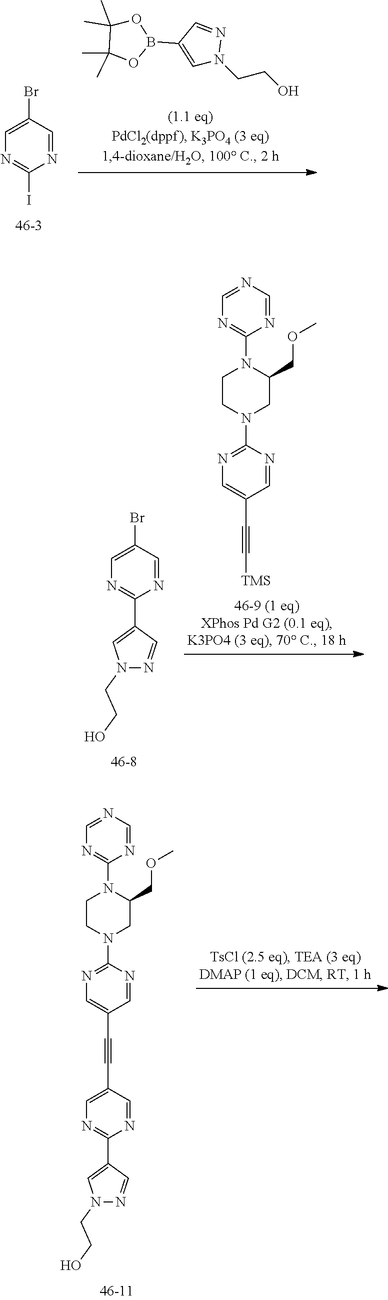 [SMILES: CC1(C)OB(c2cnn(CCO)c2)OC1(C)C ; Brc1cnc(I)nc1 ; OCCn1cc(-c2ncc(Br)cn2)cn1 ; COC[C@H]1CN(c2ncc(C#C[Si](C)(C)C)cn2)CCN1c1ncncn1 ; COC[C@H]1CN(c2ncc(C#Cc3cnc(-c4cnn(CCO)c4)nc3)cn2)CCN1c1ncncn1]

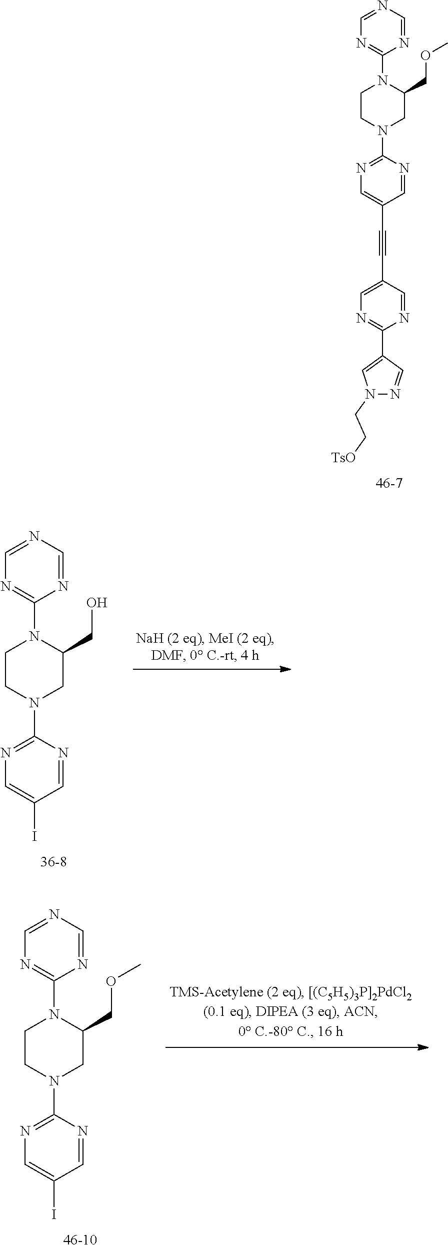 [SMILES: COC[C@H]1CN(c2ncc(C#Cc3cnc(-c4cnn(CCOS(=O)(=O)c5ccc(C)cc5)c4)nc3)cn2)CCN1c1ncncn1 ; OC[C@H]1CN(c2ncc(I)cn2)CCN1c1ncncn1 ; COC[C@H]1CN(c2ncc(I)cn2)CCN1c1ncncn1]

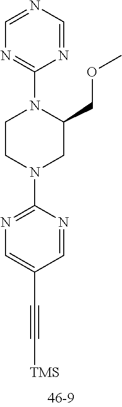 [SMILES: COC[C@H]1CN(c2ncc(C#C[Si](C)(C)C)cn2)CCN1c1ncncn1]

### Synthesis of 46-8: 2-(4-(5-bromopyrimidin-2-yl)-1H-pyrazol-1-yl)ethan-1-ol

To a stirred solution of 5-bromo-2-iodopyrimidine (46-3) (1 g. 3.51 mmol) in 1,4-dioxane (8 mL) and H₂O (2 mL) at room temperature. The reaction mixture was degassed and purged with argon for 10 min. Then to this reaction mixture were added 2-(4-(4,4,5,5-tetramethyl-1,3,2-dioxaborolan-2-yl)-1H-pyrazol-1-yl)ethan-1-ol (0.919 g, 3.86 mmol), tripotassium phosphate (2.235 g, 10.53 mmol), 1,1′-bis(diphenylphosphino)ferrocene]dichloropalladium(II) (0.257 g, 0.351 mmol) at room temperature. The reaction mixture was again degassed and purged with argon gas for 2 min. The reaction mixture was stirred at 100° C. for 12 h. The reaction mixture was quenched with water (5 mL) and extracted with EtOAc (2×20 mL). Combined organic layer was washed with brine (10 mL), dried over anhydrous Na₂SO₄, filtered and concentrated under reduced pressure. Crude compound was purified by silica gel column and compound eluted with 40% EtOAc in pet ether. Pure fractions were combined and concentrated under reduced pressure to afford 46-8. M/Z (ESI): 270.90 [M+H]⁺.

### Synthesis of 46-10: (R)-2-(4-(5-iodopyrimidin-2-yl)-2-(methoxymethyl)piperazin-1-yl)-1,3,5-triazine

To a stirred solution of 36-8 (1 g, 2.505 mmol) in DMF (10 mL) were added methyl iodide (0.711 g, 5.01 mmol), 60% NaH in mineral oil (0.200 g, 5.01 mmol) at 0° C. The reaction mixture was stirred at room temperature for 4 h. The reaction mixture was quenched with water (8 mL) and extracted with EtOAc (2×50 mL). Combined organic layer was washed with brine (10 mL), dried over anhydrous Na₂SO₄, filtered and concentrated under reduced pressure. Crude compound was purified by silica gel column and compound eluted with 30% EtOAc in pet ether. Pure fractions were combined and concentrated under reduced pressure to afford 46-10. M/Z (ESI): 414.18 [M+H]⁺.

### Synthesis of 46-9: (R)-(R)-2-(2-(methoxymethyl)-4-(5-((trimethylsilyl)ethynyl)pyrimidin-2-yl)piperazin-1-yl)-1,3,5-triazine

To a stirred solution of 46-10 (680 mg, 1.646 mmol) in ACN (7 mL) were added DIPEA (0.862 mL, 4.94 mmol) at room temperature. The reaction mixture was degassed and purged with argon for 20 min. Then to this reaction mixture was added trimethylsilylacetylene (323 mg, 3.29 mmol) and XPhos Pd G2 (129 mg, 0.165 mmol) at room temperature. The reaction mixture was stirred at 80° C. for 16 h under nitrogen atmosphere. The reaction mixture was quenched with water (10 mL) and extracted with EtOAc (2×50 mL). Combined organic layer was washed with brine (2×10 mL), dried over anhydrous Na₂SO₄, filtered and concentrated under reduced pressure. Crude compound was purified by Biotage using 100 g silica (100-200 mesh) cartridge and compound eluted with 50% EtOAc in pet ether. Pure fractions were combined and concentrated under reduced pressure to afford 46-9. M/Z (ESI): 384.32 [M+H]⁺.

### Synthesis of 46-11: (R)-2-(4-(5-((2-(3-(methoxymethyl)-4-(1,3,5-triazin-2-yl)piperazin-1-yl)pyrimidin-5-yl)ethynyl)pyrimidin-2-yl)-1H-pyrazol-1-yl)ethan-1-ol

To a stirred solution of 46-9 (450 mg, 1.173 mmol) in ACN (4 mL) and DMF (1.5 mL) were added 46-8 (379 mg, 1.408 mmol) and tripotassium phosphate (747 mg, 3.52 mmol) at room temperature. The reaction mixture was degassed and purged with argon for 15 min. Then to this reaction mixture was added XPhos Pd G2 (92 mg, 0.117 mmol) at room temperature. The reaction mixture was stirred at 70° C. for 18 h under nitrogen atmosphere in a sealed tube. The reaction mixture was quenched with water (7 mL) and extracted with EtOAc (2×40 mL). Combined organic layer was washed with brine (2×10 mL), dried over anhydrous Na₂SO₄, filtered and concentrated under reduced pressure. Crude compound was purified by Biotage using 40 g silica (100-200 mesh) cartridge and compound was eluted with 10% MeOH in DCM. Pure fractions were combined and concentrated under reduced pressure. Obtained compound was further re-purified by prep-HPLC purification (conditions: Mobile Phase—10 mM Ammonium Bicarbonate IN H₂O: MeCN COLUMN—X-Bridge PACK, C18 (25×150) mm. 5p Flow-20.0 ml/min Gradient Method 0/20,9.5/47.3,9.55/100,12/100,12.05/20,15/20) to afford 46-11. M/Z (ESI): 500.25 [M+H]⁺.

### Synthesis of 46-7: (R)-2-(4-(5-((2-(3-(methoxymethyl)-4-(1,3,5-triazin-2-ylpiperazin-1-yl)pyrimidin-5-yl)ethynyl)pyrimidin-2-yl)-1H-pyrazol-1-yl)ethyl 4-methylbenzenesulfonate

To a stirred solution of 46-11 (120 mg, 0.240 mmol) in DCM (2 mL) were added TEA (0.100 mL, 0.721 mmol), DMAP (29.3 mg, 0.240 mmol) and Ts-Cl (114 mg, 0.601 mmol) at 0° C. The reaction mixture was stirred at room temperature for 1 h under nitrogen atmosphere. The reaction mixture was quenched with water (40 mL) and extracted with EtOAc (2×70 mL). Combined organic layer was washed with brine (2×40 mL), dried over anhydrous Na₂SO₄, filtered and concentrated under reduced pressure. Crude compound was purified by Biotage using 80 g silica (230-400 mesh) cartridge and compound eluted with 3% MeOH in DCM. Pure fractions were combined and concentrated under reduced pressure. Obtained compound was further re-purified by pre-HPLC purification (conditions: Mobile Phase—10 mM Ammonium Bicarbonate IN H₂O: MeCN COLUMN—X-Bridge, C18 (19×150) mm, 5μ Flow-15.0 ml/mm Gradient Method—0/45, 2/45, 12/80, 12.01/100, 15/100, 15.01/45, 18/45). Pure fractions were combined and concentrated under reduced pressure and lyophilized to afford 46-7. M/Z (ESI): 654.18 [M+H]⁺.

¹H NMR (400 MHz, DMSO-d₆) δ (ppm)=8.92 (s, 2H), 8.65 (d, J=4.8 Hz, 4H), 8.35 (s, 1H), 7.99 (d, J=0.4 Hz, 1H), 7.62 (d, J=8.4 Hz, 2H). 7.30-7.39 (m, 2H). 4.95-5.06 (m, 1H), 4.77 (d, J=13.6 Hz, 1H), 4.54-4.67 (m, 2H), 4.44 (q, J=3.7 Hz, 4H), 3.45 (d, J=7.2 Hz, 2H), 3.33-3.40 (m, 1H), 3.15-3.28 (m, 5H), 2.31 (s, 3H).

### Synthesis of 48-6: (R)-2-(2-((4-(5-((6-(oxazol-5-yl)pyridin-3-yl)ethynyl)pyrimidin-2-yl)-1-(1,3,5-triazin-2-yl)piperazin-2-yl)methoxy)ethoxy)ethyl 4-methylbenzenesulfonate

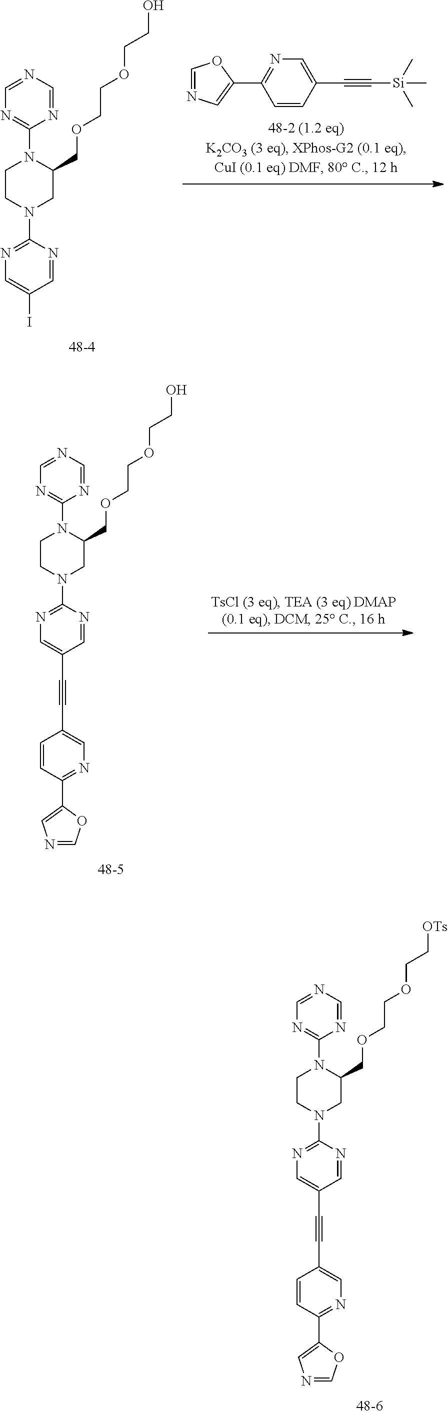 [SMILES: OCCOCCOC[C@H]1CN(c2ncc(I)cn2)CCN1c1ncncn1 ; C[Si](C)(C)C#Cc1ccc(-c2cnco2)nc1 ; OCCOCCOC[C@H]1CN(c2ncc(C#Cc3ccc(-c4cnco4)nc3)cn2)CCN1c1ncncn1 ; Cc1ccc(S(=O)(=O)OCCOCCOC[C@H]2CN(c3ncc(C#Cc4ccc(-c5cnco5)nc4)cn3)CCN2c2ncncn2)cc1]

### Synthesis of 48-5: (R)-2-(2-((4-(5-((6-(oxazol-5-yl)pyridin-3-yl)ethynyl)pyrimidin-2-yl)-j-(1,3,5-triazin-2-yl)piperazin-2-yl)methoxy)ethoxy)ethan-1-ol

The stirred solution of 48-4 (180 mg, 0.369 mmol) in DMF (2 mL) was purged with argon gas for 10 min. Then to this reaction mixture were added 48-2 (107 mg, 0.443 mmol), K₂CO₃ (153 mg, 1.108 mmol), copper(I) iodide (7.03 mg. 0.037 mmol) and chloro(2-dicyclohexylphosphino-2′,4′,6′-triisopropyl-1,1′-biphenyl)[2-(2′-amino-1,1′-biphenyl)]palladium(II) (29.1 mg, 0.037 mmol) at room temperature. The reaction mixture was again purged with argon for another 10 min. The reaction mixture was stirred at 80° C. for 12 h. Reaction mixture was diluted with water (100 mL) and extracted with EtOAc (3×100 mL). Combined organic layer was dried over Na₂SO₄ and concentrated under reduced pressure. Crude compound was triturated with diethyl ether (2×30 mL) and dried under reduced pressure to afford (5075688-0321-002) (160 mg, 0.275 mmol, 74.4% yield) as a pale yellow solid. From obtained compound (5075688-0321-002) (160 mg) 30 mg was again purified by SFC purification. Pure fractions were combined and lyophilized to afford 48-5. M/Z (ESI): 530.27 [M+H]⁻.

Analytical SFC Conditions:

| | |
|---|---|
| Column/dimensions | YMC-Diol(250 × 4.6 × 5 μ) |
| % CO2 | 55% |
| % Co solvent | 45% (100% Acetonitrile) |

Preparative SFC Conditions

| | |
|---|---|
| Column/Dimensions | YMC DIOL (250 × 10 × 5 μ) |
| % CO2 | 60% |
| % Co solvent | 40% (100% Acetonitrile) |
| Instrument details | Make/Model: SEPIATEC-50 |

¹H NMR (400 MHz, DMSO-d₆) δ (ppm)=8.77 (dd, J=0.8, 2.4 Hz, 1H), 8.65 (s, 4H), 8.59 (s, 1H), 8.06 (dd, J=2, 8.4 Hz, 1H), 7.88 (s, 1H), 7.83-7.81 (m, 1H), 4.96-4.94 (m, 1H), 4.78 (d, J=13.6 Hz, 1H), 4.60-4.51 (m, 3H), 3.52 (d, J=7.2 Hz, 2H), 3.49-3.44 (m, 2H), 3.43-3.39 (m, 5H), 3.36-3.31 (m, 2H), 3.26-3.25 (m, 1H), 3.26-3.16 (m, 1H).

### Synthesis of 48-6: (R)-2-(2-((4-(5-((6-(oxazol-5-yl)pyridin-3-yl)ethynyl)pyrimidin-2-yl)-1-(1,3,5-triazin-2-yl)piperazin-2-yl)methoxy)ethoxy)ethyl 4-methylbenzenesulfonate

To a stirred solution of 48-5 (130 mg, 0.245 mmol) in DCM (1 mL) were added TEA (0.103 mL, 0.736 mmol), DMAP (3.00 mg, 0.025 mmol) and p-TsCl (140 mg, 0.736 mmol) at 0° C. The reaction mixture was stirred at 25° C. for 16 h. Reaction mixture was diluted with water (100 mL) and extracted with EtOAc (3×100 mL). Combined organic layer was dried over Na₂SO₄ and concentrated under reduced pressure. Crude compound was purified by prep-HPLC purification. Pure fractions were combined and lyophilized to afford 48-6. M/Z (ESI): 684.20 [M+H]⁻.

### Prep HPLC Method;

Mobile Phase—10 mM Ammonium Bicarbonate IN H₂O: MeCN
COLUMN—X-Bridge C18 (10×250 mm), 5μ Flow-7 ml/min
Gradient Method-0/52,2/52,7.5/55.5,10/55.5,10.05/100,12/100,12.05/52,16/52.

¹H NMR (400 MHz, DMSO-d₆) δ (ppm)=8.79-8.76 (m, 1H), 8.63 (s, 4H), 8.59 (s, 1H), 8.06 (dd, J=2.1, 8.3 Hz, 1H), 7.88 (s, 1H), 7.83 (dd, J=0.8, 8.3 Hz, 1H), 7.79-7.75 (m, 2H), 7.45 (d, J=8.0 Hz, 2H), 4.97-4.90 (m, 1H), 4.77 (br d, J=13.5 Hz, 1H), 4.63-4.51 (m, 2H), 4.08-4.05 (m, 2H), 3.53-3.47 (m, 4H), 3.43-3.39 (m, 2H), 3.37 (br d, J=2.4 Hz, 3H), 3.29-3.16 (m, 2H), 2.39 (s, 3H).

### Synthesis of 52-7: (R)-2-(2-((4-(5-((2-(1-methyl-1H-pyrazol-4-yl)pyrimidin-5-yl)ethynyl)pyrimidin-2-yl)-1-(pyrimidin-2-yl)piperazin-2-yl)methoxy)ethoxy)ethyl4-methylbenzenesulfonate

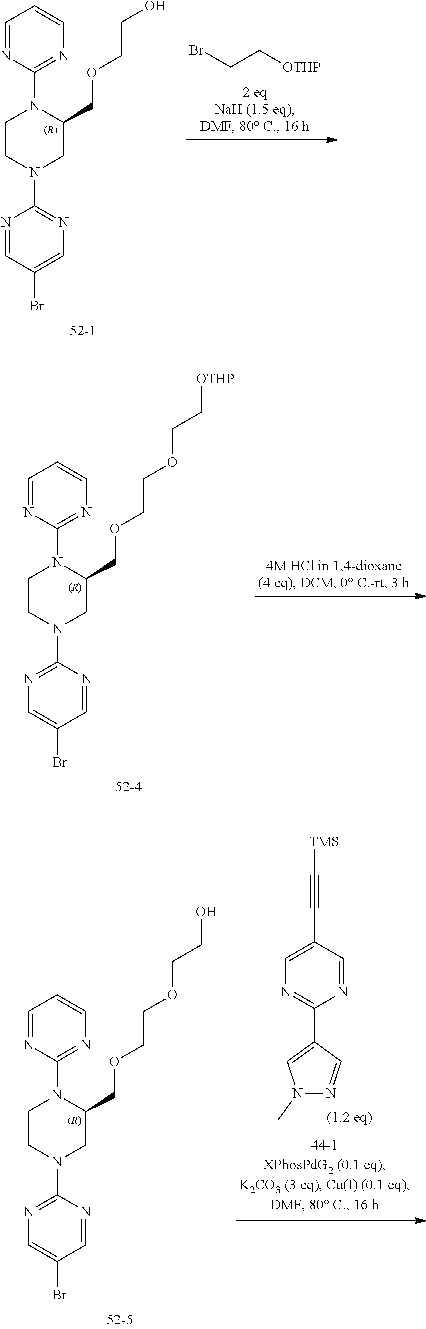

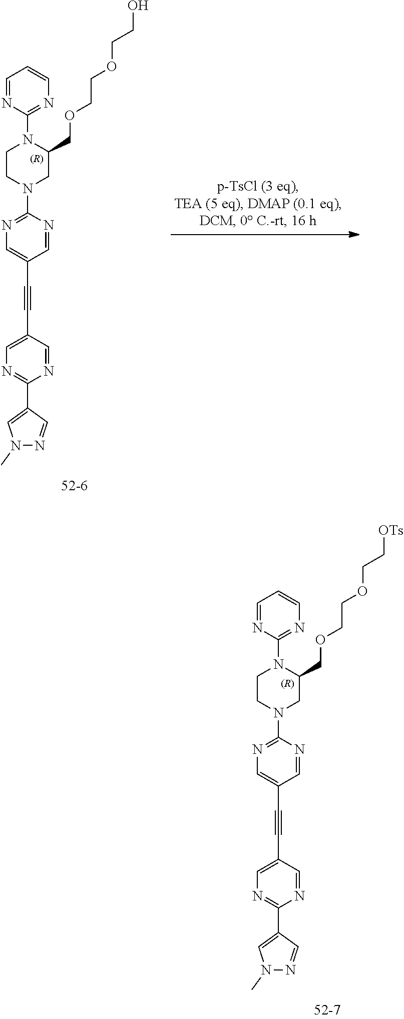

### Synthesis of 52-4: 5-bromo-2-((3R)-4-(pyrimidin-2-yl)-3-((2-(2-((tetrahy dro-2H-pyran-2-yl)oxy)ethoxy)ethoxy)methyl)piperazin-1-yl)pyrimidine

To a stirred solution of 52-1 (2.0 g, 5.06 mmol) in DMF (40 mL) was added 60% in oil NaH (0.304 g, 7.59 mmol) at 0° C. and stirred at 0° C. for 30 min. Then 2-(2-bromoethoxy)tetrahydro-2 h-pyran (2.116 g, 10.12 mmol) was added to the reaction mixture at 0° C. The reaction mixture was stirred at 80° C. for 16 h. Reaction mixture was quenched with water (150 mL) and extracted with EtOAc (3×200 mL). Combined organic layer was dried over Na₂SO₄, filtered and concentrated under reduced pressure to afford 52-4. M/Z (ESI): 525.11 [M+H]⁻.

### Synthesis of 52-5: (R)-2-(2-((4-(5-bromopyrimidin-2-yl)-1-(pyrimidin-2-yl)piperazin-2-yl)methoxy)ethoxy)ethan-1-ol

To a stirred solution of 52-4 (2 g, 3.82 mmol) in DCM (30 mL) was added 4M 1,4-dioxane hydrochloride (3.82 mL, 15.28 mmol) at 0° C. The reaction mixture was stirred at 25° C. for 3 h. Reaction mixture was concentrated under reduced pressure. The residue was diluted with aq. NaHCO₃ solution and extracted with EtOAc (3×80 mL). Combined organic layer was dried over Na₂SO₄, filtered and concentrated under reduced pressure. Crude compound was purified by Biotage using 25 g silica gel and compound eluted with 35% EtOAc in pet ether. Pure fractions were combined and concentrated under reduced pressure to afford 52-5. M/Z (ESI): 439.07 [M+H]⁻.

### Synthesis of 52-6: (R)-2-(2-((4-(5-((2-(1-methyl-1H-pyrazol-4-yl)pyrimidin-5-yl)ethynyl)pyrimidin-2-yl)-1-(pyrimidin-2-yl)piperazin-2-yl)methoxy)ethoxy)ethan-1-ol

To a stirred solution of 52-5 (800 mg, 1.821 mmol) in DMF (8 mL) were added 44-1 (607 mg, 2.367 mmol), K₂CO₃ (755 mg, 5.46 mmol), copper(I) iodide (34.7 mg, 0.182 mmol) and chloro(2-dicyclohexylphosphino-2′,4′,6′-triisopropyl-1,1′-biphenyl)[2-(2′-amino-1,1′-biphenyl)]palladium(II) (143 mg, 0.182 mmol) at room temperature and degassed with argon gas for 10 min at room temperature. The reaction mixture was stirred under nitrogen atmosphere at 80° C. for 16 h in a sealed tube. Reaction mixture was quenched with water (50 mL) and extracted with EtOAc (2×50 mL). Combined organic layer was washed with brine (2×30 mL), dried over Na₂SO₄, filtered and concentrated under reduced pressure. Crude compound was purified by Biotage using 100 g silica reverse phase RP GOLD column and compound eluted with 50% ACN in water. Pure fractions were combined and concentrated under reduced pressure to afford 52-6. M/Z (ESI): 543.71 [M+H]⁺.

### Synthesis of 52-7: (R)-(R)-2-(2-((4-(5-((2-(1-methyl-1H-pyrazol-4-yl)pyrimidin-5-yl)ethynyl)pyrimidin-2-yl)-1-(pyrimidin-2-yl)piperazin-2-yl)methoxy)ethoxy)ethyl 4-methylbenzenesulfonate

To a stirred solution of 52-6 (90 mg, 0.166 mmol) in DCM (1 mL) were added TEA (0.116 mL, 0.829 mmol), 4-dimethylaminopyridine (2.026 mg, 0.017 mmol) and p-TsCl (95 mg, 0.498 mmol) at 0° C. The reaction mixture was stirred at 25° C. for 16 h. Reaction mixture was quenched with water (40 mL) and extracted with EtOAc (2×40 mL). Combined organic layer was washed with brine (20 mL), dried over anhydrous Na₂SO₄, filtered and concentrated under reduced pressure. Crude compound was purified by Biotage using 100 g silica reverse phase RP GOLD column and compound eluted with 70% ACN in water. Pure fractions were combined and concentrated under reduced pressure to afford 52-7. M/Z (ESI): 697.37 [M+H]⁺.

¹H NMR (400 MHz, DMSO-d₆) δ=8.89 (s, 2H), 8.61 (s, 2H), 8.36-8.47 (m, 3H), 8.05 (d, J=0.4 Hz, 1H), 7.77 (d, J=8.4 Hz, 2H), 7.44 (d, J=8.0 Hz, 2H), 6.67 (t, J=4.8 Hz, 1H), 4.83-4.91 (m, 1H), 4.78 (d, J=13.6 Hz, 1H), 4.47-4.57 (m, 2H), 4.08 (t, J=4.4 Hz, 2H), 3.92 (s, 3H), 3.50-3.56 (m, 2H), 3.34-3.49 (m, 7H), 3.22-3.29 (m, 2H), 2.39 (s, 3H).

The compounds contained in Table 3 were synthesized by analogous methods from synthetic sequences above as indicated in the last column in Table 3. Commercially available reagents were substituted where necessary to produce the examples below.

TABLE 3

| | | | Observed | |
|---|---|---|---|---|
| Ex | | | Mass | Exact |
| No. | Structure | Chemical Name | (M + H) | Mass |
| 38-11 | 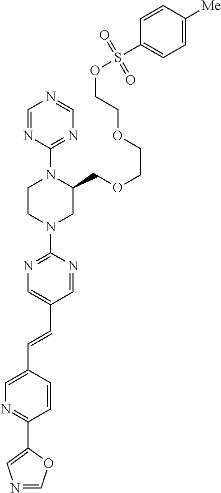 [SMILES: Cc1ccc(S(=O)(=O)OCCOCCOC[C@H]2CN(c3ncc(/C=C/c4ccc(-c5cnco5)nc4)cn3)CCN2c2ncncn2)cc1] | (R,E)-2-(2-((4-(5-(2-(6- (oxazol-5-yl)pyridin-3- yl)vinyl)pyrimidin-2-yl)-1- (1,3,5-triazin-2-yl)piperazin- 2-yl)methoxy)ethoxy)ethyl 4-methylbenzenesulfonate | 686.34 | 685.24 |
| 49-4 | 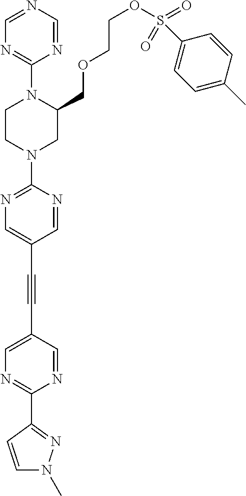 [SMILES: Cc1ccc(S(=O)(=O)OCCOC[C@H]2CN(c3ncc(C#Cc4cnc(-c5ccn(C)n5)nc4)cn3)CCN2c2ncncn2)cc1] | (R)-2-(2-((4-(5-((2-(1- methyl-1H-pyrazol-4- yl)pyrimidin-5- yl)ethynyl)pyrimidin-2-yl)- 1-(pyrimidin-2-yl)piperazin- 2-yl)methoxy)ethoxy)ethyl 4-methylbenzenesulfonate | 654.28 | 653.23 |
| 51-2 | 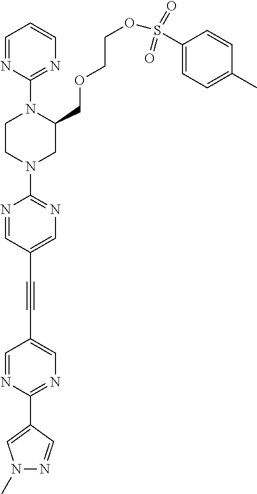 | (R)-2-((4-(5-((2-(1-methyl- 1H-pyrazol-4-yl)pyrimidin- 5-yl)ethynyl)pyrimidin-2- yl)-1-(pyrimidin-2- yl)piperazin-2- yl)methoxy)ethyl 4- methylbenzenesulfonate | 653.34 | 652.23 |

### Synthesis of [³H]-1000: [³H]-(S)-6-(1H-imidazol-1-yl)-N-(2-(2-methyl-4-(pyridin-2-yl)piperazin-1-yl)pyrimidin-5-yl)nicotinamide

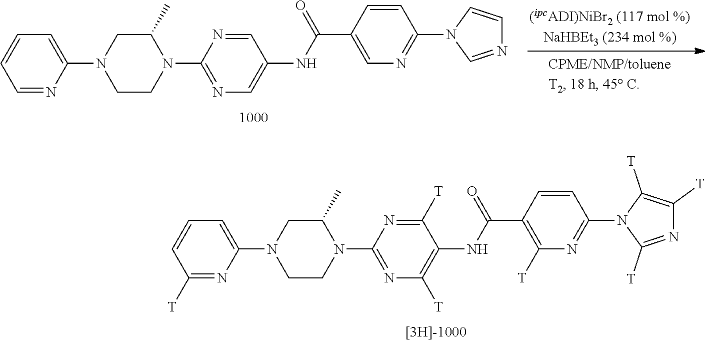

### Synthesis of Intermediate ST-A: (S)-2-(2-methyl-4-(pyridin-2-yl)piperazin-1-yl)pyrimidin-5-amine

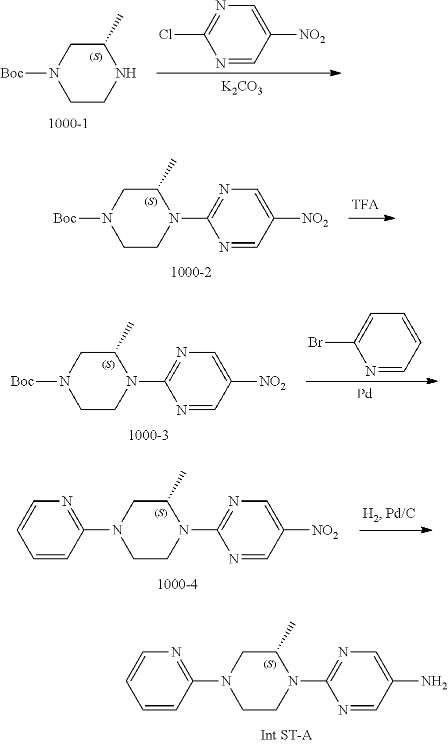

### Synthesis of 1000-2: (S)-tert-butyl 3-methyl-4-(5-nitropyrimidin-2-yl)piperazine-1-carboxylate

To a solution of (S)-tert-butyl 3-methylpiperazine-1-carboxylate (1000-1, 13.6 g, 67.9 mmol) in DMF (150 mL) was added K₂CO₃ (14.08 g, 102 mmol) and 2-chloro-5-nitropyrimidine (12.46 g, 78 mmol). The mixture was stirred for 12 h at 25° C. under N₂ balloon. TLC showed the starting material was consumed completely. Water (450 mL) was added and the mixture was stirred at 25° C. (rt) for 30 min. The precipitated solid was collected by filtration, washed with water (100 mL×3) and dried to give the 1000-2 as a solid.

¹H NMR (500 MHz, Chloroform-d): 6=9.07 (s, 2H), 5.06 (br s, 1H), 4.67 (br s, 1H), 3.91-4.29 (m, 2H), 3.28-3.36 (m, 1H), 3.13 (br s, 1H), 2.83-3.01 (m, 1H), 1.45-1.52 (m, 9H), 1.26 (d, J=6.5 Hz, 3H)

### Synthesis of 1000-3: (S)-2-(2-methylpiperazin-1-yl)-5-nitropyrimidine

To a solution of 1000-2 (21 g, 64.9 mmol) in DCM (160 mL) was added TFA (40 mL) at 0° C. The mixture was stirred for 2 h at 25° C. TLC showed most of the starting material was consumed completely. The mixture was concentrated under reduced pressure to give the crude product (S)-2-(2-methylpiperazin-1-yl)-5-nitropyrimidine (25 g, 78 mmol) as an oil. The product was diluted with DCM (200 mL) and H₂O (160 mL). Then the Na₂CO₃ was added to solution to adjust pH to 7-8. The solution was extracted with DCM (200 mL*2). The organic layer was dried over Na₂SO₄, filtered and concentrated to give 1000-3 as a solid.

¹H NMR (400 MHz, DMSO-d₆): δ=9.44 (s, 1H), 8.92~9.10 (m, 1H), 5.13~5.24 (m, 1H), 4.82 (d, J=14.4 Hz, 1H), 3.31~3.49 (m, 3H), 3.26 (d, J=7.2 Hz, 1H), 3.06 (d, J=8.8 Hz, 1H), 1.52 (s, 1H), 1.33 (d, J=7.2 Hz, 3H). MS (ESI) m/z: 224.0 [M+H]⁺.

### Synthesis of 1000-4: (S)-2-(2-methyl-4-(pyridin-2-yl)piperazin-1-yl)-5-nitropyrimidine

To a solution of 1000-3 (2.5 g, 11.20 mmol) in Dioxane (50 mL) was added 2-bromopyridine (3.72 g, 23.52 mmol), Cs₂CO₃ (14.96 g, 45.9 mmol) and chloro(2-dicyclohexylphosphino-2′,6′-dimethoxy-1,1′-biphenyl)[2-(2′-amino-1,1′-biphenyl)]palladium(II) (0.968 g, 1.344 mmol). The mixture was stirred for 12 h at 110° C. under N₂ balloon. TLC showed most of the starting material was consumed completely. The mixture was filtered and concentrated. The residue was extracted with EtOAc (3*50 mL) and H₂O (60 mL). The combined organic extracts were washed with brine (100 mL), dried over anhydrous Na₂SO₄ and filtered. The filtrate was concentrated under reduced pressure. The residue was purified using a silica gel column eluting with 15~30% EtOAc/PE to give the 1000-4 as a solid.

¹H NMR (400 MHz, Chloroform -d): 6=9.10 (s, 2H). 8.17~8.24 (m, 1H), 7.47-7.59 (m, 1H), 6.62~6.71 (m, 2H), 5.11 (dt, J=6.4, 3.2 Hz, 1H), 4.74 (dt, J=13.6, 3.6 Hz, 1H), 4.24 (d, J=12.8 Hz, 1H), 4.13 (d, J=13.2 Hz, 1H), 3.53~3.63 (m, 1H), 3.37 (dd, J=13.2, 4.0 Hz, 1H), 3.12 (td, J=12.0, 3.6 Hz, 1H), 1.34 (d, J=6.8 Hz, 3H). MS (ESI) m/z: 301.0 [M+H]⁺.

### Synthesis of (S)-2-(2-methyl-4-(pyridin-2-yl)piperazin-1-yl)pyrimidin-5-amine (Int ST-A)

To a solution of 1000-4 (2.5 g, 8.32 mmol) in MeOH (40 mL) was added Pd/C (0.2 g, 1.879 mmol). The mixture was stirred for 2 h at 25° C. under H₂ balloon. TLC showed most of the starting material was consumed completely. The mixture was filtered, and the filter cake was washed with MeOH (3*200 mL). The combined organic extracts were concentrated under reduced pressure to give Int ST-A as an oil.

¹H NMR (400 MHz, Chloroform -d): 5=8.19 (dd, J=4.8, 1.2 Hz, 1H), 8.01 (s, 2H), 7.45~7.50 (m, 1H), 6.66 (d, J=8.8 Hz, 1H), 6.60 (dd, J=6.8, 5.2 Hz, 1H), 4.76-4.85 (m, 1H), 4.32~4.40 (m, 1H), 4.19~4.26 (m, 1H), 4.10 (dt, J=12.8, 2.0 Hz, 1H), 3.23~3.41 (m, 2H), 3.15 (s, 2H). 3.01-3.08 (m, 1H), 1.22 (d, J=6.8 Hz, 3H). MS (ESI) m/z: 271.1 [M+H]1.

### Synthesis of Compound 1000: (S)-6-(1H-imidazol-1-yl)-N-(2-(2-methyl-4-(pyridin-2-yl)piperazin-1-yl)pyrimidin-5-yl)nicotinamide

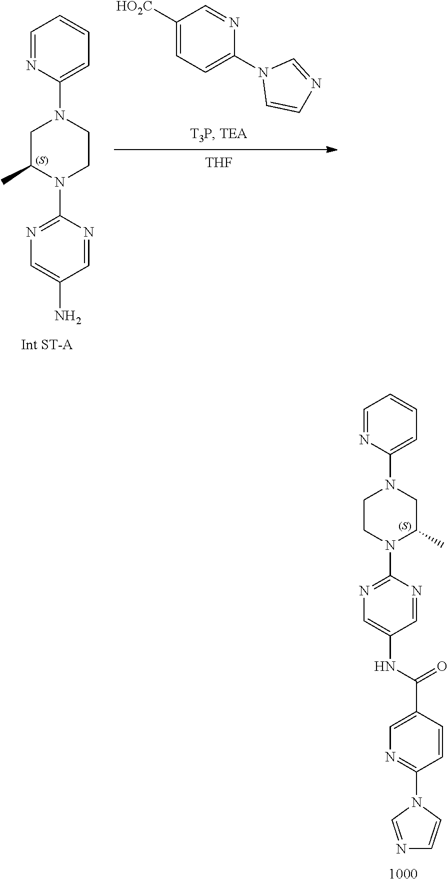

To a stirred solution of Int ST-A (150 mg, 0.555 mmol), 6-(1H-imidazol-1-yl)nicotinic acid (157 mg, 0.832 mmol) in THF (30 ml) was added TEA (0.193 ml, 1.387 mmol), 1-propanephosphonic anhydride (0.495 ml, 0.832 mmol) at 25° C. and stirred for 16 h at 25° C. The reaction mixture was quenched with ice cold water (10 mL), extracted with ethyl acetate (2×100 mL), combined organic layer washed with brine solution (2×10 mL), dried over sodium sulfate, filtered, concentrated under reduced pressure and crude compound purified by Prep HPLC (mobile phase—10 mM Ammonium Bicarbonate in H₂O: MeCN column—X-Select C18 (19×250) mm 5u Flow-18 ml/min gradient method-0/45, 6.9/76, 6.95/100, 9/100, 9.05/45, 12/45). Pure fractions concentrated and lyophilized to afford 1000 as a pale yellow solid. M/Z (ESI): 442.14 [M+H]⁺.

¹H NMR (400 MHz, DMSO-d₆) δ: 10.43 (s, 1H), 9.04 (d, J=2.0 Hz, 1H), 8.71 (s, 2H), 8.66 (s, 1H), 8.52 (dd, J=8.7, 2.3 Hz, 1H), 8.12 (dd, J=4.9, 1.2 Hz, 1H), 8.06 (t, J=1.2 Hz, 1H), 8.01 (d, J=8.6 Hz, 1H), 7.55 (ddd, J=8.6, 7.0, 2.2 Hz, 1H), 7.18 (s, 1H), 6.86 (d, J=8.8 Hz, 1H), 6.60-6.67 (m, 1H), 4.85 (dt, J=6.4, 3.2 Hz, 1H), 4.41-4.49 (m, 1H), 4.17-4.31 (m, 2H), 3.28 (br d, J=3.7 Hz. 1H), 3.19 (br dd, J=13.1, 3.8 Hz, 1H), 2.90-3.01 (m, 1H), 1.16 (d, J=6.6 Hz, 3H).

In a glove box. Compound 1000 (1.33 mg, 3.0 μmol) was dissolved in CPME (75 μL) and NMP (25 μL). The nickel precatalyst (^{ipe} ADI)NiBr₂ (6.73 mg) was dissolved in CPME (670 μL) and treated with NaHBEt₃ in toluene (1 M, 25 μL) then stirred for 5 minutes. The substrate solution (100 μL) was combined with the active catalyst solution (200 μL, 3.5 μmol) in a tritiation vessel and secured with a portable Swagelok® valve. The valve was attached to the Trisorber and subjected to two freeze -pump-thaw cycles before 102 mm tritium gas was introduced. The reaction was thawed, then placed in an oil bath at 45° C. and stirred overnight. After capture of spent tritium on the waste bed, the reaction was transferred into a vial with 10 mL saturated aqueous sodium bicarbonate. The mixture was extracted three times with dichloromethane. The combined organic layers were dried with sodium sulfate and evaporated. The residue was dissolved in EtOH for LSC and radio-HPLC analysis. Crude yield: 120 mCi; RCP: 67%. The material was purified by HPLC. The collected fractions were diluted with an equal volume of water, concentrated on a pair of C18 cartridges and eluted with EtOH. Yield: approx. 20 mL ethanol solution @3.18 mCi/mL. The Specific Activity was determined to be 44.9 Ci/mmol by mass spectrometry; MW for C₂₃H₁₇T₇N₉O [M+H]⁺: 456.3, found: 456.0.

### HPLC Analytic Conditions

Method: 10-95% B in 12 minutes, hold 3 min, 6 min re-equilibration
Column: Gemini NX C18, 4.6×50 mm. 3.5 mm @40° C.
Flow Rate: 1 mL/min
Injection Volume: 1.0 μL
Detection: UV @294 nm
Mobile Phase A: 0.05 M pH 10 TEAA in H₂O
Mobile Phase B: CH₃CN
Product Elution Time: 6.32 minutes

### HPLC Preparatory Separation Conditions

Method: Isocratic (A:B=65:35)
Column: Gemini NX C18, 10×250 mm @40° C.
Flow Rate: 5 mL/min
Injection Volume: 0.5 mL
Detection: UV @295 nm
Mobile Phase A: 0.05 M pH 10 TEAA in H₂O
Mobile Phase B: CH₃CN

### Synthesis of [³H]-2: [³H]-(R,E)-5-(2-(6-(1H-imidazol-1-yl)pyridin-3-yl)vinyl)-2-(3-(methoxymethyl)-4-(pyrimidin-2-yl)piperazin-1-yl)pyrimidine

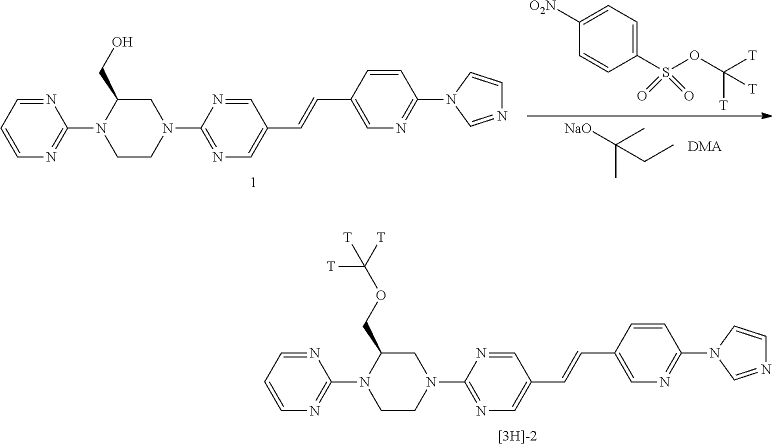

Ex. No. 1 (4.55 mg, 10 μmol) was combined with dimethylacetamide (0.1 mL) under nitrogen in a 1 mL crimp-sealed V-vial. Upon addition of sodium pentoxide (1.4 M, 6.9 μL, 9.7 μmol), the yellow suspension darkened to orange, but the solids were not fully soluble. After stirring 30 min at room temperature, the suspension was transferred on to 50 mCi [³H]methyl nosylate (0.6 μmol) in a separate 1 mL crimp-sealed V-vial under nitrogen. The vial was heated in an oil bath overnight. The reaction was partitioned between sat. aq. NaHCO₃ and CH₂Cl₂. The aqueous was extracted three more times with CH₂Cl₂. The combined organic extracts were dried over Na₂SO₄ and evaporated to afford a crude material with 24.5 mCi at 57% radiochemical purity by RP-HPLC analysis. [³H]-2 was purified by semi-prep HPLC. The collected fractions were diluted with an equal volume of water, concentrated on a pair of C18 cartridges and eluted with EtOH. Yield: 20 mL ethanol soln @0.28 mCi/mL. The Specific Activity was determined to be 63.0 Ci/mmol by mass spectrometry; MW for C₂₄H₂₃T₃N₉O [M+H]⁺: 462.2, found: 462.0.

### HPLC Analytic Conditions

Method: 10-95% B in 12 minutes, 3 min hold, 6 min re-equilibration
Column: Gemini NX C18, 4.6×50 mm, 3.5 mm @, 40° C.
Flow Rate: 1 mL/min
Injection Volume: 3.0 L
Detection: UV @4 335 nm
Mobile Phase A: 0.10% TFA in H₂O
Mobile Phase B: 0.1% TFA CH₃CN
Product Elution Time: 4.43 minutes

### HPLC Preparatory Separation Conditions

Method: Isocratic (A:B=55:45)
Column: Gemini NX C18, 10×250 mm a, 40° C.
Flow Rate: 5 mL/min
Injection Volume: 0.6 mL
Detection: UV (@340 nm
Mobile Phase A: 0.05 M pH 10 TEAA in H₂O
Mobile Phase B: CH₃CN

### Assay Procedures

### Acquisition of Human Post-Morten Tissue Samples for In Vitro Binding Assays

Frozen human brain tissues from PD patients were purchased from Analytic Biological Services Inc. The samples were post-mortem tissues from donors with clinical diagnosis of late stage of PD. Alpha-synuclein, tau and amyloid burden was determined through a combination of immunohistochemistry on frozen thin coronal sections, as well as alpha Lisa-based quantification of protein levels in a detergent insoluble protein fraction. A tissue sample of temporal cortex was identified from one donor as having moderate to high alpha-synuclein burden, low amyloid and minimal to no tau pathology. The detergent insoluble fraction of temporal cortex from this patient was used to support homogenate binding studies.

### Preparation Detergent Insoluble Fraction of Human Brain Tissue for In Vitro Binding Studies

Grey matter was dissected out of the temporal cortex tissue with a dissecting blade and minced with fine dissecting scissors. To prepare insoluble fractions, minced tissue was homogenized in ice cold TBS-TX buffer (50 mM Tris+150 mM NaCl+1% Triton×100+1 mM EDTA+1 tablet/10 mL of Complete Protease inhibitor+1 tablet/10 mL PHOSSTOP phosphatase inhibitor tablet) with glass Dounce tissue grinder. Homogenates were centrifuged at 100,000×g for 45 minutes. The pellet was resuspended in TBS-TX buffer, using a Polytron at highest setting for 30 seconds at 4° C. Homogenates were centrifuged at 100,000×g for 45 minutes and the pellet was resuspended in TBS-TX buffer. A BCA protein assay was performed on the final homogenate to determine the protein concentration. Homogenates were aliquoted in 0.5 ml/tube and stored at −70° C. until use.

### Procedure for Alpha-Svnuclein Tissue Homogenate Binding Assay (Assay 1)

For displacement a-synuclein binding assay, compounds and control were solvated in dimethyl sulfoxide (DMSO) and transferred using focused acoustic energy by an Echo 655 liquid handling instrument (Beckman Coulter, Indianapolis, IN) into designated wells of uniquely bar coded 96-well v-bottom low binding polypropylene microplates (Thermo Scientific, 249946). Compound dose response curves were prepared in a 10-point, 3-fold fashion within columns 2-11 of the microplate from high to low compound concentrations. The final assay concentration of dose response curves when starting at 1 mM ranged from 1.2 μM to 0.061 nM (0.12% DMSO final assay concentration, 270 nL/well). Controls included no inhibitor (DMSO only) dispensed into wells A1-D1, A12-D12 for minimum efficacy signal and Compound 1000 at a final assay concentration of 12 μM into wells E1-H1, E12-H12 for maximum efficacy signal. Liquid-handling steps for dispensing insoluble fractions of PD brain homogenates and radioligand were performed using a Bravo automated liquid handling platform equipped with a 96LT disposable tip head (Agilent Technologies, Santa Clara. CA). Insoluble fractions of PD brain homogenates were diluted to 50 μg/mL in the Assay Buffer, and 200 μL was dispensed to the assay plate for a final concentration of 10 μg/well. 25 μL of (9X) [³H]-1000 was dispensed to the assay plate for final assay concentration of 3.0 nM. Sealed assay plates were incubated at room temperature for 90 minutes with gentle agitation. The incubation was terminated by rapid filtration through UniFilter-96 GF/C microplates (pre-treated for 30 minutes with 0.2% Polyethylenimine at 4° C.) by using a FilterMate Harvester (PerkinElmer). The microplates were subsequently washed four times with a total volume of 3.75 mL using ice-cold Dulbecco's Phosphate-Buffered Saline (DPBS, Gibco 14190136) before drying 90 minutes at 47° C. with a vacuum oven (Fisher Scientific Isotemp 285A) or overnight at room temperature. The bottom of each UniFilter-96 GF/C microplate was adhesively sealed (PerkinElmer 6005199) prior to the addition of 50 μL MicroScint-20 liquid scintillation cocktail (PerkinElmer 6013621) to each well. A clear adhesive seal (TopSeal-A PLUS, PerkinElmer 6050185) was then applied to the top of each microplate and counted 1 minute/well on MicroBeta² system (PerkinElmer. Model: 2450-0120). Data was analyzed using IDBS ActivityBase XE Runner (version 9.6.0.148) to determine Kᵢ values shown in Data Table 1 (K_{d} value 0.90 nM, ligand concentration 3.0 nM).

### Competitive Radioligand Binding to Pathological Aggregated Beta Amyloid in AD Tissue (Assay 2):

Frozen human brain samples of Alzheimer's disease (AD) were purchased from Analytic Biological Services Inc. The samples were post-mortem tissue from donors with clinical diagnosis of AD and much of the white matter was dissected out of the frontal cortex in order to enrich the tissue preparations for gray matter. Brain homogenates of gray matter enriched frontal cortex were prepared by homogenizing the tissue in ice cold Phosphate Buffered Saline (PBS), pH 7.4 at 80 mg wet weight tissue per 1 ml for 45 seconds at 4° C., on setting 16 of Polytron. The homogenate was further diluted with ice cold PBS to 30 mg wet weight tissue per 1 ml and homogenized for an additional minute as described above. Homogenates were aliquoted in 5 ml/tube and stored at −70° C. until use.

Radioligand [³H]-105, prepared as described in ACS Med. Chem. Lett., Vol. 2, pages 498-502, was used in this assay.

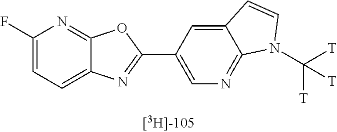

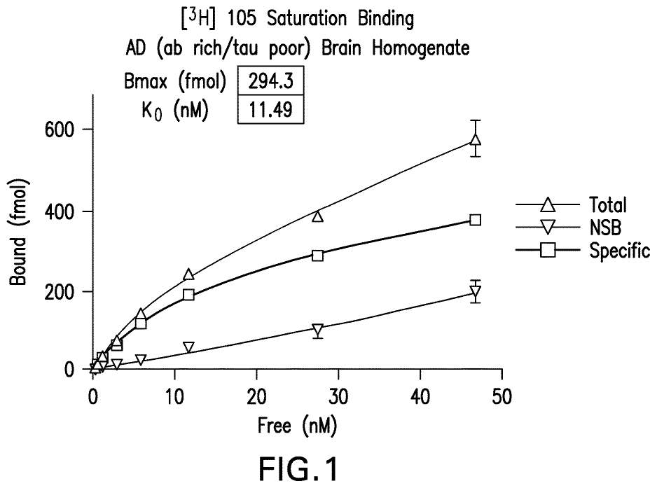

For hot saturation binding assay, various concentrations of radioligand, [³H]-105 were prepared in Assay Buffer (PBS plus 0.1% BSA) plus 20% DMSO ranging from 3.9 to 500 nM. 25 μl of radioligand was added to 200 μl of crude brain homogenates (diluted to 0.5 mg/ml in Assay Buffer) for final concentration of radioligand ranging from 0.39 to 50 nM and final crude brain homogenates of 100 ug wet weight/assay well (incubation, filtration, and determination of amount of radioligand used in assay are described below). Self-block with unlabeled compound was used to determine non-specific binding. Saturation data was analyzed using Graphpad/Prism software. FIG. 1 depicts high affinity saturation binding of [³H]-105 to AD tissue homogenate enriched in aggregated beta-amyloid pathology. FIG. 1 shows an example of hot saturation binding of [³H]-105, where the radioligand shows high affinity for aggregated beta amyloid (abeta) in AD brain homogenates with measured dissociation constant of 11 nM. This data supports the use of this ligand in radioligand binding assays to screen for binding to aggregated beta-amyloid.

For Assay 2 unlabeled test compounds were dissolved in DMSO at 10 mM. Dilutions of tests compounds to various concentrations were made in 100% DMSO at 1000× final assay concentration and 0.225 μl aliquots were dispensed into assay plates. Brain homogenates were diluted to 0.5 mg/ml from original 30 mg/ml volume in Assay Buffer, and 200 μl were added to the assay plate for a final concentration of 100 ug wet weight/assay well. [³H]-105 was prepared at 10× final concentration in Assay Buffer plus 20% DMSO and 25 μl was added to the assay plate for final assay concentration of 3.0 nM. The plates were incubated at 37° C. for 90 minutes. Unbound and bound ligand were separated by filtration of bound onto GF/B filter plates (pre-treated for 30 min with 0.1% PEI) using a Packard Filtermate and washing away unbound with 2.5 mil ice cold 5 mM Tris at pH 7.4. Filter plates were dried for 1 hr at 57° C. and 50 μl Microscint was added to each well of the plate. The plates were counted for ³H cpm for 1 min per well using PerkinElmer TopCount. Data was analyzed using IDBS Activity Base to determine Kᵢ values shown below in Data Table 1 (K_{d} value 11.0 nM, ligand concentration 3.5 nM).

Radioligand Binding Data in triton-extracted
alpha-synuclein from PD Tissue (Assay 1) and
Aβ-rich AD Tissue (Assay 2) - Data Table 1

| | α-synuclein Tissue Kᵢ | Aβ Tissue Kᵢ | Selectivity ratio |
|---|---|---|---|
| Ex. No. | (Assay 1, nM) | (Assay 2, nM) | (Assay 2/Assay 1) |
| 1 | 1.6 | 295 | 184 |
| 2 | 0.9 | 12000 | 13333 |
| 3 | 0.5 | 4000 | 8000 |
| 4 | 1.2 | 100 | 83 |
| 5 | 1.6 | 12000 | 7500 |
| 6 | 1.8 | 180 | 100 |
| 7 | 1.1 | 480 | 436 |
| 8 | 1.1 | 12000 | 10909 |
| 9 | 0.8 | 12000 | 15000 |
| 10 | 4.9 | 12000 | 2449 |
| 11 | 1.1 | 12000 | 10909 |
| 12 | 1.1 | 4000 | 3636 |
| 13 | 1.0 | 12000 | 12000 |
| 14 | 1.3 | 12000 | 9231 |
| 15 | 1.0 | 12000 | 12000 |
| 16 | 1.6 | 450 | 281 |
| 17 | 0.9 | 12000 | 13333 |
| 18 | 0.5 | 12000 | 24000 |
| 19 | 1.4 | 12000 | 8571 |
| 20 | 1.7 | 12000 | 7059 |
| 21 | 1.3 | 13000 | 10000 |
| 22 | 0.7 | 1300 | 1857 |
| 23 | 1.2 | 12000 | 10000 |
| 24 | 0.5 | 120 | 240 |
| 25 | 0.9 | 360 | 400 |
| 26 | 3.1 | 12000 | 3871 |
| 27 | 1.8 | 80 | 44 |
| 28 | 1.3 | 4000 | 3077 |
| 29 | 1.0 | 180 | 180 |
| 30 | 1.0 | 12000 | 12000 |
| 31 | 0.5 | 1300 | 2600 |
| 32 | 0.9 | 12000 | 13333 |
| 33 | 1.8 | 12000 | 6667 |
| 34 | 1.8 | 12000 | 6667 |
| 35 | 2.0 | 4000 | 2000 |
| 36 | 0.94 | 12000 | 12000 |
| 37 | 0.62 | 1200 | 1935 |
| 38 | 2 | 1200 | 600 |
| 39 | 0.52 | 4000 | 7692 |
| 40 | 0.42 | 1200 | 2857 |
| 41 | 0.8 | 1200 | 1500 |
| 42 | 0.78 | 1200 | 1538 |
| 43 | 1.22 | ND | ND |
| 44 | 0.57 | 1200 | 2105 |
| 45 | 2.038 | 1200 | 589 |
| 46 | 1.292 | 1200 | 929 |
| 47 | 0.6938 | ND | ND |
| 48 | 0.8969 | ND | ND |
| 49 | 2.91 | ND | ND |
| 50 | 0.449 | 1200 | 2673 |
| 51 | 1.834 | 1200 | 654 |
| 52 | 1.4 | 1200 | 857 |
| 53 | 0.32 | ND | ND |
| 54 | 0.91 | ND | ND |
| 55 | 6.67 | ND | ND |
| 56 | 0.64 | ND | ND |
| 57 | 4.413 | 32 | 7 |
| 58 | 1.67 | ND | ND |
| 59 | 5.059 | 1200 | 237 |
| 60 | 2.232 | 1200 | 538 |
| 61 | 1.49 | 1200 | 805 |
| 62 | 1.48 | 1200 | 811 |
| 63 | 0.9135 | 1200 | 1314 |
| 64 | 2.713 | 1200 | 442 |
| 65 | 1.098 | 1200 | 1093 |
| 66 | 1.7 | 1200 | 706 |
| 67 | 1.503 | 1200 | 798 |
| 68 | 2.557 | 1200 | 469 |
| 69 | 6.574 | 1200 | 183 |
| 70 | 2.545 | 1200 | 472 |
| 71 | 4.089 | 1200 | 293 |
| 72 | 1.944 | 1200 | 617 |
| 73 | 9.165 | ND | ND |
| 74 | 1.941 | 1200 | 618 |
| 75 | 0.9148 | 1200 | 1312 |
| 76 | 1.146 | ND | ND |
| 77 | 1.701 | 1200 | 705 |
| 78 | 1.593 | 1200 | 753 |
| 79 | 5.415 | ND | ND |
| 80 | 1.512 | 1200 | 794 |
| 81 | 1.179 | ND | ND |
| 82 | 1.428 | 1200 | 840 |
| 83 | 2.392 | ND | ND |
| 84 | 2.29 | ND | ND |
| 85 | 1.05 | ND | ND |
| 86 | 2.59 | ND | ND |
| 87 | 0.89 | ND | ND |
| 88 | 0.99 | ND | ND |
| 89 | 2.59 | ND | ND |
| 90 | 1.82 | ND | ND |
| 91 | 2.44 | ND | ND |
| 92 | 0.83 | ND | ND |
| 93 | 3.06 | ND | ND |
| 94 | 0.76 | ND | ND |
| 95 | 1.3 | ND | ND |
| 96 | 5.51 | ND | ND |
| 97 | 4.31 | ND | ND |
| 98 | 1.96 | ND | ND |

ND = Not Determined

### In Vitro Binding of [³H]-2 in Human PD Brain Tissue (Sections and Homogenates)

To assess presence of alpha synucleinopathy (Lewy body, LB and Lewy neurites, LN) in the tested human brain samples, the adjacent human PD brain slices were used for Autoradiography (ARG) and Immunihistochemistry (1HC) studies. ARG was done using radio-labelled compounds which bind to LB and LN of PD brain sections. 1HC was performed with antibodies for LB & LN (LB509), Aβ (6E10) and p-tau (AT8). Tissue homogenate binding was performed using human PD brain homogenates of cerebral cortex. Human brains from donor without neurological disorder were used as control in the same study.

### Procedures of In Vitro Autoradiography:

The frozen human brain samples of Parkinson's disease (PD) were provided by Banner Sun Health Institute (USA) and Sydney Brain Bank (Australia) through collaboration with Michael J Fox Foundation (MJFF). Additional PD and non-PD brain samples were purchased from vendors (Analytic Biological Services Inc., ABS, and Discovery Life Science, DLS). Frozen brain slices (14 m thickness) were prepared using a cryostat (Leica CM3050) and kept in sequential order. The tissue slices were placed on Superfrost Plus glass slides (Cat. #5075-FR, Brain Research Laboratories, USA), dried at room temperature, and stored in a slide box at −70° C. before use. [³H]-2 was synthesized by Radio Compound Labelling Synthesis Group at Merck. The specific activity of [³H]-2 is 62.95 Ci/mmol. The final concentrations of radioligand for in vitro autoradiography was 3 nM. On the day of a binding experiment, adjacent slices were selected from each brain sample interest for in vitro autoradiographic study and were designated as total binding and non-displaceable binding (NDB). These slices were thawed at room temperature for 15 minutes in a biosafety hood. A single concentration of [³H]-2 was applied in the study. Total binding of radioligand in a brain slice was defined in the absence of competitor, and non-displaceable binding (NDB) was determined in the presence of competitor (1.0 μM unlabeled selfblock). The brain slides were first pre-incubated at room temperature for fifteen minutes in PBS buffer, pH 7.4. The slices were then transferred to fresh buffer containing radioligand or radioligand plus competitor as described above and incubated at room temperature for ninety minutes. Incubation was terminated by washing the slices three times in ice cold (4° C.) wash buffer (PBS, pH 7.4) with each wash lasting three minutes. After washing, the slices were briefly rinsed in ice cold (4° C.) deionized water, and then dried completely by an air blower at room temperature. The slices were placed against Fuji Phosphor Image Plates (TR2025) in a sealed cassette for exposure at room temperature. After three weeks exposure, the plates were scanned in Amersham Typhoon Imager, and the scanned images were analyzed using MCID 7.1 software. [³H]-microscales (Amersham Biosciences, GE), were used for quantification of radioligand binding density. All the slice binding assays were done in the laboratory designated for studies using human tissues.

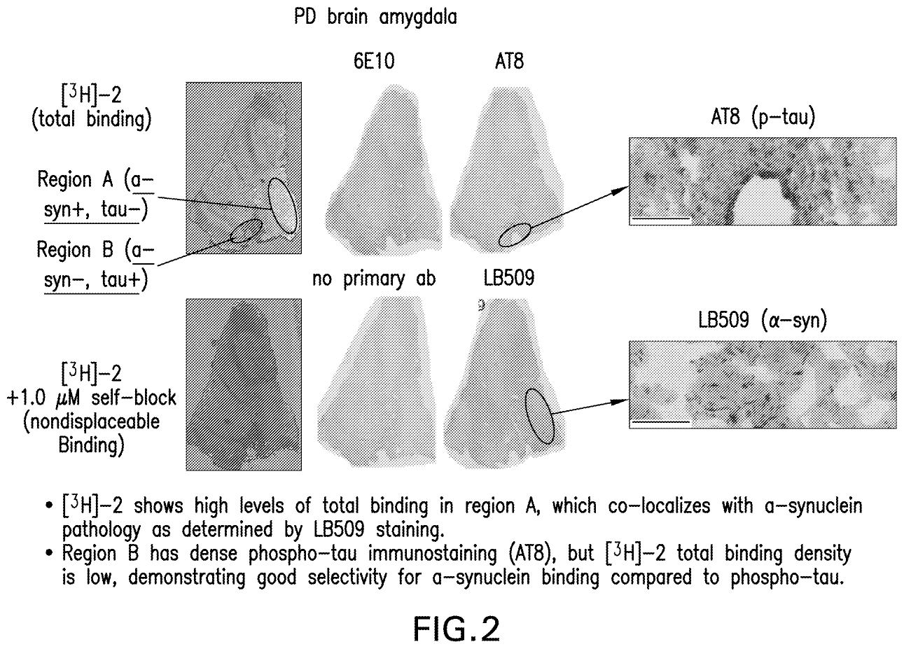

FIG. 2 depicts specific [³H]-2 binding to alpha-synucleinopathy of a PD brain amygdala section. Based on immunoreactivity (IR) of LB509 (al-syn) and AT8 (tau) of 1HC study, the figure shows positive LB509 IR with minimal AT8 IR in region A. In contrast, region B on FIG. 2 shows positive AT8 IR with minimal LB509 IR. The autoradiographic image of [³H]-2 binding only matches to the distinct LB509 IR pattern in region A but not to the AT8 IR pattern in region B by 1HC, indicating specific binding of [³H]-2 to alpha-synucleinopathy of PD brain.

### Procedures of Tissue Homogenate Binding:

The frozen human brain samples of Parkinson's disease (PD) were provided by Banner Sun Health Institute (USA) and Sydney Brain Bank (Australia) through collaboration with Michael J Fox Foundation (MJFF). Additional PD and non-PD brain samples were purchased from vendors (Analytic Biological Services Inc., ABS, and Discovery Life Science, DLS). They are post-mortem tissue from donors with clinical diagnosis of PD or non-PD. Brain homogenates of cerebral cortex were prepared by homogenizing the cortex in ice cold Phosphate Buffered Saline (PBS), pH 7.4, for 30 seconds at 4° C., on setting 6 of Polytron. The final concentration of brain homogenates was 30 mg wet tissue per 1 mL buffer. Homogenates were aliquoted in 1 mL/tube and stored at −70° C. prior to use.

[³H]-2 was synthesized by Radio Compound Labelling Synthesis Group at Merck. The specific activities of [³H]-2 is 62.95 Ci/mmol. For hot saturation binding assay, various concentrations of radioligand were used, ranging from 20 nM to 0.2 nM. Brain homogenates were diluted from original 30 mg/mL volume to final concentration of 2.2 mg/mL with assay buffer (Tris, pH 7.5, 0, 1% BSA), and 250 μl per assay tube was used in assay. Unlabeled test compounds were dissolved in DMSO at 1 mM. Dilution of test compound to various concentrations was made with assay buffer containing 2% DMSO. Total binding was defined in the absence of competing compound, and non-displaceable binding was determined in the presence of 1 μM unlabeled self-block. Compound dilutions (1OX) were added into the assay tube (25 μL each/per tube, separately) containing 200 μL brain homogenate dilution, and pre-incubate the tubes at room temperature for 30 minutes, then radioligand dilutions (1OX) were added into the assay tube (25 μL each/per tube, separately) to a final volume of 250 μL per tube.

Incubation was carried out at 37° C. for 120 minutes, and then the assay samples were filtered onto GF/C filters using Skatron 12 well harvester, washing on setting 5-5-5 (~3×2 ml) ice cold buffer (Tris, pH 7.5). GF/C filter papers for Skatron harvester were pre-soaked in 0.1% BSA for 1 hour at room temperature before use. Filters were punched into scintillation vials. Add liquid scintillation fluid (2 mL Ultima Gold) into each vial, allow to soak into filters for 4 hours and counted in on Perkin Elmer Tri-Carb 2900TR for 1 minute. The data analysis was done with Prism software. All assays were done in either duplicate or triplicate, depending on assay setting, in the laboratory designated for studies using human tissues.

Data Table 2- In Vitro Binding data for [³H]2 in
human Parkinson's Disease tissue homogenates

| [³H]-2 | PD Cortex (n = 5) |
|---|---|
| Bₘₐₓ (nM) | 17.0 ± 5.2 |
| K_{d} (nM) | 0.37 ± 0.17 |
| Bₘₐₓ / K_{d} | 46 |

### Radiochemical Synthesis of [¹¹C]-2

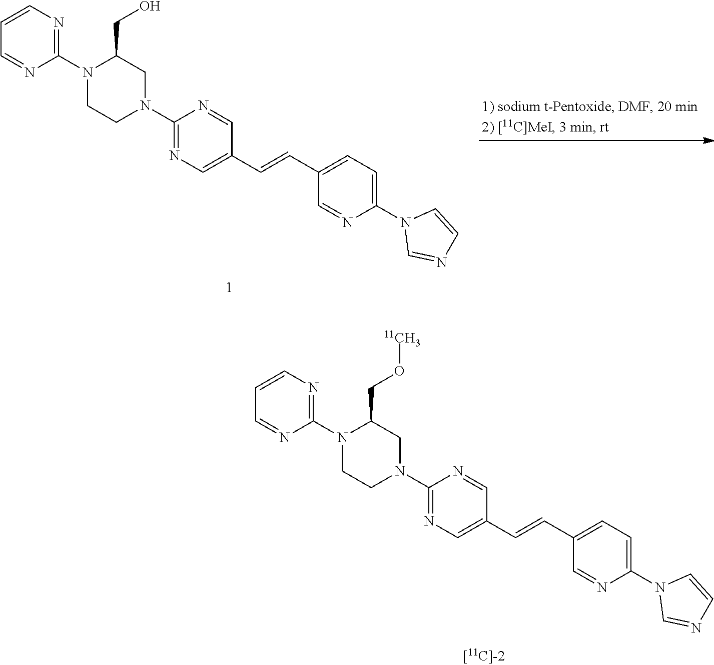 [SMILES: OC[C@H]1CN(c2ncc(/C=C/c3ccc(-n4ccnc4)nc3)cn2)CCN1c1ncccn1 ; [11CH3]OC[C@H]1CN(c2ncc(/C=C/c3ccc(-n4ccnc4)nc3)cn2)CCN1c1ncccn1]

Sodium tert-butoxide 2.5M (1.3 μL, 3.3 μmol) in dimethylformamide (50 μL) was added slowly onto Ex. No. 1 (0.41 mg, 0.93 μmol) in dimethylformamide (250 μL) under Argon at rt. After 15 minutes [¹¹C]methyl iodide was bubbled in the reaction mixture at rt. The solution was kept at rt for 3 minutes, water (700 μL) was added, and the crude was injected into the semi-preparative HPLC column. The product was purified using a Luna, 5u, C18, 250×10 mm (Phenomenex) at a flow rate of 5 mL/min. The mobile phase was acetonitrile/0.10% formic acid from 20 to 50% in 15 min. The radioactive fraction eluting between 10 and 11 minutes was collected, diluted with 20 mL of water for injection, and loaded into a Waters Sep-Pak Classic C18 cartridge (Waters, Milford, MA-USA). The Sep-Pak was rinsed with 10 nL of water and then elated wit ethanol (0.5 mL) into 10 mL sterile Nil and diluted to the desired formulation. The final product was tested for chemical and radiochemical purity by means of an analytical HPLC system (Agilent) using a Gemini, 5p, C18, 150×4.6 mm (Phenomenex) at a flow rate of 1 mL/min. The mobile phase was a mixture consisting of acetonitrile/0.1% trifluoroacetic acid in water from 5 to 90% in 7 min. Ex. No. 2 concentration was determined by means of an ultraviolet detector (254 nm). Confirmation of the identity of the product was determined by co-injection of a sample of Ex. No. 2, and radiochemical purity was determined using a sodium iodide detector (Bioscan). The retention time for [¹¹C]-2 was 5.9 min.

### [¹¹C]-2 Imaging in Rhesus Monkey

A fasted rhesus monkey (7-11 kg) is anesthetized with ketamine I.M. (15 mpk) and the monkey is placed in the PET camera bed. An I.V, catheter is inserted into the right saphenous vein. For arterial sampling, the right femoral area is aseptically prepared and an arterial catheter is placed and fixed with sutures.

Subsequent anesthesia is maintained with Propofol. Induction dose is 5 mg/kg I.V., followed by an infusion at 0.4-0.6 mg/kg/min for the duration of the scanning procedure. The animal is intubated and positioned inside the camera gantry supine, head first. Animal is maintained on ventilated medical grade air:oxygen gas mixture at approximately 23 respirations per minute for the duration of the study. Ventilation I:E ratio, volume and rate of respiration is adjusted to maintain CO₂ levels about 40 mmHg and SpO₂ levels 95 to 100%. A temperature probe, pulse oximeter, non-invasive blood pressure cuff, and end tidal CO₂ monitor are connected. Body temperature is maintained by placing K-module heating pads on dorsal and ventral sides of animal. General fluid therapy is maintained with 10 ml/kg/hr Lactated Ringer's, IV throughout scanning procedure. Another line is placed in lower saphenous artery for sampling and connected to an Instech automated blood sampling system. An aliquot of [¹¹C]-2 is injected IV over 2 min via a syringe pump, with positron emission imaging beginning at the time of injection and continuing for 90 minutes.

Whole blood samples are collected via arterial catheter into Heparin tubes for determination of radioactivity in whole blood and plasma. Samples are centrifuged and 20 μl whole blood and plasma are counted 20, 40, 80, 100, 120, and 150 seconds post PET ligand injection. Samples of blood (0.8 ml) are taken for metabolite correction and determination of radioactivity in plasma and whole blood at 3, 5, 15, 30, 60, and 90 minutes.

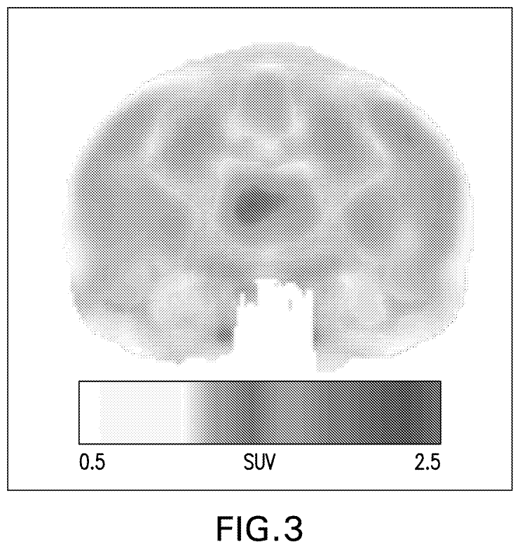

FIG. 3 shows a coronal slice of a PET image of [¹¹C]-2 in rhesus monkey brain. The image is averaged over 30-90 minutes post injection and overlaid on a brain MRI template. This demonstrates suitable distribution of [¹¹C]-2 in monkey brain.

### Radiochemical Synthesis of [¹⁸F]-Ligands

### Radiochemical Synthesis of [¹⁸F]-38

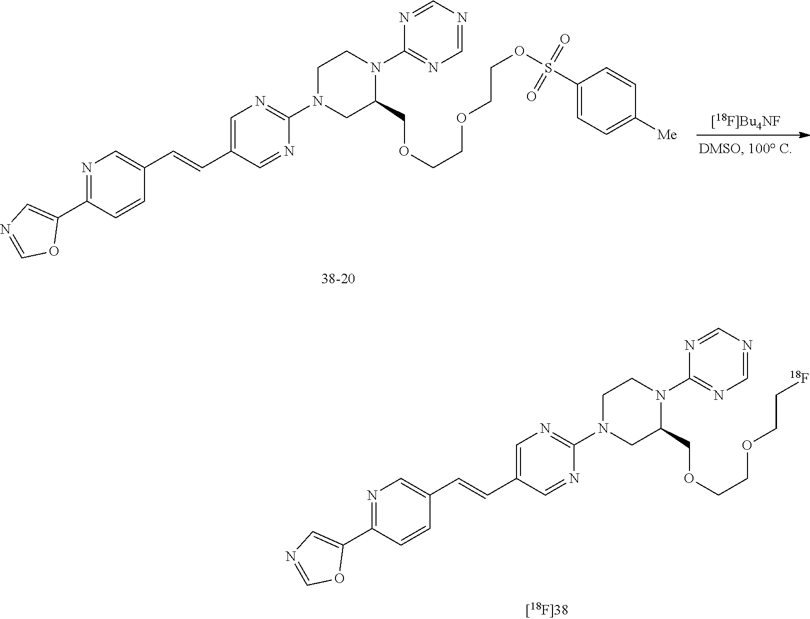 [SMILES: Cc1ccc(S(=O)(=O)OCCOCCOC[C@H]2CN(c3ncc(/C=C/c4ccc(-c5cnco5)nc4)cn3)CCN2c2ncncn2)cc1 ; [18F]CCOCCOC[C@H]1CN(c2ncc(/C=C/c3ccc(-c4cnco4)nc3)cn2)CCN1c1ncncn1]

[¹⁸F]Fluoride was concentrated on an anion exchange resin which was pretreated by flushing with EtOH (10 mL) followed by 0.5M K₃PO₄ in H₂O (10 mL) and H₂O (10 mL) before use.

The [¹⁸F]fluoride containing anion exchange resin was eluted with tetrabutylammonium mesylate (6.8 mg, 20 μmol) in CH₃CN/H₂O 1:1 (1.0 mL), followed by CH₃CN (0.5 mL) into a vented 2.5 mL v-shaped vial and dried under argon flow using conventional heating at 100° C. Additional aliquots of CH₃CN (2×0.5 mL) were added for azeotropic drying. To the vial containing dried [¹⁸F]Bu₄NF was added a solution of 38-20 (0.9 mg, 1.3 mol) in DMSO (0.5 mL). The reaction mixture was heated at 100° C. for 10 min followed by transfer to a vial containing H₂O (0.8 mL) at room temperature for dilution, mixing and injection onto a semi-prep HPLC column. The product was purified using a Gemini C6-Pheny1Hexyl, 5 m, 250×10 mm HPLC column (Phenomonex) with a flowrate of 5 ml/min and a mobile phase of 37% CH₃CN/10 mM Na₂HPO₄ pH 7.4. The radioactive fraction that eluted between 20.6 and 21.6 min was collected into a round bottom flask containing 10% captisol in H₂O (0.5 mL), evaporated under negative pressure to remove CH₃CN and transferred to a 10 mL sterile vial. The final product was tested for chemical and radiochemical purity by means of an analytical HPLC system (Agilent) using a Luna PFP(2), 3 μm, 150×3.Omm HPLC column (Phenomonex) with a flowrate of 0.7 ml/min and a mobile phase of CH₃CN/H₂O at a gradient of 40-50%. Concentration of [¹⁸F]38 was determined by means of an ultraviolet detector (254 nm). Confirmation of the identity of the product was determined by coinjection of a sample of compound 38 and radiochemical purity was determined using a sodium iodide detector (Bioscan). The retention time for compound [¹⁸F]38 was 5.4 min.

### Radiochemical Synthesis of [¹⁸F]44

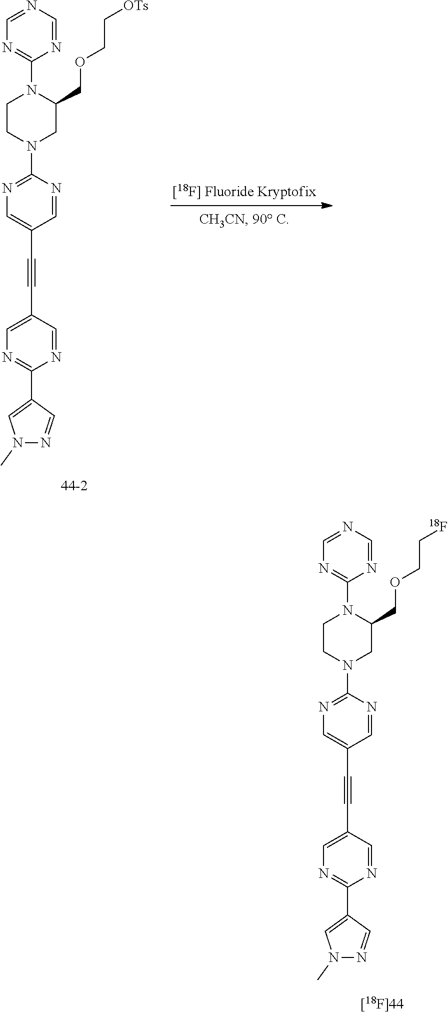 [SMILES: Cc1ccc(S(=O)(=O)OCCOC[C@H]2CN(c3ncc(C#Cc4cnc(-c5cnn(C)c5)nc4)cn3)CCN2c2ncncn2)cc1 ; Cn1cc(-c2ncc(C#Cc3cnc(N4CCN(c5ncncn5)[C@@H](COCC[18F])C4)nc3)cn2)cn1]

[¹⁸F]Fluoride was concentrated on an anion exchange resin and eluted prior to use. The [¹⁸F]fluoride containing anion exchange resin was eluted with Kryptofix 222 (7 mg, 19 μmol) and K₂CO₃ (2.1 mg, 15 μmol) in acetonitrile/water (80/20, 0.7 ml) and transferred to a vented 4 ml vial. The fluoride was dried under argon flow at 90° C. Additional aliquots of acetonitrile (2×0.5 ml) were added for azeotropic drying at 90° C.

A solution of 44-2 (0.51 mg, 0.78 μmol) in CH₃CN (0.5 mL) was added to the 4 ml vial containing the dry [¹⁸F]fluoride, the vent line was removed, and the reaction mixture was heated at 90° C. (60 W) for 15 min. After cooling down to <50° C., the reaction was diluted with HPLC eluent (−2 mL), mixed and injected into the semi-preparative HPLC column. The product was purified using Gemini. C6-Phenyl. 110A, 150×10 mm (Phenomenex), at a flow rate of 5 mL/min. The mobile phase was acetonitrile-10% H₂O/Na₂HPO₄ (10 mM) from 30 to 70% in 15 min.

The radioactive fraction eluting between 15 and 16 minutes was collected in a flask containing a 30% B-cyclodextrin solution (1 mL), evaporated under negative pressure diluted with saline and transferred into a sterile container. The final product was tested for chemical and radiochemical purity by means of an analytical HPLC system (Agilent) using a ONYX Monolithic, 5 g, C18, 100×3 mm (Phenomenex) at a flow rate of 1.5 mL/min. The mobile phase was a mixture consisting of acetonitrile/0.1% formic acid in water from 5 to 90% in 7 min. Concentration of [¹⁸F]44 was determined by means of an ultraviolet detector (254 nm). Confirmation of the identity of the product was determined by coinjection of a sample of compound 44, and radiochemical purity was determined using a sodium iodide detector (Bioscan). The retention time for compound [¹⁸F]44 was 4.1 min.

### Radiochemical Synthesis of [¹⁸F]46

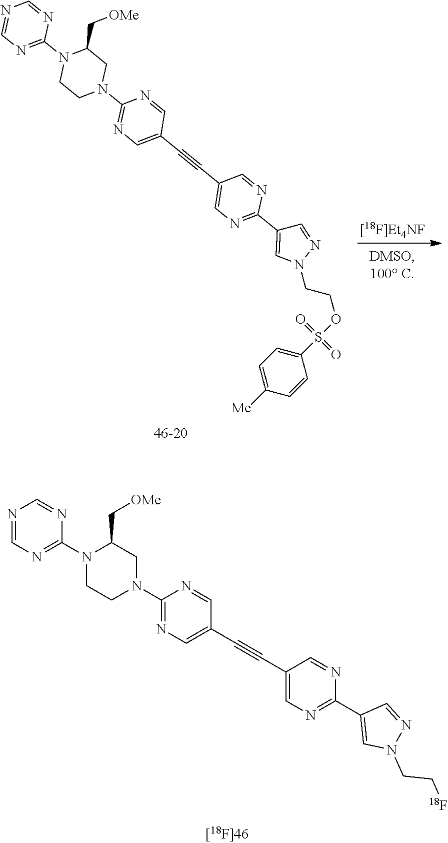 [SMILES: COC[C@H]1CN(c2ncc(C#Cc3cnc(-c4cnn(CCOS(=O)(=O)c5ccc(C)cc5)c4)nc3)cn2)CCN1c1ncncn1 ; COC[C@H]1CN(c2ncc(C#Cc3cnc(-c4cnn(CC[18F])c4)nc3)cn2)CCN1c1ncncn1]

The [¹⁸F]fluoride containing anion exchange resin was eluted with tetraethylammonium bicarbonate (4.2 mg. 22 μmol) in CH₃CN/H₂O 1:1 (1.0 mL), followed by CH₃CN (0.5 mL) into a vented 2.5 mL v-shaped vial and dried under argon flow using conventional heating at 100° C. Additional aliquots of CH₃CN (2×0.5 mL) were added for azeotropic drying. To the vial containing dried [¹⁸F]Et₄NF was added a solution of 46-21 (1.0 mg. 1.4 μmol) in DMSO (0.5 mL). The reaction mixture was heated at 100° C. for 10 min followed by transfer to a vial containing H₂O (0.8 mL) at room temperature for dilution, mixing and injection onto a semi-prep HPLC column. The product was purified using a Gemini C6-Pheny1Hexyl, 5 m, 250×10 mm HPLC column (Phenomonex) with a flowrate of 5 ml/min and a mobile phase of 35% CH₃CN/10 mM Na₂HPO₄ pH 7.4. The radioactive fraction that eluted between 24.1 and 24.6 min was collected into a round bottom flask containing 10% captisol in H₂O (0.5 mL), evaporated under negative pressure to remove CH₃CN and transferred to a 10 mL sterile vial. The final product was tested for chemical and radiochemical purity by means of an analytical HPLC system (Agilent) using a Luna PFP(2), 3 μm, 150×3.Omm HPLC column (Phenomonex) with a flowrate of 1.0 ml/min and a mobile phase of CH₃CN/H₂O at a gradient of 40-50%. Concentration of [¹⁸F]46 was determined by means of an ultraviolet detector (254 nm). Confirmation of the identity of the product was determined by coinjection of a sample of compound 46, and radiochemical purity was determined using a sodium iodide detector (Bioscan). The retention time for compound [¹⁸F]46 was 4.8 min.

The compounds contained in Table 4 were synthesized by analogous methods and precursors from synthetic sequences described above. Commercially available reagents were substituted where necessary to produce the examples below.

### General Methods:

[¹⁸F]Fluoride was concentrated on an anion exchange resin and eluted prior to use. Unless specifically stated, the [¹⁸F]fluoride containing anion exchange resin was eluted with Kryptofix 222 (7 mg, 19 μmol) and K₂CO₃ (2.1 mg, 15 μmol) in acetonitrile/water (80/20, 0.7 ml) and transferred to a vented 4 ml vial. The fluoride was dried under argon flow at 90° C. Additional aliquots of acetonitrile (2×0.5 ml) were added for azeotropic drying at 90° C.

### Column Descriptions;

Column 1: Phenomenex Gemini 5 μm C6-Phenyl 110 A 250×10 mm
Column 2: Onyx Monolithic C18 100×3.0 mm
Column 3: Agilent Zorbax Eclipse XDB-C18 9.4×250 mm 5p
Column 4: Phenomenex Gemini 5μ C18 150×4.6 mm

| | | Semi- | | Semi- | | | Analytical |
|---|---|---|---|---|---|---|---|
| ¹⁸F | Reaction | prep | | prep rt | Analytical | Analytical | rt |
| example | conditions | column | Semi-prep conditions | (min) | column | conditions | (min) |
| | 140° C. | Column | 30 to 70 in 15AD_5 mL | 14.5 | Column | A: ACN, | 3.8 |
| 29 | (MW). | 3 | A: CH3CN, D: | | 4 | D: 0.1% | |
| | 5 min. | | Na2HPO4 10 mM | | | HCOOH | |
| | DMF | | | | | 05 to 90 | |
| | | | | | | AB in 7 | |
| | | | | | | min, 1.5 | |
| | | | | | | ml/min | |
| 36 | 90° C., | Column | 40 to 70 in 15AD_5 mL | 13 | Column | A: ACN, | 5.2 |
| | 15 min, | 3 | A: CH3CN, D: | | 4 | D: 0.1% | |
| | CH₃CN | | Na2HPO4 10 mM | | | HCOOH | |
| | | | | | | 05 to 90 | |
| | | | | | | AB in 7 | |
| | | | | | | min, 1.5 | |
| | | | | | | ml/min | |
| 41 | 90° C., | Column | 40 to 70 in 15AD_5 mL | 12 | Column | A: ACN, | 4.4 |
| | 15 min, | 3 | A: CH3CN, D: | | 2 | D: 0.1% | |
| | CH₃CN | | Na2HPO4 10 mM | | | HCOOH | |
| | | | | | | 05 to 70 | |
| | | | | | | AD in 7 | |
| | | | | | | min, 1.5 | |
| | | | | | | ml/min | |
| 43 | 90° C., | Column | 40 to 70 in 15AD_5 mL | 12.5 | Column | A: ACN, | 4.4 |
| | 15 min, | 3 | A: CH3CN, D: | | 2 | D: 0.1% | |
| | CH₃CN | | Na2HPO4 10 mM | | | HCOOH | |
| | | | | | | 05 to 70 | |
| | | | | | | AD in 7 | |
| | | | | | | min, 1.5 | |
| | | | | | | ml/min | |
| 47 | 70° C., | Column | 40 to 70 in 15AD_5 mL | 15 | Column | A: ACN, | 4.3 |
| | 15 min, | 3 | A: CH3CN, D: | | 2 | D: 0.1% | |
| | CH₃CN | | Na2HPO4 10 mM | | | HCOOH | |
| | | | | | | 05 to 70 | |
| | | | | | | AD in 7 | |
| | | | | | | min, 1.5 | |
| | | | | | | ml/min | |
| 48 | 90° C., | Column | 40 to 70 in 15AD_5 mL | 14 | Column | A: ACN, | 4.3 |
| | 15 min, | 3 | A: CH3CN, D: | | 4 | D: 0.1% | |
| | CH₃CN | | Na2HPO4 10 mM | | | HCOOH | |
| | | | | | | 05 to 90 | |
| | | | | | | AB in 7 | |
| | | | | | | min, 1.5 | |
| | | | | | | ml/min | |
| 52 | 90° C., | Column | 30 to 70 in 15AD_5 mL | 18.5 | Column | A: ACN, | 4.6 |
| | 15 min, | 1 | A: 90% CH3CN- | | 2 | D: 0.1% | |
| | CH₃CN | | 10% H2O, D: | | | HCOOH | |
| | | | Na2HPO4 10 mM | | | 05 to 90 | |
| | | | | | | AD in 7 | |
| | | | | | | min, 1.5 | |
| | | | | | | ml/min | |
| 53 | 90° C., | Column | 40 to 70 in 15AD_5 mL | 14.2 | Column | A: ACN, | 4.2 |
| | 15 min, | 3 | A: CH3CN, D: | | 2 | D: 0.1% | |
| | CH₃CN | | Na2HPO4 10 mM | | | HCOOH | |
| | | | | | | 05 to 70 | |
| | | | | | | AD in 7 | |
| | | | | | | min, 1.5 | |
| | | | | | | ml/min | |
| 54 | 70° C., | Column | 30 to 70 in 15AD_5 mL | 15 | Column | A: ACN, | 4.1 |
| | 15 min, | 1 | A: 90% CH3CN- | | 2 | D: 0.1% | |
| | CH₃CN | | 10% H2O, D: | | | HCOOH | |
| | | | Na2HPO4 10 mM | | | 05 to 90 | |
| | | | | | | AD in 7 | |
| | | | | | | min, 1.5 | |
| | | | | | | ml/min | |

While the invention has been described and illustrated wxith reference to certain particular embodiments thereof, those skilled in the art will appreciate that various adaptations, changes, modifications, substitutions, deletions, or additions of procedures and protocols may be made without departing from the spirit and scope of the invention. It is intended, therefore, that the invention be defined by the scope of the claims that followv and that such claims be interpreted as broadly as is reasonable.

1. A compound of Formula I: 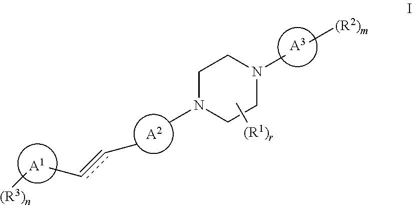
or a pharmaceutically acceptable salt thereof wherein; [symbol]
may be absent or may represent a bond;
R is independently selected from H or —C₁₋₆alkyl, where said alkyl is optionally substituted with one to three groups from —C₁₋₆alkyl, OR^{a} or halo;
R^{a} is independently selected from H, —C₁₋₆alkyl, —(CH₂)ₚOR, —(CH₂)ₚhalo, or —(CH₂)ₚO(CH₂)ₚhalo;
R^{b} is independently selected from H, —C₁₋₆alkyl, heterocyclyl, heteroaryl, —(CH₂)ₚOR, —CN, —(CH₂)ₜhalo, —(CH₂)ₛNR₂ or —O(CH₂)ₚhalo;
R^{c} is independently selected from H, halo, OR, or —C₁₋₆alkyl, where said alkyl is optionally substituted with one to three groups from —C₁₋₆alkyl, OR^{a} or halo;
R¹ is selected —(CH₂)ₛOR, —(CH₂)ₛNR₂, —(CH₂)ₛ[O(R^{c}₂)ₚ]ₓ—R^{c}, or —(CH₂)ₛhalo;
R² is independently selected from H, OR, CN, halo or —C₁₋₆alkyl, where said alkyl is optionally substituted with one to three groups from —C₁₋₆alkyl, OR^{a} or halo;
R³ is independently selected from —C₁₋₆alkyl, heteroaryl or heterocyclyl, wherein said alkyl, heteroaryl or heterocyclyl is optionally substituted with one to three groups from OR^{a} or R^{b};
Ring A¹ is selected from pyridinyl, imidazo-pyrimidinyl, triazinyl, pyrimidinyl, imidazo-pyridinyl, pyrazinyl or pyridazinyl;
Ring A² is selected from pyrimidinyl, pyridinyl, pyrazinyl or phenyl wherein said pyrimidinyl, pyridinyl, pyrazinyl or phenyl is optionally substituted with one to three groups from —C₁₋₆alkyl, OR^{a} or halo;
Ring A³ is selected from pyridinyl, pyrazinyl, pyrimidinyl, pyrrolopyrazinyl, triazinyl, indolyl, imidazolyl, oxadiazolyl, triazolyl, thiazolyl, isoxazolyl, oxazolyl, 3,4-dihydro-2H-pyrido[3,2,b][1,4]oxazine or phenyl;
m is selected from 0, 1, or 2;
n is selected from 1, 2, or 3;
p is independently selected from 1, 2 or 3;
r is selected from 1, 2 or 3;
s is independently selected from 0, 1, 2, 3, 4, 5 or 6;
t is independently selected from 0, 1, 2, 3, 4, 5 or 6; and
x is independently selected from 1, 2, 3, 4, 5 or 6.

2. The compound of claim 1, having the structure of Formula IA: 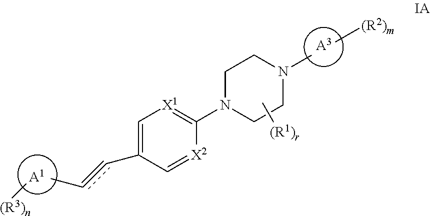
or a pharmaceutically acceptable salt thereof wherein; [symbol]
may be absent or may represent a bond;
R is independently selected from H or —C₁₋₆alkyl, where said alkyl is optionally substituted with one to three groups from —C₁₋₆alkyl, OR^{a} or halo;
R^{a} is independently selected from H, —C₁₋₆alkyl, —(CH₂)ₚOR, —(CH₂)ₚhalo, or —(CH₂)ₚO(CH₂)ₚhalo;
R^{b} is independently selected from H, —C₁₋₆alkyl, heterocyclyl, heteroaryl, —(CH₂)ₚOR, —CN, —(CH₂)ₜhalo, —(CH₂)ₛNR₂ or —O(CH₂)ₚhalo;
R^{c} is independently selected from H, halo, OR, or —C₁₋₆alkyl, where said alkyl is optionally substituted with one to three groups from —C₁₋₆alkyl, OR^{a} or halo;
R¹ is selected —(CH₂)ₛOR, —(CH₂)ₛNR₂, —(CH₂)ₛ[O(R^{c}₂)ₚ]ₓ—R^{c} or —(CH₂)ₛhalo;
R² is independently selected from H, OR, CN, halo or —C₁₋₆alkyl, where said alkyl is optionally substituted with one to three groups from —C₁₋₆alkyl, OR^{a} or halo;
R³ is independently selected from —C₁₋₆alkyl, heteroaryl or heterocyclyl, wherein said alkyl, heteroaryl or heterocyclyl is optionally substituted with one to three groups from OR^{a} or R^{b};
X¹ is N or CH;
X² is N or CH;
Ring A¹ is selected from pyridinyl, imidazo-pyrimidinyl, triazinyl, pyrimidinyl, imidazo-pyridinyl, pyrazinyl or pyridazinyl;
Ring A³ is selected from pyridinyl, pyrazinyl, pyrimidinyl, pyrrolopyrazinyl, triazinyl, indolyl, imidazolyl, oxadiazolyl, triazolyl, thiazolyl, isoxazolyl, oxazolyl, 3,4-dihydro-2H-pyrido[3,2,b][1,4]oxazine or phenyl;
m is selected from 0, 1, or 2;
n is selected from 1, 2, or 3;
p is independently selected from 1, 2 or 3;
r is selected from 1, 2 or 3;
s is independently selected from 0, 1, 2, 3, 4, 5 or 6;
t is independently selected from 0, 1, 2, 3, 4, 5 or 6; and
x is independently selected from 1, 2, 3, 4, 5, or 6.

3. The compound of claim 1 having the structure Formula IB 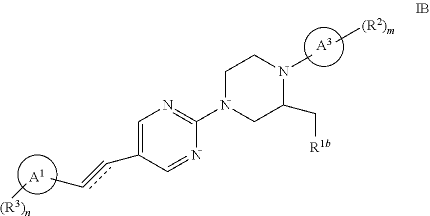
or a pharmaceutically acceptable salt thereof wherein; [symbol]
may be absent or may represent a bond;
R is independently selected from H or —C₁₋₆alkyl, where said alkyl is optionally substituted with one to three groups from —C₁₋₆alkyl, OR^{a} or halo;
R^{a} is independently selected from H, —C₁₋₆alkyl, —(CH₂)ₚOR, —(CH₂)ₚhalo, or —(CH₂)ₚO(CH₂)ₚhalo;
R^{b} is independently selected from H, —C₁₋₆alkyl, heterocyclyl, heteroaryl, —(CH₂)ₚOR, —CN, —(CH₂)ₜhalo, —(CH₂)ₛNR₂ or —O(CH₂)ₚhalo;
R^{c} is independently selected from H, halo, OR, or —C₁₋₆alkyl, where said alkyl is optionally substituted with one to three groups from —C₁₋₆alkyl, OR^{a} or halo;
R^{1b} is selected from —[O(R^{c}₂)ₚ]ₓ—R^{c}, OR, NR₂ or halo;
R² is independently selected from H, OR, CN, halo or —C₁₋₆alkyl, where said alkyl is optionally substituted with one to three groups from —C₁₋₆alkyl, OR^{a} or halo;
R³ is independently selected from —C₁₋₆alkyl, imidazolyl, morpholinyl, triazolyl, oxazolyl, triazinyl, pyrazolyl, thiazolyl, pyrazinyl, isoxazolyl, azetidinyl, pyrrolidinyl, tetrahydrotriazolopyrazinyl, piperidinyl or pyrimidinyl, wherein said alkyl, imidazolyl, morpholinyl, triazolyl, oxazolyl, triazinyl, pyrazolyl, thiazolyl, pyrazinyl, isoxazolyl, azetidinyl, pyrrolidinyl, tetrahydrotriazolopyrazinyl, piperidinyl or pyrimidinyl is optionally substituted with one to three groups from OR^{a} or R^{b};
Ring A¹ is selected from pyridinyl, imidazo-pyrimidinyl, triazinyl, pyrimidinyl, imidazo-pyridinyl, pyrazinyl or pyridazinyl;
Ring A³ is selected from pyridinyl, pyrazinyl, pyrimidinyl, pyrrolopyrazinyl, triazinyl, indolyl, imidazolyl, oxadiazolyl, triazolyl, thiazolyl, isoxazolyl, oxazolyl, 3,4-dihydro-2H-pyrido[3,2,b][1,4]oxazine or phenyl;
m is selected from 0, 1, or 2;
n is selected from 1, 2, or 3;
p is independently selected from 1, 2 or 3;
s is independently selected from 0, 1, 2, 3, or 4;
t is independently selected from 0, 1, 2, 3, 4, 5 or 6; and
x is independently selected from 1, 2, 3, 4, 5 or 6.

4. The compound of claim 1, having the structure of Formula IC 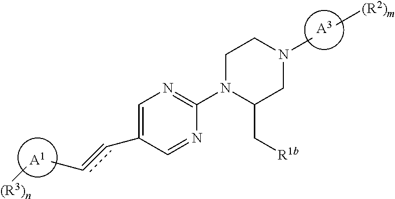
or a pharmaceutically acceptable salt thereof wherein; [symbol]
may be absent or may represent a bond;
R is independently selected from H or —C₁₋₆alkyl, where said alkyl is optionally substituted with one to three groups from —C₁₋₆alkyl, OR^{a} or halo;
R^{a} is independently selected from H, —C₁₋₆alkyl, heterocyclyl, heteroaryl, —(CH₂)ₚOR, —(CH₂)ₚhalo, or —(CH₂)ₚO(CH₂)ₚhalo;
R is independently selected from H, —C₁₋₆alkyl, —(CH₂)ₚOR, —CN, —(CH₂)ₜhalo, —(CH₂)ₛNR₂ or —O(CH₂)ₚhalo;
R^{c} is independently selected from H, halo, OR, or —C₁₋₆alkyl, where said alkyl is optionally substituted with one to three groups from —C₁₋₆alkyl, OR^{a} or halo;
R^{1b} is selected from —[O(R^{c}₂)ₚ]ₓ—R^{c}, OR, NR₂ or halo;
R² is independently selected from H, OR, CN, halo or —C₁₋₆alkyl, where said alkyl is optionally substituted with one to three groups from —C₁₋₆alkyl, ORaor halo;
R³ is independently selected from —C₁₋₆alkyl, imidazolyl, morpholinyl, triazolyl, oxazolyl, triazinyl, pyrazolyl, thiazolyl, pyrazinyl, isoxazolyl, azetidinyl, pyrrolidinyl, tetrahydrotriazolopyrazinyl, piperidinyl or pyrimidinyl, wherein said alkyl, imidazolyl, morpholinyl, triazolyl, oxazolyl, triazinyl, pyrazolyl, thiazolyl, pyrazinyl, isoxazolyl, azetidinyl, pyrrolidinyl, tetrahydrotriazolopyrazinyl, piperidinyl or pyrimidinyl is optionally substituted with one to three groups from OR^{a} or R^{b};
Ring A¹ is selected from pyridinyl, imidazo-pyrimidinyl, triazinyl, pyrimidinyl, imidazo-pyridinyl, pyrazinyl or pyridazinyl;
Ring A³ is selected from pyridinyl, pyrazinyl, pyrimidinyl, pyrrolopyrazinyl, triazinyl, indolyl, imidazolyl, oxadiazolyl, triazolyl, thiazolyl, isoxazolyl, oxazolyl, 3,4-dihydro-2H-pyrido[3,2,b][1,4]oxazine or phenyl;
m is selected from 0, 1, or 2;
n is selected from 1, 2, or 3;
p is independently selected from 1, 2 or 3;
s is independently selected from 0, 1, 2, 3, or 4;
t is independently selected from 0, 1, 2, 3, 4, 5 or 6; and
x is independently selected from 0, 1, 2, 3, 4, 5 or 6.

5. The compound of claim 1, or a pharmaceutically acceptable salt thereof, wherein Ring A¹ is selected from pyrimidinyl or pyridinyl;
Ring A³ is selected from pyridinyl, pyrazinyl, pyrimidinyl, or triazinyl;
m is selected from 0 or 1; and
n is selected from 1 or 2.

6. The compound of claim 1, or a pharmaceutically acceptable salt thereof, wherein Ring A¹ is pyridinyl or pyrimidinyl.

7. The compound of claim 1, or a pharmaceutically acceptable salt thereof, wherein Ring A² is pyrimidinyl.

8. The compound of claim 1, or a pharmaceutically acceptable salt thereof, wherein Ring A³ is selected from pyridinyl, pyrimidinyl, pyrazinyl or triazinyl.

9. The compound of claim 1, or a pharmaceutically acceptable salt thereof, wherein R¹ is —(CH₂)SOR or —(CH₂)ₛ[O(R^{c}₂)ₚ]ₓ—R^{c}.

10. The compound of claim 1, or a pharmaceutically acceptable salt thereof, wherein R³ is independently selected from —C₁₋₆alkyl, imidazolyl, morpholinyl, triazolyl, oxazolyl, triazinyl, pyrazolyl, or isoxazolyl, wherein said alkyl, imidazolyl, morpholinyl, triazolyl, oxazolyl, triazinyl, pyrazolyl, or isoxazolyl is optionally substituted with one to three groups from OR^{a} or R^{b}.

11. A compound selected from

| Ex | |
|---|---|
| No. | Chemical Name |
| 1 | (R,E)-(4-(5-(2-(6-(1H-imidazol-1-yl)pyridin-3-yl)vinyl)pyrimidin-2-yl)-1-(pyrimidin- |
| | 2-yl)piperazin-2-yl)methanol; |
| 2 | (R,E)-5-(2-(6-(1H-imidazol-1-yl)pyridin-3-yl)vinyl)-2-(3-(methoxymethyl)-4- |
| | (pyrimidin-2-yl)piperazin-1-yl)pyrimidine; |
| 3 | (R,E)-5-(2-(6-(1H-imidazol-1-yl)pyridin-3-yl)vinyl)-2-(3-((2-fluoroethoxy)methyl)-4- |
| | (pyrimidin-2-yl)piperazin-1-yl)pyrimidine; |
| 4 | (R,E)-5-(2-(6-(1H-imidazol-1-yl)pyridin-3-yl)vinyl)-2-(3-((fluoromethoxy)methyl)-4- |
| | (pyrimidin-2-yl)piperazin-1-yl)pyrimidine; |
| 5 | (R,E)-5-(2-(6-(4H-1,2,4-triazol-4-yl)pyridin-3-yl)vinyl)-2-(2-(methoxymethyl)-4- |
| | (pyridin-2-yl)piperazin-1-yl)pyrimidine; |
| 6 | (R,E)-5-(2-(6-(1H-imidazol-1-yl)pyridin-3-yl)vinyl)-2-(2-((2-methoxyethoxy)methyl)- |
| | 4-(pyridin-2-yl)piperazin-1-yl)pyrimidine; |
| 7 | (R,E)-(2-(4-(5-(2-(6-(1H-imidazol-1-yl)pyridin-3-yl)vinyl)pyrimidin-2-yl)-2- |
| | (methoxymethyl)piperazin-1-yl)pyrimidin-5-yl)methanol; |
| 8 | (R,E)-5-(2-(6-(1H-imidazol-1-yl)pyridin-3-yl)vinyl)-2-(4-(5-(fluoromethyl)pyrimidin- |
| | 2-yl)-3-(methoxymethyl)piperazin-1-yl)pyrimidine; |
| 9 | (R,E)-5-(2-(6-(1H-imidazol-1-yl)pyridin-3-yl)vinyl)-2-(2-(methoxymethyl)-4-(5- |
| | (methoxymethyl)pyrimidin-2-yl)piperazin-1-yl)pyrimidine; |
| 10 | (R)-(4-(5-((6-(1H-imidazol-1-yl)pyridin-3-yl)ethynyl)pyrimidin-2-yl)-1-(pyrimidin-2- |
| | yl)piperazin-2-yl)methanol; |
| 11 | (R)-5-((6-(1H-imidazol-1-yl)pyridin-3-yl)ethynyl)-2-(3-(methoxymethyl)-4- |
| | (pyrimidin-2-yl)piperazin-1-yl)pyrimidine; |
| 12 | (R)-5-((6-(1H-imidazol-1-yl)pyridin-3-yl)ethynyl)-2-(4-(6-fluoropyrimidin-4-yl)-3- |
| | (methoxymethyl)piperazin-1-yl)pyrimidine; |
| 13 | (R,E)-5-(2-(6-(1H-imidazol-1-yl)pyridin-3-yl)vinyl)-2-(4-(6-fluoropyrimidin-4-yl)-2- |
| | (methoxymethyl)piperazin-1-yl)pyrimidine; |
| 14 | (R,E)-5-(2-(6-(1H-imidazol-1-yl)pyridin-3-yl)vinyl)-2-(4-(6-chloropyrimidin-4-yl)-2- |
| | (methoxymethyl)piperazin-1-yl)pyrimidine; |
| 15 | (R,E)-(1-(5-(2-(6-(1H-imidazol-1-yl)pyridin-3-yl)vinyl)pyrimidin-2-yl)-4-(6- |
| | fluoropyrimidin-4-yl)piperazin-2-yl)methanol; |
| 16 | (R,E)-4-(5-(2-(2-(4-(4-fluoropyridin-2-yl)-2-(methoxymethyl)piperazin-1- |
| | yl)pyrimidin-5-yl)vinyl)pyridin-2-yl)morpholine; |
| 17 | (R,E)-5-(2-(6-(1H-imidazol-1-yl)pyridin-3-yl)vinyl)-2-(4-(4-fluoropyridin-2-yl)-2- |
| | (methoxymethyl)piperazin-1-yl)pyrimidine; |
| 18 | (R,E)-5-(2-(6-(1H-imidazol-1-yl)pyridin-3-yl)vinyl)-2-(2-(methoxymethyl)-4- |
| | (pyridin-2-yl)piperazin-1-yl)pyrimidine; |
| 19 | (R,E)-6-(2-(2-(2-(methoxymethyl)-4-(pyridin-2-yl)piperazin-1-yl)pyrimidin-5- |
| | yl)vinyl)-2-methylimidazo[1,2-a]pyrimidine; |
| 20 | (R,E)-2-(5-(2-(2-(2-(methoxymethyl)-4-(pyridin-2-yl)piperazin-1-yl)pyrimidin-5- |
| | yl)vinyl)pyridin-2-yl)oxazole; |
| 21 | (R,E)-7-(4-(5-(2-(6-(1H-pyrazol-1-yl)pyridin-3-yl)vinyl)pyrimidin-2-yl)-3- |
| | (methoxymethyl)piperazin-1-yl)pyrrolo[1,2-a]pyrazine; |
| 22 | (R,E)-5-(2-(6-(1H-imidazol-1-yl)pyridin-3-yl)vinyl)-2-(3-(methoxymethyl)-4- |
| | (pyridin-2-yl)piperazin-1-yl)pyrimidine; |
| 23 | (R,E)-(1-(5-(2-(6-(1H-imidazol-1-yl)pyridin-3-yl)vinyl)pyrimidin-2-yl)-4-(pyrazin-2- |
| | yl)piperazin-2-yl)methanol; |
| 24 | (R,E)-(1-(5-(2-(6-(1H-imidazol-1-yl)pyridin-3-yl)vinyl)pyrimidin-2-yl)-4-(pyrimidin- |
| | 4-yl)piperazin-2-yl)methanol; |
| 25 | (R,E)-5-(2-(6-(1H-imidazol-1-yl)pyridin-3-yl)vinyl)-2-(2-(methoxymethyl)-4-(5- |
| | methylpyrimidin-2-yl)piperazin-1-yl)pyrimidine; |
| 26 | (R,E)-5-(5-(2-(2-(2-(methoxymethyl)-4-(pyridin-2-yl)piperazin-1-yl)pyrimidin-5- |
| | yl)vinyl)pyridin-2-yl)oxazole; |
| 27 | (R,E)-5-(2-(6-(1H-imidazol-1-yl)pyridin-3-yl)vinyl)-2-(4-(5-fluoropyrimidin-2-yl)-3- |
| | (methoxymethyl)piperazin-1-yl)pyrimidine; |
| 28 | (R,E)-5-(2-(6-(1H-imidazol-1-yl)pyridin-3-yl)vinyl)-2-(2-(methoxymethyl)-4- |
| | (pyrimidin-2-yl)piperazin-1-yl)pyrimidine; |
| 29 | (R,E)-5-(2-(6-(1H-imidazol-1-yl)pyridin-3-yl)vinyl)-2-(4-(2-fluoropyridin-4-yl)-2- |
| | (methoxymethyl)piperazin-1-yl)pyrimidine; |
| 30 | (R,E)-5-(2-(6-(1H-imidazol-1-yl)pyridin-3-yl)vinyl)-2-(2-((2-fluoroethoxy)methyl)-4- |
| | (pyrimidin-2-yl)piperazin-1-yl)pyrimidine; |
| 31 | (R,E)-5-(2-(6-(1H-imidazol-1-yl)pyridin-3-yl)vinyl)-2-(2-((2-fluoroethoxy)methyl)-4- |
| | (pyridin-4-yl)piperazin-1-yl)pyrimidine; |
| 32 | (R,E)-5-(2-(6-(1H-imidazol-1-yl)pyridin-3-yl)vinyl)-2-(4-(6-fluoropyridin-3-yl)-2- |
| | (methoxymethyl)piperazin-1-yl)pyrimidine; |
| 33 | (R,E)-(1-(5-(2-(6-(1H-imidazol-1-yl)pyridin-3-yl)vinyl)pyrimidin-2-yl)-4-(6- |
| | fluoropyridin-3-yl)piperazin-2-yl)methanol; |
| 34 | (R,E)-4-(4-(5-(2-(6-(1H-imidazol-1-yl)pyridin-3-yl)vinyl)pyrimidin-2-yl)-2- |
| | (methoxymethyl)piperazin-1-yl)-2-fluoropyrimidine; and |
| 35 | (R,E)-5-(2-(6-(1H-imidazol-1-yl)pyridin-3-yl)vinyl)-2-(4-(5-(2- |
| | fluoroethoxy)pyrimidin-2-yl)-3-(methoxymethyl)piperazin-1-yl)pyrimidine; |
| 36 | (R,E)-5-(5-(2-(2-(3-((2-fluoroethoxy)methyl)-4-(pyrimidin-2-yl)piperazin-1- |
| | yl)pyrimidin-5-yl)vinyl)pyridin-2-yl)oxazole |
| 37 | (R,E)-5-(2-(6-(1-(2-fluoroethyl)-1H-pyrazol-4-yl)pyridin-3-yl)vinyl)-2-(3- |
| | (methoxymethyl)-4-(pyrimidin-2-yl)piperazin-1-yl)pyrimidine |
| 38 | (R,E)-5-(5-(2-(2-(3-((2-(2-fluoroethoxy)ethoxy)methyl)-4-(1,3,5-triazin-2- |
| | yl)piperazin-1-yl)pyrimidin-5-yl)vinyl)pyridin-2-yl)oxazole |
| 39 | 1-((2-((R)-4-(5-((E)-2-(6-(1H-imidazol-1-yl)pyridin-3-yl)vinyl)pyrimidin-2-yl)-2- |
| | (methoxymethyl)piperazin-1-yl)pyrimidin-5-yl)oxy)-3-fluoropropan-2-ol |
| 40 | (R,E)-2-(3-((2-fluoroethoxy)methyl)-4-(pyrimidin-2-yl)piperazin-1-yl)-5-(2-(6-(4- |
| | methyl-1H-imidazol-1-yl)pyridin-3-yl)vinyl)pyrimidine |
| 41 | (R,E)-5-(5-(2-(2-(3-((2-fluoroethoxy)methyl)-4-(1,3,5-triazin-2-yl)piperazin-1- |
| | yl)pyrimidin-5-yl)vinyl)pyridin-2-yl)oxazole |
| 42 | (R,E)-5-(5-(2-(2-(3-((2-fluoroethoxy)methyl)-4-(pyrimidin-4-yl)piperazin-1- |
| | yl)pyrimidin-5-yl)vinyl)pyridin-2-yl)oxazole |
| 43 | (R,E)-2-(2-((2-fluoroethoxy)methyl)-4-(5-(2-(2-(1-methyl-1H-pyrazol-4-yl)pyrimidin- |
| | 5-yl)vinyl)pyrimidin-2-yl)piperazin-1-yl)-1,3,5-triazine |
| 44 | (R)-2-(2-((2-fluoroethoxy)methyl)-4-(5-((2-(1-methyl-1H-pyrazol-4-yl)pyrimidin-5- |
| | yl)ethynyl)pyrimidin-2-yl)piperazin-1-yl)-1,3,5-triazine |
| 45 | (R)-5-((6-(4H-1,2,4-triazol-4-yl)pyridin-3-yl)ethynyl)-2-(4-(6-fluoropyrimidin-4-yl)- |
| | 3-(methoxymethyl)piperazin-1-yl)pyrimidine |
| 46 | (R)-2-(4-(5-((2-(1-(2-fluoroethyl)-1H-pyrazol-4-yl)pyrimidin-5-yl)ethynyl)pyrimidin- |
| | 2-yl)-2-(methoxymethyl)piperazin-1-yl)-1,3,5-triazine |
| 47 | (R)-2-(4-(5-((6-(1-(2-fluoroethyl)-1H-pyrazol-4-yl)pyridin-3-yl)ethynyl)pyrimidin-2- |
| | yl)-2-(methoxymethyl)piperazin-1-yl)-1,3,5-triazine |
| 48 | (R)-5-(5-((2-(3-((2-(2-fluoroethoxy)ethoxy)methyl)-4-(1,3,5-triazin-2-yl)piperazin-1- |
| | yl)pyrimidin-5-yl)ethynyl)pyridin-2-yl)oxazole |
| 49 | (R)-2-(2-((2-fluoroethoxy)methyl)-4-(5-((2-(1-methyl-1H-pyrazol-3-yl)pyrimidin-5- |
| | yl)ethynyl)pyrimidin-2-yl)piperazin-1-yl)-1,3,5-triazine |
| 50 | (R)-2-(2-((2-fluoroethoxy)methyl)-4-(5-((6-(1-methyl-1H-pyrazol-4-yl)pyridin-3- |
| | yl)ethynyl)pyrimidin-2-yl)piperazin-1-yl)-1,3,5-triazine |
| 51 | (R)-2-(3-((2-fluoroethoxy)methyl)-4-(pyrimidin-2-yl)piperazin-1-yl)-5-((2-(1-methyl- |
| | 1H-pyrazol-4-yl)pyrimidin-5-yl)ethynyl)pyrimidine |
| 52 | (R)-2-(3-((2-(2-fluoroethoxy)ethoxy)methyl)-4-(pyrimidin-2-yl)piperazin-1-yl)-5-((2- |
| | (1-methyl-1H-pyrazol-4-yl)pyrimidin-5-yl)ethynyl)pyrimidine |
| 53 | (R)-2-(3-((2-fluoroethoxy)methyl)-4-(pyrimidin-4-yl)piperazin-1-yl)-5-((2-(1-methyl- |
| | 1H-pyrazol-4-yl)pyrimidin-5-yl)ethynyl)pyrimidine |
| 54 | (R)-2-(1-(2-fluoroethyl)-1H-pyrazol-4-yl)-5-((2-(3-(methoxymethyl)-4-(pyrimidin-4- |
| | yl)piperazin-1-yl)pyrimidin-5-yl)ethynyl)pyrimidine |
| 55 | (R)-5-(5-((2-(3-((2-fluoroethoxy)methyl)-4-(1,3,5-triazin-2-yl)piperazin-1- |
| | yl)pyrimidin-5-yl)ethynyl)pyrimidin-2-yl)oxazole |
| 56 | (R)-2-(4-(5-((2-(1-(fluoromethyl)-1H-pyrazol-4-yl)pyrimidin-5-yl)ethynyl)pyrimidin- |
| | 2-yl)-2-(methoxymethyl)piperazin-1-yl)-1,3,5-triazine |
| 57 | (R)-2-(4-(5-((6-(1H-imidazol-1-yl)pyridin-3-yl)ethynyl)-6-fluoropyrazin-2-yl)-2- |
| | (methoxymethyl)piperazin-1-yl)pyrimidine |
| 58 | (R)-5-((6-(4H-1,2,4-triazol-4-yl)pyridin-3-yl)ethynyl)-4-fluoro-2-(3- |
| | (methoxymethyl)-4-(pyrimidin-4-yl)piperazin-1-yl)pyrimidine |
| 59 | (R)-5-((6-(3-fluoro-4H-1,2,4-triazol-4-yl)pyridin-3-yl)ethynyl)-2-(3- |
| | (methoxymethyl)-4-(pyrimidin-2-yl)piperazin-1-yl)pyrimidine |
| 60 | (R)-2-(4-(5-((6-(1H-imidazol-1-yl)pyridin-3-yl)ethynyl)pyrimidin-2-yl)-2- |
| | (methoxymethyl)piperazin-1-yl)-4-fluoropyrimidine |
| 61 | (R)-4-(4-(5-((6-(1H-imidazol-1-yl)pyridin-3-yl)ethynyl)pyrimidin-2-yl)-2- |
| | (methoxymethyl)piperazin-1-yl)-2-fluoropyrimidine |
| 62 | (S)-5-(5-((2-(3-((2-fluoroethoxy)methyl)-4-(1,3,5-triazin-2-yl)piperazin-1- |
| | yl)pyrimidin-5-yl)ethynyl)pyridin-2-yl)oxazole |
| 63 | 3-(((R)-4-(5-((6-(1H-imidazol-1-yl)pyridin-3-yl)ethynyl)pyrimidin-2-yl)-1- |
| | (pyrimidin-2-yl)piperazin-2-yl)methoxy)-2-fluoropropan-1-ol |
| 64 | (R)-5-(5-((2-(4-(2-fluoropyridin-4-yl)-3-(methoxymethyl)piperazin-1-yl)pyrimidin-5- |
| | yl)ethynyl)pyridin-2-yl)oxazole |
| 65 | 1-(4-(6-fluoropyrimidin-4-yl)-1-(5-((6-(oxazol-5-yl)pyridin-3-yl)ethynyl)pyrimidin-2- |
| | yl)piperazin-2-yl)-N-methylmethanamine |
| 66 | (R)-5-(5-((2-(4-(5-(2-fluoroethoxy)pyrimidin-2-yl)-3-(methoxymethyl)piperazin-1- |
| | yl)pyrimidin-5-yl)ethynyl)pyridin-2-yl)oxazole |
| 67 | 1-fluoro-3-(((R)-4-(5-((6-(oxazol-5-yl)pyridin-3-yl)ethynyl)pyrimidin-2-yl)-1-(1,3,5- |
| | triazin-2-yl)piperazin-2-yl)methoxy)propan-2-ol |
| 68 | (R)-5-((6-(3-fluoroazetidin-1-yl)pyridin-3-yl)ethynyl)-2-(3-(methoxymethyl)-4- |
| | (pyrimidin-2-yl)piperazin-1-yl)pyrimidine |
| 69 | (R)-5-((6-(1H-imidazol-1-yl)pyridin-3-yl)ethynyl)-2-(4-(6-fluoropyrazin-2-yl)-3- |
| | (methoxymethyl)piperazin-1-yl)pyrimidine |
| 70 | (R)-2-(4-(5-((6-(4H-1,2,4-triazol-4-yl)pyridin-3-yl)ethynyl)pyrimidin-2-yl)-2-((2- |
| | fluoroethoxy)methyl)piperazin-1-yl)-1,3,5-triazine |
| 71 | (R)-5-((6-(4H-1,2,4-triazol-4-yl)pyridin-3-yl)ethynyl)-2-(3-((2-fluoroethoxy)methyl)- |
| | 4-(pyrimidin-2-yl)piperazin-1-yl)pyrimidine |
| 72 | 5-((6-((S)-3-fluoropyrrolidin-1-yl)pyridin-3-yl)ethynyl)-2-((R)-3-(methoxymethyl)-4- |
| | (pyrimidin-2-yl)piperazin-1-yl)pyrimidine |
| 73 | (R)-1-(5-((2-(3-((2-fluoroethoxy)methyl)-4-(1,3,5-triazin-2-yl)piperazin-1- |
| | yl)pyrimidin-5-yl)ethynyl)pyrimidin-2-yl)-1H-pyrazol-4-amine |
| 74 | (R)-5-(5-((2-(3-((3-fluoropropoxy)methyl)-4-(1,3,5-triazin-2-yl)piperazin-1- |
| | yl)pyrimidin-5-yl)ethynyl)pyridin-2-yl)oxazole |
| 75 | (R)-7-(5-((2-(3-((2-fluoroethoxy)methyl)-4-(1,3,5-triazin-2-yl)piperazin-1- |
| | yl)pyrimidin-5-yl)ethynyl)pyridin-2-yl)-5,6,7,8-tetrahydro-[1,2,4]triazolo[4,3- |
| | a]pyrazine |
| 76 | (R)-5-((6-(4-(2H-1,2,3-triazol-2-yl)piperidin-1-yl)pyridin-3-yl)ethynyl)-2-(3- |
| | (methoxymethyl)-4-(pyrimidin-2-yl)piperazin-1-yl)pyrimidine |
| 77 | (R)-2-(4-(5-((6-(1-(2-(2-fluoroethoxy)ethyl)-1H-pyrazol-4-yl)pyridin-3- |
| | yl)ethynyl)pyrimidin-2-yl)-2-(methoxymethyl)piperazin-1-yl)-1,3,5-triazine |
| 78 | (R)-2-(2-((2-(2-fluoroethoxy)ethoxy)methyl)-4-(5-((2-(1-methyl-1H-pyrazol-4- |
| | yl)pyrimidin-5-yl)ethynyl)pyrimidin-2-yl)piperazin-1-yl)-1,3,5-triazine |
| 79 | (R)-5-((2-(3-((2-(2-fluoroethoxy)ethoxy)methyl)-4-(1,3,5-triazin-2-yl)piperazin-1- |
| | yl)pyrimidin-5-yl)ethynyl)-2,5′-bipyrimidine |
| 80 | (R)-2-(4-(5-((6-(1H-imidazol-1-yl)pyridin-3-yl)ethynyl)-4-fluoropyrimidin-2-yl)-2- |
| | ((2-methoxyethoxy)methyl)piperazin-1-yl)-1,3,5-triazine |
| 81 | (R)-2-(4-(5-((6-(6-fluoropyrimidin-4-yl)pyridin-3-yl)ethynyl)pyrimidin-2-yl)-2- |
| | (methoxymethyl)piperazin-1-yl)pyrimidine-5-carbonitrile |
| 82 | (R)-2-(3-((2-(2-(2-fluoroethoxy)ethoxy)ethoxy)methyl)-4-(pyrimidin-2-yl)piperazin- |
| | 1-yl)-5-((2-(1-methyl-1H-pyrazol-4-yl)pyrimidin-5-yl)ethynyl)pyrimidine |
| 83 | (R)-2-(3-(13-fluoro-2,5,8,11-tetraoxatridecyl)-4-(pyrimidin-2-yl)piperazin-1-yl)-5-((2- |
| | (1-methyl-1H-pyrazol-4-yl)pyrimidin-5-yl)ethynyl)pyrimidine |
| 84 | (R)-2-(4-(5-((2-(1-(2-fluoroethyl)-1H-pyrazol-4-yl)pyrimidin-5-yl)ethynyl)pyrimidin- |
| | 2-yl)-3-(methoxymethyl)piperazin-1-yl)-1,3,5-triazine |
| 85 | (R)-2-(1-(2-fluoroethyl)-1H-pyrazol-4-yl)-5-((2-(4-(5-fluoropyridin-2-yl)-3- |
| | (methoxymethyl)piperazin-1-yl)pyrimidin-5-yl)ethynyl)pyrimidine |
| 86 | (S)-5-((2-(4H-1,2,4-triazol-4-yl)pyrimidin-5-yl)ethynyl)-2-(4-(5-fluoropyridin-2-yl)- |
| | 2-methylpiperazin-1-yl)pyrimidine |
| 87 | (S)-2-(4-(5-((2-(1-(2-fluoroethyl)-1H-pyrazol-4-yl)pyrimidin-5-yl)ethynyl)pyrimidin- |
| | 2-yl)-3-(methoxymethyl)piperazin-1-yl)-1,3,5-triazine |
| 88 | (R)-5-fluoro-2-(4-(5-((2-(1-(2-fluoroethyl)-1H-pyrazol-4-yl)pyrimidin-5- |
| | yl)ethynyl)pyrimidin-2-yl)-2-(methoxymethyl)piperazin-1-yl)pyrimidine |
| 89 | (R)-5-((2-(4H-1,2,4-triazol-4-yl)pyrimidin-5-yl)ethynyl)-2-(4-(5-fluoropyridin-2-yl)- |
| | 3-(methoxymethyl)piperazin-1-yl)pyrimidine |
| 90 | (R)-5-fluoro-2-(4-(5-((6-(1-(2-fluoroethyl)-1H-pyrazol-4-yl)pyridin-3- |
| | yl)ethynyl)pyrimidin-2-yl)-2-(methoxymethyl)piperazin-1-yl)pyrimidine |
| 91 | (R)-2-(4-(5-((2-(1-(3-fluoropropyl)-1H-pyrazol-4-yl)pyrimidin-5- |
| | yl)ethynyl)pyrimidin-2-yl)-2-(methoxymethyl)piperazin-1-yl)-1,3,5-triazine |
| 92 | (R)-5-(2-fluoroethoxy)-2-(2-(methoxymethyl)-4-(5-((6-(1-methyl-1H-pyrazol-4- |
| | yl)pyridin-3-yl)ethynyl)pyrimidin-2-yl)piperazin-1-yl)pyrimidine |
| 93 | (R)-5-(5-((2-(3-((2-fluoroethoxy)methyl)-4-(pyrimidin-2-yl)piperazin-1-yl)pyrimidin- |
| | 5-yl)ethynyl)pyrimidin-2-yl)oxazole |
| 94 | (R)-2-(4-(6-fluoropyrimidin-4-yl)-3-(methoxymethyl)piperazin-1-yl)-5-((2-(1-methyl- |
| | 1H-pyrazol-4-yl)pyrimidin-5-yl)ethynyl)pyrimidine |
| 95 | (R)-2-(2-((fluoromethoxy)methyl)-4-(5-((2-(1-methyl-1H-pyrazol-4-yl)pyrimidin-5- |
| | yl)ethynyl)pyrimidin-2-yl)piperazin-1-yl)-1,3,5-triazine |
| 96 | (S)-2-(3-((2-fluoroethoxy)methyl)-4-(5-((2-(1-methyl-1H-pyrazol-4-yl)pyrimidin-5- |
| | yl)ethynyl)pyrimidin-2-yl)piperazin-1-yl)-1,3,5-triazine |
| 97 | (R)-5-fluoro-2-(2-((2-fluoroethoxy)methyl)-4-(5-((2-(1-methyl-1H-pyrazol-3- |
| | yl)pyrimidin-5-yl)ethynyl)pyrimidin-2-yl)piperazin-1-yl)pyrimidine |
| 98 | (R)-2-(2-((2-fluoroethoxy)methyl)-4-(5-((2-(1-methyl-1H-pyrazol-5-yl)pyrimidin-5- |
| | yl)ethynyl)pyrimidin-2-yl)piperazin-1-yl)-1,3,5-triazine |

or a pharmaceutically acceptable salt thereof.

12. The compound of claim 11
(R,E)-(4-(5-(2-(6-(1H-imidazol-1-yl)pyridin-3-yl)vinyl)pyrimidin-2-yl)-1-(pyrimidin-2-yl)piperazin-2-yl)methanol;
(R,E1-5-(2-(6-(1H-imidazol-1-yl)pyridin-3-yl)vinyl)-2-(3-(methoxymethyl)-4-(pyrimidin-2-yl)piperazin-1-yl)pyrimidine,
(R,E)-5-(2-(6-(1H-imidazol-1-yl)pyridin-3-yl)vinyl)-2-(3-((2-fluoroethoxy)methyl)-4-(pyrimidin-2-yl)piperazin-1-yl)pyrimidine;
(R,E)-5-(2-(6-(1H-imidazol-1-yl)pyridin-3-yl)vinyl)-2-(3-((fluoromethoxy)methyl)-4-(pyrimidin-2-yl)piperazin-1-yl)pyrimidine;
(R)-5-((6-(1H-imidazol-1-yl)pyridin-3-yl)ethynyl)-2-(4-(6-fluoropyrimidin-4-yl)-3-(methoxymethyl)piperazin-1-yl)pyrimidine;
(R,E)-5-(2-(6-(1H-imidazol-1-yl)pyridin-3-yl)vinyl)-2-(4-(6-fluoropyrimidin-4-yl)-2-(methoxymethyl)piperazin-1-yl)pyrimidine;
(R,E)-5-(2-(6-(1H-imidazol-1-yl)pyridin-3-yl)vinyl)-2-(4-(4-fluoropyridin-2-yl)-2-(methoxymethyl)piperazin-1-yl)pyrimidine;
(R,E)-5-(2-(6-(1H-imidazol-1-yl)pyridin-3-yl)vinyl)-2-(4-(2-fluoropyridin-4-yl)-2-(methoxymethyl)piperazin-1-yl)pyrimidine;
(R,E)-5-(2-(6-(1H-imidazol-1-yl)pyridin-3-yl)vinyl)-2-(2-((2-fluoroethoxy)methyl)-4-(pyridin-4-yl)piperazin-1-yl)pyrimidine;
(R,E)-5-(2-(6-(1H-imidazol-1-yl)pyridin-3-yl)vinyl)-2-(4-(6-fluoropyridin-3-yl)-2-(methoxymethyl)piperazin-1-yl)pyrimidine;
(R,E)-4-(4-(5-(2-(6-(1H-imidazol-1-yl)pyridin-3-yl)vinyl)pyrimidin-2-yl)-2-(methoxymethyl)piperazin-1-yl)-2-fluoropyrimidine;
(R,E)-5-(5-(2-(2-(3-((2-(2-fluoroethoxy)ethoxy)methyl)-4-(1,3,5-triazin-2-yl)piperazin-1-yl)pyrimidin-5-yl)vinyl)pyridin-2-yl)oxazole;
(R,E)-2-(2-((2-fluoroethoxy)methyl)-4-(5-(2-(2-(1-methyl-1H-pyrazol-4-yl)pyrimidin-5-yl)vinyl)pyrimidin-2-yl)piperazin-1-yl)-1,3,5-triazine;
(R)-2-(2-((2-fluoroethoxy)methyl)-4-(5-((2-(1-methyl-1H-pyrazol-4-yl)pyrimidin-5-yl)ethynyl)pyrimidin-2-yl)piperazin-1-yl)-1,3,5-triazine;
(R)-2-(4-(5-((2-(1-(2-fluoroethyl)-1H-pyrazol-4-yl)pyrimidin-5-yl)ethynyl)pyrimidin-2-yl)-2-(methoxymethyl)piperazin-1-yl)-1,3,5-triazine;
(R)-5-(5-((2-(3-((2-(2-fluoroethoxy)ethoxy)methyl)-4-(1,3,5-triazin-2-yl)piperazin-1-yl)pyrimidin-5-yl)ethynyl)pyridin-2-yl)oxazole;
(R)-2-(2-((2-fluoroethoxy)methyl)-4-(5-((2-(1-methyl-1H-pyrazol-3-yl)pyrimidin-5-yl)ethynyl)pyrimidin-2-yl)piperazin-1-yl)-1,3,5-triazine;
(R)-2-(3-((2-fluoroethoxy)methyl)-4-(pyrimidin-2-yl)piperazin-1-yl)-5-((2-(1-methyl-1H-pyrazol-4-yl)pyrimidin-5-yl)ethynyl)pyrimidine;
(R)-2-(3-((2-(2-fluoroethoxy)ethoxy)methyl)-4-(pyrimidin-2-yl)piperazin-1-yl)-5-((2-(1-methyl-1H-pyrazol-4-yl)pyrimidin-5-yl)ethynyl)pyrimidine,
or a pharmaceutically acceptable salt thereof.

13. The compound of claim 11
(R,E)-(4-(5-(2-(6-(1H-imidazol-1-yl)pyridin-3-yl)vinyl)pyrimidin-2-yl)-1-(pyrimidin-2-yl)piperazin-2-yl)methanol;
(R,E)-5-(2-(6-(1H-imidazol-1-yl)pyridin-3-yl)vinyl)-2-(3-(methoxymethyl)-4-(pyrimidin-2-yl)piperazin-1-yl)pyrimidine;
(R,E)-5-(2-(6-(1H-imidazol-1-yl)pyridin-3-yl)vinyl)-2-(3-((2-fluoroethoxy)methyl)-4-(pyrimidin-2-yl)piperazin-1-yl)pyrimidine;
(R)-5-((6-(1H-imidazol-1-yl)pyridin-3-yl)ethynyl)-2-(4-(6-fluoropyrimidin-4-yl)-3-(methoxymethyl)piperazin-1-yl)pyrimidine;
(R,E)-5-(2-(6-(1H-imidazol-1-yl)pyridin-3-yl)vinyl)-2-(4-(6-fluoropyrimidin-4-yl)-2-(methoxymethyl)piperazin-1-yl)pyrimidine;
(R,E)-5-(2-(6-(1H-imidazol-1-yl)pyridin-3-yl)vinyl)-2-(4-(2-fluoropyridin-4-yl)-2-(methoxymethyl)piperazin-1-yl)pyrimidine;
(R,E)-5-(2-(6-(1H-imidazol-1-yl)pyridin-3-yl)vinyl)-2-(4-(6-fluoropyridin-3-yl)-2-(methoxymethyl)piperazin-1-yl)pyrimidine;
(R,E)-5-(5-(2-(2-(3-((2-(2-fluoroethoxy)ethoxy)methyl)-4-(1,3,5-triazin-2-yl)piperazin-1-yl)pyrimidin-5-yl)vinyl)pyridin-2-yl)oxazole;
(R)-2-(2-((2-fluoroethoxy)methyl)-4-(5-((2-(1-methyl-1H-pyrazol-4-yl)pyrimidin-5-yl)ethynyl)pyrimidin-2-yl)piperazin-1-yl)-1,3,5-triazine;
(R)-2-(4-(5-((2-(1-(2-fluoroethyl)-1H-pyrazol-4-yl)pyrimidin-5-yl)ethynyl)pyrimidin-2-yl)-2-(methoxymethyl)piperazin-1-yl)-1,3,5-triazine;
(R)-2-(3-((2-(2-fluoroethoxy)ethoxy)methyl)-4-(pyrimidin-2-yl)piperazin-1-yl)--((2-(1-methyl-1H-pyrazol-4-yl)pyrimidin-5-yl)ethynyl)pyrimidine,
or a pharmaceutically acceptable salt thereof.

14. The compound of claim 11, or a pharmaceutically acceptable salt thereof, which is labeled with an isotope selected from ²H, ³H, ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁶Br, ⁷⁷Br, ¹²³I, ¹²⁴I or ¹³¹I.

15. The compound of claim 11, or a pharmaceutically acceptable salt thereof, which is isotopically labeled with ³H, ¹¹C or ¹⁸F.

16. A pharmaceutical composition comprising a compound of claim 1, or a pharmaceutically acceptable salt thereof, and at least one pharmaceutical excipient.

17. A method of imaging alpha-synuclein deposits in a human patient, using a compound of claim 14, or a pharmaceutically acceptable salt thereof, as the imaging agent, comprising the following steps;
a) placing a human patient in a supine position in the PET camera;
b) administering about 0.1 to about 10 mCi of a compound of claim 14 to the patient; and
c) performing an emission scan of the cerebral region of the patient's head to identify aggregations of alpha-synuclein in the brain tissue of the patient.

18. A method of measuring the clinical efficacy of therapeutic agents for Parkinson's disease comprising the steps of
a) administering an isotopically-labeled compound of Formula I, according to claim 14, to the patient diagnosed with PD before treatment with said therapeutic agent,
b) measuring the amount of alpha-synuclein aggregate formation in the patient's brain tissue,
c) administering an isotopically-labeled compound of Formula I, according to claim 14, to the patient after treatment with said therapeutic agent,
d) measuring the amount of alpha-synuclein aggregate formation in the patient's brain tissue after treatment, and
e) analyzing whether said therapeutic agent stopped or decreased the progression of alpha-synuclein aggregate formation in the patient's brain tissue.

19. (canceled)

20. (canceled)
